(19) United States
(12) Patent Application Publication
(10) Pub. No.: US 2024/0255537 A1
(43) Pub. Date: Aug. 1, 2024

(54) SYSTEMS, DEVICES, AND METHODS FOR CELL PROCESSING

(71) Applicant: Cellares Corporation, South San Francisco, CA (US)

(72) Inventors: Daniele MALLEO, Menlo Park, CA (US); Wilson Wai TOY, San Francisco, CA (US); Matthias WEBER, South San Francisco, CA (US)

(21) Appl. No.: 18/532,621

(22) Filed: Dec. 7, 2023

Related U.S. Application Data
(63) Continuation of application No. PCT/US2023/080593, filed on Nov. 20, 2023.
(60) Provisional application No. 63/427,720, filed on Nov. 23, 2022.

(51) Int. Cl. G01N 35/10; G01N 35/00

(52) U.S. Cl. CPC G01N 35/1009; G01N 2035/00326

(57) ABSTRACT

Disclosed herein are cell processing and manufacturing systems, devices, and methods thereof. A system for cell processing may comprise a plurality of docking station modules each configured to perform, in cooperation with a corresponding plurality of cartridge modules, one or more cell processing operations upon cells within a cartridge.

### INCORPORATION BY REFERENCE

This application claims priority to U.S. Provisional Application Ser. No. 63/427,720 filed on Nov. 23, 2022, which is hereby incorporated by reference in its entirety.

### TECHNICAL FIELD

Devices, systems, and methods herein relate to manufacturing cell products for biomedical applications using automated systems.

### BACKGROUND

Generally, cellular therapies based on hematopoietic stem cells (HSCs), chimeric antigen receptor (CAR) T cells, NK cells, tumor infiltrating lymphocytes (TILs), T-cell receptors (TCRs), regulatory T cells (T regs), gamma delta (γδ) T cells, and others rely on manufacturing of cell products. Cell product manufacturing typically involves multiple cell processing steps. Conventional solutions for cell product manufacturing may rely on cumbersome manual operations performed in expensive biosafety cabinets and/or cleanrooms. However, skilled laboratory technicians, adequate sterile enclosures such as cleanroom facilities, and associated protocols and procedures for regulated (GMP) manufacturing may be expensive to design and use. Many current manufacturing processes may also employ numerous manual reagent preparation and instrument manipulation steps during a manufacturing protocol, and the processes may require several days or even weeks. Although, even platforms described as automated cell processing in a closed system may generally rely on pre-configured instrumentation and tubing sets that limit operational flexibility and do not reliably prevent process failure due to accidental operator/human error. Moreover, most efforts to automate cell product manufacturing have been directed to automating individual processing steps of a cell therapy manufacturing workflow, and even systems that automate several steps may lack end-to-end process flexibility, process robustness, and process scalability. These and other limitations of the previous attempts at automation of cell processing are addressed in various embodiments disclosed herein.

### SUMMARY

According to an embodiment, the present disclosure relates generally to methods and systems for processing cell products.

In some variations, the present disclosure further relates to an automated system for cell processing, comprising a cartridge comprising a plurality of cartridge modules including a bioreactor module, a counterflow centrifugal elutriation module, and at least one of an electroporation module, a magnetic-activated cell selection module, a fluorescence-activated cell selection module, or a spinoculation module, at least one sterile liquid transfer port, and a liquid transfer bus fluidically coupled to each cartridge module, and a docking station comprising a plurality of docking station modules corresponding to the plurality of cartridge modules, each of the plurality of docking station modules being independently configured to perform one or more cell processing operations upon the cartridge in coordination with a respective cartridge module, wherein the docking station is sized and shaped to receive a single cartridge, wherein the liquid transfer bus is fluidically coupled to each cartridge module by fluid conduits arranged between ports on the liquid transfer bus and respective ports of each cartridge module, wherein the fluid conduits comprise tubing or channeling, wherein the system further comprises a sterile liquid transfer device configured to facilitate transfer of liquid between the cartridge and a closed volume fluid device, wherein the closed volume fluid device is a fluid vessel, wherein the sterile liquid transfer device comprises at least one actuator, wherein the actuator actuates one of the at least one sterile liquid transfer port of the cartridge and a corresponding sterile liquid transfer port of a closed volume fluid device, wherein the system further comprises a fluid vault disposed within a housing of the system, the fluid vault being accessible by a user to insert and/or remove one or more closed volume fluid devices, wherein each of the one or more cell processing operations performed upon the cartridge are performed on cells within the cartridge, wherein the plurality of cartridge modules comprises the electroporation module, wherein the plurality of cartridge modules comprises the magnetic-activated cell selection module, wherein the plurality of cartridge modules comprises the fluorescence-activated cell selection module, wherein the plurality of cartridge modules comprises the spinoculation module, wherein the plurality of docking station modules comprises a corresponding bioreactor module, wherein the plurality of docking station modules comprises a corresponding magnetic-activated cell selection module, wherein the plurality of docking station modules comprises a corresponding fluorescence-activated cell selection module, wherein the plurality of docking station modules comprises a corresponding electroporation module, wherein the plurality of docking station modules comprises a corresponding counterflow centrifugal elutriation module, wherein the plurality of docking station modules comprises a corresponding spinoculation module, wherein the cartridge comprises a pump fluidically coupled to the liquid transfer bus, wherein the system further comprises a pump actuator configured to interface with the pump, wherein the system further comprises a waste reservoir to capture waste generated by the cartridge, and/or wherein the transfer of liquid between the cartridge and the closed volume fluid device comprises extraction of at least a portion of a cell processing sample from the cartridge.

In some variations, the present disclosure further relates to an automated cell processing method comprising performing at least two cell processing operations within a cartridge positioned in a docking station, wherein the cartridge comprises a plurality of cartridge modules, at least one sterile liquid transfer port, and a liquid transfer bus fluidically coupled to each cartridge module, and wherein the docking station comprising a plurality of docking station modules corresponding to the plurality of cartridge modules and is sized and shaped to receive a single cartridge, wherein the plurality of cartridge modules comprises a bioreactor module, a counterflow centrifugal elutriation module, and at least one of an electroporation module, a magnetic-activated cell selection module, a fluorescence-activated cell selection module, or a spinoculation module, and wherein the cell processing operations are automatic upon execution of a set of received instructions, wherein the method further comprises actuating one of the at least one sterile liquid transfer port of the cartridge and a corresponding sterile liquid transfer port of a closed volume fluid device to facilitate transfer of liquid therebetween, wherein facilitating the transfer of liquid between the cartridge and the closed volume fluid device comprises actuating the one of the at least one sterile liquid transfer port and the corresponding sterile liquid transfer port, wherein the transfer of liquid between the cartridge and the closed volume fluid device comprises extraction of at least a portion of a cell processing sample from the cartridge, wherein the plurality of cartridge modules comprises the electroporation module, wherein the plurality of cartridge modules comprises the magnetic-activated cell selection module, wherein the plurality of cartridge modules comprises the fluorescence-activated cell selection module, wherein the plurality of cartridge modules comprises the spinoculation module, wherein the plurality of docking station modules comprises a corresponding bioreactor module, wherein the plurality of docking station modules comprises a corresponding counterflow centrifugal elutriation module, wherein the plurality of docking station modules comprises a corresponding electroporation module, wherein the plurality of docking station modules comprises a corresponding magnetic-activated cell selection module, wherein the plurality of docking station modules comprises a corresponding fluorescence-activated cell selection module, wherein the plurality of docking station modules comprises a corresponding spinoculation module, and wherein the two or more cell processing performed within the cartridge are performed upon cells within the cartridge.

Additional variations, features, and advantages of the invention will be apparent from the following detailed description and through practice of the invention.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

FIG. 1A is a block diagram of an illustrative variation of a cell processing and manufacturing system. FIG. 1B is a block diagram of an illustrative variation of a cartridge.

FIG. 2A is a block diagram of an illustrative variation of a cell processing and manufacturing system. FIG. 2B is a perspective view of an illustrative variation of a cell processing station of a cell processing and manufacturing system. FIG. 2C is a side view of an illustrative variation of a cell processing station and cartridge of a cell processing and manufacturing system. FIG. 2D is a perspective view of an illustrative variation of a cell processing and manufacturing system. FIG. 2E is an interior view of an illustrative variation of a cell processing and manufacturing system. FIG. 2F is a block diagram of an illustrative variation of a cell processing and manufacturing system. FIG. 2G is a block diagram of another illustrative variation of a cell processing and manufacturing system.

FIG. 3 is a block diagram of another illustrative variation of a cell processing and manufacturing system.

FIG. 4A is a perspective view of another illustrative variation of a docking station and a cartridge of a cell processing and manufacturing system. FIG. 4B and FIG. 4C are other perspective views of another illustrative variation of a docking station and a cartridge of a cell processing and manufacturing system.

FIGS. 5A-5V provide additional perspective views of aspects of another illustrative variation of at least one of a docking station, a cartridge, and a sterile liquid transfer instrument of a cell processing and manufacturing system.

FIG. 6 is a schematic diagram of an illustrative variation of a cartridge.

FIG. 7 is a schematic diagram of another illustrative variation of a cartridge.

FIG. 8A is a side view of an illustrative variation of a cartridge. FIG. 8B is a top view of an illustrative variation of a cartridge. FIG. 8C is a side view of an illustrative variation of a cartridge. FIG. 8D is a perspective view of an illustrative variation of a cartridge.

FIG. 9 shows a cross-sectional side view of an illustrative variation of a cartridge.

FIG. 10A shows an illustrative variation of a rotary valve and an actuator. FIG. 10B shows an illustrative variation of a rotary valve docked with an actuator.

FIG. 11A is a perspective view of an illustrative variation of a cartridge comprising a CCE module in an extended configuration. FIG. 11B is a cross-sectional side view of illustrative variation of a CCE module in a retracted configuration. FIG. 11C is a cross-sectional side view of an illustrative variation of a CCE module in an extended configuration.

FIG. 12A is a perspective view of an illustrative variation of a docking station magnetic-activated cell sorting (MACS) module comprising a magnet in an ON configuration. FIG. 12B is a perspective view of an illustrative variation of a docking station MACS module comprising a magnet in an OFF configuration.

FIG. 13 is a perspective view of an illustrative variation of a cartridge and a docking station bioreactor module. FIG. 14 is a perspective view of an illustrative variation of a cartridge coupled to a docking station bioreactor module.

FIG. 15 is a block diagram of an illustrative variation of a fluid connector system.

FIG. 16A is a schematic diagram of an illustrative variation of a fluid connector. FIG. 16B is a detailed schematic diagram of the fluid connector depicted in FIG. 16A. FIG. 16C is a schematic diagram of the fluid connector depicted in FIG. 16A in a coupled configuration. FIG. 16D is a schematic diagram of the fluid connector depicted in FIG. 16A in an open port configuration. FIG. 16E is a schematic diagram of the fluid connector depicted in FIG. 16A receiving a gas. FIG. 16F is a schematic diagram of the fluid connector depicted in FIG. 16A receiving a sterilant. FIG. 16G is a schematic diagram of the fluid connector depicted in FIG. 16A in an open valve configuration. FIG. 16H is a schematic diagram of the fluid connector depicted in FIG. 16A transferring fluid between fluid devices coupled to the fluid connector. FIG. 16I is a schematic diagram of the fluid connector depicted in FIG. 16A in a closed valve configuration.

FIG. 16J is a schematic diagram of the fluid connector depicted in FIG. 16A in a closed port configuration. FIG. 16K is a schematic diagram of the fluid connector depicted in FIG. 16A in an uncoupled configuration. FIG. 16L is a schematic diagram of the fluid connector depicted in FIG. 16A uncoupled from a sterilant source.

FIG. 17A is a front perspective view of a fluid connector in a closed port configuration.

FIG. 17B is a rear perspective view of the fluid connector depicted in FIG. 17A in the closed port configuration. FIG. 17C is a rear view of the fluid connector depicted in FIG. 17B in the closed port configuration. FIG. 17D is a front perspective view of a fluid connector in an open port configuration. FIG. 17E is a rear perspective view of the fluid connector depicted in FIG. 17D in the open port configuration. FIG. 17F is a rear view of the fluid connector depicted in FIG. 17E in the open port configuration.

FIG. 18A is a side view of a fluid connector in an uncoupled configuration. FIG. 18B is a cross-sectional side view of a fluid connector in an uncoupled configuration. FIG. 18C is a side view of a fluid connector in a coupled configuration. FIG. 18D is a cross-sectional side view of a fluid connector in a coupled configuration. FIG. 18E is a side view of a fluid connector in an open port configuration. FIG. 18F is a cross-sectional side view of a fluid connector in an open port configuration. FIG. 18G is a side view of a fluid connector in an open valve configuration. FIG. 18H is a cross-sectional side view of a fluid connector in an open valve configuration.

FIG. 19 is a schematic diagram of an illustrative variation of a fluid connector system.

FIG. 20A is a schematic diagram of an illustrative variation of a fluid connector system.

FIGS. 20B and 20C are schematic diagrams of an illustrative variation of a fluid connector connection process.

FIG. 21 is a block diagram of an illustrative variation of a fluid connector system.

FIG. 22 is a block diagram of an illustrative variation of a fluid connector system.

FIG. 23 is a block diagram of an illustrative variation of a fluid connector system.

FIG. 24A is a block diagram of an illustrative variation of a fluid connector system. FIG. 24B is a schematic diagram of an illustrative variation of a fluid connector connection process.

FIG. 24C is a schematic diagram of an illustrative variation of a valve.

FIG. 25A is a block diagram of an illustrative variation of a fluid connector system. FIG. 25B is a schematic diagram of an illustrative variation of a fluid connector connection process.

FIG. 25C is a schematic diagram of an illustrative variation of a valve.

FIG. 26A is a side view of an illustrative variation of a pump actuator and pump. FIG. 26B is a side view of an illustrative variation of a pump actuator coupled to a pump.

FIG. 27 is a flowchart of an illustrative variation of a method of transferring fluid using a fluid connector.

FIG. 28 is a flowchart of an illustrative variation of a method of cell processing.

FIG. 29 is a flowchart of an illustrative variation of a method of cell processing.

FIG. 30A is a flowchart of an illustrative variation of a method of cell processing for autologous CAR T cells or engineered TCR cells. FIG. 30B is a flowchart of an illustrative variation of a method of cell processing for allogeneic CAR T cells or engineered TCR cells.

FIG. 31 is a flowchart of an illustrative variation of a method of cell processing for HSC cells.

FIG. 32 is a flowchart of an illustrative variation of a method of cell processing for TIL cells.

FIG. 33 is a flowchart of an illustrative variation of a method of cell processing for NK-CAR cells.

FIGS. 34A-34C are flowcharts of illustrative variations of methods of cell processing for T_{reg} cells.

FIG. 35 is a flowchart of an illustrative variation of a method of cell processing.

FIG. 36 is a flowchart of an illustrative variation of a method of executing a transformation model.

FIG. 37 is an illustrative variation of a graphical user interface relating to an initial process design interface.

FIG. 38 is an illustrative variation of a graphical user interface relating to creating a process.

FIG. 39 is an illustrative variation of a graphical user interface relating to an empty process.

FIG. 40 is an illustrative variation of a graphical user interface relating to adding a reagent and a consumable container.

FIG. 41 is an illustrative variation of a graphical user interface relating to a process parameter.

FIG. 42 is an illustrative variation of a graphical user interface relating to a patient weight process parameter.

FIG. 43 is an illustrative variation of a graphical user interface relating to a preprocess analytic.

FIG. 44 is an illustrative variation of a graphical user interface relating to a white blood cell count preprocess analytic.

FIG. 45 is an illustrative variation of a graphical user interface relating to process parameter calculation.

FIG. 46 is an illustrative variation of a graphical user interface relating to a completed process setup.

FIG. 47 is an illustrative variation of a graphical user interface relating to process operations activation settings.

FIG. 48 is an illustrative variation of a graphical user interface relating to a filled process operations activation settings.

FIG. 49 is an illustrative variation of a graphical user interface relating to an initial process operations.

FIG. 50 is an illustrative variation of a graphical user interface relating to dragging in process operations.

FIG. 51 is another illustrative variation of a graphical user interface relating to dragging in process operations.

FIG. 52 is an illustrative variation of a graphical user interface relating to a filled process operations.

FIG. 53 is an illustrative variation of a graphical user interface relating to product monitoring.

FIG. 54 is another illustrative variation of a graphical user interface relating to product monitoring.

FIG. 55 is a block diagram of an illustrative variation of a cell processing and manufacturing system.

FIG. 56 is a cross-sectional side view of an illustrative variation of a counterflow centrifugal elutriation (CCE) module.

FIG. 57 is a cross-sectional side view of an illustrative variation of a magnetic-activated cell selection (MACS) module.

FIG. 58 is a perspective view of an illustration variation of a CCE system.

FIGS. 59A-59B are perspective views of an illustrative variation of a CCE system. FIG. 59C is a side cross-sectional view of an illustrative variation of a CCE system. FIGS. 59D-59F are side cross-sectional views of an illustrative variation of a rotor of a CCE module.

FIG. 60A is a plan view of an illustrative variation of a rotor of a CCE module. FIGS. 60B and 60C are perspective views of an illustrative variation of a rotor of a CCE module. FIG. 60D is a side view of an illustrative variation of a rotor of a CCE module. FIG. 60E is a perspective view of an illustrative variation of a rotor in a housing. FIGS. 60F and 60G are plan schematic views of illustrative variations of a rotor of a CCE module. FIG. 60H is a side view of an illustrative variation of a rotor of a CCE module. FIG. 60I is a perspective view of another illustrative variation of a rotor of a CCE module. FIG. 60J is a perspective view of yet another illustrative variation of a rotor of a CCE module. FIG. 60K is a schematic plan view of another illustrative variation of rotor dimensions of a CCE module. FIG. 60L is an image of a set of illustrative variations of rotors of a CCE module.

FIGS. 61A-61C are schematic views of an illustrative variation of a cell separation process.

FIG. 62A is a perspective view of an illustrative variation of a MACS system in a first configuration. FIG. 62B is a perspective view of an illustrative variation of a MACS system in a second configuration. FIG. 62C is a cross-sectional side view of an illustrative variation of a MACS system. FIG. 62D is a perspective view of an illustrative variation of a MACS system in the second configuration. FIG. 62E is a plan view of an illustrative variation of a flow cell and magnet array of a MACS system. FIG. 62F is a plan view of an illustrative variation of a flow cell of a MACS system. FIG. 62G is a schematic diagram of an illustrative variation of a flow cell and magnet array.

FIGS. 63A-63E are perspective views of illustrative variations of a magnet array.

FIG. 64A is a perspective view of an illustrative variation of a flow cell. FIG. 64B is a cross-sectional side view of an illustrative variation of a flow cell. FIG. 64C is a schematic diagram of an illustrative variation of a MACS system.

FIGS. 65A-65C are schematic diagrams of an illustrative variation of a flow cell.

FIGS. 66A-66C are schematic diagrams of an illustrative variation of a cell separation process.

FIGS. 67A-67B are schematic diagrams of an illustrative variation of a cell processing and manufacturing system.

FIG. 68A is a cross-sectional perspective view of an illustrative variation of a bioreactor.

FIG. 68B is a cross-sectional side view of an illustrative variation of a bioreactor. FIG. 68C is a perspective view of an illustrative variation of an enclosure of a bioreactor. FIG. 68D is a plan view of an illustrative variation of an enclosure of a bioreactor.

FIG. 68E is a perspective view of an illustrative variation of a membrane of a bioreactor.

FIG. 68F is a side view of an illustrative variation of a membrane of a bioreactor. FIG. 68G is a perspective view of an illustrative variation of a membrane of a bioreactor. FIG. 68H is a bottom view of an illustrative variation of a membrane of a bioreactor.

FIG. 69A is a cross-sectional side view of an illustrative variation of an enclosure of a bioreactor. FIG. 69B is a cross-sectional perspective view of an illustrative variation of an enclosure of a bioreactor.

FIG. 70 is an exploded perspective view of an illustrative variation of a bioreactor.

FIG. 71A is a plan view of an illustrative variation of a bioreactor. FIG. 71B is a cross-sectional side view of an illustrative variation of a bioreactor.

FIG. 72 is a schematic diagram of an illustrative variation of an electroporation system.

FIG. 73 is an exploded perspective view of an illustrative variation of an electroporation module.

FIGS. 74A-74B are schematic diagrams of illustrative variation of an electroporation process.

FIG. 75 is a circuit diagram of an illustrative variation of an electroporation process.

FIGS. 76A-76D are plots of illustrative variations of an electroporation process.

FIG. 77A is a flowchart of an illustrative variation of a method of separating cells. FIG. 77B is a flowchart of an illustrative variation of a method of concentrating cells. FIG. 77C is a flowchart of an illustrative variation of a method of buffer exchange.

FIG. 78 is a flowchart of another illustrative variation of a method of separating cells.

FIG. 79A is a flowchart of an illustrative variation of a closed-loop method of separating cells 7900. FIG. 79B is a flowchart of an illustrative variation of a closed-loop method of elutriating cells 7910. FIG. 79C is a flowchart of an illustrative variation of a closed-loop method of harvesting cells 7920.

FIG. 80A is a flowchart of an illustrative variation of a method of separating cells. FIG. 80B is a flowchart of an illustrative variation of a method of selecting cells.

FIG. 81 is a flowchart of another illustrative variation of a method of separating cells.

FIG. 82A is a flowchart of an illustrative variation of a method of preparing a bioreactor.

FIG. 82B is a flowchart of an illustrative variation of a method of loading a bioreactor. FIG. 82C is a flowchart of an illustrative variation of a method of preparing a bioreactor. FIG. 82D is a flowchart of an illustrative variation of a method of calibration for a bioreactor. FIG. 82E is a flowchart of an illustrative variation of a method of mixing reagents. FIG. 82F is a flowchart of an illustrative variation of a method of mixing reagents. FIG. 82G is a flowchart of an illustrative variation of a method of culturing cells. FIG. 82H is a flowchart of an illustrative variation of a method of refrigerating cells. FIG. 82I is a flowchart of an illustrative variation of a method of taking a sample. FIG. 82J is a flowchart of an illustrative variation of a method of culturing cells.

FIG. 82K is a flowchart of an illustrative variation of a method of media exchange. FIG. 82L is a flowchart of an illustrative variation of a method of controlling gas. FIG. 82M is a flowchart of an illustrative variation of a method of controlling pH.

FIG. 83 is a flowchart of an illustrative variation of a method of electroporating cells.

FIG. 84 is a flowchart of another illustrative variation of a method of electroporating cells.

FIG. 85 are schematic diagrams of an illustrative variation of a fluid connector.

FIG. 86 are schematic diagrams of an illustrative variation of a fluid connector port.

FIG. 87 is a schematic diagram of an illustrative variation of a fluid connector connection process.

FIG. 88 is a schematic diagram of an illustrative variation of a fluid connector connection process.

FIG. 89 is a schematic diagram of an illustrative variation of a fluid connector.

FIG. 90A is a side view of an illustrative variation of a fluid connector. FIG. 90B is a perspective view of the fluid connector depicted in FIG. 90A. FIG. 90C is a cross-sectional side view of the fluid connector depicted in FIG. 90A.

FIG. 91A is a side view of an illustrative variation of a fluid connector. FIG. 91B is a perspective view of the fluid connector depicted in FIG. 91A. FIG. 91C is a cross-sectional side view of the fluid connector depicted in FIG. 91A.

FIG. 91D is a side view of an illustrative variation of a fluid connector. FIG. 91E is a perspective view of the fluid connector depicted in FIG. 91D. FIG. 91F is a cross-sectional side view of the fluid connector depicted in FIG. 91D.

FIG. 92A is a side view of an illustrative variation of a fluid connector. FIG. 92B is a transparent side view of the fluid connector depicted in FIG. 92A. FIG. 92C is a perspective view of the fluid connector depicted in FIG. 92A. FIG. 92D is a cross-sectional side view of the fluid connector depicted in FIG. 92A.

FIG. 93A is a perspective view of an illustrative variation of a fluid connector. FIG. 93B is a transparent perspective view of the fluid connector depicted in FIG. 93A.

FIG. 94A is a perspective view of an illustrative variation of a fluid connector. FIG. 94B is a transparent perspective view of the fluid connector depicted in FIG. 94A.

FIG. 95A is a perspective view of an illustrative variation of a fluid connector. FIG. 95B is a transparent perspective view of the fluid connector depicted in FIG. 95A. FIG. 95C is a detailed side view of a port in an open port configuration. FIG. 95D is a detailed side view of a port in a closed port configuration.

FIG. 96A is a plan view of an illustrative variation of a fluid device. FIG. 96B is a side view of an illustrative variation of a fluid device coupled to a robot. FIG. 96C is a perspective view of an illustrative variation of a fluid device held by a robot.

FIG. 97A is a perspective view of an illustrative variation of a MACS module. FIG. 97B is a cross-sectional perspective view of an illustrative variation of a MACS module. FIG. 97C is a cross-sectional side view of an illustrative variation of a MACS module.

FIG. 98 is a flowchart of an illustrative variation of a method of cell processing.

FIG. 99 is a flowchart of an illustrative variation of a method of cell processing.

FIG. 100 is a flowchart of an illustrative variation of a method of cell processing.

FIG. 101 is a flowchart of an illustrative variation of a method of cell processing.

FIG. 102 is a schematic diagram of an illustrative variation of a cell processing and manufacturing system.

FIGS. 103A and 103B are perspective views of an illustrative variation of a sterile liquid transfer device.

### DETAILED DESCRIPTION

### Definitions

The term “a” or “an” refers to one or more of that entity, i.e., may refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used m conjunction with a range or series of values, the term “about” applies to the endpoints of the range or each of the values enumerated in the series, unless otherwise indicated. As used in this application, the terms “about” and “approximately” are used as equivalents.

As used herein, the term “sterile” is used as a non-limiting description of some variations, an optional feature providing advantages in operation of certain systems and methods of the disclosure. Maintaining sterility is typically desirable for cell processing but may be achieved in various ways, including but not limited to providing sterile reagents, media, cells, and other solutions; sterilizing cartridge(s) and/or cartridge component(s) after loading (preserving the cell product from destruction); and/or operating the system in a sterile enclosure, environment, building, room, or the like. Such user or system performed sterilization steps may make the cartridge or cartridge components sterile and/or preserve the sterility of the cartridge or cartridge components.

The term “and” as used herein is interchangeably used with “or” unless expressly stated otherwise.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” “below,” and words of similar import, when used in this application, shall refer to this application as a whole and not to any particular portions of the application.

### Cell Processing and Manufacturing System

Systems and methods for processing and manufacturing cell products for biomedical applications are described herein. In variations, these systems may include a cell processing and manufacturing system (CPMS) comprising a cartridge including a cell product and a cell processing station including at least a controller and at least one novel docking station configured to receive a cartridge. In some variations, the CPMS may perform automated manufacturing of cell products. Briefly, the cell processing station may also include a pump, a reagent vault, a fluid source, a fluid connector, at least one sensor, and a sterile liquid transfer device. The docking station of the cell processing station may include one or more docking station modules (e.g., at least one docking station module, or a plurality of docking station modules) and may be configured to receive the cartridge. The controller of the cell processing station may include a processor, a memory, a display, an input device, and a communication device for executing methods described herein. The cartridge of the CPMS may include a liquid transfer bus, a sensor (e.g., at least one sensor), a fluid connector, and a one or more modules (e.g., at least one cartridge module or a plurality of cartridge modules). The one or more cartridge modules may include a counterflow centrifugal elutriation module, a bioreactor, an electroporation module, a spinoculation module, a fluorescence-activated cell selection module, and a cell separation module, which may be a magnetic-activated cell selection module. The docking station may be configured to receive a single cartridge and the cartridge may be stationary within the docking station. Further, the one or more docking station modules may be configured to engage and/or interface with the cartridge and/or a corresponding module of the cartridge to perform cell processing steps on the cell product, such that the CPMS performs cell processing steps on the cell product. In some variations, a plurality of cell processing steps may be performed within a single cartridge.

Moreover, methods for processing and manufacturing cell products for biomedical applications may include a method of processing a solution containing a cell product. For instance, the method may include the cell processing steps of digesting tissue using an enzyme reagent to release a select cell population into solution, enriching cells using a docking station CCE module, washing cells using the docking station CCE module, selecting cells in the solution using a docking station selection module, sorting cells in the solution using a docking station sorting module, differentiating or expanding the cells in a cartridge bioreactor module, activating cells using an activating reagent, electroporating cells, transducing cells using a vector, and finishing a cell product.

Advantageously, in some variations, the multitude of cell processing steps may be performed on a cartridge positioned within a docking station of a cell processing and manufacturing system, without having to move or transport the cartridge out of the docking station.

### Cell Processing Station (CPS)

The CPMS introduced above may generally include a cell processing station having a docking station including a plurality of docking station modules, each independently configured to perform one or more cell processing operations upon a cartridge within the docking station. The docking station modules may include one or more of a bioreactor module, a cell selection module (e.g., a docking station magnetic-activated cell selection module), a sorting module (e.g., a fluorescence activated cell sorting (FACS) module), an electroporation module, a counterflow centrifugal elutriation (CCE) module, a reagent vault, and the like. In variations, the docking station modules of the CPS may be configured to interface with corresponding cartridge modules of the cartridge to perform cell processing upon a cell product within the cartridge.

The CPS may advantageously include a docking station that is configured to integrate the functionality of a variety of cell processing tasks into a single structure, thereby allowing a cell product to be generated in an end-to-end automated fashion without intervention by a user to change settings, transfer cell products, and the like. In particular, the single structure may be a cartridge configured to undergo a complete end-to-end automation of a multitude of processes (e.g., washing, enrichment, elutriation, magnetic cell separation, sorting, activation, genetic modification, electroporation, spinoculation, formulation, expansion, harvest/formulation and cryopreservation) while docked in a docking station to generate a cell product. In some variations, the cartridge may include two or more bioreactor modules for generating two or more cell products via a split process. In combination with a cartridge of the present disclosure, cell processing tasks may be performed on cells within the cartridge in coordination with corresponding modules of the cartridge, according to a predetermined processing schedule, in order to generate a processed cell product.

In some variations, the cell processing station of the present disclosure may be a standalone unit, featuring a standard power option, which may be moved with ease throughout a lab space (e.g., on rollers or casters) as needed. In particular, the cell processing and manufacturing system of the present disclosure, which may include the mobile cell processing station having the docking station and the cartridge housed therein, may be compact and easily implemented within any lab space, as an expensive installation is not required. Accordingly, the cell processing and manufacturing system of the present disclosure may enable users to perform process development studies prior to making larger investments in production and to scale up. Moreover, because a standalone unit may not require that the cartridge be moved between modules and/or instruments during cell processing, design and developmental risks for cell processing may be reduced.

### Cartridge

The cartridge may include a plurality of cartridge modules (also referred to herein as cartridges, consumable cartridges, consumables, and cell processing devices) such as a bioreactor, a counterflow centrifugal elutriation (CCE) module, a magnetic cell sorter (e.g., magnetic-activated cell selection module (MACS)), an electroporation device (e.g., electroporation module (EP)), a sorting module (e.g., fluorescence activated cell sorting (FACS) module), an acoustic flowcell module, a centrifugation module, a microfluidic enrichment module, combinations thereof, and the like. The cell processing and manufacturing systems described herein may reduce operator intervention and increase throughput by automating cartridge (and cell product) processing. For example, the automated cell processing and manufacturing systems may facilitate sterile liquid transfers between one or more cartridges and the cell processing station (e.g., one or more docking station modules). Additionally, or alternatively, the automated cell processing and manufacturing systems may facilitate sterile liquid transfers between one or more cartridges and other components of the system such as a fluid connector (e.g., sterile liquid transfer port), a reagent vault, a sampling vessel (e.g., sterile liquid transfer device, and combinations thereof, as described in detail herein).

The cartridge may be configured to be portable and facilitate automated and sterile cell processing. For example, the cartridge may be configured to be moved by a user from a location outside of the CPS to a reception location of the docking station within the cell processing station.

In some variations, the cartridge may further include one or more of a sterile liquid transfer port, a liquid transfer bus (also referred to herein as a fluidic bus) fluidically coupled to each module, and a pump fluidically coupled to the liquid transfer bus.

In some variations, the cartridge may optionally provide a self-contained device capable of performing one or more cell processing steps via one or more modules. The modules may be integrated into a fixed configuration within the cartridge. Additionally. or alternatively, the cartridge modules may be configurable or moveable within the cartridge, permitting various cartridges to be assembled from shared modules. Similarly stated, the cartridge may be a single, closed unit with fixed components for each cartridge module; or the cartridge may contain configurable modules coupled by configurable fluidic, mechanical, optical, and electrical connections. In some variations, one or more sub-cartridges, each containing a set of modules, may be configured to be assembled to perform various cell processing workflows. Moreover, the cartridge modules may each be provided in a distinct housing or may be integrated into a cartridge or sub-cartridge with other modules. While the modules may generally be described herein as distinct groups of components for the sake of simplicity, the modules (and their respective components) may be arranged in any suitable configuration. For example, the components of different modules may be interspersed with each other within the CPS such that each module is defined by the set of connected components that collectively perform a predetermined function. However, the components of each cartridge module may or may not be physically grouped within the cartridge. In some variations, multiple cartridges may be used to process a single cell product through transfer of the cell product from one cartridge to another cartridge of the same or different type and/or by splitting cell product into more cartridges and/or pooling multiple cell products into fewer cartridges.

Generally, each of the docking station modules of the system may interface with its respective module or modules on the cartridge. For example, an electroporation module on the cartridge (if present) may interface with the docking station electroporation module to perform an electroporation step on the cell product—and may also interface with common components, such as components of a fluidic bus line (e.g., pumps, valves, sensors, etc.). An advantage of such split module designs is that expensive components (e.g., motors, sensors, heaters, lasers, etc.) may be retained in the docking station module of the system. The use of disposable cartridges may eliminate the need, in such variations, to sterilize cartridges between use.

Various materials may be used to construct the cartridge and the cartridge housing, including metal, plastic, rubber, and/or glass, or combinations thereof. The cartridge, its components, and its housing may be molded, machined, extruded, 3D printed, or any combination thereof. The cartridge may contain components that are commercially available (e.g., tubing, valves, fittings); these components may be attached or integrated with custom components or devices. The housing of the cartridge may constitute an additional layer of enclosure that further protects the sterility of the cell product. In some variations, the cartridge may be designed to be single-use or disposable.

Generally, an operator may load or unload the cartridge in an environment of class ISO-5 or better, utilizing aseptic technique to ensure that sterility of the contents of the cartridge is maintained when the cartridge is opened. In some variations, the operator may perform loading or unloading of the cartridge using manual aseptic connections (e.g., sterile tube welding).

### Reagent Vault

The system may generally include one or more a reagent vaults where reagents are stored. Nonlimiting examples of the one or more reagents may include cell culture media, buffer, cytokines, proteins, enzymes, polynucleotides, transfection reagents, non-viral vectors, viral vectors, antibiotics, nutrients, cryoprotectants, solvents, cellular materials, and pharmaceutically acceptable excipients. Additionally or alternatively, waste may be stored in the reagent vault. In some variations, in-process samples extracted from one or more cartridges may be stored in the reagent vault. The reagent vault may comprise one or more controlled temperature compartments (e.g., freezers, coolers, water baths, warming chambers, or others, at e.g. about −80° C., about −20° C., about 4° C., about 25° C., about 30° C. about 37° C., and about 42° C.). Temperatures in these compartments may be varied during the cell manufacturing process to heat or cool reagents. In variations of the methods of the disclosure, a robot (or manually with the aid of an operator) may engage each of the cartridge and the reagent vault. The reagent vault may interface with one or more sterile liquid transfer ports on the cartridge such that reagent or material may be dispensed into the cartridge. Optionally, fluid may be added or removed from the cartridge before, during, or after reagent addition or removal. In some variations, the system comprises a sterile liquid transfer instrument, similarly configured to transfer fluid into or out of the cartridge in an automated, manual, or semi-automated fashion. An operator may stock the sterile liquid transfer instrument with reagents manually. The reagent vault may have automated doors to permit access for sterile liquid transfer devices and/or other reagent vessels, optionally each under independent closed loop temperature control. The devices and vessels may be configured for pick-and-place movement by a robot. In some variations, the reagent vault may include one or more sample pickup areas. For example, a robot may be configured to move one or more reagents to and from one or more of the sample pickup areas.

In some variations, the reagent vault may be sized to house a plurality of sterile liquid transfer devices required to support a single patient process. The sterile liquid transfer devices may be manually provided to and or replaced within the reagent vault on a predetermined schedule (e.g., every 3 days or as required by the cell processing task).

### Cell Selection System

The CPMS described herein may include a cell selection system configured to separate cells based on predetermined criteria. For example, cells may be separated based on physical characteristics such as size and/or density using, for example, counterflow centrifugal elutriation. Cells may also be separated based on the presence of predetermined antigens of a cell using, for example, magnetic-activated cell selection. In some variations, a CPMS having modules (e.g. cartridge modules, docking station modules) for these separation methods may facilitate one or more cell processing steps including, but not limited to, cell concentration, cell dilution, cell washing, buffer replacement, and magnetic separation. The cell selection systems described herein may increase throughput and cell yields output, in a compact and portable structure. For example, prior to magnetically separating cells, a suspension of cells may be mixed with magnetic reagents in excess or at a predetermined concentration (e.g., cells/mL). Likewise, after magnetically separating cells, the cells may be washed in a solution (e.g., suitable buffered solution).

In some variations, a cell separation system (or cell separator) may include a rotor configured for counterflow centrifugal elutriation of cells in a fluid, a first magnet configured to magnetically rotate the rotor and separate the cells from the fluid in the rotor, a flow cell in fluid communication with the rotor and configured to receive the cells from the rotor, and a second magnet configured to magnetically separate the cells in the flow cell.

In some variations, a CCE module may be integrated into a cartridge to enable a cell processing and manufacturing system to separate cells based on cell size and/or density. In some variations, a cell separation system may comprise a housing comprising a rotor configured to separate cells from a fluid (e.g., separate cells of different size and/or density from cells that remain in the fluid), and a magnet configured to magnetically rotate the rotor. The housing may be configured to move relative to the magnet or vice versa (e.g., move the magnet relative to the housing). The CCE modules described herein may provide cell separation within a compact and portable housing where the magnet may be disposed external to the housing (e.g., magnet disposed within a corresponding CCE module of a docking station).

In some variations, a compact rotor that may aid cartridge integration may include input and output fluid conduits extending from the rotor towards opposing sides of a rotor housing. For example, a rotor may include a first side having a first fluid conduit and a second side having a second fluid conduit where the second side is opposite the first side. An elutriation chamber (e.g., cone) may be coupled between the first fluid conduit and the second fluid conduit.

In some variations, a method of separating cells from a fluid may include moving a rotor towards a magnet, the rotor defining a rotational axis, flowing the fluid through the rotor, rotating the rotor (e.g., magnetically) about the rotational axis using the magnet while flowing the fluid through the rotor, and moving the rotor away from the magnet.

In some variations, a method of separating cells from a fluid may include flowing the fluid comprising the cells into a flow cell. A set of the cells may be labeled with magnetic particles. The set of cells may be magnetically attracted towards a magnet array for a dwell time, and the set of cells may flow out of the flow cell after the dwell time.

In some variations, a flow cell may include an elongate cavity having a cavity height and a magnet array comprising a plurality of magnets, each of the magnets spaced apart by a spacing distance. A predetermined ratio between the cavity height to the spacing distance may optimize magnetic separation of the cells in the flow cell.

### Electroporation

In some variations, an electroporation module (EP), or electroporator, as described herein may be configured to facilitate one or more of transduction and transfection of cells. As described in more detail herein, the EP may be configured to physically separate a first volume of fluid (e.g., first batch) comprising cells from a second volume of fluid (e.g., second batch, third batch) comprising cells using a gas (e.g., air gap). The EP may also be configured to apply an electroporation signal (e.g., voltage pulse, waveform) separately to each discrete batch of fluid to improve electroporation efficiency and thus increase throughput. In some variations, active electric field compensation may similarly improve electroporation efficiency and throughput.

In some variations, a cell processor may include a fluid conduit configured to receive a first fluid comprising cells and a second fluid (e.g., gas, oil), a set of electrodes coupled to the fluid conduit, a pump coupled to the fluid conduit, and a controller comprising a processor and memory. The controller may be configured to generate a first signal to introduce the first fluid into the fluid conduit using the pump, generate a second signal to introduce the second fluid into the fluid conduit such that the second fluid separates the first fluid from a third fluid, and generate an electroporation signal to electroporate the cells in the fluid conduit using the set of electrodes.

In some variations, a method of electroporating cells may include receiving a first fluid comprising cells in a fluid conduit, receiving a second fluid comprising a gas in the fluid conduit to separate the first fluid from a third fluid, and applying an electroporation signal to the first fluid to electroporate the cells.

In some variations, a method of electroporating cells may include receiving a first fluid comprising cells in a fluid conduit, applying a resistance measurement signal to the first fluid using a set of electrodes, measuring a resistance between the first fluid and the set of electrodes, and applying an electroporation signal to the first fluid based on the measured resistance.

### Bioreactor

In some variations, a bioreactor may include an enclosure comprising a base and a sidewall, and a gas-permeable membrane coupled to one or more of the base and the sidewall of the enclosure. The gas-permeable membrane may aid cell culture. In some variations, a cell processing and manufacturing system may include the bioreactor and an agitator coupled to the bioreactor. The agitator may be configured to agitate the bioreactor based on orbital motion.

### Fluid Connector

Notably, traditional cell processing systems lack automated, multi-use sterile fluid connector solution for cell therapy production where a set of sterile fluid connectors are capable of multiple connection and disconnection cycles with the given system. For example, conventional sterile fluid connectors may typically be single-use devices and may therefore be expensive and labor intensive with respect to manufacturing and performing a cell processing procedure. Generally, the fluid connectors described herein may include a plurality of sealed enclosures between a sterile portion (e.g., fluid connector lumen or cavity) and an external (e.g., non-sterile) ambient environment, thereby facilitating aseptic control of a fluid connector and devices coupled thereto. The fluid connectors described herein may be a durable component that may be reused for multiple cycles while maintaining sterility and/or bioburden control. For example, the fluid connector may be sterilized using a sterilant without harming the cell product or other biological material.

In some variations, a sterile manufacturing system as described herein may utilize one or more sterile fluid connectors and have a configuration suitable to be manipulated by a robot, such as a robotic arm and the like. The sterile fluid connectors described herein may enable the transfer of fluids in an automated, sterile, and metered manner for automating cell therapy manufacturing. Automating cell therapy manufacturing may in turn provide lower per patient manufacturing costs, a lower risk of process failure, and the ability to meet commercial scale patient demand for cell therapies. In some variations, sterile fluid connectors may increase one or more of sterility, efficiency, and speed by removing a human operator from the manufacturing process. An automated and integrated sterilization process as described herein may be applied to the fluid connector to maintain sterility of the system. For example, the fluid connector may maintain sterility through multiple connection/disconnection cycles between separate sterile closed volume fluid devices (e.g., enclosure, container, vessel, cartridge, docking station module, cartridge module, bioreactor, enclosed vessel, sealed chamber). Accordingly, the systems, devices, and methods described herein may reduce the complexity of a sterilization process, reduce energy usage, and increase sterilization efficiency.

In some variations, a fluid connector may include a first connector configured to mate with a second connector (e.g., male connector and female connector). Respective proximal ends of the connectors may be configured to connect (e.g., be in fluid communication, form a fluid pathway) with respective fluid devices in order to transfer one or more of fluid (e.g., liquid and/or gas) and biological material (e.g., cell product) between the fluid devices. The distal ends of the connectors may include ports configured to mate with each other. The fluid connector may also include a sterilant port configured to facilitate sterilization of a chamber within the distal ends of the first and second connectors. The fluid connector may be sterilized before or after connection as desired to ensure sterility. In this manner, the fluid connector may be reused for multiple connection and disconnection cycles.

In some variations, a cell processing and manufacturing system utilizing the fluid connectors described herein may include a robot configured to operate the fluid connector and a controller configured to control the robot to manipulate (e.g., move, connect, open, close, disconnect) the first and second connectors together (without human interaction) while maintaining sterility of the fluid connector and a plurality of fluid devices, thereby further reducing the risk of contamination. The fluid devices may be one or more of an instrument, a docking station module, a cartridge, a cartridge module, and the like.

### Cell Processing Control

Systems and methods for manufacturing cell products for biomedical applications using automated systems are described herein. Conventional semi-automated solutions for cell processing may not allow users to define biological processes. Instead, users may select from a limited set of predefined machine processes and process-control parameters. Additionally, the conventional semi-automated solutions may not provide scalable manufacturing solution for cell therapy production. For example, cell therapy manufacturing may traditionally be executed batchwise (i.e. one product will be manufactured in a single room/suite, with required processing tools located inside). This may either be guided by a technician following a standard operating procedure (SOP), or in some cases, processing tools (e.g., Miltenyi Prodigy, Lonza Cocoon) may carry out a series of processing steps for a single patient product on a single multi-functional processing tool. However, existing solutions (e.g., Miltenyi Prodigy) may not allow users to define biological processes. Furthermore, the manual labor required of conventional solutions may increase the risk of product contamination and human error.

In some variations of the present disclosure, a set of cell therapy biological manufacturing processes may be transformed into a set of machine instructions suitable for automated execution using the systems described herein. For example, a method of transforming user-defined cell processing operations into cell processing steps to be executed by a processor of an automated cell processing and manufacturing system may include receiving an ordered input list of cell processing operations, and executing a transformation model on the ordered input list to create an ordered output list of cell processing steps capable of being performed by the system. As used herein, a transform model may refer to an algorithm, process, or transformation configured to translate a set of cell processing steps into a set of machine or hardware instructions for the system. Advantageously, in some variations, the multitude of cell processing steps may be performed on a cartridge positioned in a docking station of a cell processing and manufacturing system, without having to move or transport the cartridge out of the docking station. In some variations, the docking station modules may be controlled to perform cell processing steps on each cell product. In this manner, the systems and methods enable biologists to define manufacturing processes in biological terms and have the system transform this biological model (e.g., process definition) into a set of machine-executed instructions.

The end-to-end closed system automation described herein may reduce process failure rates and cost. For example, end-to-end automation may reduce manufacturing time (e.g., dwell times) and increase throughput as compared to conventional manual methods. That is, a plurality of processes (e.g., 10 or more processes) may be executed simultaneously. The methods described herein may further reduce opportunities for contamination and user error. Thus, the systems, apparatuses, and methods described herein may increase one or more of cell processing automation, repeatability, reliability, process flexibility, instrument throughput, process scalability, and reduce one or both of labor costs, and process duration.

### I. SYSTEM

Described here are systems and apparatuses configured to perform cell processing steps to manufacture a cell product (e.g., cell therapy product). In some variations, the CPS of the CPMS may include a docking station having a plurality of docking station modules each independently configured to perform one or more cell processing operations upon a cartridge (e.g., fluid device) of the CPMS. The use of a controller may facilitate one or more of automation, efficiency, and sterility of a cell processing and manufacturing system.

In some variations, the cell processing and manufacturing system may comprise a cell processing station that comprises an enclosure.

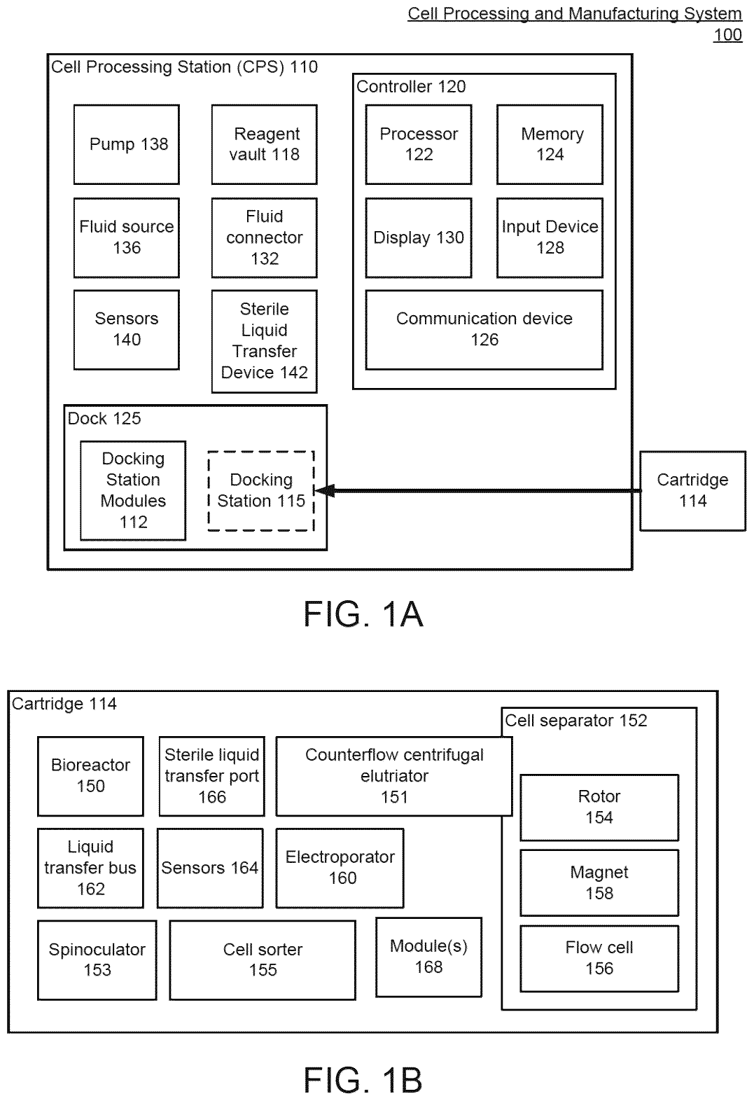

FIG. 1A is a block diagram of a cell processing and manufacturing system (CPMS) 100 including a cell processing station (CPS) 110 and a cartridge 114 (also referred to herein as a consumable). In some variations, the CPS 110 may include one or more of a controller 120, a dock 125, a pump 138, a reagent vault 118, a fluid connector 132, a sterilant source 134, a fluid source 136, at least one sensor 140, and a sterile liquid transfer device 142. In some variations, the controller 120 may comprise one or more of a processor 122, a memory 124, a communication device 126, an input device 128, and a display 130. In some variations, the dock 125 may include a plurality of docking station modules 112 and a docking station 115 configured to receive the cartridge 114, where each of the plurality of docking station modules 112 may correspond to a cartridge module of the cartridge 114.

In some variations, a CPS may include a fully, or at least partially, enclosed housing inside which one or more cell processing steps are performed in a fully, or at least partially, automated process. In some variations, the CPS may be an open system lacking an enclosure, which may be configured for use in a clean room, biosafety cabinet, or other sterile location. In some variations, the CPS may be configured to perform sterile liquid transfers into and out of the cartridge in a fully or partially automated process. For example, one or more fluids may be stored in the sterile liquid transfer device 142. In some variations, the sterile liquid transfer device 142 may be a portable consumable that may be moved within the CPMS 100. The sterile liquid transfer devices and fluid connectors described herein enable the transfer of fluids in an automated, sterile, and metered manner for automating cell therapy manufacturing. In some variations, the enclosure of the CPS may be configured to meet International Organization for Standardization (ISO) standard ISO7 or better (e.g., ISO6 or ISO5). An advantage of meeting ISO7 or better standards is that the system may be used in a facility that does not meet ISO7 standards (i.e. that lack a clean room or other sufficiently filtered air space). Optionally, the facility may be an ISO8 or ISO9 facility. In some variations, a CPS may include a volume of less than about 800 m³, less than about 700 m³, less than about 600 m³, less than about 500 m³, less than about 300 m³, less than about 250 m³, less than about 200 m³, less than about 150 m³, less than about 100 m³, less than about 50 m³, less than about 25 m³, less than about 10 m³, and less than about 5 m³, including all ranges and sub-values in-between.

In some variations, the cartridge 114 may receive cell product from different donors or contain cell product intended for different recipients. Cell product from a single donor may be split between multiple cartridges 114 if necessary to generate enough product for therapeutic use, or when a donor provides cell product for several recipients (e.g., for allogeneic transplant). In some variations, the multiple cartridges 114 may be placed sequentially in a docking station 115 (e.g., a first cartridge may be processed in the docking station and then a second cartridge may be processed in the docking station). In yet other variations, a first cartridge may be placed in a docking station 115 of a first cell processing station 110 and a second cartridge may be placed in a docking station 115 of a second cell processing station 110. The cell product for a single recipient may advantageously be split between multiple cartridges 114, if necessary, and processed within separate CPSs, to generate enough product for therapeutic use for the recipient. Similarly, the cell product for the single recipient may be split between multiple cartridges 114, if necessary, and processed within separate CPSs, to generate several cell products with unique genetic modifications, and then optionally recombined in certain ratios, for therapeutic use for the recipient. In other variations, the cell product from a single donor may be split between two separate bioreactors within the same cartridge, whereby they undergo separate processes. The cell products may then be combined and used as part of a therapeutic treatment.

As illustrated in FIG. 1B, a cartridge 114 may include a plurality of cartridge modules including one or more of a bioreactor 150, a cell separator 152, an electroporator 160, a spinoculator 153, a cell sorter 155, a counterflow centrifugal elutriator 151, and other modules 168, as appropriate and as described in more detail herein. It should also be appreciated that one or more of the plurality of cartridge modules may use shared equipment in order to accomplish a task of the module. The cartridge 114 may also include a liquid transfer bus 162 (also referred to herein as a fluidic bus), at least one sensor 164, and a fluid connector 166, as described in more detail herein. A cell separator 152 may include one or more of a rotor 154, flow cell 156, and magnet 158. In some variations, the magnet 158 may include one or more magnets and/or magnet arrays. For example, the cell separation system 152 may include a first magnet configured to magnetically rotate a rotor 154 and a second magnet (e.g., magnet array) configured to magnetically separate cells in flow cell 156.

### Cell Processing Station (CPS)

In some variations, a CPS 110 may include at least a partially enclosed enclosure (e.g., housing) in which one or more automated cell processing steps are performed. For example, the CPS 110 may be configured to transfer sterile liquid into and out of a cartridge 114 in a fully or partially automated process. In some variations, a CPS 110 may not have an enclosure and be configured for use in a clean room, a biosafety cabinet, or other suitably clean or sterile location. In some variations, the CPS 110 may be part of a CPMS 100 that includes a feedthrough access biosafety cabinet, quality control instrumentation, pump, consumable (e.g., fluid device), fluid connector, consumable feedthrough, and sterilization system (e.g., sterilant source and/or generator, fluid source, heater/desiccator, aerator).

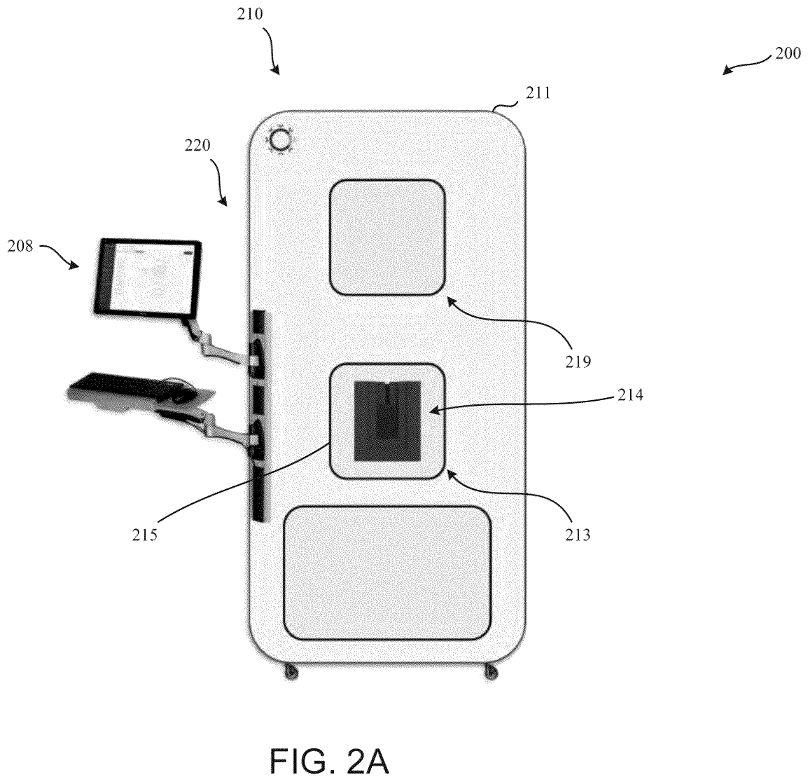

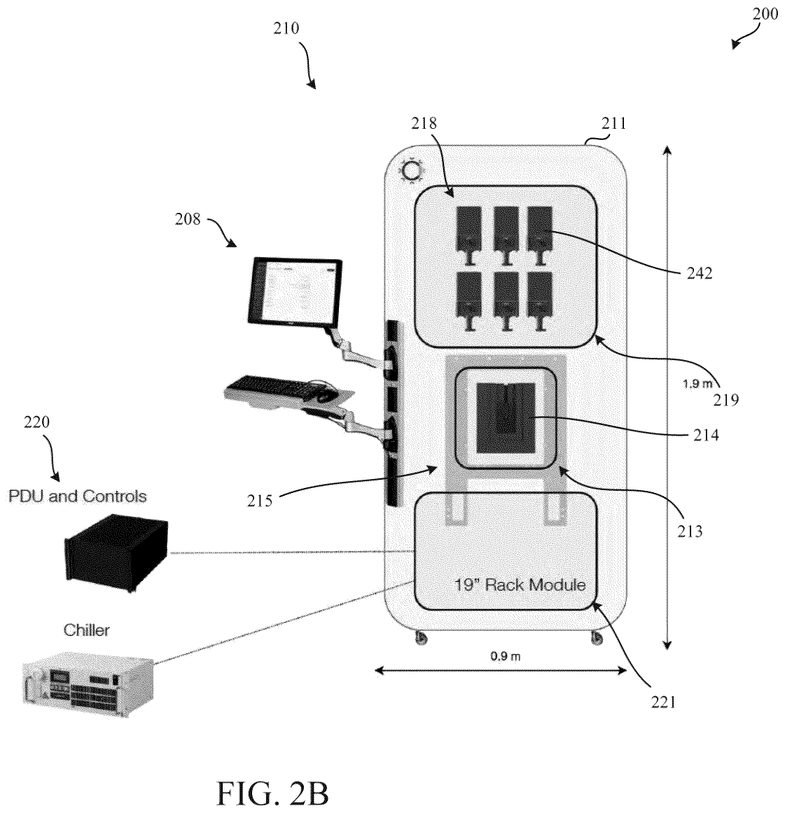

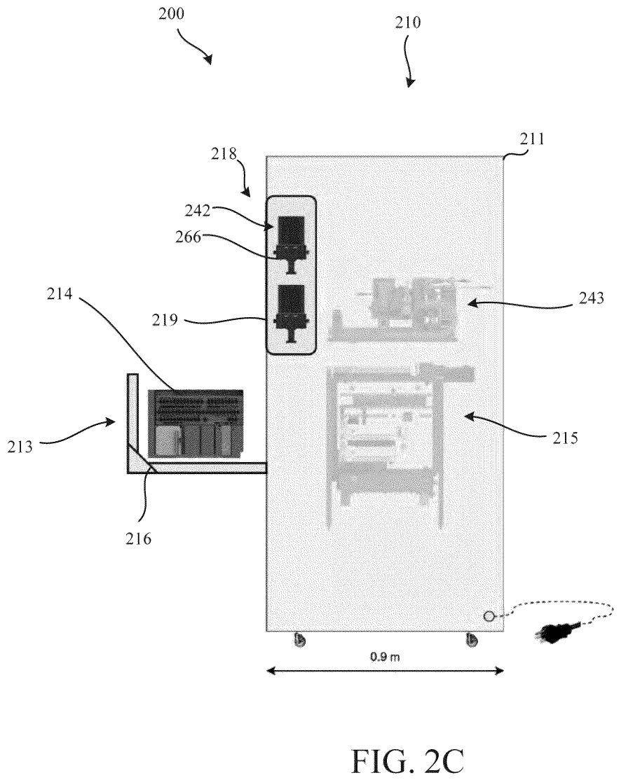

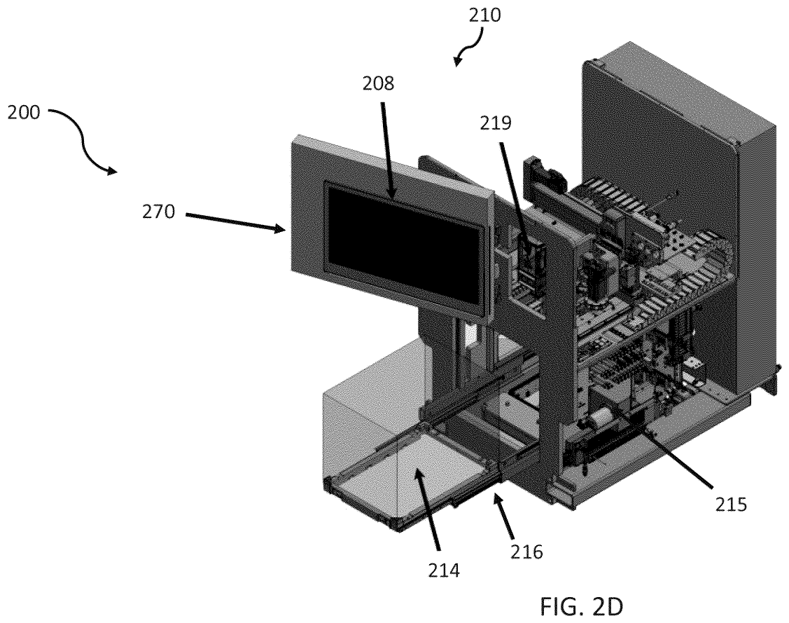

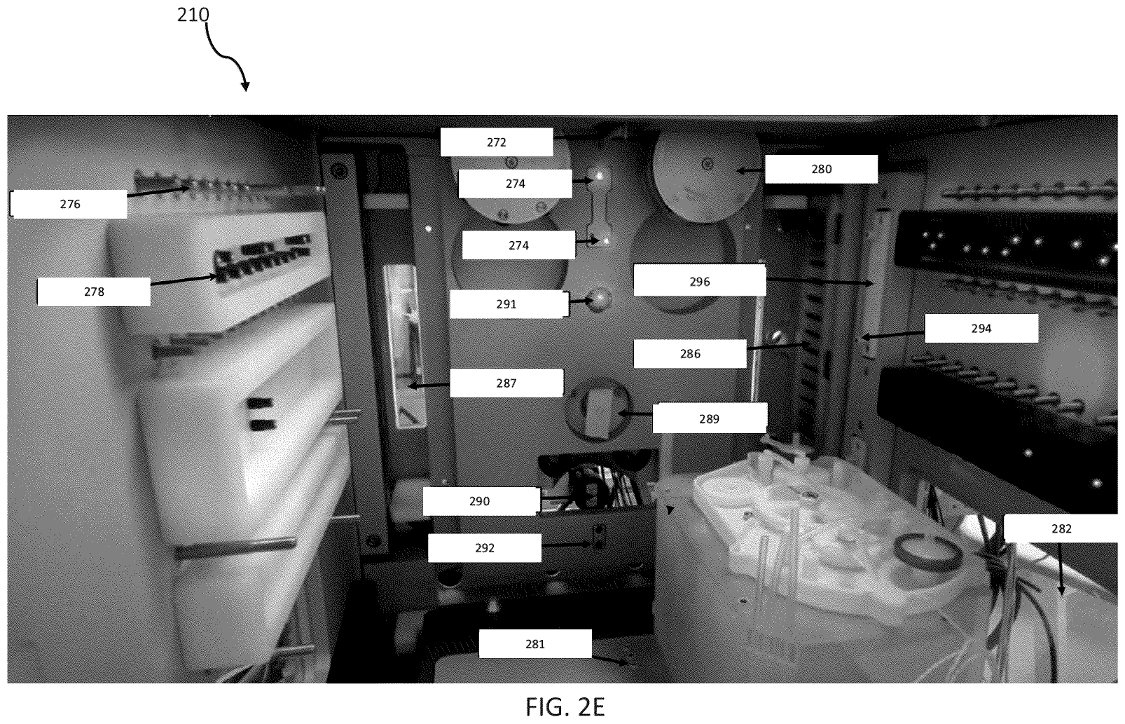

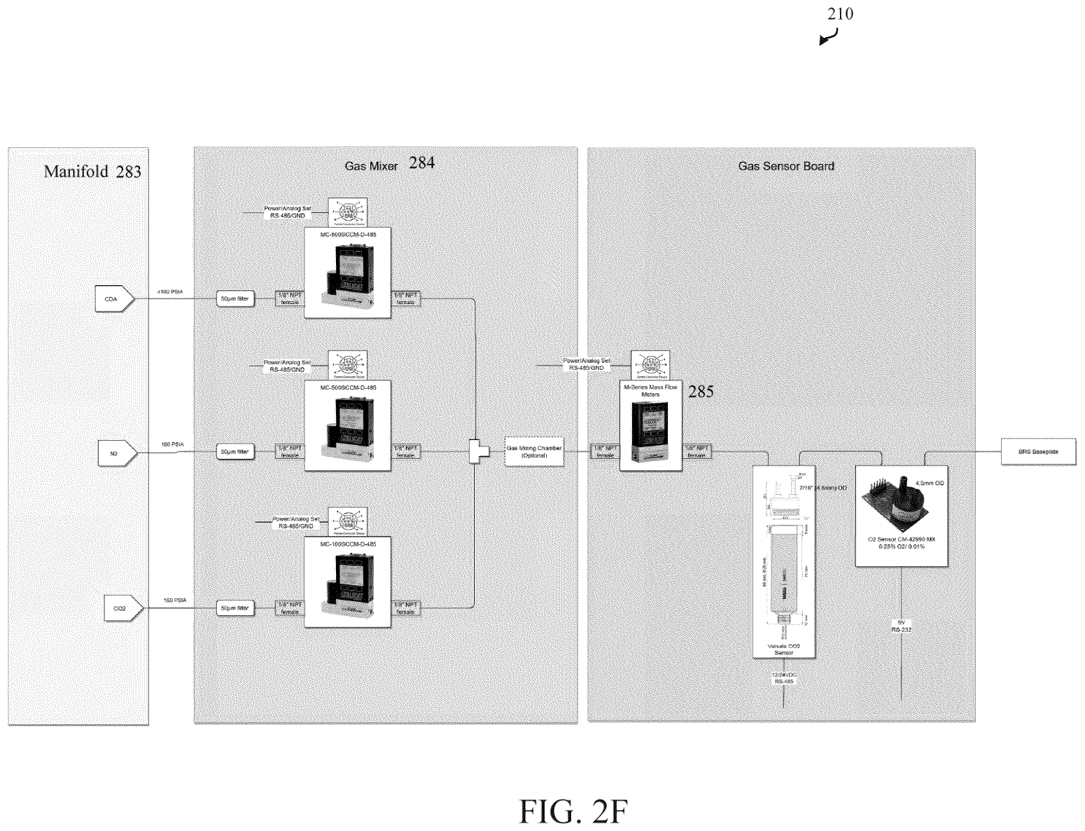

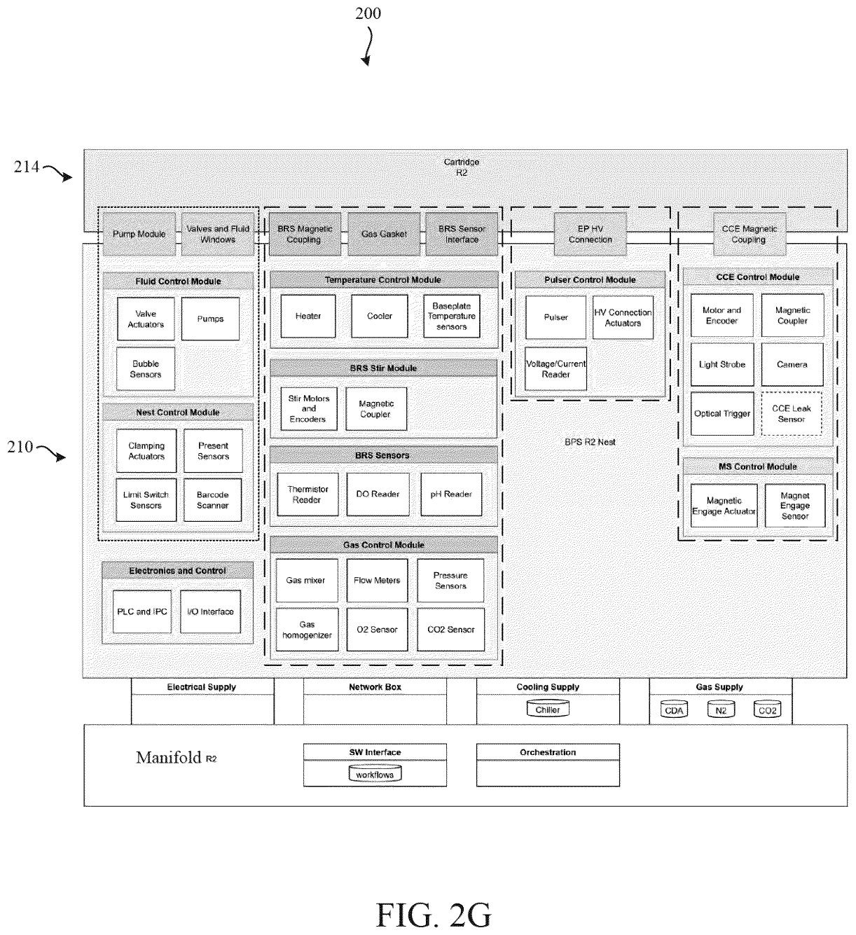

FIG. 2A is a block diagram of a cell processing and manufacturing system 200 including a cell processing station (CPS) 210 and a cartridge 214. The CPS 210 may include an enclosure 211 having four walls, a base, and a top. The CPS 210 may be divided into an interior zone with a feedthrough access 213, and, optionally, quality control (QC) instrumentation. An air filtration inlet (not shown) may provide high-efficiency particulate air (HEPA) filtration to provide ISO7 or better air quality in the interior zone. This air filtration may maintain sterile cell processing in an ISO8 or ISO9 manufacturing environment. The CPS 210 may also have an air filter on the air outlet to preserve the ISO rating of the room. In some variations, the CPS 210 may further include a novel docking station 215 having a plurality of docking station modules, such as a bioreactor, a cell selector (e.g., MACS), an electroporator (EP), a counterflow centrifugal elutriator (CCE), a cell sorter (e.g., fluorescence activated cell sorting (FACS)), a spinoculator, and the like. In variations, the CPS 210 may also include a sterile liquid transfer instrument, a reagent vault, and a sterilization system. The reagent vault may be accessible by a user through a sample pickup port 219.

In some variations of methods according to the disclosure, a human operator may load a cartridge 214, which may be empty or include any combination of the modules described herein, into the feedthrough 213. The cartridge 214 may be pre-sterilized, or after passing through the feedthrough 213 and into the docking station 215, may be sterilized using ultraviolet radiation (UV) or chemical sterilizing agents provided as a vapor, spray, or wash. In some variations, the cartridge 214 may be pre-loaded with an input cell product by the user. After positioning the sterilized, loaded cartridge 214 into the docking station 215 via the feedthrough 213, the user may initiate automated cell processing using a computer processor in the computer server rack (e.g., a controller 220). Advantageously, in some variations, the cartridge may undergo an automated end-to-end process to generate a desired cell product, all while remaining in a docked position in the docking station 215. At the end of cell processing, the cartridge 214, now having the processed cell product, may be retrieved by the user from the docking station 215 via the feedthrough 213. In some variations, an outer surface of the enclosure 202 may include an input/output device 208 (e.g., display, touchscreen) communicatively coupled to the controller 220.

FIG. 2B is a perspective view of the CPMS 200 of FIG. 2B. In some variations, the CPS 210 may have a height of more than about a meter, such as between about 1 m and about 1.1 m, between about 1 m and about 1.2 m, between about 1 m and about 1.3 m, between about 1 m and about 1.4 m, between 1 m and about 1.5 m, between about 1 m and about 1.6 m, between about 1 m and about 1.7 m, between about 1 m and about 1.8 m, between about 1 m and about 1.9 m, between about 1 m and about 2 m, between about 1 m and about 2.5 m, between about 1 m and about 3 m, between about 1 m and about 5 m, between about 3 m and about 10 m, between about 5 m and about 20 m, between about 10 m and about 30 m, between about 20 m and 100 m, and more than about 100 m, including all values and ranges in-between. For example, in some variations, the CPS may have a height of about 1.9 m. In some variations, the CPS 210 may have one or both of a length and width of more than about 1 meter, such as between about 1 m and about 5 m, between about 3, and about 10 m, between about 5 m and about 20 m, between about 10 m and about 30 m, between about 20 m and 100 m, and more than about 100 m, including all values and ranges in-between. Oppositely, in some variations, the CPS 210 may have one or both of a length and width of less than about 1 meter, such as between about 0.2 m and about 0.9 m, between about 0.3 m and about 0.9 m, between about 0.4 m and about 0.5 m, between about 0.5 in and about 0.9 m, between about 0.6 m and about 0.9 m, between about 0.7 m and about 0.9 m, or between about 0.8 m and about 0.9 m. For example, in some variations, the CPS may have one or both of a length and width of about 0.9 m.

As in FIG. 2B, the CPMS 200 may include a cartridge 214 and a CPS 210 having an enclosure 211. The cartridge 214 may be fed through a feedthrough 213 of the enclosure to be arranged within a docking station 215 of the CPS 210. The CPS 210 may also include a sample pickup port 219, which may also be a reagent vault 218 having one or more sterile liquid transfer devices 242. In variations, the CPS 210 may further include a rack module 221 for housing one or more of a controller 220, a power distribution unit (PDU), and a chiller, where the controller 220 may be controllable at least by a user interface of an input/output device 208. In some variations, the CPS 210 may be mobile. For instance, the CPS 210 may be outfitted with wheels or casters for repositioning and relocation. As the CPS 210 may be easily transportable, it may be possible to transport it into any room in a facility (e.g., a hospital), or to transport two or more CPSs into a single room, if desired.

FIG. 2C is a side view of a cell processing and manufacturing system 200 depicting a cartridge 214 (e.g., any of the cartridges described herein) being introduced into a CPS 210 (e.g., any of the CPSs described herein). Each inserted cartridge 214 may undergo one or more cell processing operations. As shown in FIG. 2C, the cartridge 214 may be positioned on a docking station drawer 216 that is movable through a feedthrough 214 of an enclosure 211 of the CPS 210 to position the cartridge 214 within a docking station 215 of the CPS 210. The CPS 210 may further include a built-in reagent vault 218 configured to store one or more sterile liquid transfer devices 242 which may be inserted, removed, and/or exchanged via sample pickup port 219 (e.g., by an operator or by a robot of the CPS). In some variations, the docking station 215 may be outfitted with, in addition to the docking station modules configured to interface with corresponding modules of the cartridge 214, a sterile liquid transfer instrument 243. The sterile liquid transfer instrument 243, which may optionally be separate from but releasably couplable to the cartridge 214, may be configured to transfer fluids from and/or to sterile liquid transfer devices 242 of the reagent vault 218 (or other fluid vessels similarly configured with a fluid connector, as will be described later, in order to be controllable by the sterile liquid transfer instrument 243 to exchange fluids with the cartridge 214). In some variations, the sterile liquid transfer instrument 243 may include a robot controllable to interact with each of the sterile liquid transfer devices 242 of the reagent vault 218 (which may be referred to herein generally as fluid transfer devices 242) and with the cartridge 214 in order to supply and remove fluids from modules of the cartridge 214, as required by cell processing operations.

Similarly, FIG. 2D is a perspective view of a CPMS 200 depicting a cartridge 214 positioned on a docking station drawer 216 of a CPS 210. As shown, the drawer 216 may include a telescoping feature that may extend from the docking station 215 (e.g., exterior to the CPS to receive the cartridge 214) and retract into the docking station 215 (e.g., upon receiving the cartridge 214) such that the entirety of the drawer 216 may be enclosed within the docking station 215. Also depicted in FIG. 2D is a sample pickup port 219 and a door 270 having an input/output device 208. In some variations, the door 270 may be movable relative to the sample pickup port 219. In particular, the door 270 may be configured to be repositioned such that it inhibits or allows access to the sample pickup port 219. The door 270 may be repositionable manually and/or automatically (e.g., by an operator and/or by the controller 220). For example, in some variations, the door 270 may be slidable (e.g., translatable along a mount) both toward (and in front of) and away from the sample pickup port 219. Accordingly, the door 270 may provide temporary access to sterile liquid transfer devices and/or other reagent vessels at the sample pickup port 219. Moreover, in some variations, the door 270 may support, or may be, input/output device 208. For example, at least a portion of the door 270 may include the input/output device 208 configured to directly receive user input (e.g., via a touchscreen and/or button controls).

As described above, the CPS 210 may generally include at least one docking station module (e.g., docking station modules 112 in FIG. 1A) configured to engage the cartridge 214 and/or interface with a corresponding module of the cartridge 214. An interior view of a CPS 210 having several exemplary docking station modules is depicted in FIG. 2E. For example, in some variations, the CPS 210 may include a nest control module for ensuring that the cartridge is properly oriented within the docking station 215 and/or connecting the cartridge 214 to the CPS 210. The nest control module may include one or both of electrical interface 272 and cartridge presence sensors 274. The electrical interface 272 may be, for example, a PCB with pogo pins configured to physically and electrically interface with a corresponding electrical portion of the cartridge 214. Moreover, the presence sensors 274 may include one or both of a cartridge seated sensor and a cartridge presence sensor. A cartridge seated sensor may be a diffuse reflective sensor configured to direct a beam across the cartridge 214 and detect any light reflected at the sensor. When the beam is reflected, the cartridge may be mispositioned. Accordingly, the cartridge seated sensor may be electrically coupled to the controller 220 and/or the input/output device 208 to indicate to an operator (e.g., visually, audibly, haptically, etc.) whether the cartridge 214 is oriented correctly within the CPS 210. Therefore, an operator may have an opportunity to reorient the cartridge 214 within the CPS 210 prior to clamping the cartridge 214 inside the CPS 210. Similarly, the cartridge presence sensor may be a diffuse reflective sensor (as previously described) which may be used to determine whether a cartridge is present in the docking station 215. In some variations, the nest control module may include one or more additional or alternative components to ensure that the cartridge 214 is properly oriented within and connected to the CPS 210. Nonlimiting examples of the one or more additional or alternative components may include such as clamping actuators (e.g., for clamping the cartridge 214 within the docking station 215), limit switch detectors (e.g., for sensing physical contact between the cartridge and the docking station 215), and barcode scanners.

Moreover, in some variations, the CPS may include a fluid control module having one or more of valve actuators 276, sensors 278, and pump mechanism 280. The valve actuators 276 may be pinch valve actuators that control fluid (e.g., liquid, gas) flow through the fluidic bus (e.g., to and from the cartridge 214, as described herein throughout) by adjusting an amount of pressure applied to the valves via pneumatic cylinders. The sensors 278 may detect bubbles in one or more fluid pathways of the fluidic bus. For example, the sensors 278 may be bubble sensors that use fiber optics to visualize changes in the amplitude of the sensor output, which may inform whether air, liquid, or air and liquid (i.e., bubbles) are flowing through the fluidic bus. The pump mechanism 280 may be configured to move fluid through the cartridge 214. For example, the pump mechanism 280 may include one or more pump rollers coupled to one or more motors that enable the one or more pump rollers to act as peristaltic pumps to move fluid through the cartridge 214. The pump mechanism 280 may engage with the cartridge 214 via tubing to form one or more fluid pathways. In some variations, the pump mechanism 280 may be considered a discrete module (i.e., a pump module) that is configured to engage with a plurality of cartridge modules. That is, any modules requiring gas or fluid flow (e.g., gas and/or liquid flow) may be engaged with the pump module.

Additionally, or alternatively, the CPS may include a bioreactor module for cultivating various cell types (described herein throughout). Within the docking station 215, the bioreactor module may include one or both of ports 281 and viewing window 282 (also referred to herein as a fluid window). The ports 281 may be supply supports (e.g., gas, fluid, or solid supply ports) configured to feed nutrients (e.g., CO2, N2, and/or air) into the bioreactor such that cells may be properly cultured. The viewing window 282 may, for example, allow a camera to detect a liquid level of one or more containers of the bioreactor.

Referring briefly to FIG. 2F, in some variations, the bioreactor module may include a manifold 283 external to the CPS 210. The manifold 283 may be configured to supply gas to the CPS 210 by housing external sources for clean dry air (CDA), nitrogen (N2), and carbon dioxide (CO2), etc., and providing the gases to a gas mixer 284 of the CPS 210. Accordingly, the bioreactor module may formulate the gases for supply to the bioreactor within the cartridge 214. In some variations, the supplied gases may be filtered and then each provided to one or more mass flow meters 285 (depicted on the input side of the bioreactor in FIG. 2F). The one or more mass flow meters may be considered a first flow meter configured to combine the gases and provide the resulting gas to a second mass flow meter before being evaluated by a carbon dioxide sensor, an oxygen sensor, and then provided to the bioreactor (e.g., via a baseplate of the bioreactor). The mass flow meters 285 may be configured to provide information about mass flow of gas (e.g., volume per second of each gas) and ensure that a proper cell growth environment is provided. That is, cell growth requires a specific mixture of gases (e.g., CO2, O2) and the mixture of gases required may change depending on which growth state the cells are in.

Referring again to FIG. 2E, the CPS may include a magnetic-activated cell selection module (MACS) for magnetically selecting cells having a predetermined property or component, such as an antigen (described herein throughout). Accordingly, the MACS may include magnets 286, which may engage with an actuator and the cartridge 214 to carry out magnetic selection of the cells. Additionally, in some variations, the MACS may include viewing window 287 (also referred to herein as fluid window), which may allow a camera (e.g., an external camera) to view a liquid volume and/or a degree of cell separation that occurred during magnetic selection.

As another example, the CPS may include a counterflow centrifugal elutriation (CEE) module configured to separate cells based on predetermined characteristics such as size and/or density (described herein throughout). The CEE module may include one or more of magnetic coupler 289, camera 290, tachometer 291, and sensor 292. The magnetic coupler 289 may include a motor having a magnet coupled thereto, where the magnet is configured to pair with a mating magnet within the cartridge 214. Thus, when the magnets are close enough to attract each other, the motor of the magnetic coupler 289 may spin to operate the CCE module. Next, the camera 290 may be configured to view an elution chamber of the CEE (e.g., a bicone or funnel where cell separation occurs) within the cartridge 214. The tachometer 291 may be configured to calculate the RPMs of the CCE when it is rotating. In particular, the tachometer 291 may include a sensor configured to direct a beam at a rotor of the CEE within the cartridge 214, and the rotor may be reflective (e.g., may include reflective tape or coating) such that the tachometer 291 may calculate the RPMs of the CEE relative to the rotor. In some variations, the tachometer 291 may be paired with the camera 290 such that images may be taken to visualize cells in the CCE within the cartridge 214. Moreover, the sensor 292 may be a leak detection sensor configured to use fiber optics to detect a leak in the CEE rotor. The sensor 292 may be configured to shut down the CEE process when a leak occurs. In some variations, the sensor 292 may be electrically coupled to the controller and/or the input/output device 208 to indicate to an operator (e.g., visually, audibly, haptically, etc.) whether a leak is occurring.

As yet another example, the CPS may include an electroporation (EP) module for facilitating intracellular delivery (i.e., transfection by electroporation) of macromolecules (described herein throughout). The EP module may include one or both of a sensor 294 and an actuator 296. The sensor 294 may be a liquid volume sensor configured to use fiber optics detect a volume of fluid within a flow cell of the EP module. Moreover, the actuator 296 may be a high voltage actuator configured to form a high voltage coupling between the cartridge 214 and the CPS 210.

In some variations, one or more components of the modules described above with respect to FIG. 2E may be shared among the modules. For example, the electrical interface 272 described with respect to the nest control module may additionally, or alternatively, be a component of one or more docking station modules described above (e.g., the fluid control module, the bioreactor module, the magnetic cell selection module, the CEE module, and the EP module) and/or one or more docking station modules described in more detail below (e.g., the electronics and control module).

FIG. 2G is a block diagram of a CPMS 200 described herein throughout. In particular, the block diagram of FIG. 2G shows components of a CPS 210 of the CPMS 200, which may be grouped in various combinations to form docking station modules, as described above with respect to FIGS. 2E and 2F. For example, the CPS 210 may include one or more of electronics and control module, the nest control module, the fluid control module, a gas control module, bioreactor sensors, a bioreactor stir module, a temperature control module, a pulser control module, a CCE control module, and magnetic separation (MS) control module. In some variations, one or more of a pump mechanism, valves, viewing windows, magnetic couplings (e.g., for CEE and/or bioreactor modules), a gas gasket, a bioreactor sensor interface, an electroporator (EP) high voltage (HV) connection, and a CCE magnetic coupling may enable the docking station 215 of the CPS 210 to interface with the cartridge 214. In variations, the CPS 210 may also include, or be operatively coupled to, a manifold (e.g., a gas manifold) which may be either integrated with or separate from the CPS 210, an electrical supply, a network box, a cooling supply, a gas supply, and software for automating cell processing operations within the cartridge 24 (e.g., SW Interface, Orchestration). In some variations, the electronics and control module may include one or more of a programmable logic controller (PLC), and/or interprocess communication (IPC), as well as one or more of the controller 220, the input/output device 208, and electrical interface 272. Accordingly, the electronics and control module may allow for user interaction with the CPS 210 and may thereby coordinate at least a subset of operations and processes of the CPMS 200.

Moreover, the dotted lines in FIG. 2F depict how components of the CPS 210 may be grouped to form docking station modules configured to interface with the cartridge 214 and/or corresponding modules of the cartridge 214, as described above with respect to FIG. 2E. For instance, the fluid control module, which may include valve actuators, pumps, and/or bubble sensors, may interface and/or engage with the fluidic bus, the exterior of cartridge 214, a fluid connector of the cartridge 214, and/or fluid windows of the cartridge 214. As another example, the nest control module may engage an electrical portion of the cartridge 214 and/or one or more exterior portions of the cartridge 214 (e.g., for clamping the cartridge 214 within the docking station 215).

As yet another example, a bioreactor module of the cartridge 214 may be controllable via the magnetic coupling, the gas gaskets, and/or the bioreactor sensor interface. In some variations, this control may be by or in coordination with one or more of the temperature control module, the bioreactor stir module, the bioreactor sensors, and the gas control module. In variations, the temperature control module may include one or more of a heater, a cooler, baseplate temperature sensors, and the like. In variations, the bioreactor stir module may include one or more of stir motors and encoders, a magnetic coupler, and the like. In variations, the bioreactor sensors may include one or more of a thermistor reader, a dissolved oxygen (DO) reader, a pH reader, and the like. In variations, the gas control module may include one or more of a gas mixer, flow meters, pressure sensors, a gas homogenizer, an oxygen sensor, a carbon dioxide sensor, and the like (as described with respect to FIG. 2G). Similarly, an electroporator module of the cartridge 214 may be controllable via the EP HV connection by the pulser control module, where, in some variations, the pulser control module may include one or more of a pulser, an HV connection actuator, a voltage and/or current reader, and the like. Analogously, the CCE module and/or magnetic cell separation module and the like of the cartridge 214 may be controllable via the CCE magnetic coupling by the CCE control module and the MS control module, respectively (among others). In some variations, the CCE control module may include one or more of a motor and encoder, a magnetic coupler, a light strobe, a camera, an optical trigger, and a CCE leak sensor. In some variations, the MS control module may include one or more of a magnetic engage actuator, a magnetic engage sensor, and the like.

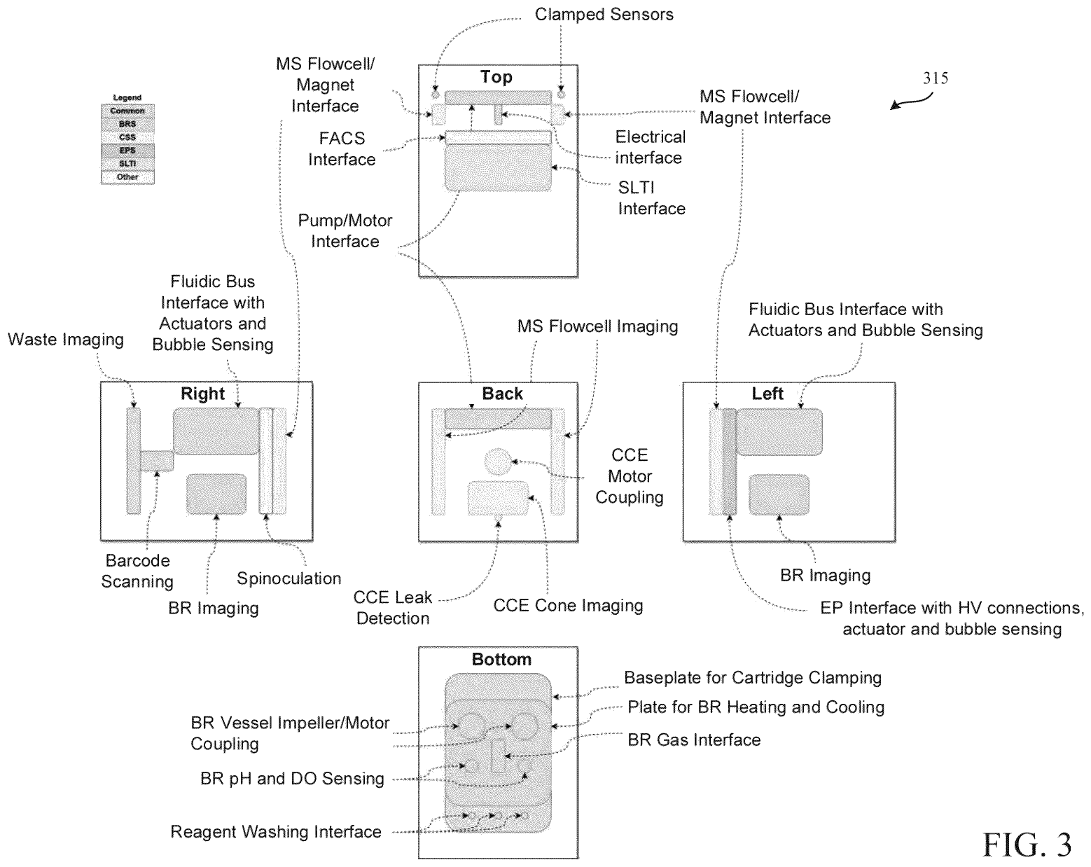

FIG. 3 provides a two-dimensional (2D) schematic, in view of the cartridge modules, modules, instruments, and docking station modules described above, of a docking station 315 of the CPS. Particularly, the 2D schematic of FIG. 3 represents the docking station 315 of the CPS in a flattened version, such that relative locations of interfaces between corresponding modules are shown and assuming the front of the docking station 315 is open so as to receive a cartridge therein. In other words, assuming an instance when the docking station 315 is a rectangular prism, panels of FIG. 3 are labeled “Bottom”, “Back”, “Top”, “Left”, and “Right” and modules (e.g., cartridge modules and/or docking station modules) and instruments are identified based on their positioning on the docking station 315. The 2D schematic of FIG. 3 illustrates how the CPS may advantageously perform multiple functions on a cartridge docked within the docking station 315 by carefully utilizing the panels to perform different cell processing steps in a partially or fully enclosed system, which no other system may do. FIG. 3 includes reference to a sterile liquid transfer instrument (SLTI), a bioreactor (BR), and a magnetic separator (MS), such as a MACS.

For instance, a magnetic separator (MS) and a barcode, or barcode scanner (interfaced with the nest control module) are interfaced on a left wall of the docking station 315. The top wall of the docking station 315 comprises an electroporator (EP) box and a bubble sensor reader (of the fluid control module). The right wall of the docking station 315 comprises and an electroporator (EP) high voltage (HV) interface and a fluid bus, which interfaces with the pump module and the valves and fluid windows to supply to and remove fluids from each of the cartridge modules within the cartridge. The back wall of the docking station 315 comprises strobes, a CCE motor, and a camera, which are included within the CCE control module to interface with the CCE module of the cartridge. The bottom wall of the docking station 315 comprises a bioreactor tub, a gas mixer, and a gas sensor, which are configured to interface with the bioreactor module of the cartridge, and a clamp plate configured to secure the cartridge on a drawer of the docking station 315.

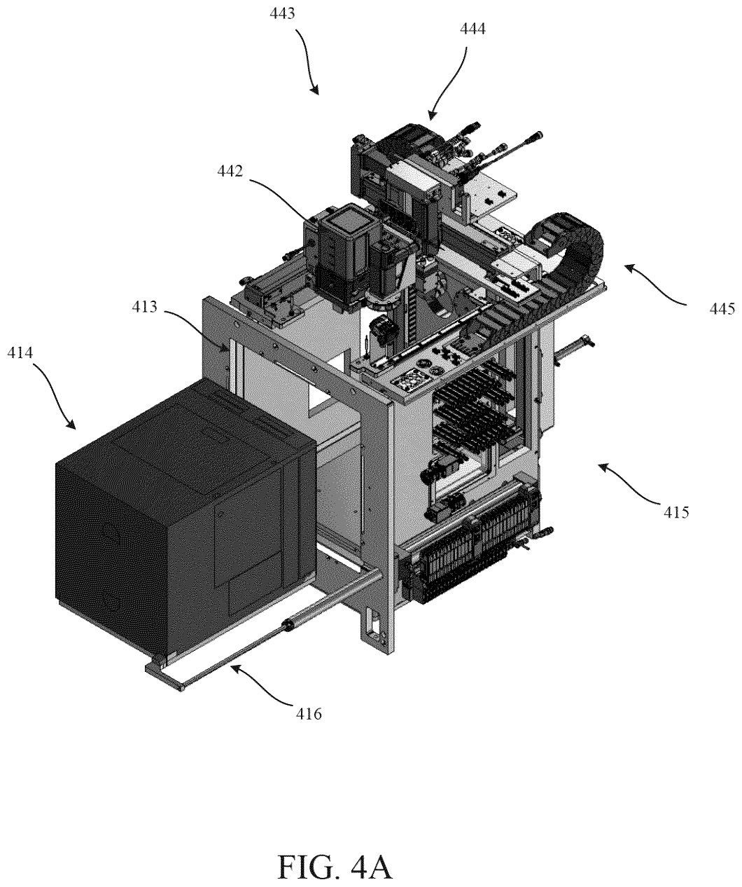

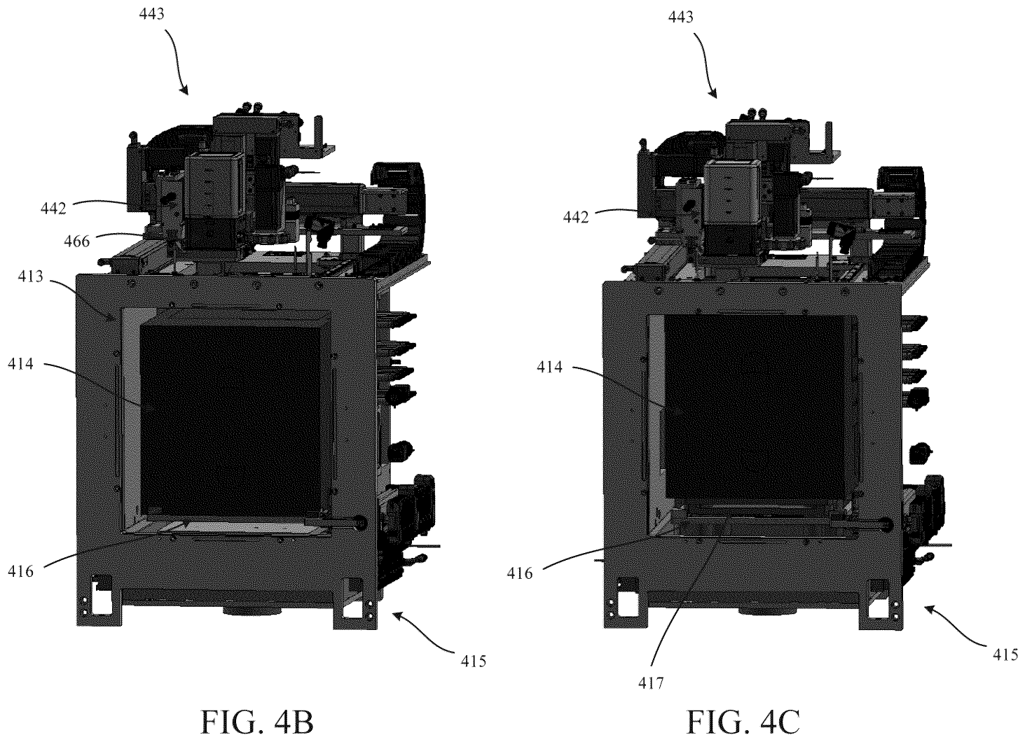

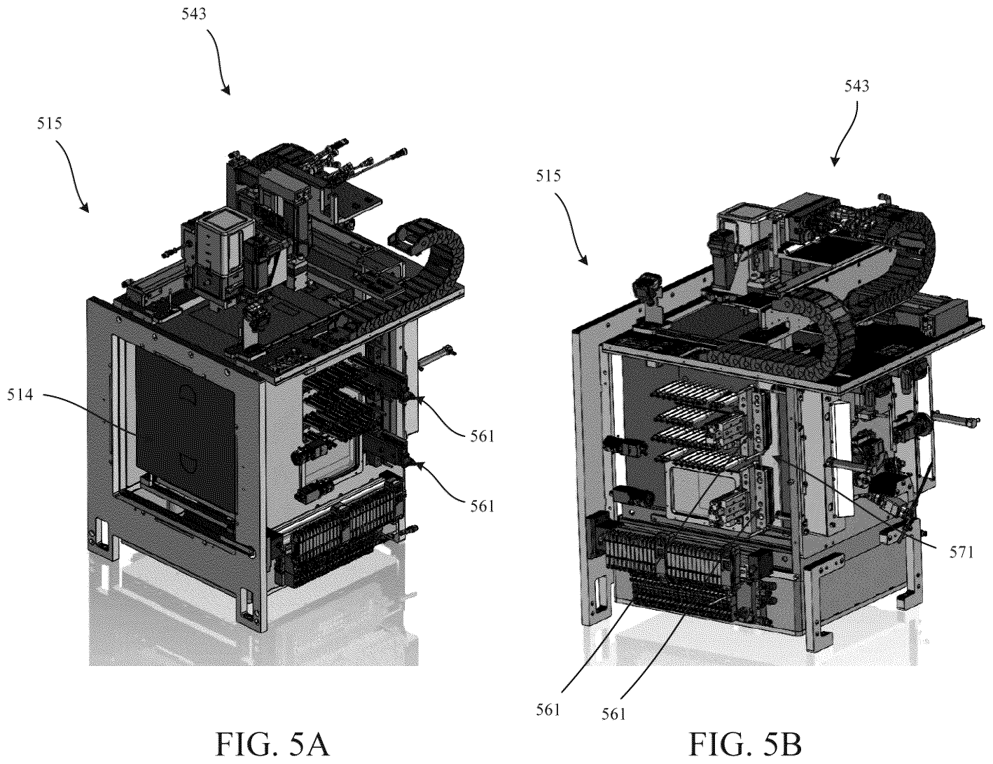

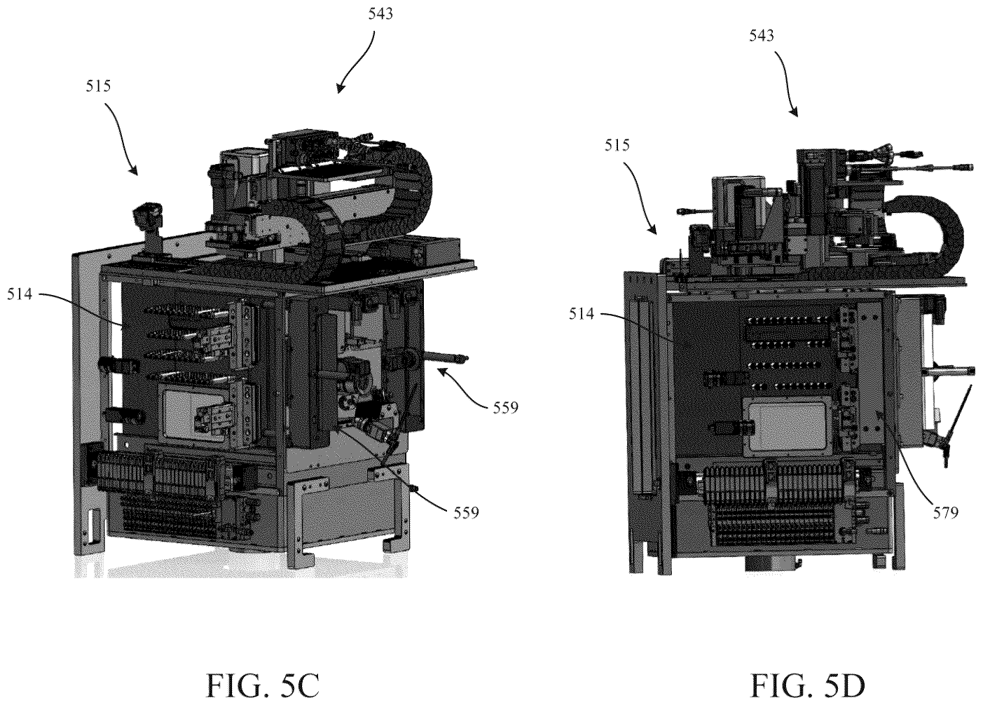

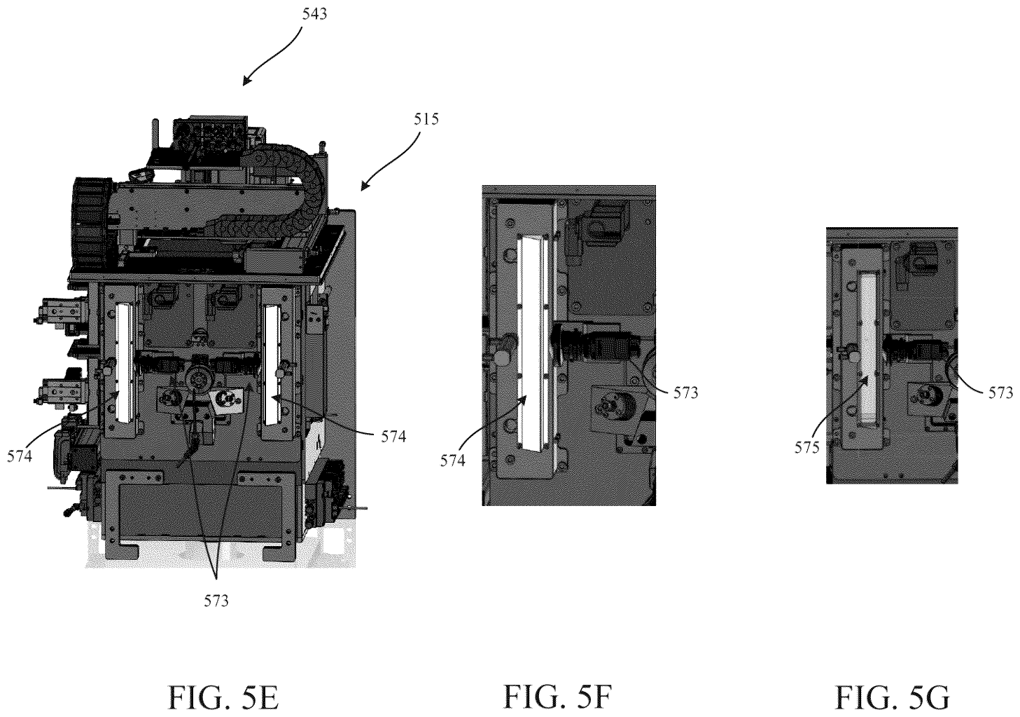

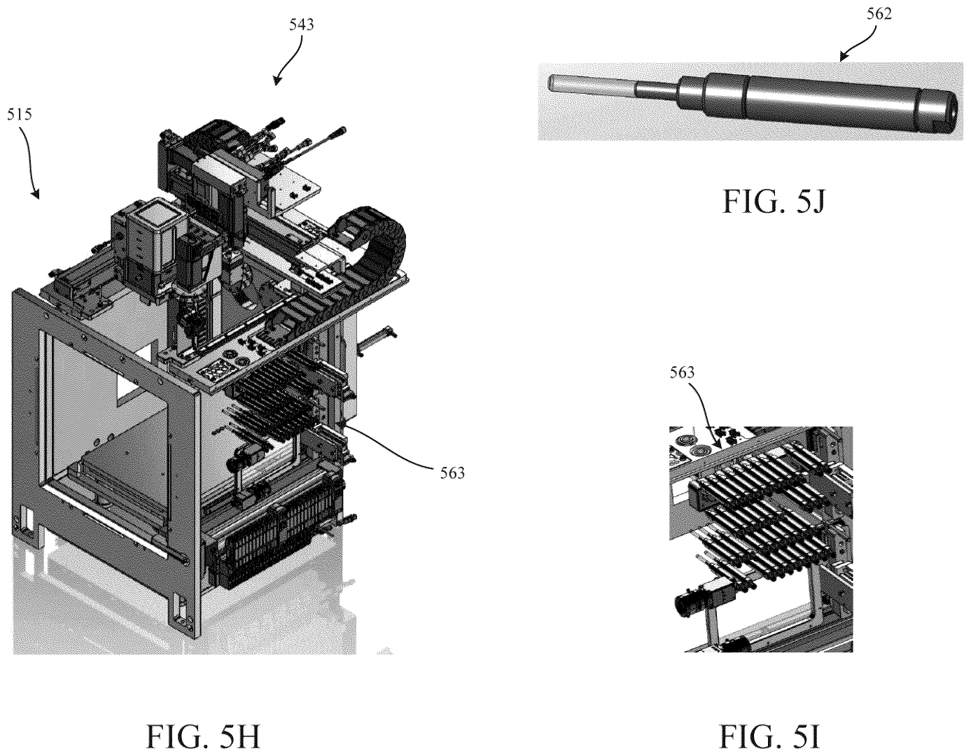

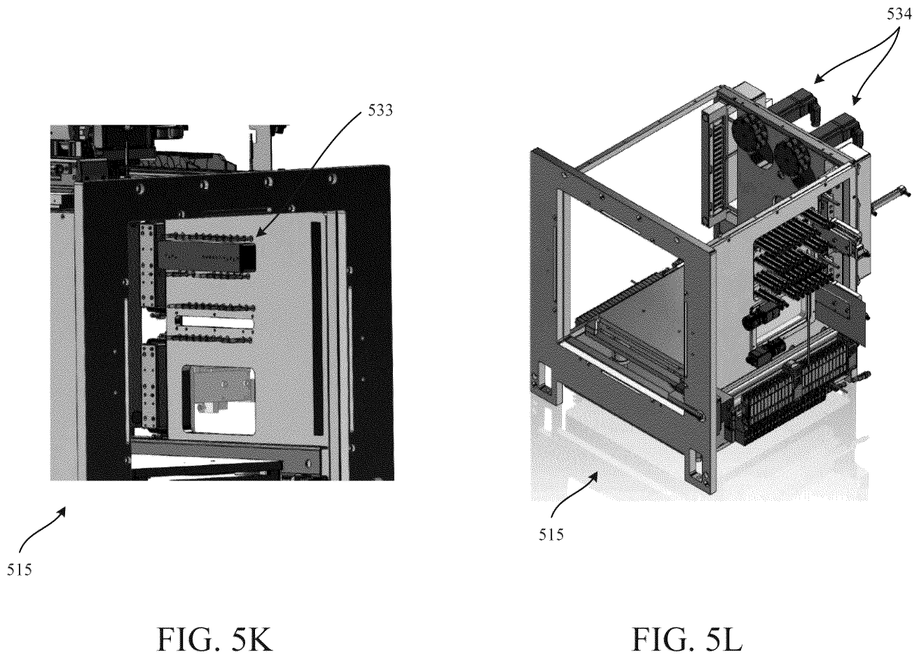

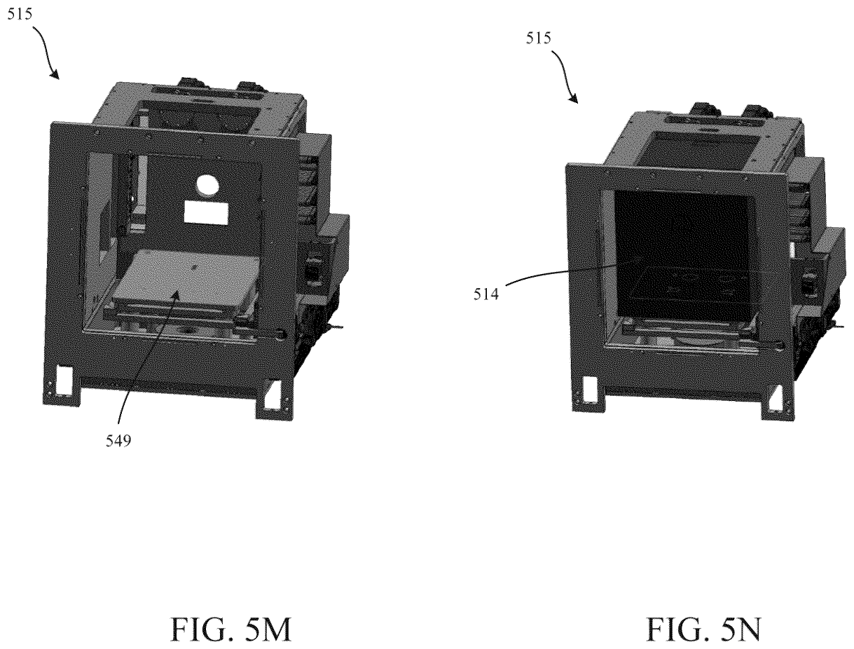

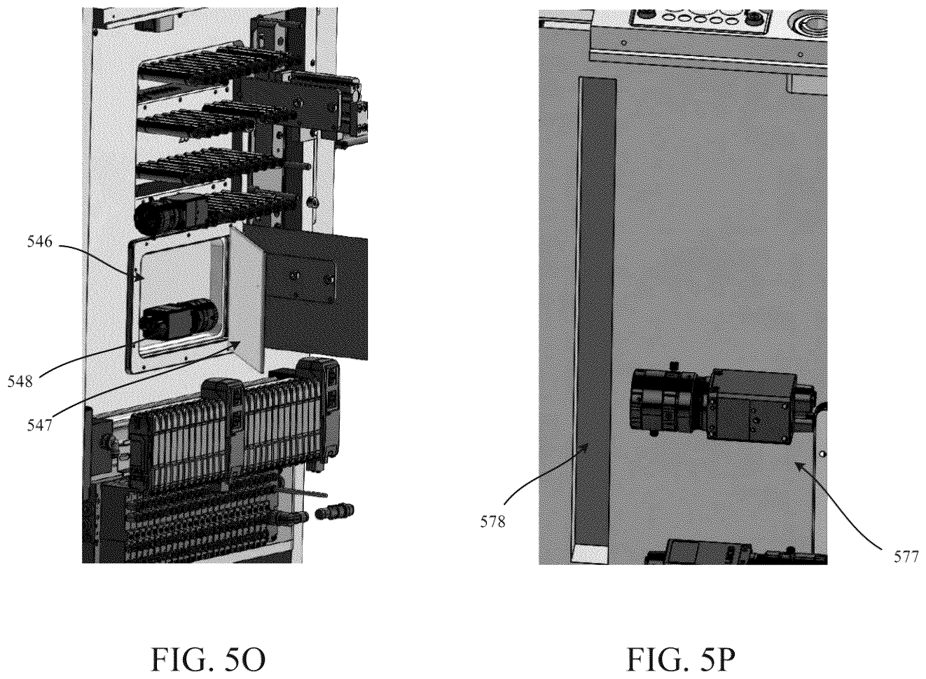

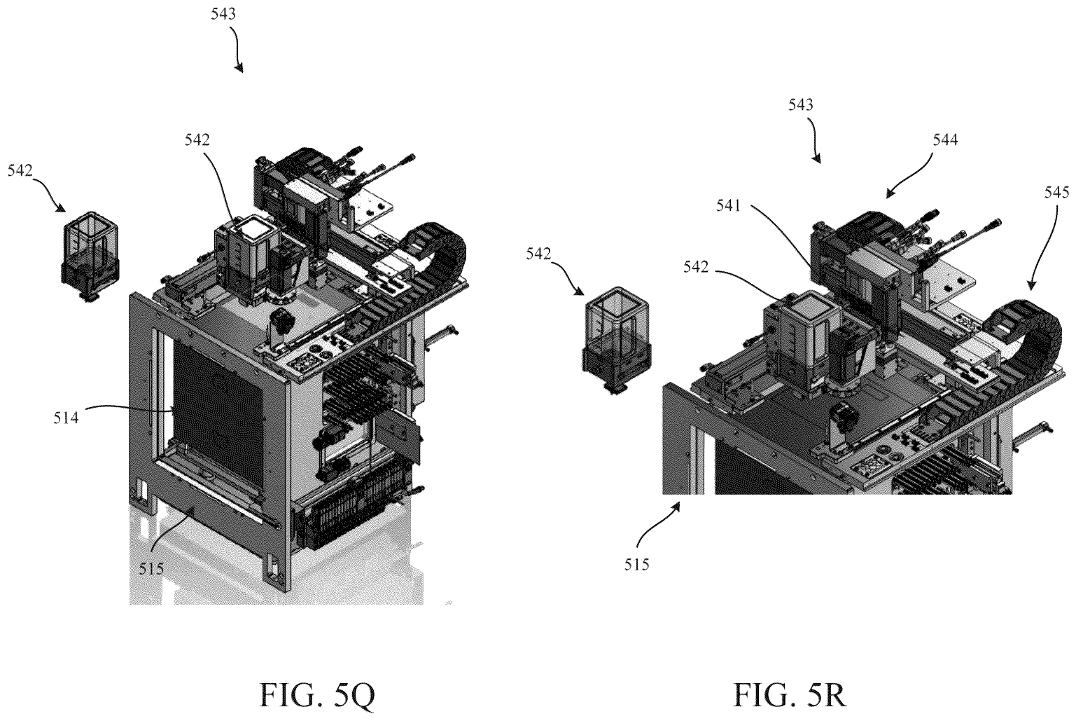

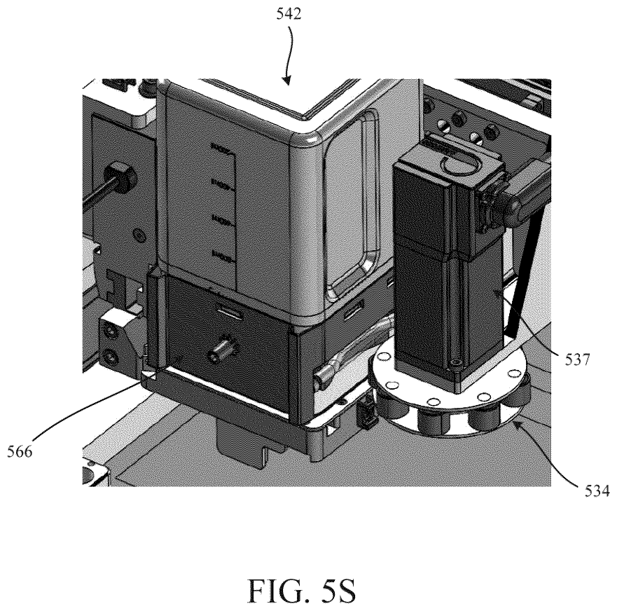

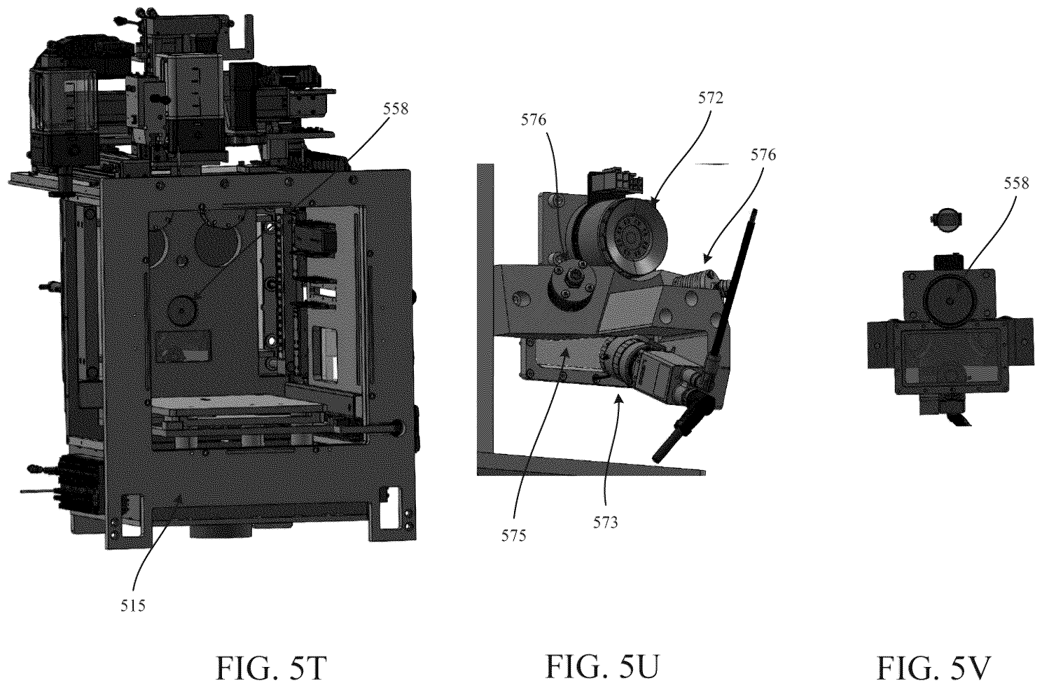

The 2D schematic of FIG. 3, in view of the preceding Drawings, will be described in further detail below with reference to FIG. 4A-5V.

FIG. 4A provides a perspective view of a rendering of a docking station 415, a sterile liquid transfer instrument 443 coupled thereto, and a cartridge 414 (e.g., a consumable) disposed on a docking station drawer 416. As shown in FIG. 4A, the cartridge 414 is yet to be moved into a chamber of the docking station 415 via feedthrough 413. In variations, the sterile liquid transfer instrument 443 comprises a first robot 444 and a second robot 445 configured to provide two axis range of motion to the sterile liquid transfer instrument 443. In variations, and as shown in FIG. 4A, a sterile liquid transfer device 442 may be coupled to the sterile liquid transfer instrument 443 in a position so as to be proximate to the cartridge 414 when the cartridge 414 is within the docking station 415. Accordingly, the sterile liquid transfer instrument 443 may advantageously be used to supply or extract media (e.g., fluids and liquids) to and from the cartridge while the cartridge is docked within the docking station 415. In variations, the docking station drawer 416 is a telescoping drawer, as will be exploited in FIG. 4B.

As shown in FIG. 4B, the cartridge 414 may be retracted into the docking station 415 on the docking station drawer 416 and via the feedthrough 413. The cartridge 414 may then be lifted from the docking station drawer 416 by lift 417 (shown in FIG. 4C) and the cartridge 414 may be positioned into contact with and, in variations, clamped to a top internal surface of the docking station 415. In variations, bringing the cartridge 414 into contact with the top internal surface of the docking station 415 allows the cartridge 414 to be engaged by a sterile liquid transfer instrument 443. For instance, after engagement between the cartridge 414 and the sterile liquid transfer instrument 443, a sterile liquid transfer device 442 may supply fluids or extract fluids to or from the cartridge 414 via a sterile liquid transfer port 466 of the sterile fluid transfer device 442.

FIGS. 5A-5V illustrate various aspects of the CPMS and arrangements of components relative to the docking station of the CPS, including docking station modules and, in variations, cartridge modules. The panels and associated features shown in FIGS. 5A-5V may be part of the same assembly shown in FIGS. 4A-4C in accordance with some variations.

FIG. 5A and FIG. 5B illustrate aspects of an electroporation assembly of the CPMS of the present disclosure. Each of FIG. 5A and FIG. 5B depict a cartridge 514 within a docking station 515, wherein the docking station 515 is coupled to a sterile liquid transfer instrument 543 on a top surface thereof. As shown in FIG. 5A, the docking station 515 comprises a docking station electroporation module, wherein electroporator actuators 561 thereof are shown. An aspect 571 of a corresponding cartridge electroporation module is shown in FIG. 5B, where it may be appreciated that the electroporation actuators 561 of the docking station electroporation module interface with the aspect 571 of the cartridge electroporation module.

FIG. 5C and FIG. 5D illustrate aspects of a magnetic cell sorting assembly of the CPMS of the present disclosure. Each of FIG. 5C and FIG. 5D depict a cartridge 514 within a docking station 515, wherein the docking station 515 is coupled to a sterile liquid transfer instrument 543 on a top surface thereof. In variations, the docking station 515 comprises a docking station magnetic-activated cell selection (MACS) module and the cartridge 514 comprises a cartridge MACS module. FIG. 5C shows a docking station MACS module 559, wherein actuators of the docking station MACS module 559 are in a retracted position. FIG. 5D shows an aspect 579 of the MACS assembly 559, wherein the actuators of the docking station MACS module 559 are in an extended position and are configured to supply magnetism to a corresponding cartridge MACS module of the cartridge 514. FIG. 5E through FIG. 5G provide additional details regarding the docking station MACS module 559. In variations, the docking station MACS module 559 comprises at least one camera 573 for viewing the corresponding cartridge MACS module of the cartridge 514 through a viewing window 575. In variations, the at least one camera 573 is positioned such that a mirror 574 is deployed to allow viewing of the corresponding cartridge MACS module of the cartridge 514 through the viewing window 575.

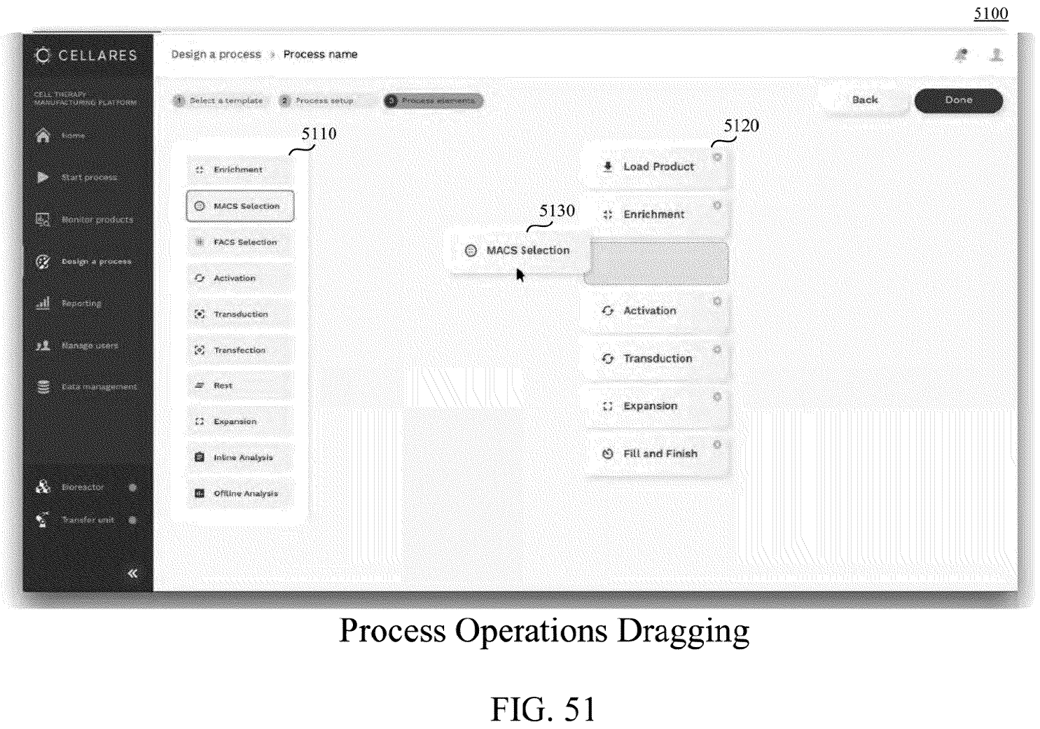

FIG. 5H through FIG. 5 illustrates liquid transfer bus actuators 563 of a docking station 515 having a sterile liquid transfer instrument 543 thereon, where the liquid transfer bus actuators 563 are configured to interface with a liquid transfer bus of a cartridge arranged within the docking station 515 to direct fluids within the cartridge. FIG. 51 provides for closer inspection of the liquid transfer bus actuators 563, and FIG. 5J provides for closer inspection of a single actuator 562 thereof. In variations, actuators of the liquid transfer bus actuators 563 may serve to compress corresponding pinch valves on the cartridge to allow flow to be directed to different places within the cartridge.

Working together with the liquid transfer bus actuators of FIG. 5H through FIG. 5J, FIG. 5K illustrates bubble sensors 533 arranged on an internal surface of a docking station 515 and configured to detect any bubbles that may exist within the liquid transfer bus.

Fluids may be pumped throughout a cartridge by motors 534 attached to pump rollers arranged on a back wall of a docking station 515. Together, this permits the pump rollers to be used as peristaltic pumps to move fluid through the cartridge.

FIG. 5M and FIG. 5N illustrate engagement between a bioreactor baseplate 549 of a docking station 515 and a cartridge 514 arranged therein. In variations, the bioreactor baseplate 549 may be formed integrally with the lift (described previously), which enables the cartridge 514 to be lifted from a docking station drawer and brought into contact with a top internal surface of the docking station 515. The bioreactor baseplate 549, in variations, is an aspect of a docking station bioreactor module configured to interact with a cartridge bioreactor module of the cartridge 514. To this end, the docking station bioreactor module interacts with bioreactors of the cartridge bioreactor module via motors within the docking station bioreactor module to, for instance, drive impellers within the bioreactors to agitate fluids and suspend liquids therein. Moreover, via the bioreactor baseplate 549, pre-mixed gases may be provided from the docking station bioreactor module to the bioreactors of the cartridge bioreactor module to support cell growth.

FIG. 5O illustrates aspects of a docking station bioreactor module of the present disclosure. As shown in FIG. 5O, the docking station bioreactor module may comprise a camera 548, a viewing window 546, and a mirror 547. The camera 548 is able to monitor fluid level within bioreactors of the cartridge bioreactor module through the viewing window 546. In variations, the camera 548 is positioned such that a mirror 547 is deployed to allow viewing of a corresponding cartridge bioreactor module through the viewing window 546.

Similarly, FIG. 5P illustrates aspects of a docking station waste module configured to evaluate waste volume level within the cartridge. To this end, the docking station waste module may comprise a camera 577, a viewing window 578, and, optionally, a mirror (not shown). The camera 577 is able to monitor waste fluid level within the cartridge through the viewing window 578. In variations, the camera 548 is positioned such that the mirror is deployed to allow viewing of waste levels within the cartridge through the viewing window 546. In variations, the cartridge may be directly plumbed to a waste tank, which may be emptied, cleaned and/or replaced with each process.

FIG. 5Q through FIG. 5S illustrate a sterile liquid transfer instrument 543 coupled to a top surface of a docking station 515. The sterile liquid transfer instrument 543 may comprise a first robot 544 and a second robot 545 which provide, when actuated with a gantry 541, at least two axes of movement relative to the docking station 515. As before, a sterile liquid transfer device 542 may be coupled to the gantry 541 of the sterile liquid transfer instrument 543 in order to be positioned such that engagement with the cartridge 514 is possible. To this end, as shown in FIG. 5S, engagement may include supplying or retracting fluids from the cartridge 514 via a sterile liquid transfer port 566 of the sterile liquid transfer device 542, wherein such supplying and retracting is performed by a motor 537 in combination with pump rollers 534, thereby providing peristaltic fluid flow into and out of the sterile fluid transfer device 542.

FIG. 5T through FIG. 5V illustrates aspects of a docking station CCE module. As shown in FIG. 5T, the docking station CCE module may be arranged in a rear (i.e. back) wall of a docking station 515 and may comprise at least a magnet 558 actuated by a motor 572 and configured to magnetically couple to a cartridge CCE module to rotate a CCE compartment thereof. As the CCE compartment rotates, the CCE compartment may be imaged by imaging components of the docking station CCE module, which include camera lights 576, a viewing window 575, and a camera 573.

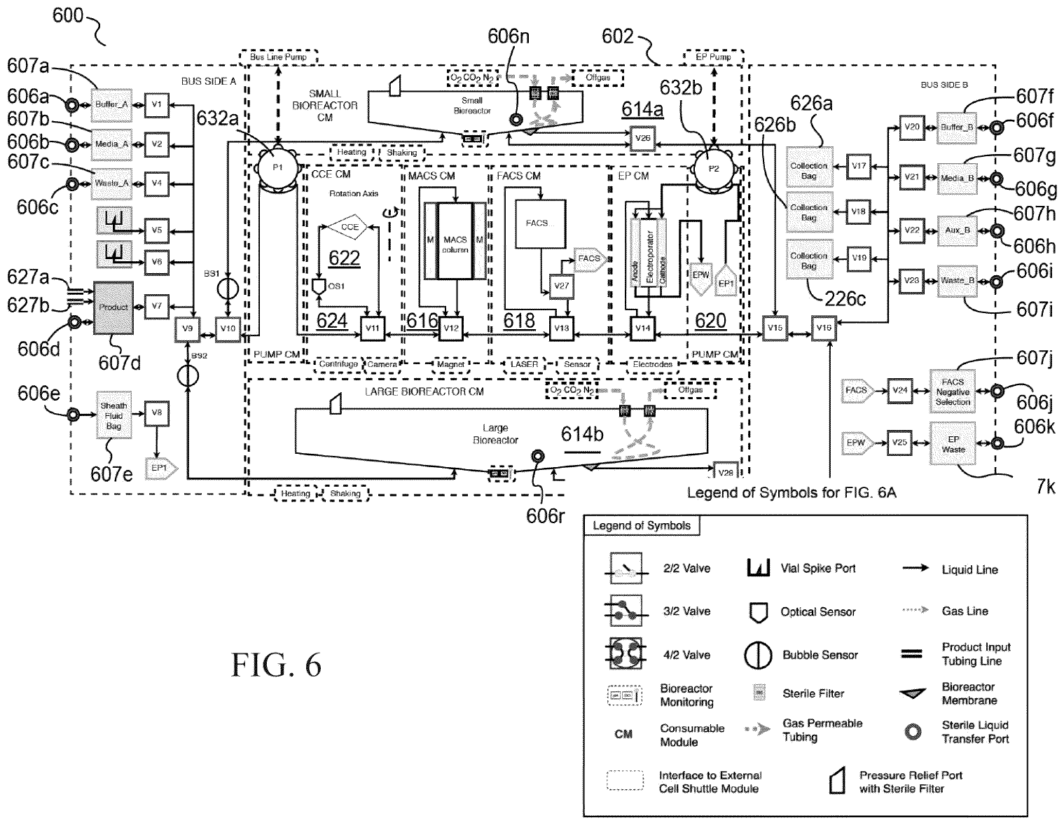

FIG. 6 is a schematic illustration of a cartridge 600 that may be a consumable produced from materials at a cost that make recycling or limited use practical. The cartridge 600 may comprise a liquid transfer bus 624 fluidically coupled to a small bioreactor module 614a, a large bioreactor module 614b, a cell selection module 616, a cell sorting module 618, an electroporation module 620, and a counterflow centrifugal elutriation (CCE) module 622. In some variations, the cell selection module 616 may be a magnetic-activated cell selection (MACS) module. The cell sorting module 618 may comprise a fluorescence activated cell sorting (FACS) module. The cartridge 60 may comprise a housing 602 that renders the cartridge self-contained, and optionally protects the contents from contamination. Sterile liquid transfer ports (SLTPs), 606a-606k may be fluidically coupled to reservoirs 607a-607k, and each independently be a flexible bag or a rigid container. In some variations, flexible bags may be configured to hold large volumes and to permit transfer of fluid without replacing transferred fluid with liquid or gas to maintain the pressure in the reservoir, as the bag may collapse when fluid is transferred out and expand when fluid is transferred in.

In some variations, the liquid transfer bus 624 may comprise valves V1 to V28 and corresponding tubing that fluidically links the valves to one another and to each of the modules. Valves shown coupled to four fluidic lines are 4/2 (4 port 2 position) valves and valves shown coupled to three fluidic lines are 3/2 (3 port 2 position) valves. Internal flow paths of the valves are indicated in the legend. The cartridge may further comprise a first pump 632a and a second pump 632b, each of which expose tubing on the exterior of the housing 602 to permit each pump to interface with pump actuators (e.g., rotors) in some docking station modules in the CPS. The liquid transfer bus 624 may be fluidically coupled to reservoir 607d and a product bag which is fluidically coupled to SLTP 606d and to product input tubing lines 627a-627b. An operator may input a cell product into reservoir 607d by connecting product input tubing line 627a or 627b to an external source of cells (e.g., a bag of cells collected from a donor). SLTP 606d may be configured to enable a system according to the disclosure (e.g., CPS) to add fluid to the reservoir 607d in an automated fashion. For example, one or more fluid-carrying containers such as reservoirs 607a-607k, bags, etc. may receive fluid via an SLTP. Additionally or alternatively, the SLTP may be configured to enable periodic sampling of one or more of the fluid-carrying containers. The cartridge may further comprise collection bags 626a-626c, fluidically coupled to the liquid transfer bus 624 via valves V17-V19. The cartridge 600 may be configured to permit an operator to remove the collection bags 626a-626c after completion of cell processing by the system.

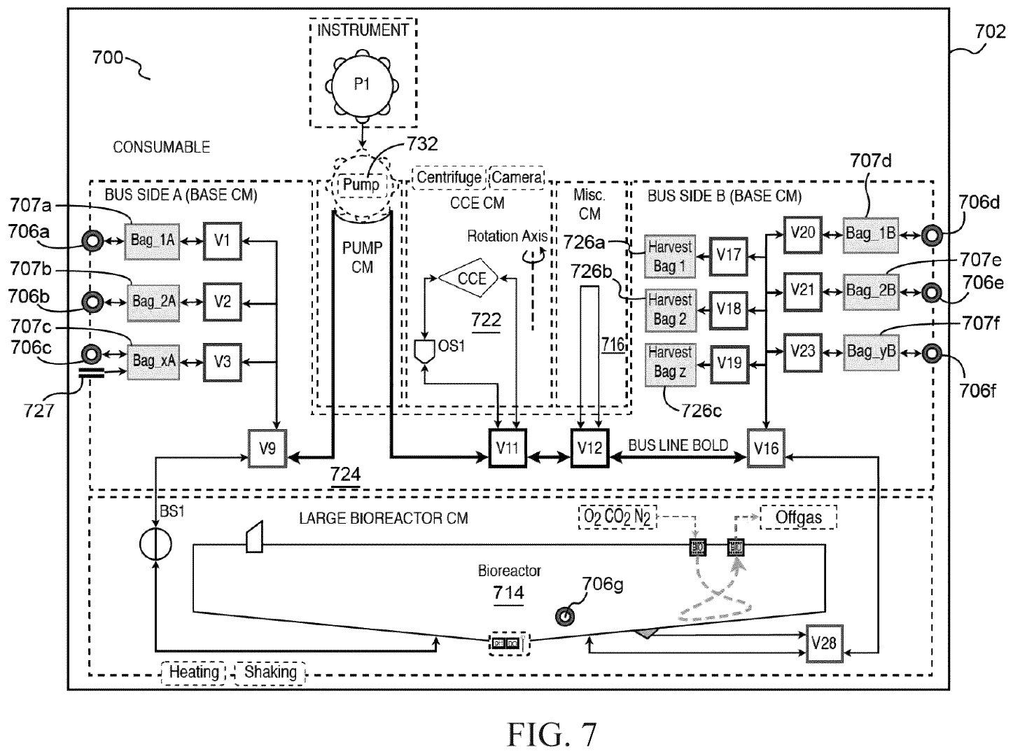

FIG. 7 is a schematic diagram of another variation of a cartridge 700. For example, cartridge 700 may comprise a reduced feature set compared to cartridge 600. The cartridge 700 may comprise a liquid transfer bus 724 fluidically coupled to a bioreactor module 714, a counterflow centrifugal elutriation (CCE) module 722, and at least one module 716 selected from a cell selection module, a cell sorting module, an electroporation module, a spinoculation module, or any other cell processing module. The cartridge 700 may comprise a housing 702 and sterile liquid transfer ports (SLTPs) 706a-706f (e.g., fluid connector) fluidically coupled to reservoirs 707a-707f, which may each independently be a flexible bag or a rigid container. SLTP 706g is fluidically coupled to the bioreactor module 714 to permit direct access by a system or an operator to the bioreactor. Reservoir 707c may be fluidically coupled to SLTP 706c and product input tubing line 727. In some variations, the liquid transfer bus 724 may comprise 14 valves V1-V3, V9, V11-V12, V17-V23 and V28 and tubing that fluidically couples the values to one another and/or each of the modules. The cartridge may further comprise collection bags 726a-726c fluidically coupled to the liquid transfer bus 724 via valves V17-V19. The cartridge may further comprise a pump 732 which exposes the tubing on the exterior of the housing 702 to permit each pump to interface with a pump actuator in the system (e.g., cell processing station).

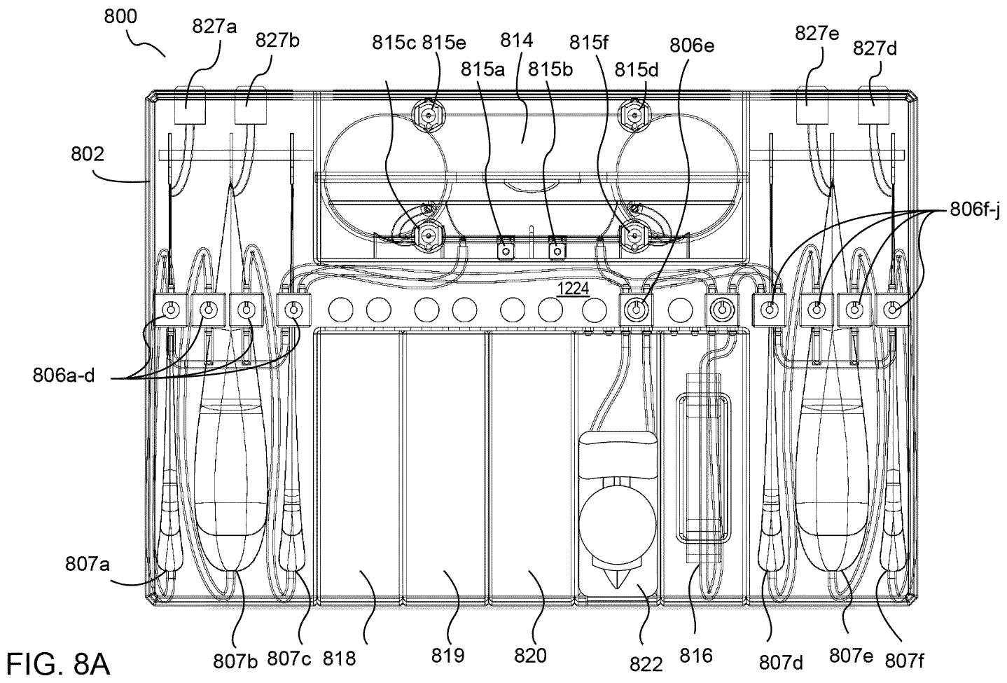

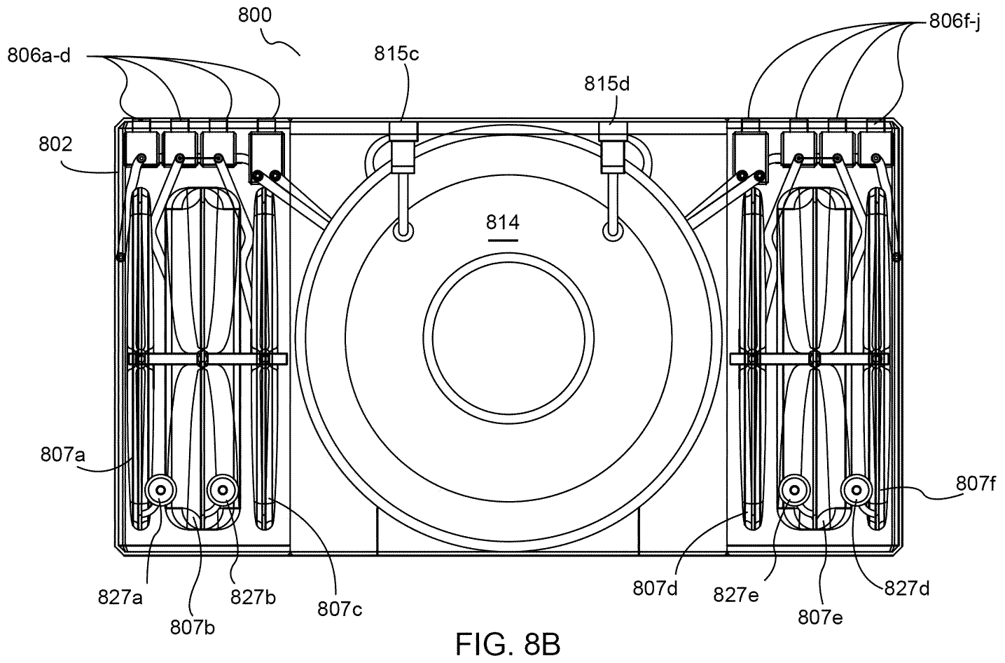

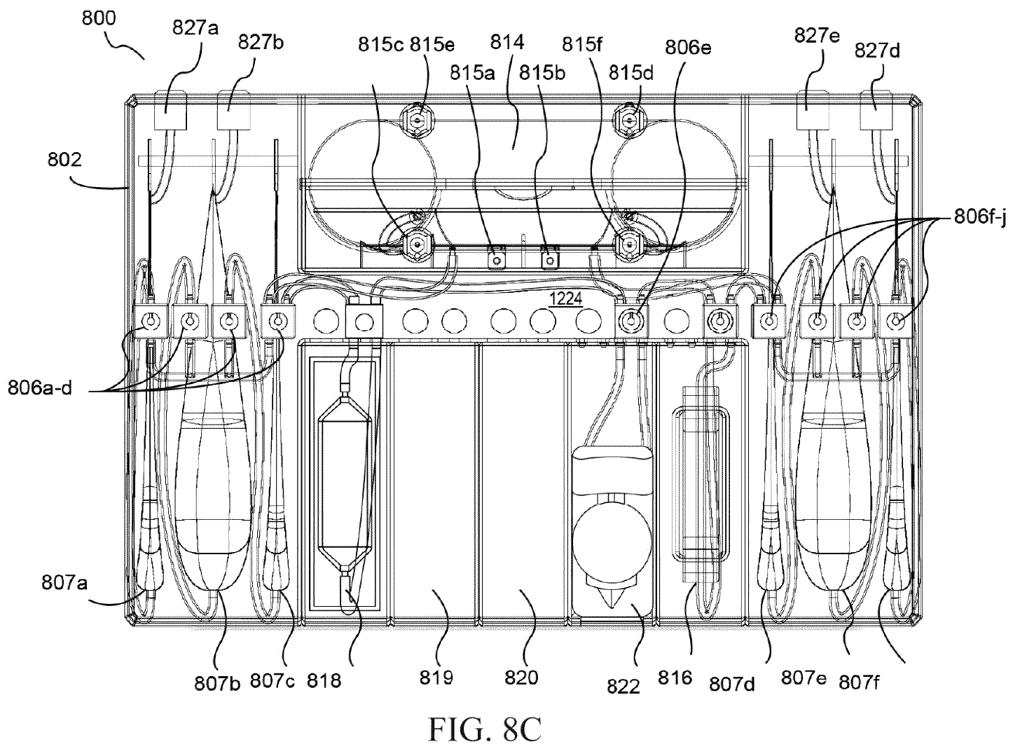

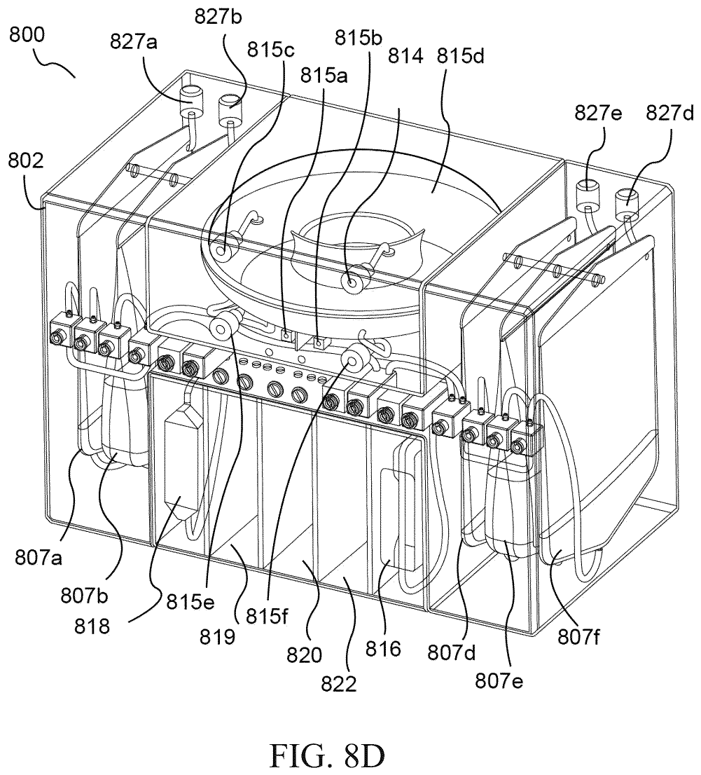

A side and top view of another variation of a cartridge is shown in respective FIGS. 8A and 8B. In some variations, a cartridge 800 may comprise a bioreactor 814, a pump 816, and a counterflow centrifugal elutriation (CCE) module 822. The cartridge 800 may comprise blanks 818, 819, and 820 configured to house additional module(s) such as a cell selection module, cell sorting module, an electroporation module, a small bioreactor module, a spinoculation module, and the like. In some variations, a blank may define an empty volume of the cartridge reserved to house a module at another time. In some variations, the cartridge 800 may comprise two or more additional bioreactors and/or reservoirs in blanks 818, 819, 820. Along the near surface of the cartridge 800 may be sterile liquid transfer ports 806a-806j fluidically connected to reservoirs 807a-807f. Reservoirs 807b and 807e may comprise fluid (e.g., buffer or media). Along the top surface are product input tubing lines 827a-827d, which may be fluidically connected to reservoirs 807a, 807b, 807e, and 807f, respectively. A liquid transfer bus 824 may fluidically connect the SLTPs, reservoirs, and product input tubing lines to the modules via tubing.

In some variations, the housing 802 may have external dimensions of about 225 mm×about 280 mm×385 mm, about 225 mm×about 295 mm×385 mm, and about 450 mm×about 300 mm×about 250 mm, including all values and sub-ranges in-between. In some variations, the cartridge 800 may be about 10%, about 20%, about 30% or more smaller in volume, including all ranges and sub-values in-between. In some variations, the cartridge 800 may be about 10%, about 20%, about 30%, about 50%, about 100%, about 200%, or more in volume, including all ranges and sub-values in-between. Of course, external dimensions of the cartridge 800 are dependent in part on internal dimensions of the docking station of the CPS, as the cartridge 800 must be sized for receipt within the docking station and engagement with corresponding docking station module interfaces arranged proximate internal walls of the docking station.

In some variations, a cartridge 800 as shown in the side view of FIG. 8C and perspective view of FIG. 8D may comprise a MACS module 818. For example, the bioreactor module 814 may comprise ports 815a-815f including a pH and DO sensors (ports 815a and 815b), a gas input line 815c, an output line 815d each having a sterile filter behind the connector, and a coolant input line 815e and output line 815f from the docking station bioreactor module interface when it interfaces with cartridge bioreactor module 814 (for heat exchange). For example, the gas input line 815c may be configured for gas transfer into a fluid (e.g., through headspace gas control or a gas-permeable membrane).

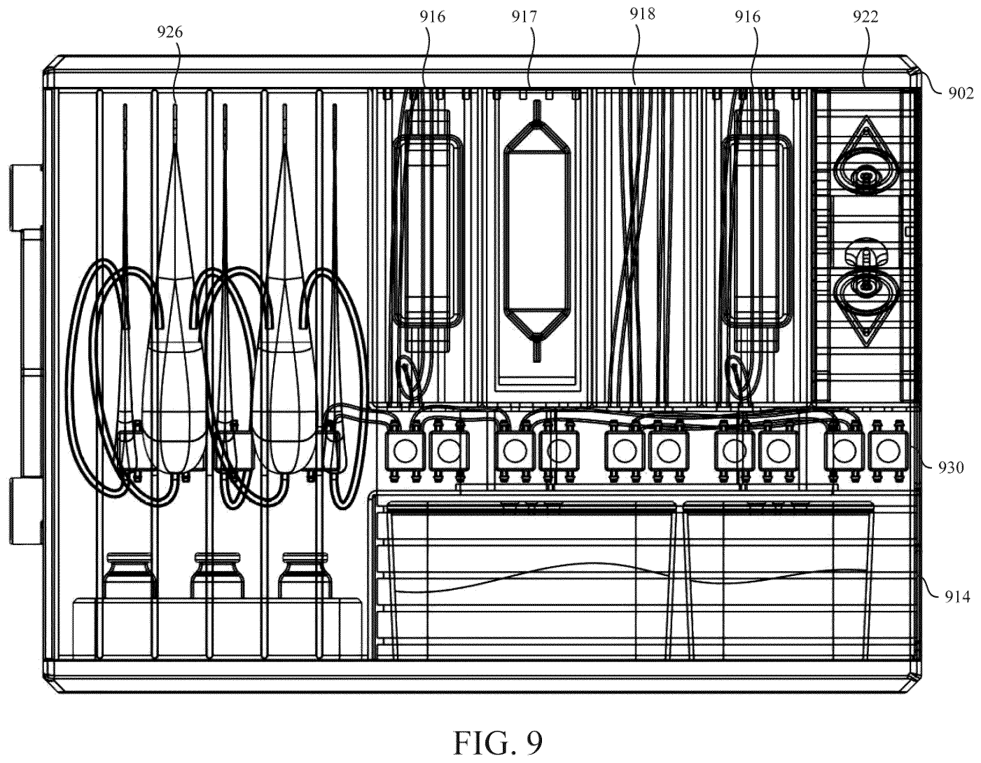

FIG. 9 shows a cross-sectional side view of a cartridge 900. In some variations, a cartridge 900 may comprise an enclosure (e.g., housing), a bioreactor 914, one or more pumps 916, valve 930, cell selection module 917, and a counterflow centrifugal elutriation (CCE) module 922. In some variations, the cell selection module 616 may be a magnetic-activated cell selection (MACS) module 917. The cartridge may further comprise collection bags 926. The cartridge 900 may optionally comprise blanks configured to house additional module(s) such as a cell selection module, a cell sorting module, an electroporation module 918, and the like. In some variations, the cartridge 900 may comprise one or more bioreactors and/or reservoirs in the blanks.

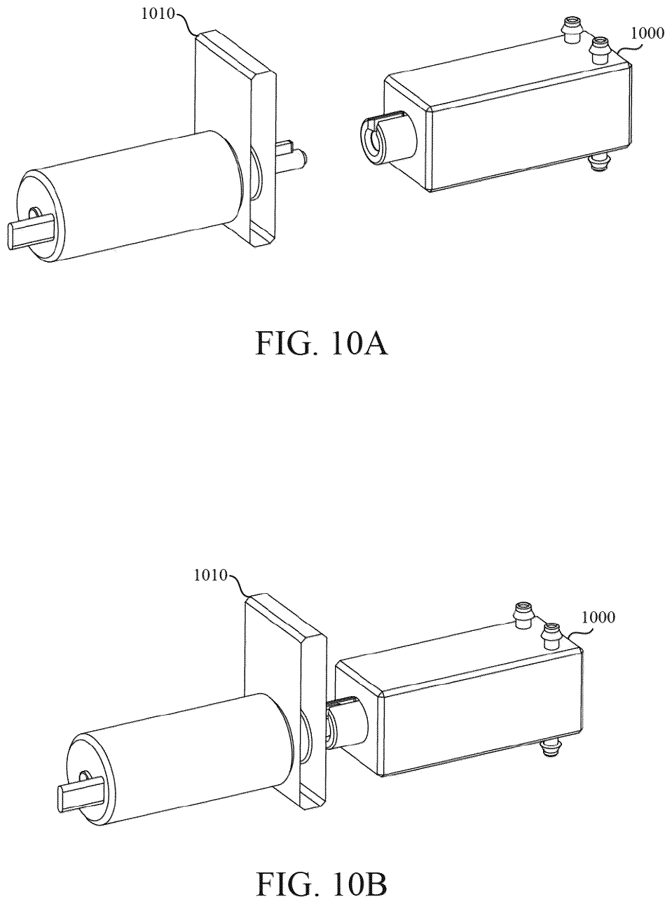

In some variations, a cartridge may comprise one or more valves. In some variations, the valve 1000 on the cartridge may be configured to receive an actuator 1010 provided by a docking station module (as shown in FIG. 10A) of the docking station of the CPS. As the cartridge is inserted into the docking station, the valve 1000 may be configured to dock with the actuator 1010 of the docking station module (as shown in FIG. 10B), such that rotation of the actuator 1010 may cause switching of the valve 1000 from one position to another position. In some variations, the valves may be constructed to pinch a section of soft tubing. The pinch valves may comprise a closed configuration, and an external actuator may be configured to interface with the pinch valve (e.g., utilizing a solenoid with linear motion) to open or close the valve. The valves themselves may be configured to be disposable whereas the actuators may be integrated into a docking station module configured to process cartridges repeatedly.

### Counterflow Centrifugal Elutriation

Counterflow centrifugal elutriation (CCE) is a technique used to separate cells based on characteristics such as size and/or density. Counterflow centrifugal elutriation combines centrifugation with counterflow elutriation where centrifugation corresponds to the process of sedimentation under the influence of a centrifugal force field and counterflow elutriation corresponds to the process of separation by washing. Separation takes place in a cone (e.g., bicone, funnel) shaped elutriation chamber. Particles (e.g., cells) conveyed in a fluid into the elutriation chamber are acted upon by two opposing forces; centrifugal force driving the fluid away from an axis of rotation; and fluid velocity driving the fluid towards the axis of rotation (e.g., counterflow). By varying the flow rate and the centrifugal force, the separation of particles (e.g., cells) may be achieved. For example, as described in more detail herein, particles may be separated based on properties such as size and density.

Counterflow centrifugal elutriation may perform multiple operations useful for cell therapy manufacturing workflows including, but not limited to, cell washing, cell concentration, media/buffer replacement, transduction, and separation of white blood cells from other blood components (e.g., platelets, and red blood cells). In some variations, a fluid source (e.g., apheresis bag) for a cell separation process may comprise a suspension of white blood cells, red blood cells, platelets, and plasma. In order to separate immune cells of interest, white blood cells may be isolated and subsequently magnetically tagged for magnetic separation. A white blood cell separation step may be performed in a CCE module to separate cells based on size and density, while magnetic separation may be performed in a MACS module. In some variations, a CCE module may be integrated into a cartridge to enable a cell processing and manufacturing system to separate cells based on one or more of a progression through a cell cycle (e.g., G₁/M phase cells being larger than G₀, S, or G2 phase cells) and cell type (e.g., white blood cells from red blood cells and/or platelets).

Generally, a rotor configured to spin may comprise an elutriation chamber (e.g., cone, bicone). A fluid comprising a suspension of cells may be pumped under continuous flow into the rotor. As cells are introduced into the cone (e.g., bicone), the cells migrate according to their sedimentation rates to positions in the gradient where the effects of the two forces upon them are balanced. Smaller cells having low sedimentation rates (e.g., platelets) may be quickly washed toward the axis of rotation with increased flow velocity. Such smaller cells may be output (e.g., washed out) of the cone. Relatively larger (or denser) cells (e.g., red blood cells) flow through the cone relatively more slowly and reach equilibrium at an elutriation boundary where the centrifugal force and the drag force are in balance, and the fluid velocity is relatively low because the cone has widened. The largest or densest cells (e.g., white blood cells) remain near the inlet to the chamber where centrifugal force and fluid velocity are high. By increasing the flow rate in gradual steps, successive fractions of increasingly large or dense cells (e.g., platelets→red blood cells→white blood cells) may be output from the rotor. Continued incremental increases in fluid flow rate will eventually elutriate all cells from the cone.

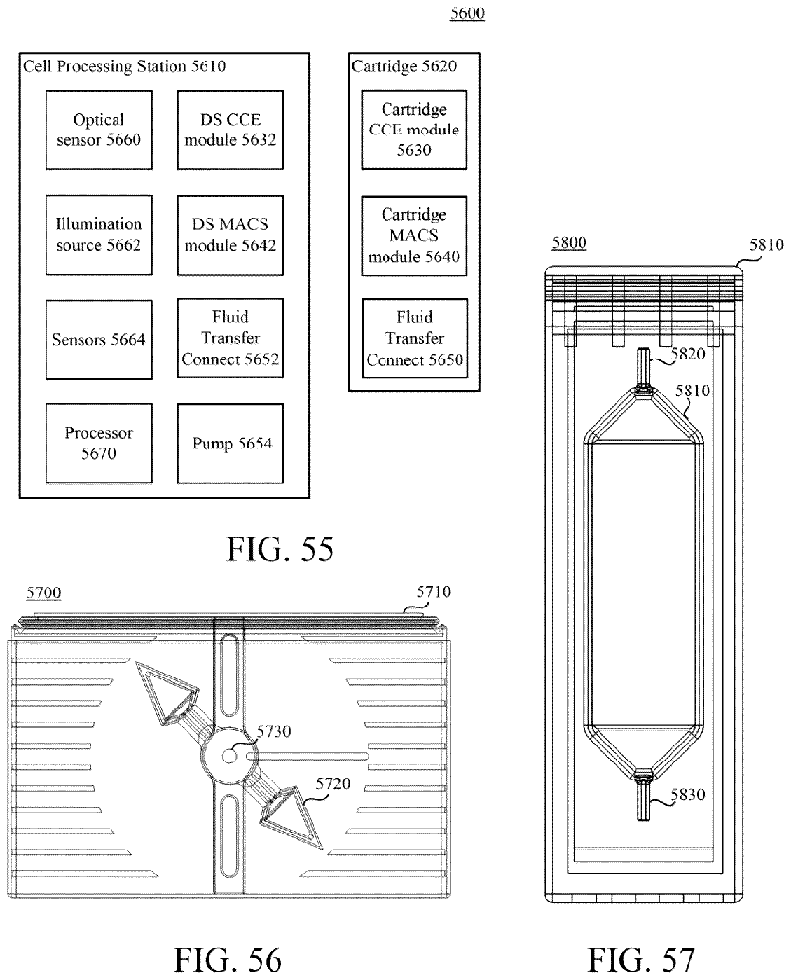

Returning now to the Drawings, FIG. 55 is a block diagram of a cell separation system 5600 comprising a cell processing station (CPS) 5610 and a cartridge 5620. In some variations, the CPS 5610 may comprise one or more of a docking station (DS) counterflow centrifugal elutriation (CCE) module 5632 (e.g., first magnet), a DS magnetic-activated cell selection (MACS) module 5642 (e.g., magnet array, second magnet), a fluid transfer connect 5652, which may be a sterile liquid transfer port or a connection to a liquid transfer bus of the cartridge, a pump 5654, an imaging system comprising an optical sensor 5660 and an illumination source 5662, a sensor 5664, and a processor 5670. In some variations, the cartridge 5620 may comprise one or more of a cartridge CCE module 5630 (e.g., rotor), a cartridge MACS module 5640 (e.g., flow cell), and a fluid transfer connect 5650, which may be a sterile liquid transfer port or the liquid transfer bus of the cartridge, and which may correspond to the fluid transfer connect 5652 of the CPS 5610. For example, a cartridge for cell processing may comprise a liquid transfer bus and a plurality of modules, each module fluidically linked to the liquid transfer bus.

In some variations, the imaging system (e.g., optical sensor 5660, illumination source 5622) may be configured to generate image data corresponding to one or more of the cartridge CCE module 5630 and cartridge MACS module 5640. For example, image data of fluid flow through a rotor of a cartridge CCE module 5630 may be analyzed and used to control a flow rate of fluid and/or rotation rate of the rotor, as described in more detail herein. In some variations, the optical sensor 5660 may be a CMOS/CCD sensor having, for example a resolution of about 100 μm, a working distance of between about 40 mm and about 100 mm, and a focal length of less than about 8 mm. The optical sensor 5660 may be configured to operate synchronously with the illumination source 5662. In some variations, the optical sensor 5660 may comprise one or more of a colorimeter, turbidity sensor, and optical density sensor. In some variations, the illumination source 5662 may operate as a strobe light configured to output light pulses synchronized to a rotation rate of a rotor of the CCE module 5630.

In some variations, the sensor 5664 may comprise one or more of an optical density sensor configured to measure an intensity of fluid, a leak detector configured to detect moisture and/or leaks, an inertial sensor configured to measure vibration, a pressure sensor configured to measure pressure in a fluidic line (e.g., photoelectric sensor), a bubble sensor configured to detect the presence of a bubble in a fluid conduit, colorimetric sensor, vibration sensor, and the like.

In some variations, the fluid transfer connect 5652 may comprise one or more valves, configured to control fluid flow between the CPS 5610 and the cartridge 5620. The processor 5670 may correspond to the controller (e.g., processor and memory) described in more detail herein. The processor 5670 may be configured to control one or more of the DS CCE module 5632, the DS MACS module 5642, the pump 5654, fluid transfer connect 5652 (e.g., valves), the optical sensor 5660, the illumination source 5662, and the sensors 5664.

In some variations, a CPMS 5600 for cell processing may comprise a cartridge 5600 comprising a rotor of a CCE module 5630 configured for counterflow centrifugal elutriation of cells in a fluid. A first magnet of a docking station CCE module 5632 may be configured to magnetically rotate the rotor and separate the cells from the fluid in the rotor. The cartridge may further comprise a flow cell of a MACS module 5640 coupled to the rotor and configured to receive the cells from the rotor. A second magnet of a docking station MACS module 5642 may be configured to magnetically separate the cells in the flow cell.

In some variations, an illumination source 5662 may be configured to illuminate the cells. An optical sensor 5660 may be configured to generate image data corresponding to the cells. In some variations, the CPMS 5600 may comprise one or more of an oxygen depletion sensor, leak sensor, inertial sensor, pressure sensor, and bubble sensor. In some variations, the CPMS 5600 may comprise one or more valves and pumps.

FIG. 56 is a cross-sectional side view of a counterflow centrifugal elutriation (CCE) module 5700 comprising a housing 5710 (e.g., enclosure), a rotor 5720 configured to rotate within and relative to the housing 5710, and one or more fluid ports 5730 (e.g., fluid inlet, fluid outlet).

FIG. 57 is a cross-sectional side view of a magnetic-activated cell selection (MACS) module comprising a housing 5810 (e.g., enclosure), a first fluid port 5820 (e.g., fluid inlet), a second fluid port 5830 (e.g., fluid outlet), and a flow cell 5810 coupled in between the first fluid port 5820 and the second fluid port 5830. As described in more detail herein, the flow cell 5810 may comprise a cavity (e.g., chamber) comprising one or more channels (e.g., linear channels, laminar fluid flow channel). In some variations, the cavity of the flow cell 5810 may be substantially empty. For example, the flow cell 5810 may be absent a mesh, beads, tortuous channels, and the like. In some variations, the flow cell 5810 may have a longitudinal axis aligned perpendicular to ground. That is, the flow cell 5810 may be oriented vertically where the first fluid port 5820 is disposed at a higher elevation than the second fluid port 5830 such that gravity may aid fluid flow through the flow cell 5810.

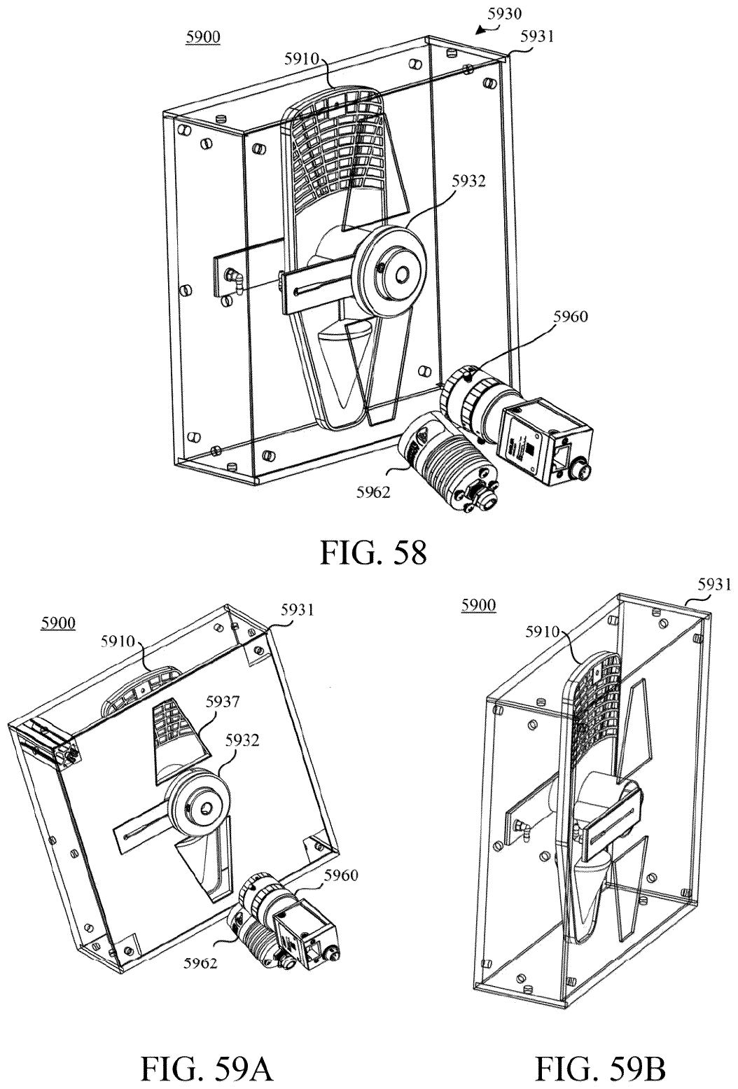

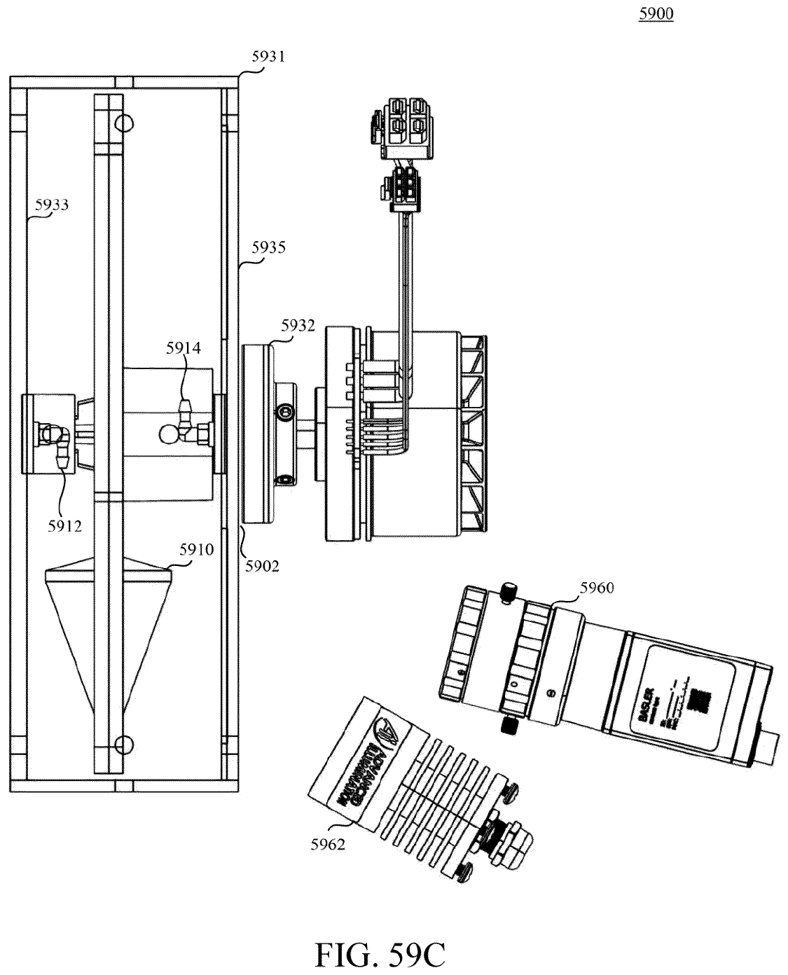

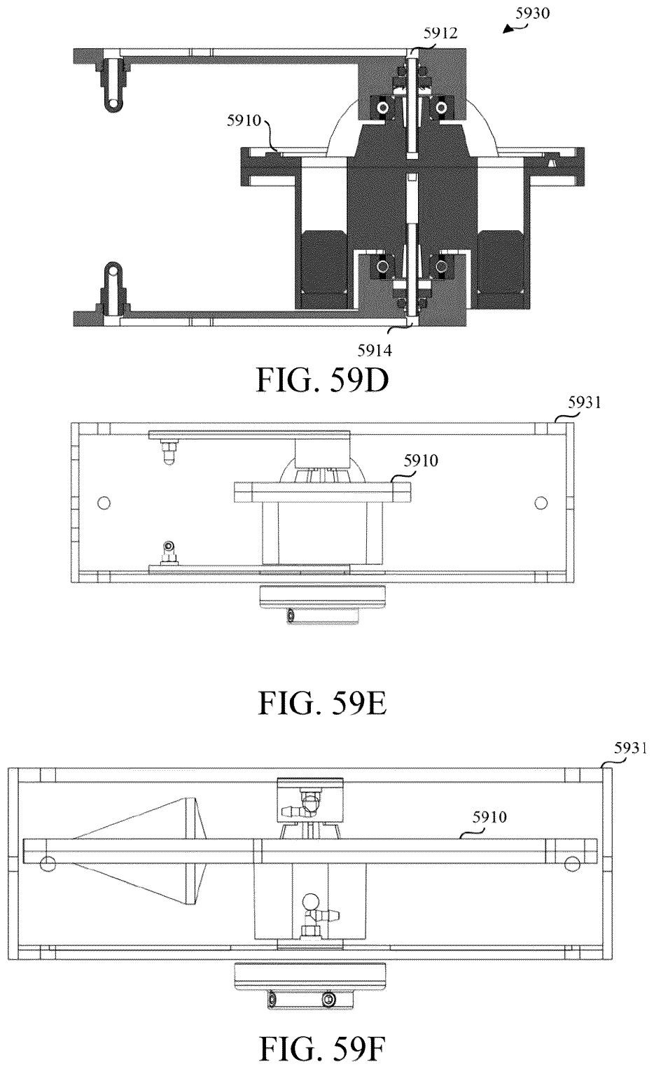

FIG. 58 is a semi-transparent perspective view of a CPMS 5900 for cell processing comprising a CCE system. FIG. 59A provides a relatively opaque perspective view of the CPMS 5900 comprising the CCE system. As shown in FIGS. 58 and 59A, the CCE system comprises a cartridge CCE module 5930 including a housing 5931 and a rotor 5910, a docking station CCE module 5932 including an optical sensor 5960 and an illumination source 5962. In some variations, the docking station CCE module 5932 may comprise a magnet configured to magnetically rotate the rotor 5910 within the cartridge CCE module 5930. One or more portions of the housing 5931 and rotor 5910 may be optically transparent to facilitate illumination by the illumination source 5962 and image data generation by the optical sensor 5960.

In some variations, the CPMS 5900 for cell processing may comprise a cartridge 5930 comprising a housing 5931 and a rotor 5910 therein configured to separate cells from a fluid. A corresponding docking station module 5932 comprising a magnet may be configured to interface with the cartridge 5930 to magnetically rotate the rotor 5910. The cartridge 5930 may be configured to move a cell product between a plurality of cartridge modules, docking station modules, and/or other instruments. In some variations, the housing 5931 may enclose the rotor 5910. In some variations, the housing 5931 may comprise one or more apertures 5937 configured to facilitate visualization (e.g., imaging) of the rotor 5910. FIGS. 58 and 59A depict a magnet 5932 in proximity, but not attached, to housing 5931. FIG. 59B is a perspective view of the rotor 5910 and housing 5931 without the magnet 5932, optical sensor 590, and illumination source 5962.

In some variations, the cartridge 5930 (e.g., housing 5931, 5910) may comprise a consumable component such as a disposable component, limited use component, single use component, and the like. In some variations, the magnet 5932 may comprise a durable component that may be re-used a plurality of times. In some variations, the magnet 5932 may be releasably coupled to the housing 5931. For example, the housing 5931 may be moved relative to the magnet 5932 to facilitate magnetic coupling between the magnet 5932 and a plurality of cartridges 5930. Additionally or alternatively, the magnet 5932 may be configured to be moved relative to the housing 5931.

FIG. 59C is a side cross-sectional view of a cartridge CCE module 5930. In some variations, the housing 5931 of the rotor 5910 may comprise a first side 5933 comprising the first fluid port 5912 (e.g., first fluid conduit) and a second side 5935 comprising the second fluid port 5914 where the second side 5935 is opposite the first side 5933. The rotor 5910 (including a cone or bicone as described in more detail herein) may be coupled between the first fluid port 5912 and the second fluid port 5914. In some variations, the cartridge CCE module 5930 may comprise an air gap 5902 between the housing 5931 and a magnet 5932. That is, the cartridge 5930 and magnet 5932 may couple in a non-contact manner. Consequently, the cartridge need not mechanically couple to the magnet 5932 to perform counterflow centrifugal elutriation. Therefore, the rotor 5910 may have a low alignment sensitivity with the magnet 5932, as well as low vibration between the rotor 5910 and the magnet 5932. Furthermore, the space between the rotor 5910 and magnet 5932 enables the second fluid port 5914 to extend toward the second side 5935 of the housing 5931, thus allowing for fluid to flow on each side of the rotor 5910.

In some variations, counterflow centrifugal elutriation may be performed by the CPMS 5900 by moving a magnet 5932 towards a rotor 5910 (or vice versa). The rotor may define a rotational axis (e.g., coaxial with the first fluid port 5912 and the second fluid port 5914). Fluid may flow through the rotor via the first fluid port 5912 and the second fluid port 5914. The magnet 5932 may magnetically rotate the rotor about the rotational axis while flowing the fluid through the rotor 5910. The rotor may move away from the magnet. For example, moving the rotor 5910 may include advancing and withdrawing the rotor 5910 relative to the magnet 5932.

In some variations, fluid may flow through first fluid port 5912 along the first side 5933 of the rotor 5910 and into the rotor 5910. After counterflow centrifugal elutriation through the rotor 5910, the fluid may flow out of the rotor 5910 through second fluid port 5914 along the second side 5935 of the rotor 5910.

In some variations, counterflow centrifugal elutriation may be visualized by optical sensor 5960 and illumination source 5962 in order to monitor and modify cell separation in real-time based on predetermined criteria in a closed loop manner in order to maximize elutriation efficiency. In some variations, an optical sensor 5960 may be configured to image any portion of the rotor through which fluid flows (e.g., first fluid conduit, second fluid conduit, third fluid conduit, first bicone, second bicone). For example, image data of one or more of the fluid and the cells in the rotor 5910 may be generated using the optical sensor 5960. In some variations, one or more of the fluid and the cells may be illuminated using the illumination source 5962. For example, an output of a cone may be imaged by an optical sensor to identify non-target cells being elutriated.

In some variations, one or more of a rotation rate of the rotor and a flow rate of the fluid may be selected based at least in part on the image data. For example, the rotor may comprise a rotation rate of up to 6,000 RPM. For example, the fluid may comprise a flow rate of up to about 150 ml/min while rotating the rotor. In some variations, the rotor may be moved towards the illumination source 5962 and the optical sensor 5960. Additionally or alternatively, the rotor 5910 may be moved away from the illumination source 5962 and the optical sensor 5960.

FIG. 59D is a side cross-sectional view of a rotor 5910 including a first fluid port 5912 (e.g., fluid conduit, inlet) and a second fluid port 5914 (e.g., fluid conduit, outlet). In some variations, the first fluid port 5912 and the second fluid port 5914 may extend in parallel with each other and/or a rotational axis of the rotor 5910. In some variations, the first fluid port 5912 and the second fluid port 5914 may be disposed on opposite sides of the rotor 5910, which may simplify fluid routing, cartridge design, and also reduce manufacturing costs. For example, the fluidic seals may be simplified since they contain only a single lumen each. Conventionally, a complicated fluid flow path (including inlet and outlet) is formed on a first side of a rotor due to a fixed mechanical coupling of a drive motor to a second side of the rotor. FIGS. 59E and 59F are cross-sectional side views of a rotor 5910 disposed within housing 5931.

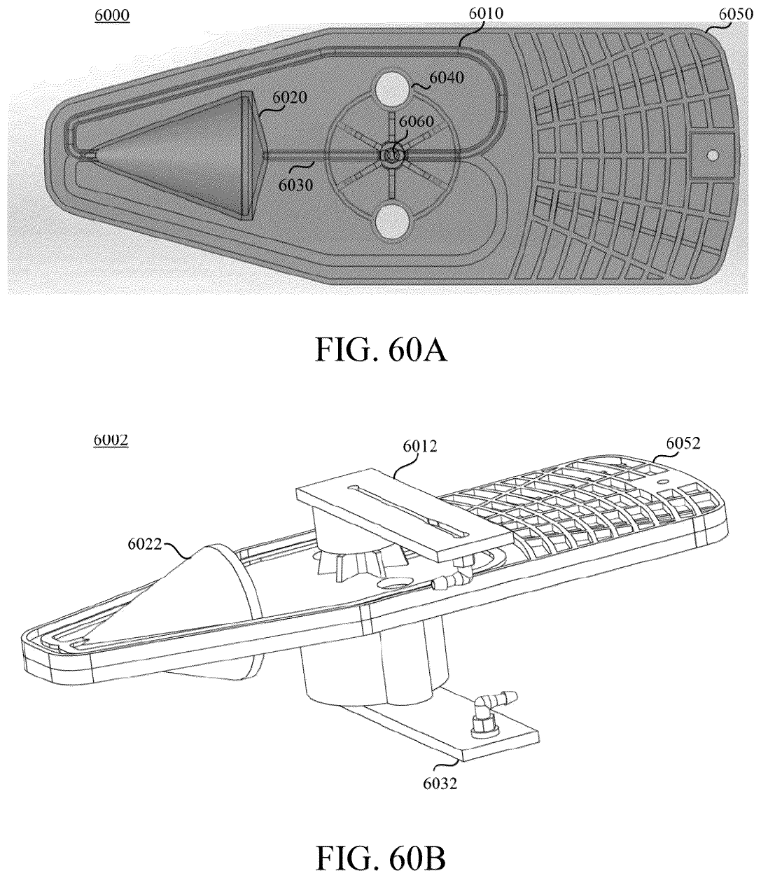

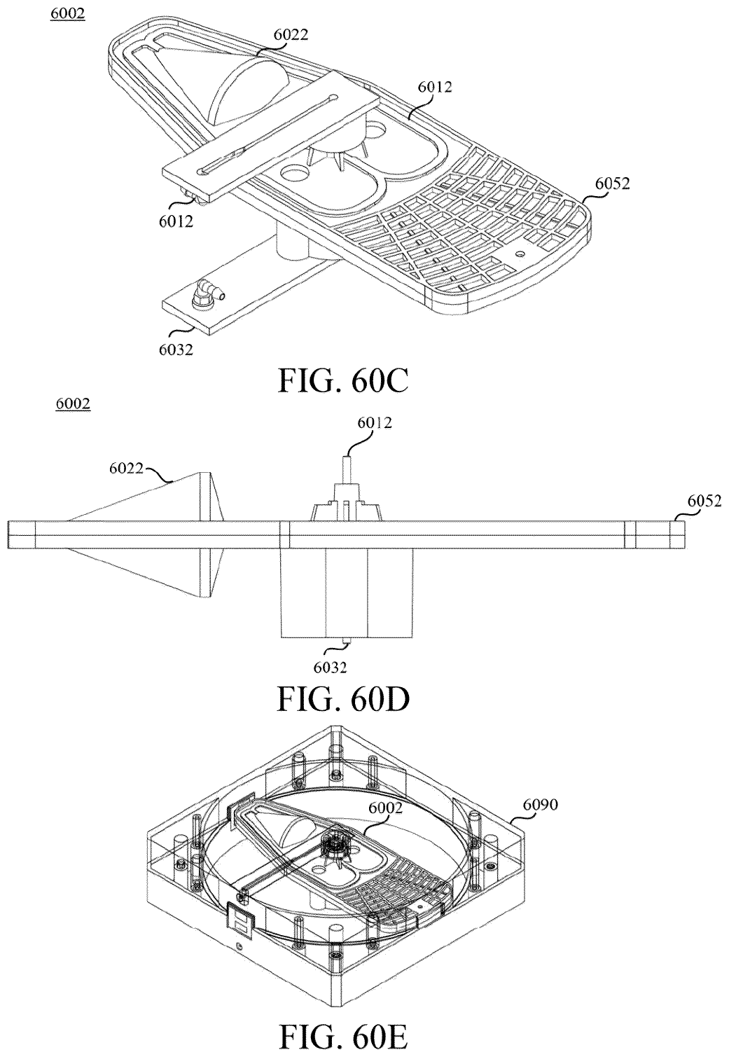

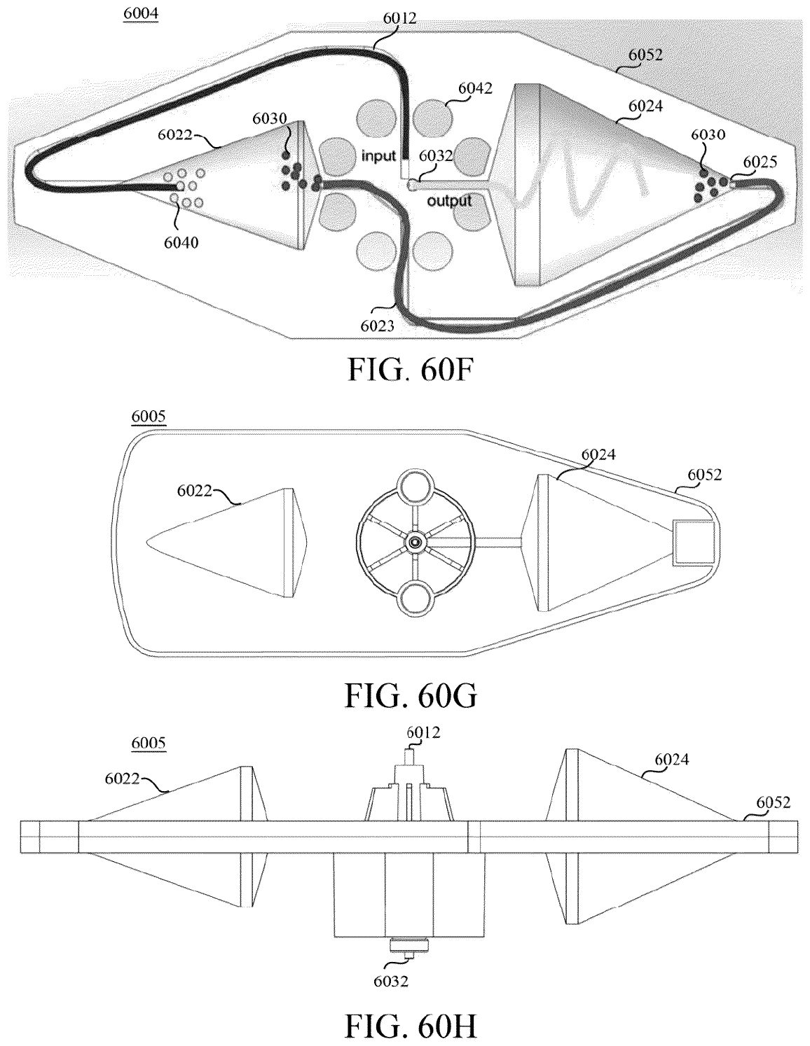

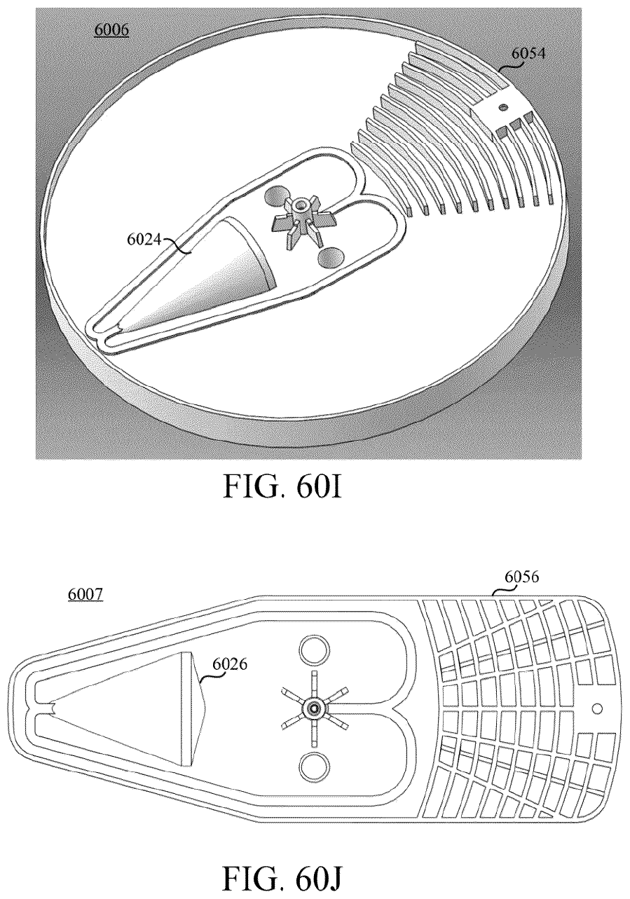

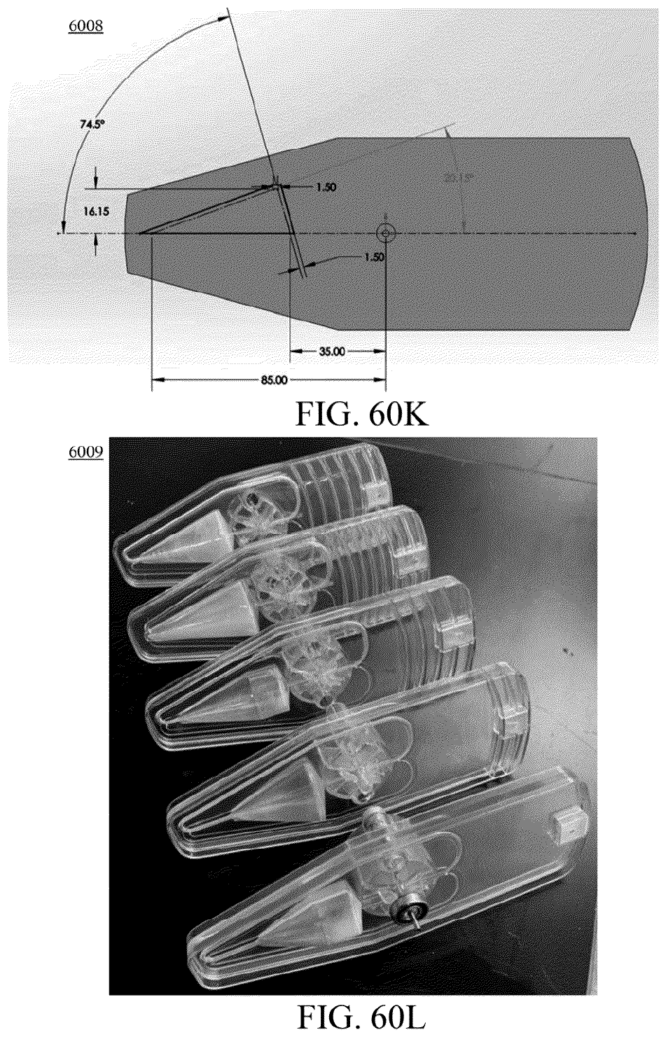

FIG. 60A is a plan view of a rotor 6000 that may be used with any of the CCE systems, CCE modules, cartridges, housings, combinations thereof, and the like described herein. The rotor 6000 may comprise a first fluid conduit 6010, a cone 6020 (e.g., bicone), a second fluid conduit 6030, a magnetic portion 6040 (e.g., magnet), and housing 6050. Fluid may flow sequentially through the first fluid conduit 6010, the cone 6020, and the second fluid conduit 6030. In some variations, the magnetic portion 6040 may comprise one or more magnets. In some variations, the rotor 6000 may define a rotation axis 6060. In some variations, at least a portion of the first fluid conduit 6010 and at least a portion of the second fluid conduit 6030 may extend parallel to the rotation axis (e.g., into and out of the page with respect to FIG. 60A). In some variations, at least a portion of the first fluid conduit 6010 and at least a portion of the second fluid conduit 6030 may be co-axial.

In some variations, the cone 6020 may comprise a bicone having a first cone including a first base and a second cone including a second base such that the first base faces the second base. In some variations, a bicone may comprise a cylinder (or some other shape) between and/or in fluid communication with the first cone and the second cone. For example, one or more cones of a rotor may comprise a generally stepped shape. For example, one or more cones may comprise stacked circular steps. In some variations, a cone of a rotor may comprise a single cone.

In some variations, at least a portion of the rotor may be optically transparent to facilitate visualization and/or imaging of the rotor 6000 and/or fluid (e.g., cells) in the rotor 6000. For example, the cone 6020 may be transparent, as well as portions of the first fluid conduit 6010 and the second fluid conduit 6030.

In some variations, the cone may comprise a volume of between about 10 ml and about 40 ml. In some variations, the cone may comprise a cone angle of between about 40 degrees and about 60 degrees.

In some variations, a cone may comprise a first cone (e.g., distal cone) and a second cone (e.g., proximal cone) where the first cone is larger than the second cone. In some variations, a first cone length may be between about 60 mm and about 90 mm. In some variations, a proximal cone length may be between about 15 mm and about 40 mm. In some variations, a cone diameter (e.g., maximum diameter of the cone) may be between about 15 mm and about 40 mm.

In some variations, the rotor 6000 may comprise an asymmetric shape. In some variations, a first portion (e.g., first end) of the rotor 6000 may comprise the cone 6020 and a second portion (e.g., second end) may comprise a paddle shape.

In some variations, the cone may comprise a length of at least about 4 cm (e.g., between about 9 cm and about 12 cm), a cone diameter of about 5 cm or less (e.g., between about 3 cm and about 5 cm), a fluid flow rate of up to about 100 ml/min (e.g., between about 60 ml/min and about 100 ml/min), and a rotation rate of less than about 3000 RPM. The shape of the first cone and the second cone may be generally linear (as opposed to convex or concave).

FIGS. 60B and 60C are perspective views, and FIG. 60D is a side view of a rotor 6002 comprising a first fluid conduit 6012, a cone 6022, a second fluid conduit 6032, and a housing 6052. FIG. 60E is a perspective view of the rotor 6002 disposed in a housing 6090.

FIG. 60F is a plan view of a rotor 6004 having two cones (e.g., two bicones) configured to elutriate cells (e.g., red blood cells, leukapheresis product) in a second cone in order to recirculate a buffer for reuse. The rotor 6004 may comprise a housing 6052, a first fluid conduit 6012, a first cone 6022 coupled to the first fluid conduit 6012, a second fluid conduit 6023 coupled to the first cone 6022, and a second cone 6024 coupled to the second conduit 6023, and a third fluid conduit 6032 coupled to the second cone 6024. The first cone 6022 may comprise a first volume, and the second cone 6024 may comprise a second volume larger than the first volume. In some variations, a ratio of a second volume to a first volume may be between about 2:1 to about 5:1. Fluid may flow sequentially through the first fluid conduit 6012, the first cone 6022, the second fluid conduit 6023, the second cone 6024, and the third fluid conduit 6032. In some variations, the rotor 6004 may comprise a magnetic portion 6042.

In some variations, the first cone 6022 may comprise a first bicone and the second cone 6024 may comprise a second bicone. In some variations, the first bicone may comprise a third cone including a first base and a fourth cone including a second base such that the first base faces the second base. In some variations, the second bicone may comprise a fifth cone including a third base and a sixth cone including a fourth base such that the third base faces the fourth base.

In some variations, a portion of the rotor 6004 may be optically transparent, such as first cone 6022, second cone 6024, and at least a portion of first fluid conduit 6012, second fluid conduit 6023, and third fluid conduit 6032. In some variations, the first fluid conduit 6012 may comprise an inlet and the third fluid conduit 6032 may comprise an outlet.

In some variations, cells may enter the first cone 6022 and red blood cells (RBCs) 6030 may be elutriated into the second cone 6024. Since the second cone 6024 is further out from an axis of rotation (center of housing 6052), the RBCs 6030 may be concentrated at an inlet 6025 of the second cone 6024 due to centrifugation. The larger volume of the second cone 6024 may further reduce the velocity of fluid (e.g., buffer), thereby reducing the force on RBCs 6030 within the second cone 6024. By recirculating the fluid (e.g., buffer), a higher concentration of RBCs may be elutriated with less fluid (e.g., buffer). In some variations, white blood cells 6040 may be harvested from the first cone 6022. An optical sensor may be configured to image the first cone 6022 to generate imaging data used to identify a boundary between the WBCs 6040 and RBCs 6030. In some variations, the recirculating fluid may be passed through a filter to remove small particles (e.g., platelets) with less fluid (e.g., buffer).

FIG. 60G is a plan view and FIG. 60H is a side view of a rotor 6005 having two cones (e.g., two bicones) configured to elutriate cells (e.g., red blood cells) in a second cone. A rotor having two cones may facilitate recirculation of buffer for reuse. The rotor 6006 may comprise a housing 6052, a first fluid conduit 6012, a first cone 6022 coupled to the first fluid conduit 6012, a second cone 6024 coupled to the first cone 6022, and a fluid conduit 6032 (e.g., outlet) coupled to the second cone 6024.

FIG. 60I is a perspective view of a rotor 6006 comprising a cone 6024 and housing 6054. FIG. 60J is a perspective view of a rotor 6007 comprising a cone 6026 and housing 6056. FIG. 60K is a schematic plan view of rotor 6008 and corresponding dimensions. FIG. 60L is an image of a set of rotors having varying dimensions.

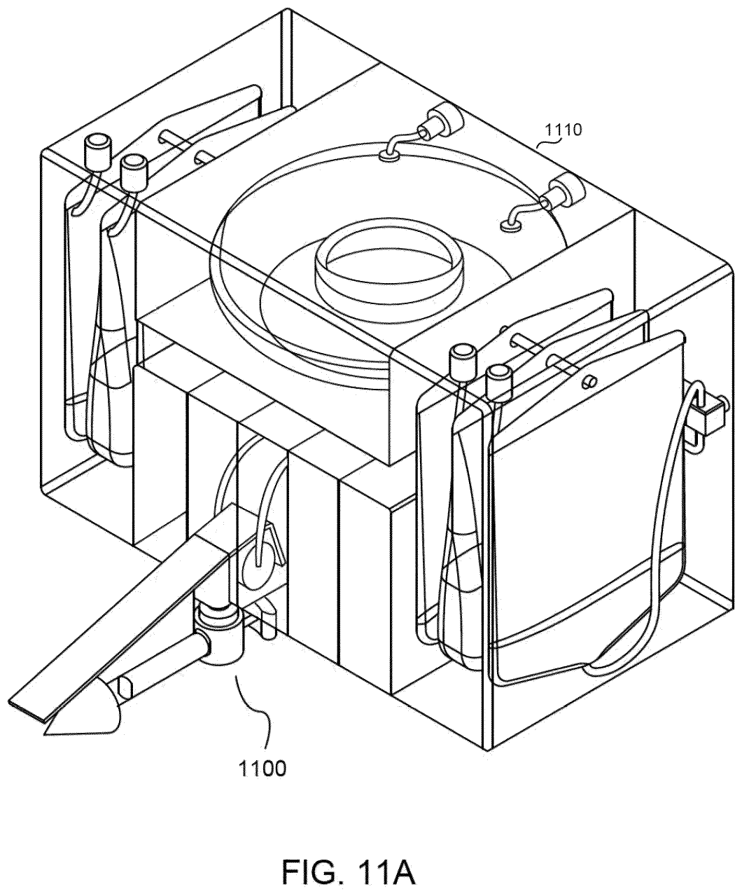

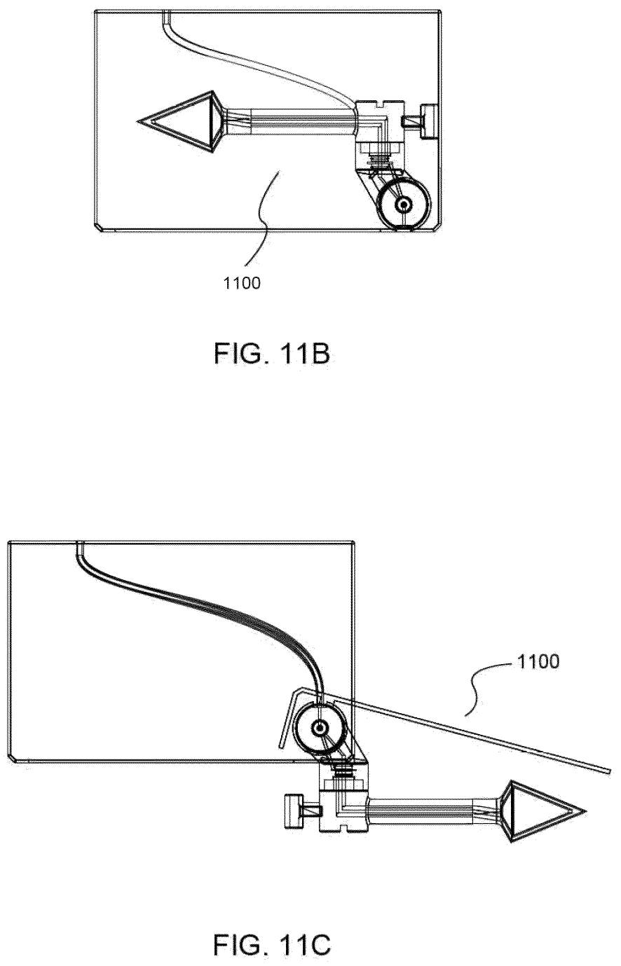

FIGS. 11A-11C depict another variation of the counterflow centrifugal elutriation (CCE) module 1100. FIG. 11A is a perspective view of a cartridge 1110 comprising a CCE module 1100 in an extended configuration configured to receive a docking station (DS) CCE module. FIGS. 11B and 11C are cross-sectional side views of a cartridge CCE module 1100 in respective retracted and extended configurations. In some variations, a cartridge CCE module may comprise a conical element having an internal surface and an external surface fixedly attached to a distal end of a linear member having an internal surface and an external surface. The proximal end of the linear member may be rotationally attached to a fulcrum in order to enable extension, retraction, and/or rotation of the linear member. For example, FIG. 11C depicts a linear member extended outside the housing of the cartridge and then rotated to generate a centrifugal force. A cell product may be conveyed between the internal surface and external surface of the linear member (optionally in tubing) to the conical element and fed into an opening at the distal end of the internal surface of the conical element, such that the flow of the cell product may run counter to the centrifugal force generated by rotation of the linear member. Cells in the cell product may be separated based on the ratio of their hydrodynamic cross section to their mass, due to the counterflow of the solution and sedimentation of cells subject to centrifugal force. The flow rate may then be increased and/or the rotation of the linear member may be decreased to permit cells to selectively return through the void in the interior surface of the linear member to the proximal end of the linear member. The selected cells may be directed into a tube that returns the selected cells to the cartridge. After an enrichment/washing step is performed, the linear member may be retracted into the housing to the retracted configuration as shown in FIG. 11B.

### Magnetic Cell Selection

Generally, the systems and methods described herein may select cells on the basis of magnetically labeled cells corresponding to cells having a predetermined antigen. For example, a cell suspension of interest may be immunologically labeled with magnetic particles (e.g., magnetic beads) configured to selectively bind to the surface of the cells of interest. The labeled cells may generate a large magnetic moment when the cell suspension is flowed through a flow cell. The flow cell may be disposed in proximity to a magnet array (e.g., permanent magnets, electromagnet) generating a magnetic field having a gradient across the flow cell to attract the labeled cells for separation, capture, recovery, and/or purification. The magnet array may be configured to generate non-uniform magnetic fields at the edges and the interfaces of the individual magnets so as to cover the full volume of the flow cell such that a magnetophoretic force equals a drag force exerted by the fluid flowing through the flow cell.

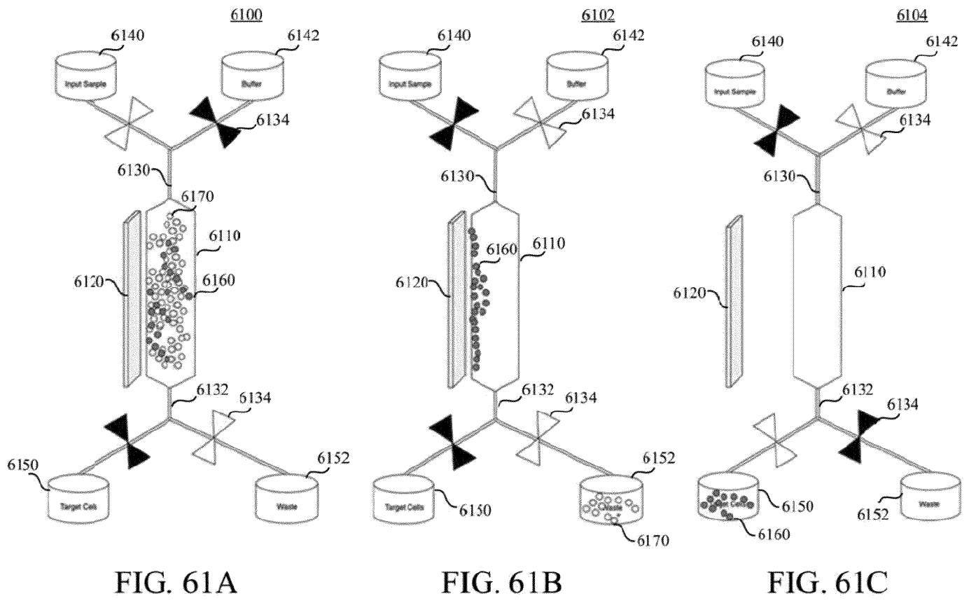

FIG. 61A-61C are schematic views of a magnetic cell separation (e.g., magnetic-activated cell selection) system and process. A magnetic cell separation system may comprise a flow cell 6110 comprising an inlet 6130 and an outlet 6132, a magnet array 6120, a first fluid source 6140 (e.g., input sample source), a second fluid source 6142 (e.g., buffer source), a third fluid source 6150 (e.g., target cell reservoir), a fourth fluid source 6152 (e.g., waste reservoir), and a set of valves 6134. As shown in step 6100, a set of cells 6160, 6170 may comprise labeled cells 6160 (e.g., magnetically labeled cells) and non-labeled cells 6170 may flow into the flow cell 6110. For example, a set of the cells 6160 may be labeled with a magnetic-activated cell selection (MACS) reagent. A MACS reagent may be incubated with the set of cells to label (e.g., attach, couple) the cells to the MACS reagent. As described in more detail herein, the magnet array 6120 may be disposed external to the flow cell 6110 such that the magnet array 6120 may be moveable relative to the flow cell 6110. For example, the magnet array 6120 may move away from the flow cell 6110 to facilitate flowing the set of cells 6160 out of the flow cell 6110. Conventional flow cells comprise tortuous paths including meshes and/or beads to capture cells. However, recovery of labeled cells from conventional flow cell configurations is difficult. By contrast, the flow cells 6110 described herein may lack tortuous paths such as beads, meshes, and the like, and therefore enable serial separations to be performed efficiently using either positive selection or negative selection. In some variations, the flow cells may comprise generally laminar channels as described in more detail herein.

At step 6102, the magnet array 6120 may magnetically attract the set of cells 6160 towards the magnet array 6120 for a predetermined dwell time and/or based on a measured quantity of magnetically separated cells. In some variations, the dwell time may be at least one minute (e.g., at least two minutes, at least three minutes, at least five minutes). The non-labeled cells 6170 are not magnetically attracted to the magnet array 6120 and may flow out of the outlet 6132 of the flow cell 6110 and into the fourth fluid source 6152. In some variations, the fluid (e.g., cells 6160, 6170) within the flow cell may be held statically within the flow cell 6110 for a dwell time before the fluid (e.g., cells 6170) flow from outlet 6132. In some variations, a longitudinal axis of the flow cell 6110 may be oriented substantially perpendicular to ground in order for fluid flow through the flow cell 6110 to be aided by gravity. At step 6104, the magnetic coupling between the magnet array 6120 and the cells 6160 may be released after the dwell time, and the cells 6160 may flow into the third reservoir 6150.

In some variations, stiction may cause cells to remain attached to a surface of a flow cell even after removal of a magnet array 6120. Therefore, a gas may be flowed through the flow cell 6110 to aid cell collection into the third reservoir 6150. Gas flow through the flow cell may provide improved cell recovery over liquid flushing through the flow cell. An interface generated by a gas (e.g., bubble, air gap) may be maintained by gravity, thereby enabling implementation of a relatively wide flowcell that further improves cell recovery relative to a horizontally oriented flow cell. The MACS modules described herein may be configured for positive selection and/or negative selection by modifying the sequence of steps.

Additionally or alternatively, an optical sensor may be configured to image a flow cell to generate imaging data used to identify a quantity of cells magnetically attracted to the magnet array. Fluid containing labeled cells may be flowed out of the flow cell when a predetermined quantity of cells have been measured by the optical sensor.

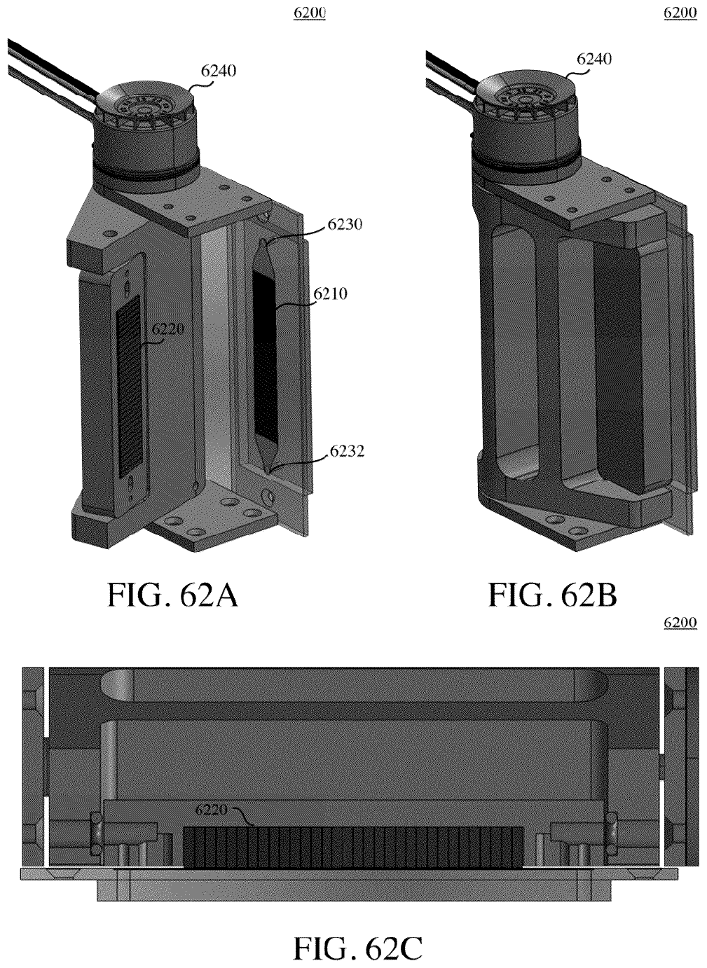

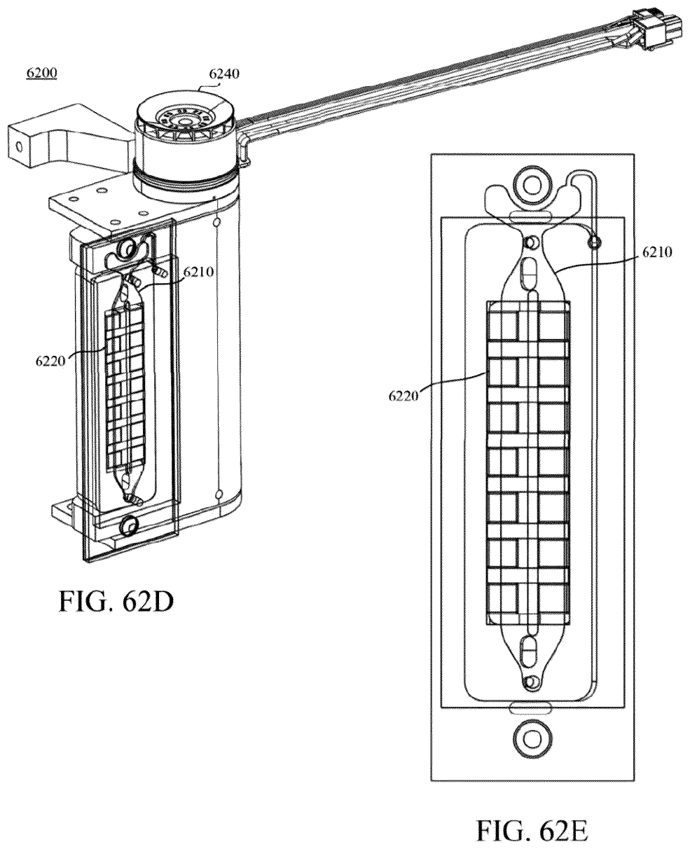

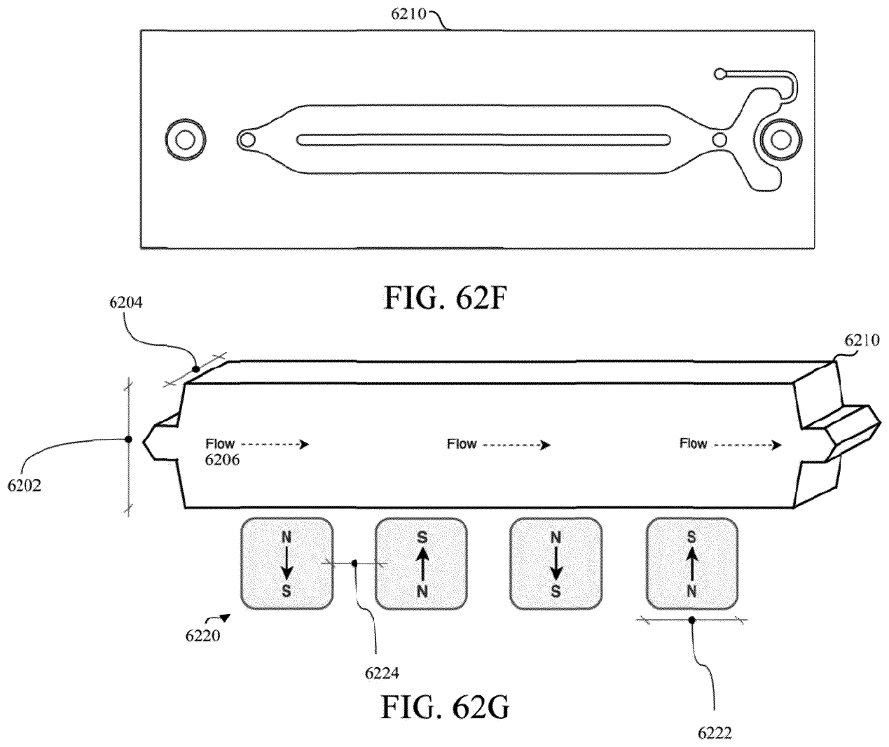

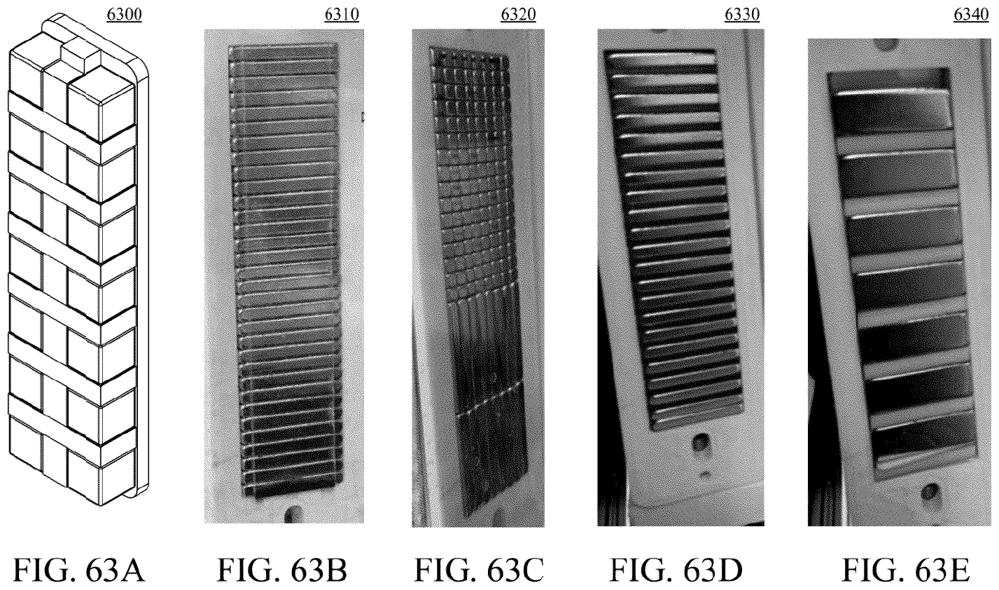

FIG. 62A is a perspective view of a cartridge MACS module 6200 in a first configuration. The cartridge MACS module 6200 may be a component of any of the cartridges described herein. For example, a cartridge for cell processing may comprise a liquid transfer bus and a plurality of modules with each module fluidically linked to the liquid transfer bus. The cartridge MACS module 6200 may comprise a flow cell 6210 comprising an elongate cavity having a cavity height, an inlet 6230, and an outlet 6232. The MACS module 6200 may further comprise a magnet array 6220 comprising a plurality of magnets. Each of the magnets may be spaced apart by a spacing distance, such as illustrated in FIGS. 62G, 63D, and 63E, although FIGS. 62A-62E illustrate a magnet array 6220 with magnets in contact with adjacent magnets.

FIG. 62G is a schematic diagram of the flow cell 6210 and magnet array 6220. In some variations, the flow cell 6210 may comprise a cavity height 6202 and a cavity width 6204. Fluid may be configured to flow through the flow cell 6210 in a first direction 6206. The magnet array 6220 may comprise a plurality of magnets with each magnet comprising a respective width 6222. In some variations, adjacent magnets may be separated by a predetermined spacing distance 6224. Each magnet pair may have the same or different spacing distance 6224. As shown in FIG. 62G, an orientation (e.g., poles) of the magnets in the magnet array 6220 may comprise a predetermined pattern. In some variations, a ratio of the cavity height 6202 to the spacing distance 6224 is between about 20:1 and about 1:20, between about 10:1 and about 1:10, between about 5:1 and about 1:5, and between about 3:1 and about 1:3, including all values and sub-ranges in-between. In some variations, an actuator 6240 (e.g., linear, rotary) may be configured to move the magnet array 6220 relative to the flow cell 6210. In some variations, an orientation (e.g., poles) of the magnets in the magnet array 6220 may comprise a predetermined pattern (e.g., Halbach array).

In some variations, the magnet array 6220 may move relative to the flow cell 6210 or vice versa. FIG. 62A illustrates the cartridge MACS module 6200 in an open configuration and FIG. 62B illustrates the cartridge MACS module 6200 in a closed configuration. FIG. 62B is a perspective view of the cartridge MACS module 6200 in a second configuration where labeled cells may be magnetically attracted towards the magnet array 6220. In the second configuration, the magnetic field lines generated by the magnet array traverse the flow channel exerting a magnetophoretic force on magnetically tagged cells that are injected into the channel. FIG. 62C is a cross-sectional side view of the cartridge MACS module 6200 including the magnet array 6220. FIG. 62D is a perspective view of a cartridge MACS module 6200 in the second configuration. FIG. 62E is a plan view of a flow cell 6210 and magnet array 6220 of a MACS system. FIG. 62F is a plan view of a flow cell 6210 of a cartridge MACS module.

FIG. 63A-63E are perspective views of a set of magnet arrays 6300, 6310, 6320, 6330, 6340. One or more of the size, strength, shape, spacing, and orientation of the magnets in a magnet array may be set to generate a magnetic field to attract magnetically-labeled cells. Additionally or alternatively, a magnet array may comprise a high-magnetic permeability material configured to enhance or reduce the field strength and field gradients within the flow cell. The material may be disposed between a magnet and flowcell. Additionally or alternatively, the material may be disposed within the flowcell and/or on one or more sides of the flowcell.

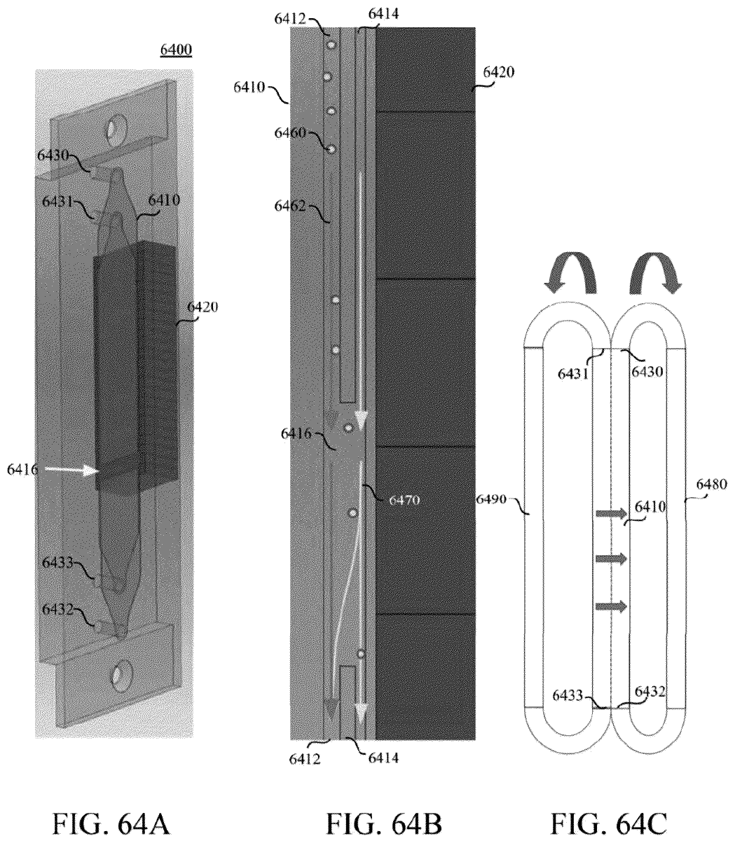

FIGS. 64A and 64B are respective perspective and cross-sectional side views of a cartridge MACS module 6400 comprising a flow cell 6410 and a magnet array 6420. The flow cell 6410 may comprise a set of linear channels 6412, 6414, 6416 comprising a first channel 6412 parallel to a second channel 6414, and a third channel 6416 in fluid communication with each of the first channel 6412 and the second channel 6416. As shown in FIG. 64B, the third channel 6416 may be disposed between the first channel 6412 and the second channel 6416 and define a volume where fluid from the first channel 6412 and the second channel 6416 interact (e.g., mix). In some variations, the flow cell 6410 may comprise a first inlet 6430 coupled to the first channel 6412 and configured to receive a first fluid 6460 (e.g., cells). A second inlet 6431 may be coupled to the second channel 6414 and configured to receive a second fluid 6470 (e.g., buffer). The flow cell 6410 may comprise a first outlet 6432 coupled to the first channel 6412 and a second outlet 6433 coupled to the second channel 6414.

The magnet array 6420 may be disposed external to the flow cell 6400 and may be moved relative to the flow cell 6400 as described herein. In some variations, a longitudinal axis of the flow cell 6410 may be perpendicular to ground such that fluid flows in a generally vertical direction.

In some variations, the first channel 6412 may have different dimensions form the second channel 6414. For example, a first cavity height of the first channel 6412 may be larger than a second cavity height of the second channel 6414. For example, a ratio of the first cavity height to a second cavity height may be between about 1:1 to about 3:7, between about 1:1 to about 2:3, and between about 2:3 to about 3:7, including all values and sub-ranges in-between. Fluid flowing through the first channel 6412 may have a slower flow rate relative to the second channel 6414 due to the larger cavity height of the first channel 6412 relative to the second channel 6414. In some variations, the third channel 6416 may comprise a ratio of a length of the third channel 6416 to a diameter of the third channel 6416 of between about 2:1 to about 6:1, between about 2:1 to about 3:1, between about 3:1 to about 4:1, between about 4:1 to about 5:1, between about 5:1 to about 6:1, and between about 3:1 to about 5:1, including all values and sub-ranges in-between.

As shown in FIG. 64B, a first fluid 6462 may flow through the flow cell 6410 generally following a first direction. The magnetically-labeled cells 6416 within the first fluid 6462 may separate from the rest of the first fluid 6462 within the third channel 6416 as the magnetic attractive forces generated by magnet array 6420 pulls the cells 6416 away from the first channel 6412 and towards the second channel 6414 (e.g., towards the magnet array 6420). Similarly, a second fluid 6470 (e.g., buffer) may flow through the second channel 6414. As the cells 6416 flow towards the magnet array 6420, they displace the second fluid 6470 flowing through the third channel 6416 such that a portion of the second fluid 6470 may flow into the first channel 6412. In this manner, magnetically-labeled cells 6416 may be magnetically separated from a first fluid 6462 and the second fluid 6470 may aid removal of the first fluid 642 not including the cells 6416.

In some variations, a set of fluidic loops may be coupled to the flow cell to enable a plurality of cell separation cycles. FIG. 64C is a schematic diagram of a cartridge MACS module comprising a flow cell 6410, a first fluid conduit 6480 coupled to an inlet 6430 of the flow cell 6410 and an outlet 6432 of the flow cell 6410. The first fluid conduit 6480 may be configured to receive the set of cells from an outlet 6432 of the flow cell 6410 for recovery and/or recirculation through the inlet 6430 of the flow cell 6410. A second fluid conduit 6490 may be coupled to the inlet 6431 of the flow cell 6410 and the outlet 6433 of the flow cell 6410 to recirculate fluid such as buffer and unrecovered magnetically-labeled cells. The second fluid conduit 6490 may be configured to receive a fluid without the set of cells from the flow cell 6410. Higher purities of labeled cells may be recovered based on a number of cycles performed. For example, a single cell separation cycle may yield about 80% cell purity, a second cell separation cycle may yield about 96% cell purity, a third cell separation cycle may yield about 99.2% cell purity, and a fourth cell separation cycle may yield about 99.84% cell purity.

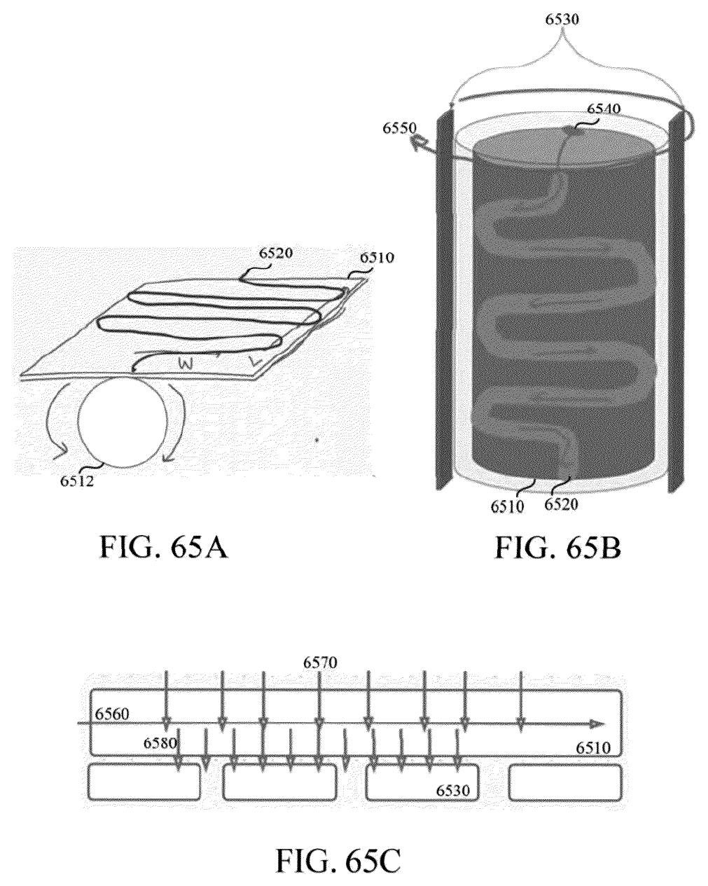

In some variations, applying a centrifugal force to a magnetic cell separation process may further attract labeled cells toward a magnetic array independently of fluid flow rate so as to maintain throughput. FIGS. 65A-65C are schematic diagrams of a cartridge MACS module 6500 utilizing centrifugal force to aid a cell separation process. FIG. 65A depicts a flattened flow cell 6510 configured to be wrapped to form a generally cylindrical shape 6512. The flow cell 6510 may comprise a curved flow path 6520.

FIG. 65B illustrates a cylindrical flow cell 6510 concentrically surrounded by (e.g., nested within) a cylindrical magnet array 6530. In FIG. 65B, only a cross-section of the magnet array 6530 is shown for the sake of clarity. The flow cell 6510 may be spaced apart from the magnet array 6530 by a predetermined spacing distance. Accordingly, the flow cell 6510 may be configured to rotate 6550 about a longitudinal axis to generate a centrifugal force on the fluid 6540 within the flow path 6520 in an outward direction towards the magnet array 6530. During a cell separation process, the fluid may be subject to set of forces depicted in FIG. 65C including a bulk fluidic force 6560 in an axial (e.g., bulk flow) direction, a centrifugal force 6570 in a radially outward direction from a center of rotation (e.g., proportional to a net particle system buoyancy), and a magnetic force 6580 extending radially outward from a center of rotation (e.g., proportional to net particle system magnetic attractiveness). In some variations, labeled cells may comprise a higher density than non-labeled cells. Therefore, centrifugal force may preferentially push the labeled cells towards the magnet 6530, further increasing the specificity and efficiency of cell separation.

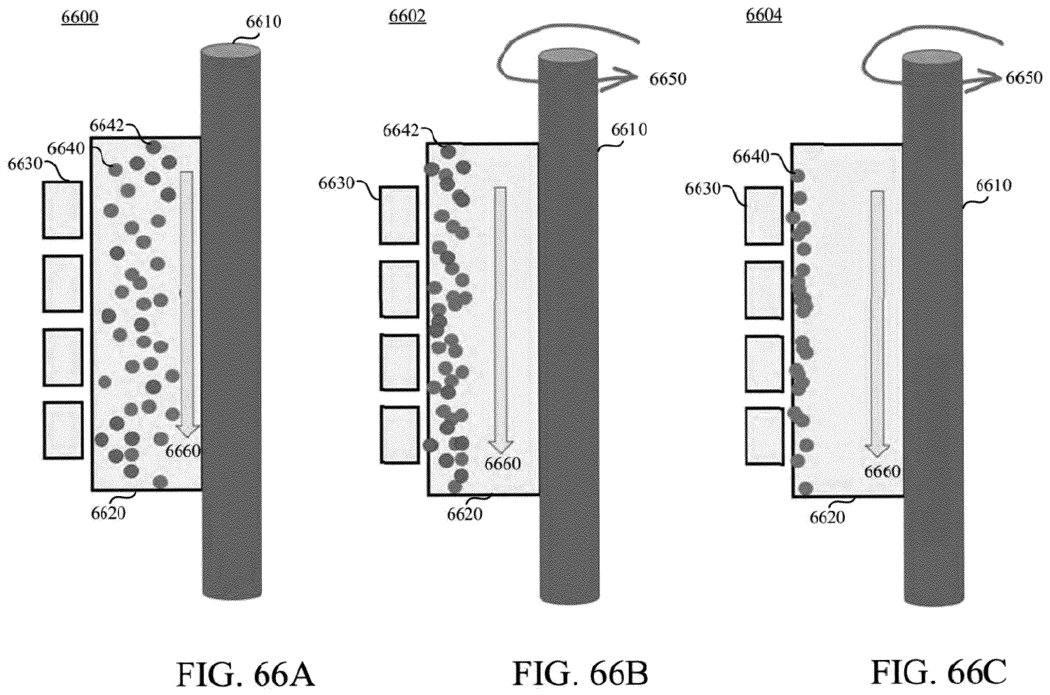

FIGS. 66A-66C are schematic diagrams of a cell separation system and process. A magnetic cell separation system may comprise a flow cell 6610 comprising a flow path 6620 (shown schematically flattened for sake of clarity), and a magnet array 6630. As shown in step 6600, a set of cells 6640, 6642 may comprise labeled cells 6640 (e.g., magnetically labeled cells) and non-labeled cells 6642 may flow into the flow path 6620 of flow cell 6610. For example, a set of the cells 6640 may be labeled with a magnetic-activated cell selection (MACS) reagent. The magnet array 6630 may be disposed external to the flow cell 6610 such that the magnet array 6630 may be moveable relative to the flow cell 6610. For example, the magnet array 6630 may move away from the flow cell 6610 to facilitate flowing the set of cells 6640 out of the flow cell 6610.

At step 6602, the flow cell 6650 may be rotated to generate centrifugal force to push the cells 6640, 6642 toward the magnet array 6630. In some variations, a longitudinal axis of the flow cell 6610 may be oriented substantially perpendicular to ground in order for fluid flow through the flow cell 6610 to be aided by gravity. At step 6604, the magnet array 6630 may magnetically attract the set of cells 6640 towards the magnet array 6630 for a predetermined dwell time as described herein. The non-labeled cells 6642 are not magnetically attracted to the magnet array 6630 and may flow out of the flow cell 6610 into, for example, a waste vessel. In some variations, the fluid (e.g., cells 6160, 6170) within the flow cell may be held statically within the flow cell 6110 for a dwell time before the fluid (e.g., cells 6170) flow from outlet 6132. In some variations, the magnetic coupling between the magnet array 6630 and the cells 6640 may be released after the dwell time, and the cells 6640 may be recovered.

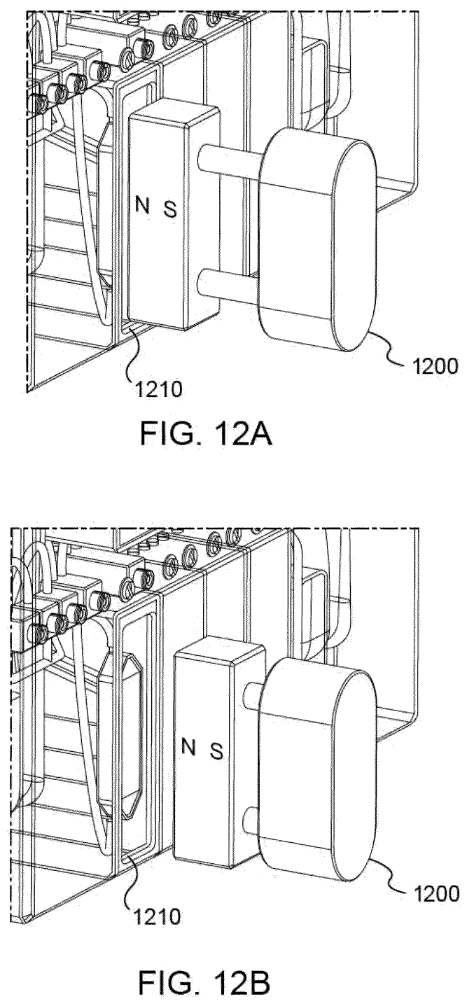

FIGS. 12A and 12B illustrate the magnet of the docking station MACS module 1200 comprising a magnet and a cartridge MACS module 1210. The magnet is shown in FIG. 12A in an ON configuration and shown in FIG. 12B in an OFF configuration.

### Bioreactor

The bioreactors described herein may comprise a vessel configured to culture mammalian cells. Generally, cell and gene therapy products may be grown in a bioreactor to produce a clinical dose which may subsequently be administered to a patient. A number of biological and environmental factors may be controlled to optimize the proliferation speed and success of cell growth. The bioreactor modules described herein enable one or more of monitoring, adjusting, and/or controlling of cell growth (e.g., to facilitate consistent and efficient cellular proliferation).

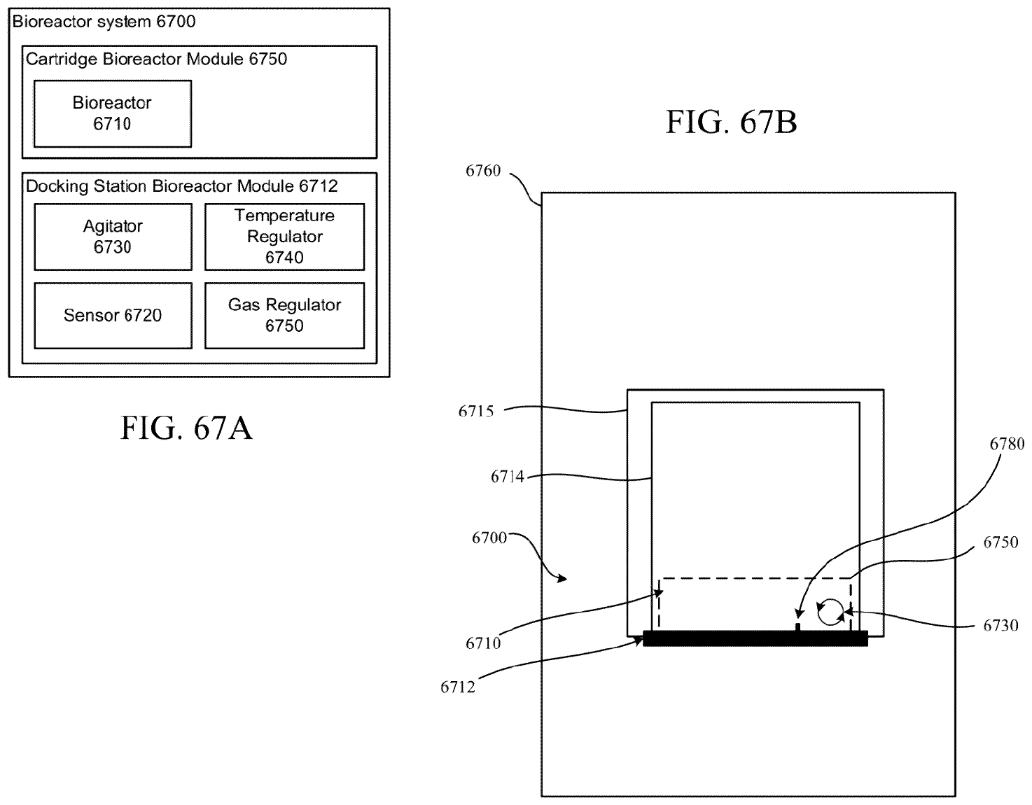

FIG. 67A is a schematic diagram of a bioreactor system 6700 comprising one or more of a cartridge bioreactor module 6750, the cartridge bioreactor module 6750 comprising a bioreactor 6710, and a docking station bioreactor module 6712 comprising one or more sensors 6720, an agitator 6730, a temperature regulator 6740, and a gas regulator 6750. In some variations, the sensor 6720 may be configured to monitor (e.g., measure, sense, determine) one or more characteristics of the cartridge bioreactor module, including of the cells in the bioreactor 6710. For example, the sensor 6720 may comprise one or more of a pH sensor, a DO sensor, a temperature sensor, a glucose sensor, a lactose sensor, a cell density sensor, a humidity sensor, combinations thereof, and the like, which may be used to probe the environment within the bioreactor 6710. One or more of the sensors may be a non-invasive optical sensor.

FIG. 67B is a schematic diagram of a cell processing and manufacturing system comprising a CPS 6760 and a bioreactor system 6700 therein, wherein the bioreactor system 6700 comprises a docking station bioreactor module 6712, a cartridge 6714, and a cartridge bioreactor module 6750. The docking station bioreactor module 6712 may comprise an agitator 6730, and a temperature regulator, sensor(s), and gas regulator (not shown). The CPS 6760 may further comprise a fluid transfer connect 6780. In some variations, the cartridge 6714 for cell processing may comprise a liquid transfer bus and, in addition to the cartridge bioreactor module 6750, a plurality of modules (e.g., CCE module, MACS module, EP module). Each module may be fluidically linked to the liquid transfer bus. In variations, the cartridge bioreactor module may comprise at least one bioreactor.

As shown in FIG. 67B, the docking station bioreactor module 6712 may be configured to interface with the cartridge bioreactor module 6750 of the cartridge 6770, wherein the cartridge module bioreactor 6750 comprises a bioreactor therein. In some variations, the docking station bioreactor module 612 may comprise the agitator 6730 configured to couple to the bioreactor of the cartridge bioreactor module 6750. The agitator 6730 may be configured to agitate, within the bioreactor, cell culture media comprising cells. In some variations, the fluid transfer connect 6780 may be configured to couple the docking station bioreactor module 6700 with the cartridge bioreactor module 6750.

As shown in FIG. 67B, the cartridge 6714 is disposed within the CPS 6760. Once the fluid transfer connect 6780 couples (e.g., to create a sterile flow path) the docking station bioreactor module 6712 of the CPS 6760 to the cartridge bioreactor module 6750 of the cartridge 6714, the cell culture medium within the bioreactor of the cartridge bioreactor module 6750 may be agitated by an agitator 6730 of the docking station bioreactor module 6712. In some variations, the fluid transfer connect 6780 may comprise a set of foldable sidewalls (e.g., like an accordion) configured to receive and dissipate the agitation of the agitator 6730 without transmitting such motion to the CPS 6760. That is, the fluid transfer connect 6780 may function as a bellows to maintain the connection between the docking station bioreactor module 6712 of the CPS 6760 and the cartridge bioreactor module 6780 without agitating the CPS 6760.

In some variations, an agitator may be configured to generate motion (e.g., orbital, rotary, linear) to the bioreactor of the cartridge bioreactor module 6750 in order to mix the culture in instances where it is required to encourage interactions with a reagent and cells. For example, orbital motion may be used to create a homogenous culture volume such that a small sample taken from the culture may be representative of the culture at large. In some variations, the agitator 6730 may comprise one or more impellers. The agitator 6730 may be configured to provide variable-intensity mixing during culture at defined periods.

In some variations, orbital motion may encourage increased interactions within the cell culture, such as in the toroidal bioreactors described herein that comprise a geometry that may encourage the continuous and gentle flow of fluid around the bioreactor, thereby aiding homogenous mixing with minimal shear stress transferred to the cells.

In some variations, the temperature regulator may be configured to control a temperature of a bioreactor and corresponding processes. The temperature regulator may be coupled to the bioreactor. For example, the temperature regulator may control a temperature of a cell culture to be between about 2° C. and about 40° C. and thereby ensure that a culture is heated to physiological conditions and cooled to slow metabolic processes (e.g., to keep cells in a dormant state) as desired. For example, the thermal regulator may comprise a circulating coolant coupled to a heat exchanger coupled to a thermal interface (e.g., heating/cooling plate of the docking station bioreactor module 6712).

In some variations, the gas regulator may be coupled to the bioreactor and configured to control a gas composition of a bioreactor and corresponding processes using one or more of clean dry air (CDA), carbon dioxide, and nitrogen, as shown in FIG. 2E. The gas regulator may be coupled to the bioreactor within the cartridge bioreactor module 6750. For example, the sensors and gas regulator may provide closed-loop gas control of the cartridge bioreactor module 6750. In some variations, CDA may comprise oxygen such as pure oxygen. In some variations, the gas regulator may comprise a manifold coupled to one or more gas sources. The manifold may include a solenoid coupled to a valve (e.g., restrictive orifice) configured to control gas flow through the bioreactor. The solenoid may be configured to pulse to control a quantity and composition of gas received through the manifold. Additionally or alternatively, one or more of a proportional valve and Mass Flow Controller (MFC) may be configured to meter and control the flow of gas to a manifold. In some variations, the gas regulator may comprise one or more sensors to measure the gas mixture and/or flow rate. Additionally or alternatively, the sensors may be configured for closed-loop control of gas flow through the gas regulator.

In some variations, measured pH from a pH sensor may be used to control a pH of the bioreactor using the gas regulator. For example, in response to the measured pH, the gas regulator may control a CO2 concentration of the gas contacting the cell culture to control the free hydrogen ions and pH of the culture. In some variations, a pH of the bioreactor may be between about 5.5 and about 8.5. One or more of CO2 composition of the gas in the bioreactor, buffer, and reagents (e.g., acid, base) may be used to regulate pH. In some variations, a dissolved oxygen concentration of the bioreactor may be between about 0% and about 21%. Nitrogen composition of the gas in the bioreactor may be used to regulate the dissolved oxygen concentration. For example, control of both the agitator in the bioreactor and the flow rate and composition of the gas contacting the cell culture may regulate the dissolved carbon dioxide concentration.

In some variations, measured dissolved oxygen from a dissolved oxygen sensor may be used to control an oxygen concentration (e.g., below atmospheric levels) of the bioreactor using the gas regulator. For example, gas regulator may control a nitrogen concentration of the gas contacting the cell culture to create hypoxic conditions.

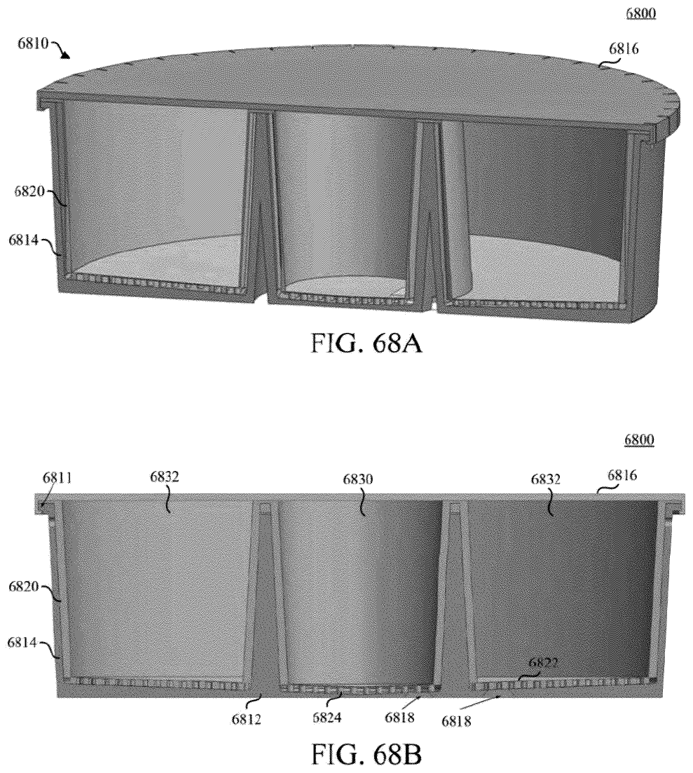

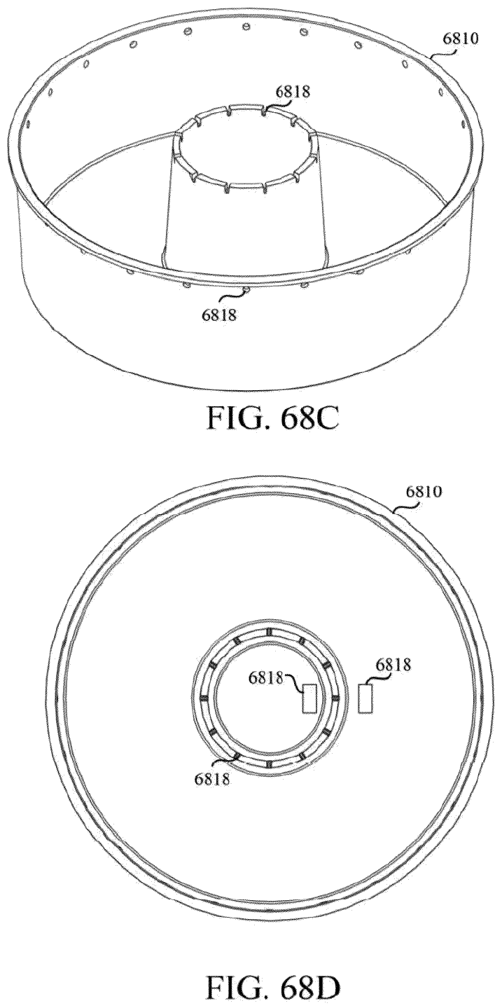

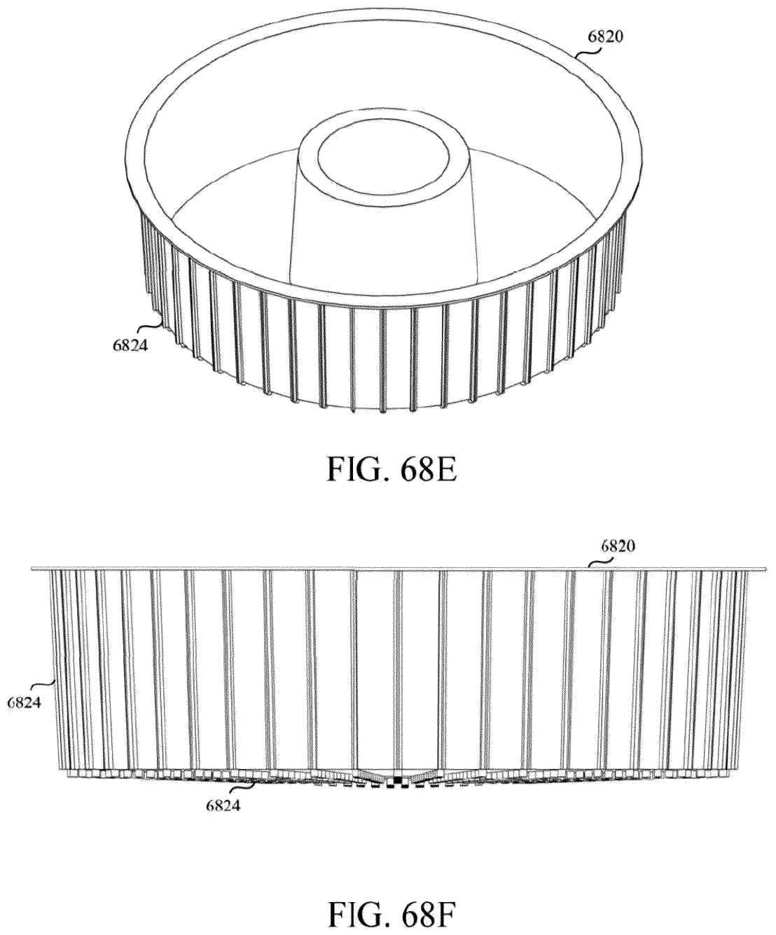

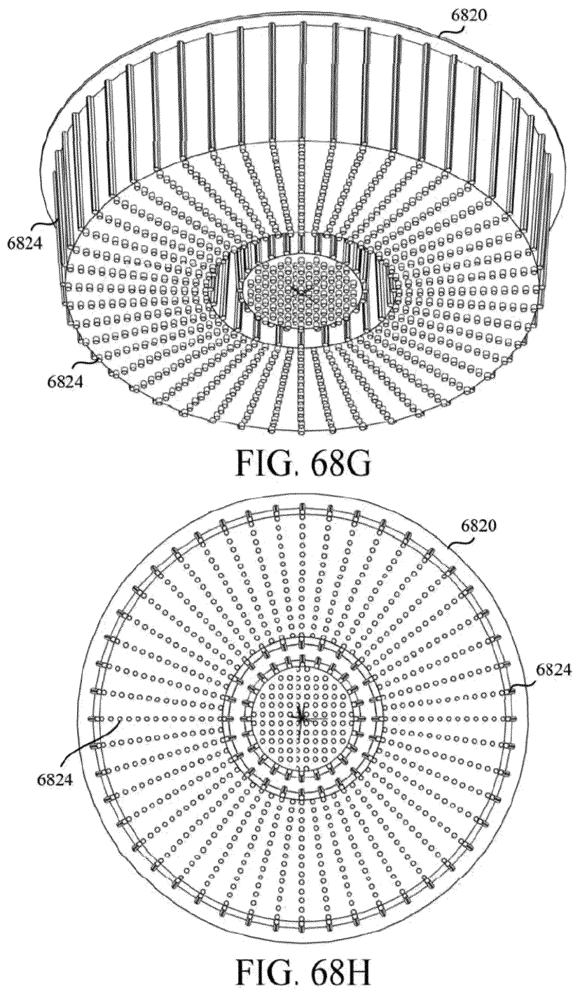

FIGS. 68A and 68B are cross-sectional perspective views of a bioreactor 6800 comprising an enclosure 6810 comprising a base 6812, a sidewall 6814, and a top 6816. A gas-permeable membrane 6820 may be coupled to one or more of the base 6812 and the sidewall 6814 of the enclosure 6810. In some variations, the enclosure 6810 may comprise a first chamber 6830 having a first volume and a second chamber 6832 having a second volume, the first chamber 6830 separated from the second chamber 6832, and the first volume smaller than the second volume. In some variations, the first chamber 6830 may be concentrically nested within the second chamber 6832. For example, nesting the chambers may enable larger overall working volume ranges (e.g., 100:1). The first chamber 6830 may comprise a well shape with an angled base surface to promote fluid pooling at a center of the first chamber 6830 during aspiration. In some variations, the base 6812 may be disposed on a thermal regulator (not shown) such as a thermoelectric element. In some variations, the enclosure 6810 may be composed of a thermally conductive material such as a metal (e.g., aluminum).

In some variations, the bioreactor 6800 may be coupled to a gas regulator (not shown) to facilitate gas transfer through the gas-permeable membrane 6820 (e.g., into and out of the culture). The gas-permeable membrane 6820 may be configured to hold a cell culture. Gas may diffuse through the surfaces of the culture that contact the gas-permeable membrane to enable increased oxygenation of the cell culture and removal of gaseous metabolic byproducts of the cell culture, and thus increase the potential for metabolic activity. For example, the gas-permeable membrane 6820 enables dissolved oxygen to diffuse into the culture in close proximity to a cell bed where the oxygen may be consumed. In some variations, the bioreactor may be coupled to both a first gas regulator to facilitate gas transfer through the gas-permeable membrane and a second gas regulator to facilitate control of headspace gas composition.

In addition to gas transfer, the bioreactors described herein may be configured to efficiently control a temperature of a cell culture using a conductive thermal interface (e.g., gas-permeable membrane 6820, enclosure 6810) along both a base and sidewall of the bioreactor.

In some variations, the first chamber 6830 may comprise a working volume of between about 10 ml and about 100 ml. In some variations, the first chamber 6830 may comprise a total volume of between about 10 ml and about 130 ml. In some variations, the second chamber 6832 may comprise a working volume of between about 100 ml and about 1000 ml. In some variations, the second chamber 6832 may comprise a total volume of between about 100 ml and about 1400 ml. In some variations, the first chamber 6830 may comprise a diameter of between about 10 mm and about 100 mm, and a height of between about 10 mm and about 100 mm. In some variations, the second chamber 6832 may comprise a diameter of between about 100 mm and about 250 mm, and a height of between about 10 mm and about 100 mm.

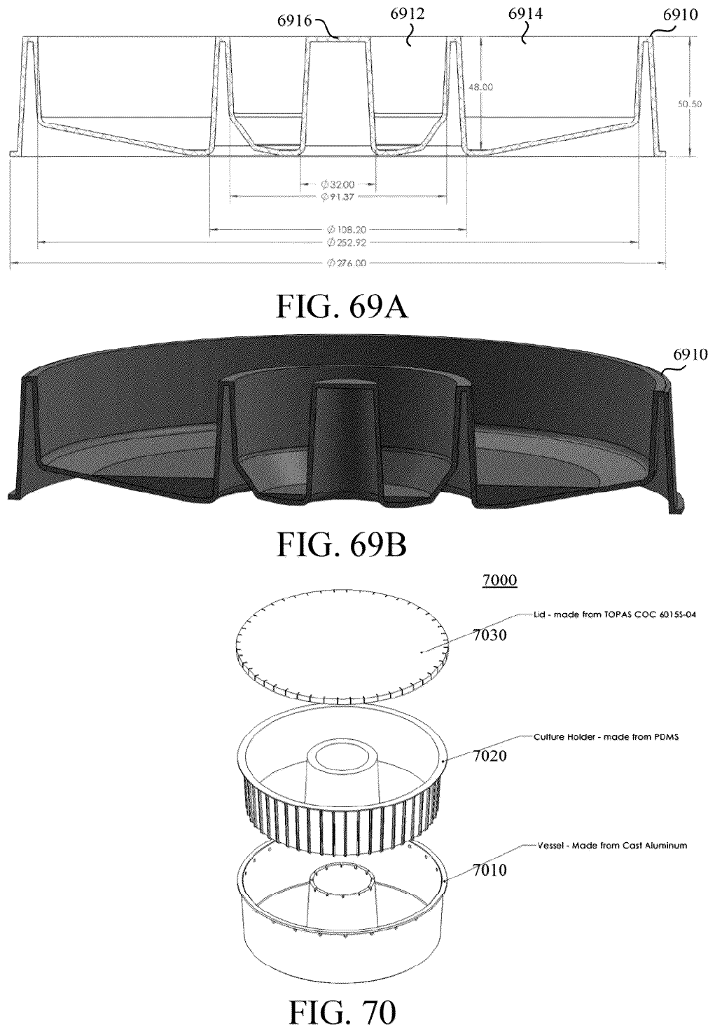

As shown in FIG. 68B, a base 6822 of the gas-permeable membrane 6820 may comprise an angle between about 3 degrees and about 10 degrees relative to the base 6812 of the enclosure 6810. Similarly, FIGS. 69A and 69B depict a sloped base. For example, due to a slope of the base 6822, the chambers 6830, 6832 are deeper towards a center of the bioreactor 6800. This may encourage cell growth towards a center of the bioreactor 6800, which may aid one or more of cell sampling, cell transfer, cell recovery, and the like. In some variations, orbital motion of the bioreactor 6800 may promote cell congregation toward a center of the bioreactor 6800, thereby increasing interaction between the cells.

In some variations, the gas-permeable membrane 680 may comprise a curved surface. In some variations, the gas-permeable membrane may comprise a set of patterned curved surfaces. For example, the set of patterned curved surfaces may comprise a radius of curvature of between about 50 mm and about 500 mm.

In some variations, the bioreactor may be configured to facilitate monitoring (e.g., temperature, pH, dissolved oxygen) and fluid flow (e.g., gas composition, fluid transfer) between the chambers. As shown in FIG. 68C, the enclosure 6810 may comprise one or more nested surfaces curved around a longitudinal axis (e.g., center) of the enclosure 6810. For example, the nested surfaces may comprise a set of concentric toroids. The enclosure 6810 may comprise a toroid shape. FIG. 68C is a perspective view and FIG. 68D is a bottom view of enclosure 6810 comprising a set of apertures 6818 (e.g., holes, openings, slits, slots). In some variations, the apertures 6818 may enable gas and/or heat transfer between the components and chambers of the bioreactor 6800. Additionally or alternatively, one or more sensors may be coupled to the apertures 6818. For example, the apertures 6818 may be coupled to a non-contact sensor (e.g., pH, DO) such as an optical sensor (not shown) configured to determine a fluorescent spot disposed on a surface of the bioreactor. In some variations, one or more of a sensor and fluid connector may be introduced through the apertures 6818.

In some variations, the gas-permeable membrane extends along the base 6812 and the sidewall 6814 of the enclosure 6810, as shown in FIG. 68B. In some variations, the gas-permeable membrane extends only along the base 6812 of the enclosure 6810. FIG. 68E is a perspective view and FIG. 68F is a side view of the gas-permeable membrane 6820 where an outer surface of the gas-permeable membrane 6820 comprises one or more projections 6824 (e.g., projections, spacers, ribs). The projections 6824 are also depicted in the perspective view of FIG. 68G and bottom view of FIG. 68H. The projections 6824 contact the enclosure 6810 and define a cavity between the enclosure 6810 and the gas-permeable membrane 6820. That is, the projections 6824 may be configured to mechanically space away the enclosure 6810 from a portion of the gas-permeable membrane 6820 to facilitate thermal transfer from the enclosure 6810 to the cell culture. In some variations the gas-permeable membrane may comprise polydimethylsiloxane (PDMS) (e.g., silicone), fluorinated ethylene propylene (FEP), polyolefin (PO), polystyrene (PS), ethyl vinyl acetate (EVA) and have a thickness of between about 0.1 mm and about 0.4 mm, between about 0.2 mm and about 0.3 mm, and about 0.25 mm, including all ranges and sub-values in-between.

FIG. 69A is a cross-sectional side view of an enclosure 6910 of a bioreactor comprising a first chamber 6912, a second chamber 6914, and a column 6916 extending along a longitudinal axis of the enclosure 6910. FIG. 69B is a cross-sectional perspective view of the enclosure 6910 showing the nested curves of the enclosure 6910. The column 6916 may be configured to promote cell culture in combination with agitation such as orbital motion.

FIG. 70 is an exploded perspective view of a bioreactor 7000 comprising an enclosure 7010, a gas-permeable membrane 7020, and a top 7030. The top 7030 may be composed of a material such as polyethylene.

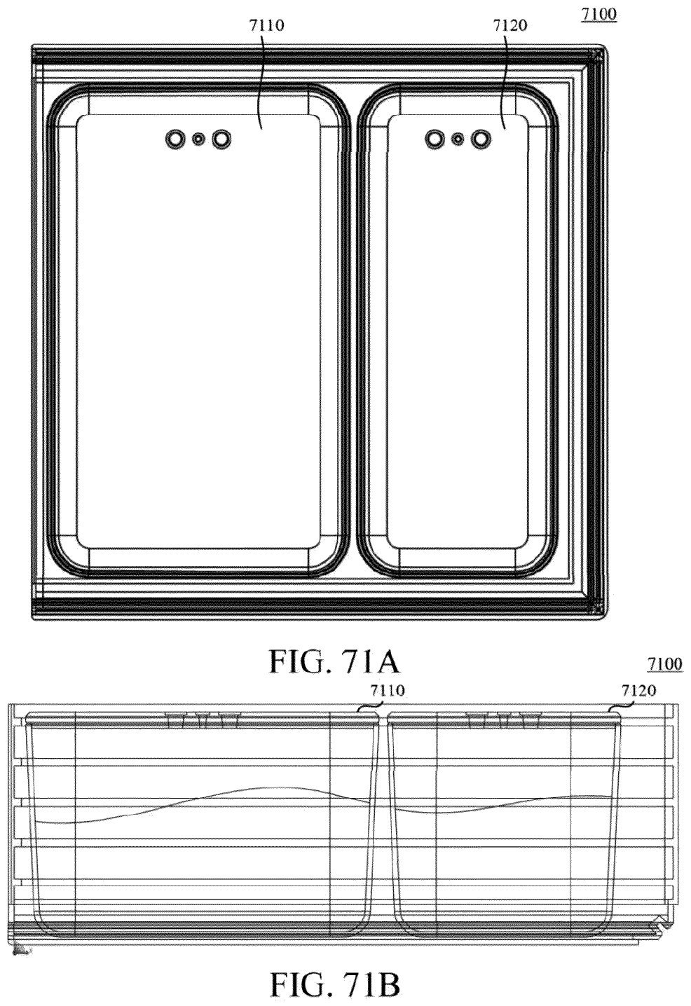

FIG. 71A is a plan view of a bioreactor 7100 comprising a first chamber 7110 and a second chamber 7120. FIG. 71B is a cross-sectional side view of the bioreactor 7100.

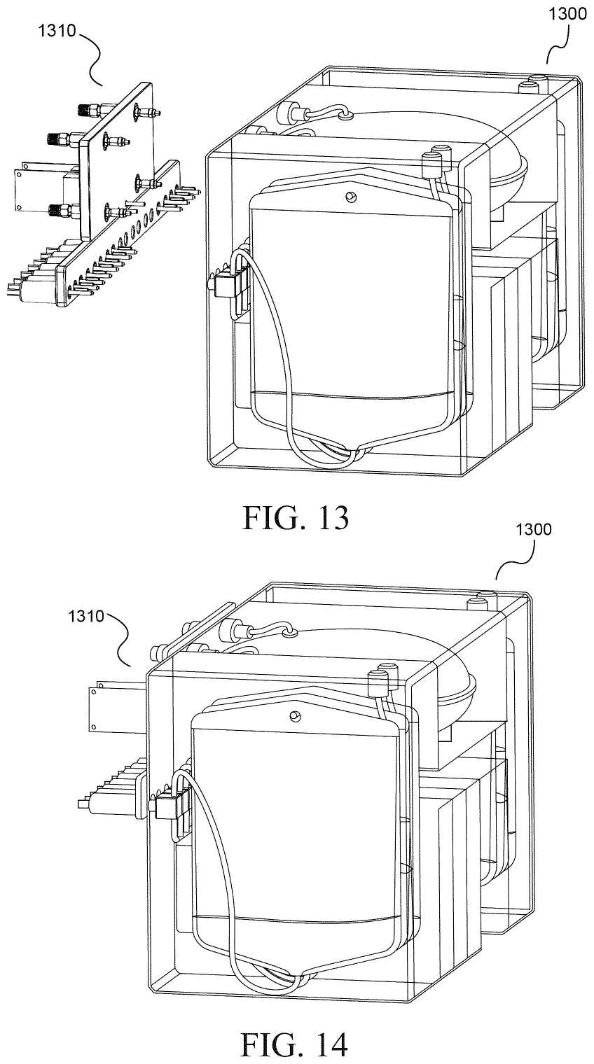

FIG. 13 and FIG. 14 are perspective views of a cartridge 1300 and docking station bioreactor module interface 1310. The docking station bioreactor module interface 1310 is coupled to the cartridge 1300 in FIG. 14.

### Electroporation Module

In some variations, an electroporation module may be configured to facilitate intracellular delivery of macromolecules (i.e., transfection by electroporation). An electroporation module may contain a continuous flow or batch mode chamber and one or more sets of electrodes for applying direct or alternating current to the chamber. An electrical discharge from one or more capacitors, or current sources, may generate sufficient current in the chamber to promote transfer of a polynucleotide, protein, nucleoprotein complex, or other macromolecule into the cells in the cell product. As with other modules described herein, one or more components used for the process step (here, electroporation) may be provided on the cartridge or in the docking station to which the cartridge interfaces. For example, the capacitor(s) and/or batteries may be provided in the docking station module on the cartridge module. The electroporation module may, in some variations, be configured to apply an electric field to a cell suspension under continuous flow in a microfluidic device, e.g., as described in Garcia et al. Sci. Rep. 6:21238 (2016).

Additionally or alternatively, intracellular delivery of macromolecules may also be achieved by other methods, such as mechanoporation. It should be understood that throughout the disclosure variations comprising an electroporation module may instead or in addition comprise a mechanoporation module, or another module configured to perform any suitable method of delivering macromolecules into cells. Mechanoporation may be achieved by, for example, applying transient, fluidic pressure to a solution containing cells, or by applying physical pressure to the cells (e.g., by microneedles). Illustrative methods of mechanoporation by passing a cell suspension through a constriction are provided, e.g., in International Patent Publication No. WO 2017/041051 and WO 2017/123663, and are incorporated by reference herein. Mechanoporation may also be achieved by applying a vortex to a cell suspension in a microfluidic device.

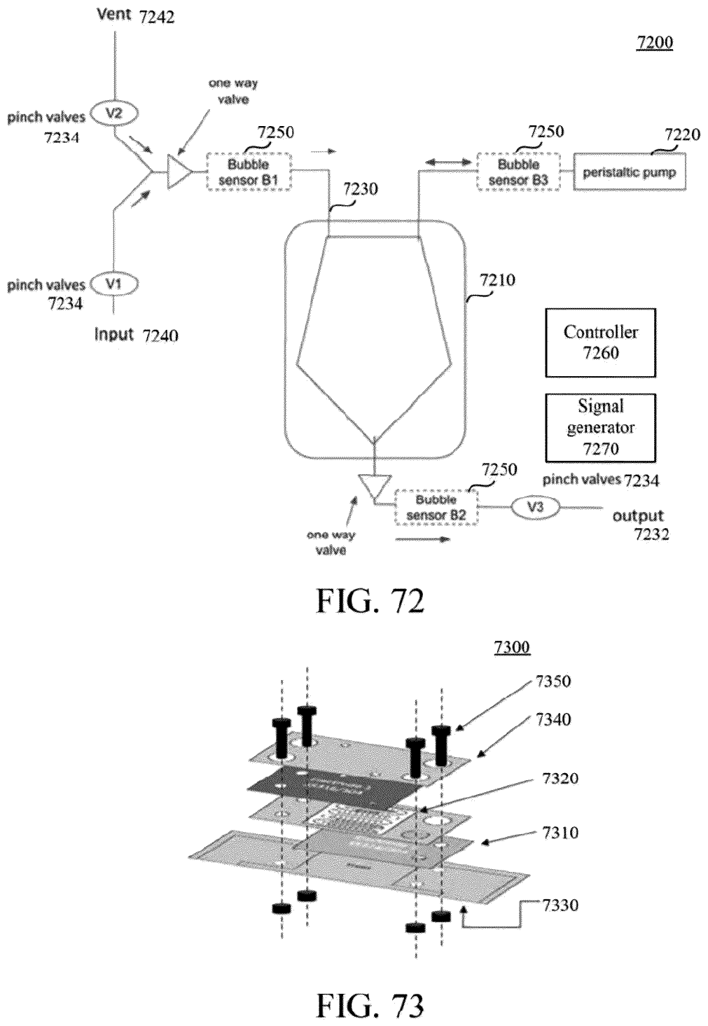

FIG. 72 is a schematic diagram of an electroporation module 7200 (e.g., electroporation system) comprising an electroporation chamber 7210 (which may comprise a fluid conduit), a pump 7220, an inlet 7230, an outlet 7232, a set of pinch valves 7234, a first fluid source 7240 (e.g., fluid reservoir, cell reservoir), a second fluid source 7242 (e.g., vent, gas source), a set of sensors 7250 (e.g., bubble sensors), and a controller (e.g., processor and memory) configured to control the module 7200, and a signal generator 7270 configured to deliver an electroporation signal (e.g., voltage pulse) to the electroporation chamber 7210.

In some variations, the fluid conduit 7210 may be configured to receive a first fluid comprising cells and a second fluid. A set of electrodes may be coupled to the fluid conduit 7210. A pump may be coupled to the fluid conduit 7210. The controller 7260 may be configured to generate a first signal to introduce the first fluid into the fluid conduit 7210 using the pump 7220, generate a second signal to introduce the second fluid into the fluid conduit 7210 such that the second fluid separates the first fluid from a third fluid, and generate an electroporation signal to electroporate the cells in the fluid conduit 7210 using the set of electrodes.

In some variations, the second fluid may comprise a gas or oil. In some variations, the controller may be configured to generate a third signal to introduce the third fluid into the fluid conduit 7210. The third fluid may be separated from the first fluid by the second fluid. In some variations, a cartridge for cell processing may comprise a liquid transfer bus and a plurality of modules such as the electroporation module 7200. Each module may be fluidically linked to the liquid transfer bus.

The set of sensors 7250 may be configured to measure fluid changes in a fluid conduit such as a change from a first fluid to a second fluid (e.g., liquid to air) in the fluid conduit. The module 7200 may further comprise a set of valves configured to ensure fluid does not backflow into the electroporation chamber 7210 and/or fluid source 7240. The electroporation chamber 7210 may comprise a cavity configured to hold a fluid to be electroporated and a set of electrodes to apply an electroporation signal to the fluid. For example, the signal generator 7270 may generate a square valve pulse as described in more detail herein.

In some variations, the electroporation module 7200 (e.g., valves 7234, pump 7220, sensors 7250, and controller 7260) may be configured to control fluid flow through the electroporation chamber 7210 in a discontinuous (e.g., batch process) manner. For example, a first batch of cells may undergo electroporation and be physically separated from a second batch of cells by an intermediate fluid such as air or fluid such as oil. Separating cell batches may reduce mixing of transfected and non-transfected cells, and further ensure fixed batch volume. That is, a fluid gap may form a visually verifiable boundary between cell batches to reduce diffusion and mixing between electroporated and non-electroporated cells. Separating cell batches may reduce the duration of time that cells are exposed to certain cytotoxic reagents (e.g., electroporation buffer), thereby increasing performance.

In some variations, a batch of cells may be electroporated when substantially static (e.g., substantially no fluid flow state). By contrast, conventional continuous flow electroporation has an upper fluid flow rate limit correlated to a transfection efficiency. In the batch processing described herein, cell batches may be transferred into and out of the electroporation chamber 7210 at a predetermined rate to increase the overall throughput of the system 7200 without a decrease in electroporation efficiency. Furthermore, the electroporation system 7200 does not utilize a precisely controlled flow rate/pulse rate such as those needed for continuous flow electroporation systems.

FIG. 73 is an exploded perspective view of an electroporation module 7300 of a cartridge, which may comprise an electrode 7310, a fluid conduit 7320 (e.g., electroporation chamber), a substrate 7330 (e.g. alloy busbar), a housing 7340, and a fastener 7350. In some variations, the fluid conduit 7320 may be configured to hold a volume of fluid between about 0.4 ml and about 3.5 ml. The electroporation module 7300 is a parallel-plate design. In some variations, the electrodes may comprise stainless steel and may be separated by an insulating gasket. In some variations, the electrodes may be polished and/or coated with nonreactive materials (e.g., gold, platinum) to reduce gradual buildup of biological matter (e.g., charged molecules, DNA, proteins) on the electrode surface.

Generally, a method of electroporating cells may comprise receiving a first fluid comprising cells in a fluid conduit, receiving a second fluid in the fluid conduit to separate the first fluid from a third fluid, applying an electroporation signal to the first fluid to electroporate the cells. In some variations, the third fluid may be received in the fluid conduit separated from the first fluid by the second fluid. In some variations, the first fluid may be substantially static when applying the electroporation signal.

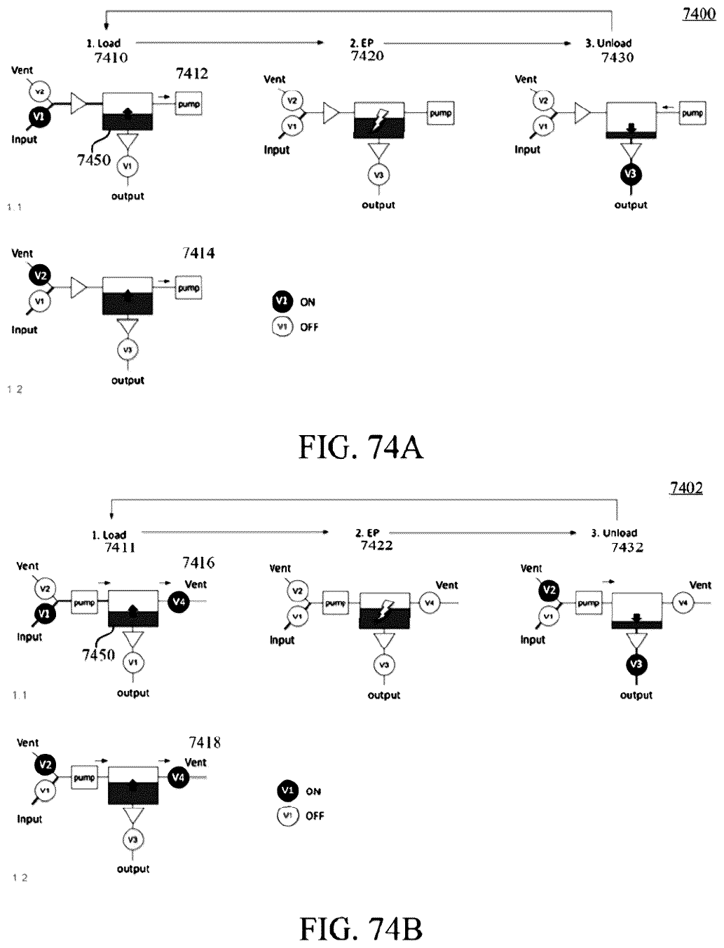

FIGS. 74A-74B are schematic diagrams of variations of an electroporation process 7400, 7402. A method 7400 may include loading cells 7410 into an electroporation chamber 7450. For example, at step 7412, a first fluid may be pumped into the electroporation by opening valve v1 and the pump generating negative pressure (valves v2 and v3 are closed). At step 7414, a second fluid (e.g., gas, oil) may separate the first fluid from a third fluid to create a first batch of cells to electroporate. For example, valves v1 and v3 may be closed with valve v2 open and the pump generating negative pressure. In some variations, a loading volume may be between about 1 ml and about 3 ml with a pumping time of between about 8 seconds and about 15 seconds (at a rate of about 20 ml/min). At step 7420, the cells of the first fluid may be electroporated with each of the valves closed and the pump off. At step 7430, the cells of the first fluid may be flowed out of the electroporation chamber 7450 to output where valves v1 and v2 are closed, valve v3 is open, and the pump generates positive pressure.

FIG. 74B depicts another configuration where a pump is disposed between an input and the electroporation chamber such that the pump may be configured to pump in a single direction. A method 7402 may include loading cells 7411 into an electroporation chamber 7450. For example, at step 7416, a first fluid may be pumped into the electroporation by opening valve v1 and v4, and the pump generating positive pressure (valves v2 and v3 are closed). At step 7418, a second fluid (e.g., gas, oil) may separate the first fluid from a third fluid to create a first batch of cells to electroporate. For example, valves v and v3 may be closed with valves v2 and v4 open, and the pump generating positive pressure. At step 7422, the cells of the first fluid may be electroporated with each of the valves closed and the pump off. At step 7432, the cells of the first fluid may be flowed out of the electroporation chamber 7450 to output where valves v1 and v4 are closed, valves v2 and v3 are open, and the pump generates positive pressure.

In some variations, an impedance/resistance across electrodes of an electroporation system may increase over time due to electrode passivation/degradation due to charged biological matter (e.g., charged molecules, DNA, proteins) attaching to the electrode surface. Active electrical field compensation may be applied to ensure a consistent electrical field strength applied to cells over multiple batches of cells. This may reduce the need for electrode surface modification to reduce passivation.

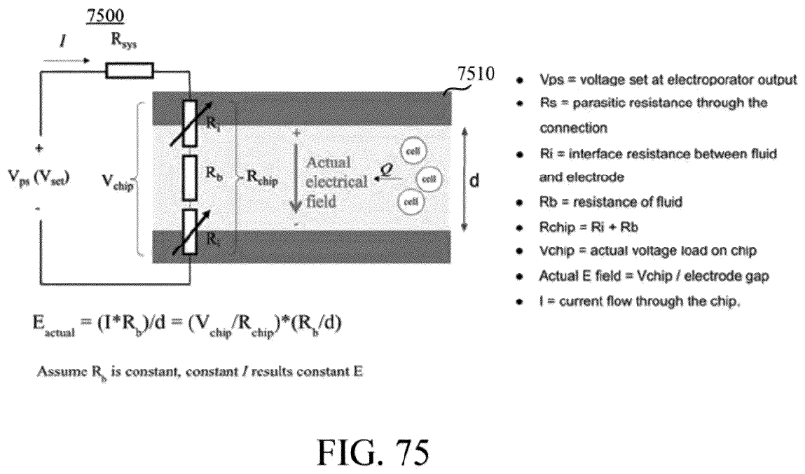

FIG. 75 is a circuit diagram of a resistor divider network for an electroporation process 7500. For example, a set of cells may be introduced into an electroporation chamber 7510 to which a voltage V_{chip} may be applied. Fluid resistance R_{b} corresponds to a fluid (e.g., cell mixture) resistance. Assuming a uniform cell distribution, the fluid resistance R_{b} should be consistent, also assuming the same volume of each fluid batch being electroporated. Rᵢ corresponds to a resistance between fluid and electrode, which increases over time through the electroporation process. In a conventional electroporation process, voltage Vₚₛ is constant. However, due to the increasing Rᵢ over time, the voltage applied to the fluid will decrease over time, leading to lower electrical field strength.

Due to variations in fluid resistance R_{b} and the low number of pulses that may be applied, interpolation to compensate for reduced electrical field strength may not accurately compensate for electrode passivation.

In some variations, a method of electroporating cells may comprise receiving a first fluid comprising cells in a fluid conduit, applying a resistance measurement signal to the first fluid using a set of electrodes, measuring a resistance between the first fluid and the set of electrodes, and applying an electroporation signal to the first fluid based on the measured resistance. In some variations, a second fluid comprising a gas may be received in the fluid conduit before applying the electroporation signal to the fluid. The first fluid may be separated from a third fluid by the second fluid.

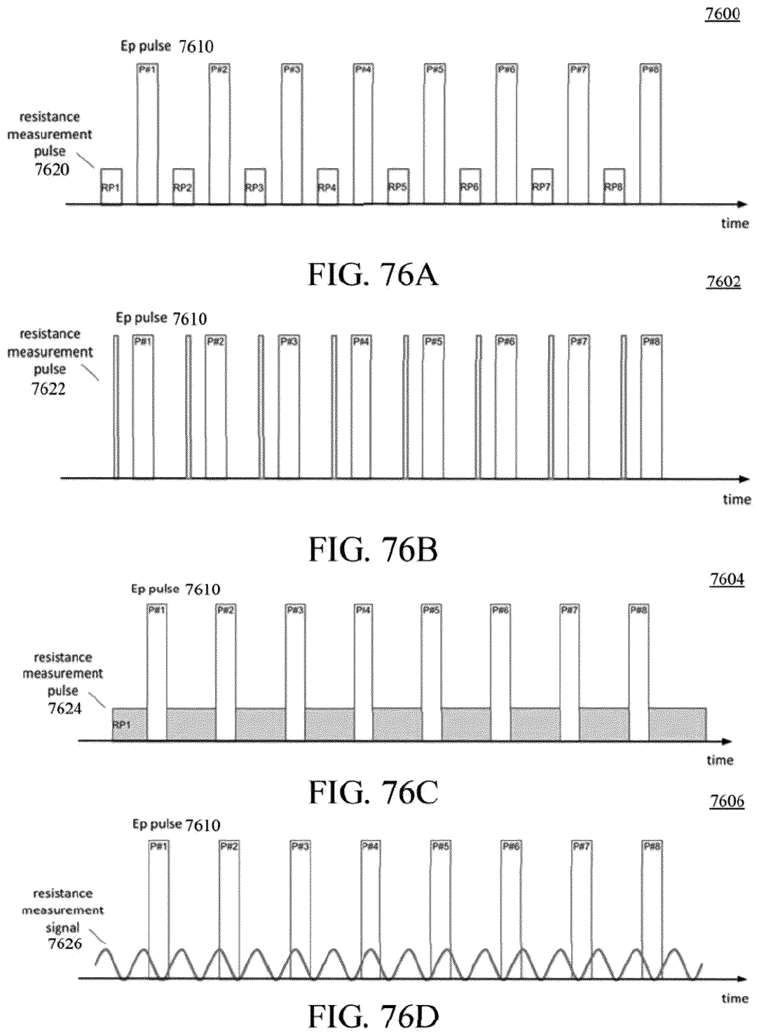

FIGS. 76A-76D are plots 7600, 7602, 7604, 7606 of measurement waveforms and electroporation waveforms. FIG. 76A depicts a first resistance measurement pulse 7620 with a low voltage and a wide pulse width. FIG. 76B depicts a second resistance measurement pulse 7622 with a high voltage and a short pulse width. FIG. 76C depicts a third resistance measurement pulse 7624 with a continuous low voltage waveform to monitor an impedance change continuously over time. FIG. 76D depicts a fourth resistance measurement pulse 7626 with a low AC voltage waveform to monitor an impedance change continuously over time. Each of the resistance measurement pulses avoid inducing electroporation in the cells by reducing voltage and/or pulse width. By monitoring the voltage current of the applied resistance measurement pulse, a change in resistance may be measured and the electroporation pulse applied to a cell batch may be compensated accordingly.

In some variations, an electroporation signal may comprise between about 1 pulse and about 50 pulses, a voltage of between about 100 V and about 700 V, a pulse width of between about 100 μs and about 1 ms, a pulse spacing between about 5 second to about 30 seconds, a resistance pulse voltage of between about 10 V and about 40 V, and a resistance pulse width of between about 10 μs and about 50 μs.

For example, an eight-batch electroporation run may receive one electroporation pulse per batch. Each electroporation pulse may have an electrical field strength between about 0.5 kV/cm and about 2.0 kV/cm. The resistance measurement pulse applied before each batch may have an electrical field strength less than about 0.2 kV/cm such that electroporation is not induced by the resistance measurement pulse.

### Sterile Liquid Transfer Device

Generally, the sterile liquid transfer devices described herein may be configured to store fluid for transfer to another component of a cell processing and manufacturing system such as a cartridge, bioreactor, and the like. In some variations, the sterile liquid transfer device may comprise a portable consumable configured to be moved using a robot. For example, a robot may be configured to move a sterile liquid transfer device from a reagent vault to an ISO 7 space to a sterile liquid transfer instrument within a cell processing and manufacturing system. The sterile liquid transfer device enables the transfer of fluids in an automated, sterile, and metered manner for automating cell therapy manufacturing.

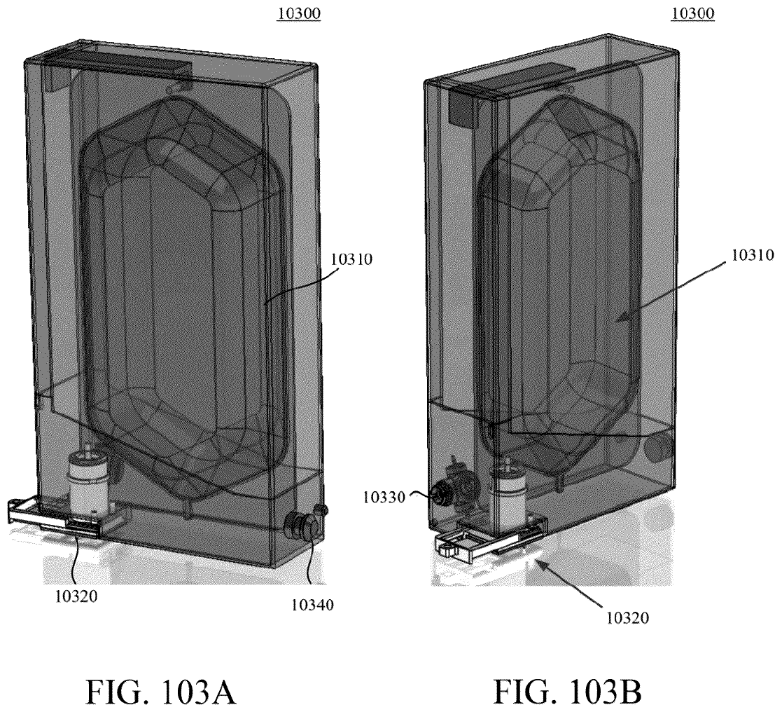

FIGS. 103A and 103B are perspective views of a sterile liquid transfer device 10300 comprising a fluid cavity 10310 (e.g., container, vessel), fluid connector 10320, and pump 10330. Fluid stored within fluid cavity 10310 may be transferred in and out of the sterile liquid transfer device 10300 through the fluid connector 10320 using the pump 10330. In some variations, the sterile liquid transfer device 10300 may comprise an engagement feature 10340 (e.g., robot mount) to facilitate robotic arm control.

### Fluid Connector

Generally, the aseptic fluid connectors described herein may form a sterile fluid pathway between at least two fluid devices to enable fluid transfer that may be one or more of sterile, fully automated, and precisely metered (e.g., precise control of a transferred fluid volume). In some variations, the robot may be configured to couple a fluid connector between at least two sterile liquid transfer devices and a plurality of cartridge modules. In some variations, the robot may be configured to operate the fluid controller to open and close a set of ports and valves of the fluid connector. The use of a robot and controller to operate the fluid connector may facilitate automation and sterility of a cell processing and manufacturing system.

In some variations, a system may comprise a robot configured to operate a fluid connector as described herein, and a controller comprising a memory and processor. The controller may be coupled to the robot. The controller may be configured to generate a port signal to couple the first port to the second port using the robotic arm, generate a first valve signal to translate the first valve relative to the second valve using the robotic arm, and generate a second valve signal to transition the first valve and the second valve to the open configuration.

In some variations, a fluid pump may be coupled to the sterilant source, and the controller may be configured to generate a first fluid signal to circulate a fluid into the chamber through the sterilant port. The controller may be configured to generate a second fluid signal to circulate the sterilant into the chamber through the sterilant port to sterilize at least the chamber. The controller may be configured to generate a third fluid signal to remove the sterilant from the chamber.

In some variations, the controller may be configured to generate a port signal to couple the first port to the second port using the robot, generate a first valve signal to translate the first valve relative to the second valve using the robot, and generate a second valve signal to transition the first valve and the second valve to the open configuration.

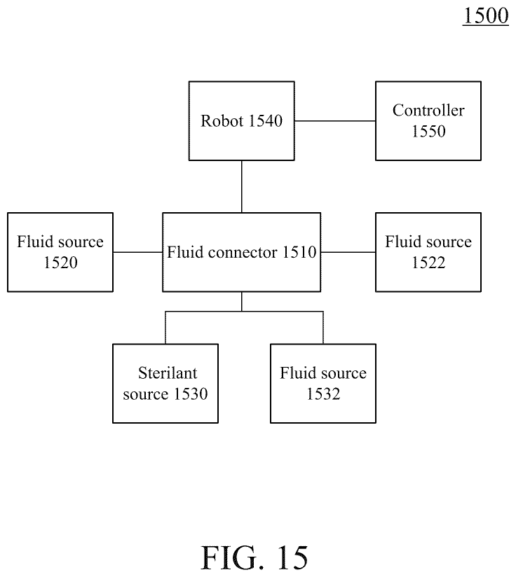

The fluid connector may further allow for a plurality of connection cycles in a sterile system and may be controlled without human intervention. For example, the fluid connector may comprise one or more of engagement features to facilitate robotic control and alignment features to ensure proper connection between connector components. FIG. 15 is a block diagram of an illustrative variation of a fluid connector system 1500 comprising a fluid connector 1510, a first fluid device 1520 (e.g. cartridge, cartridge module, sterile liquid transfer device (SLTD)), a second fluid device 1522 (e.g., cartridge, cartridge module, SLTD), sterilant source 1530, fluid source 1532, robot 1540, and controller 1550.

The fluid connector 1510 may be removably coupled (e.g., connected/disconnected, attached/detached) to each of the first fluid device 1520, second fluid device 1522, sterilant source 1532, fluid source 1532, and robot 1540. In some variations, a fluid device may comprise one or more of a cartridge and sterile liquid transfer device. For example, a sterile liquid transfer device may be in fluid communication with a cartridge via the fluid connector.

As described in more detail herein, separate portions (e.g., male connector, female connector) of the fluid connector 1510 may be removably coupled to each other. The robot 1540 may be configured to physically manipulate (e.g., removably couple) one or more of the fluid connector 1510, first fluid device 1520, second fluid device 1522, sterilant source 1530, and fluid source 1532 in a predetermined manner. For example, the robot 1540 may connect the fluid connector 1510 between the first fluid device 1520 and the second fluid device 1522. The robot 1540 may also connect the sterilant source 1530 and/or fluid source 1532 to a sterilant port of the fluid connector 1510. In some variations, the robot 1540 may control one or more valves and/or ports of the fluid connector 1510, and thereby initiate a sterilization process for one or more portions of the fluid connector 1510 using, for example, sterilant from the sterilant source 1530. The controller 1550 may be coupled to one or more of the robot 1540, sterilant source 1530, and fluid source 1532 to control one or more of fluid transfer and sterilization.

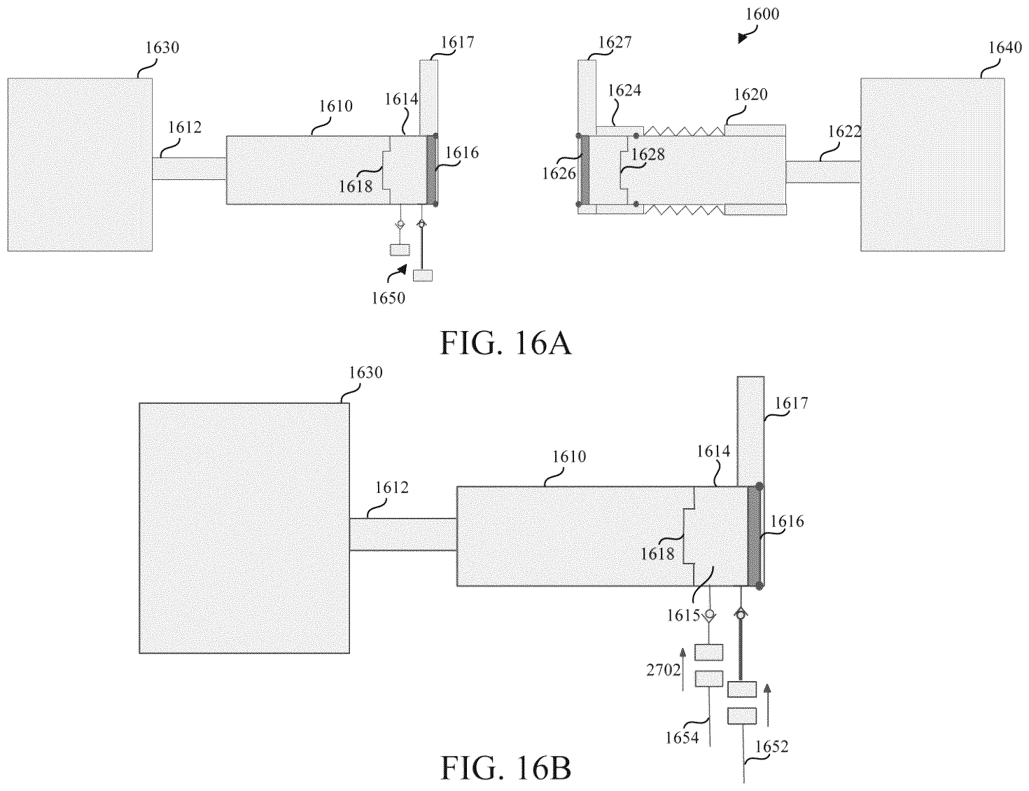

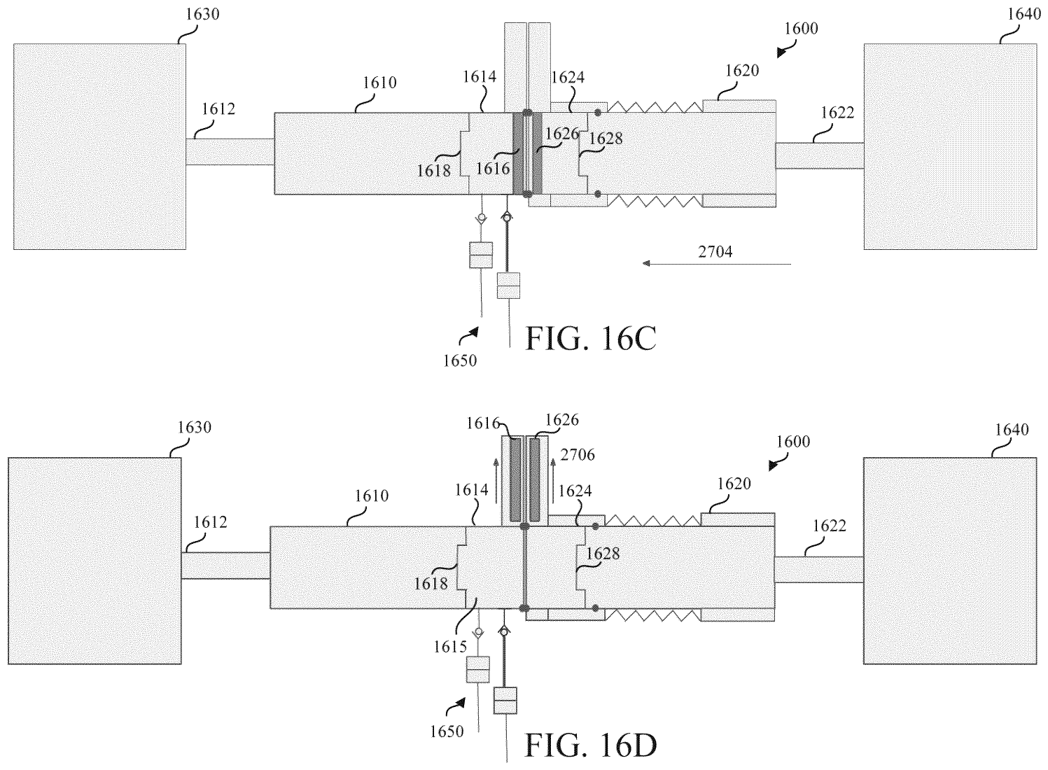

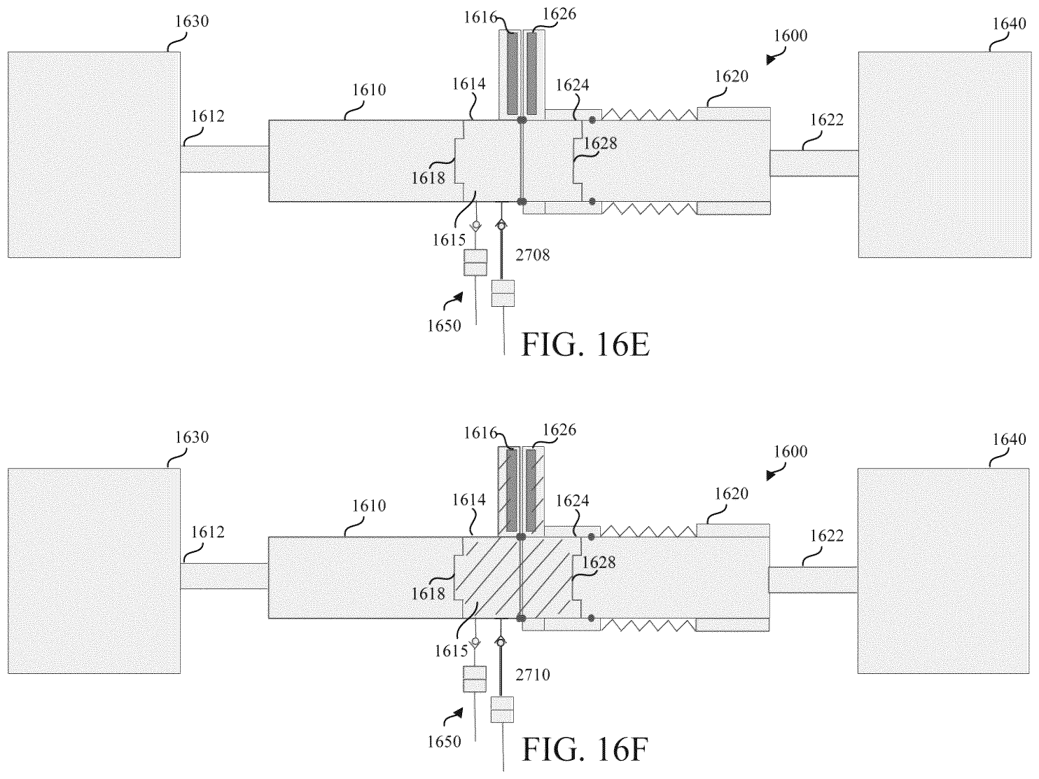

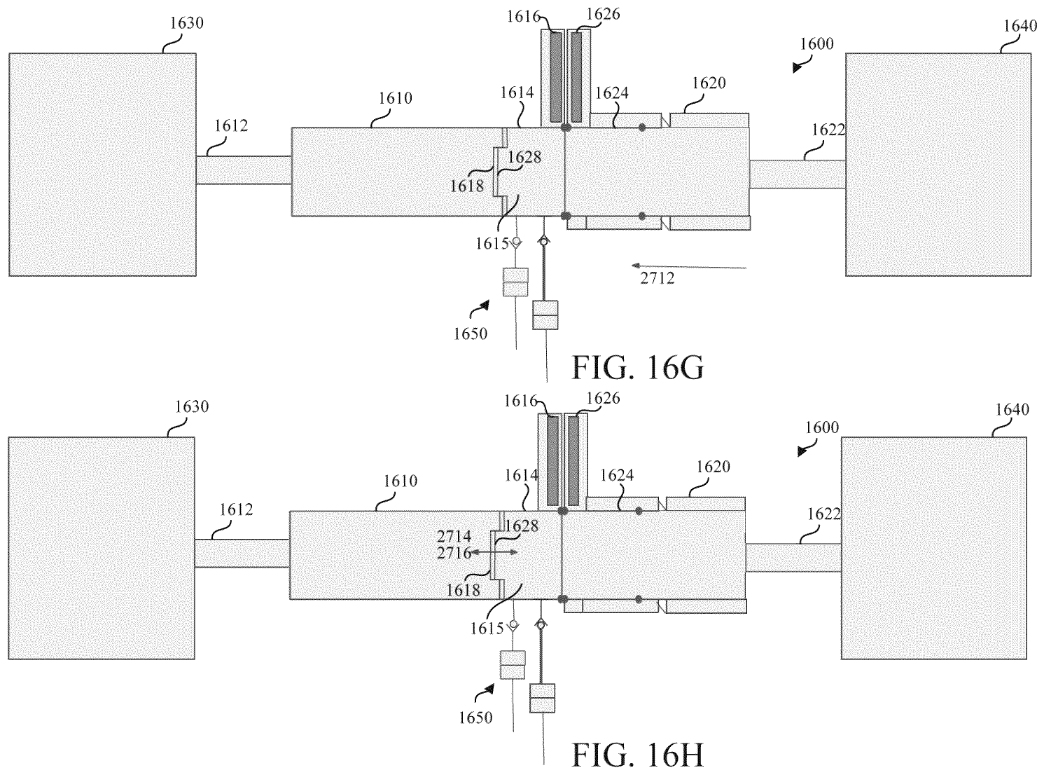

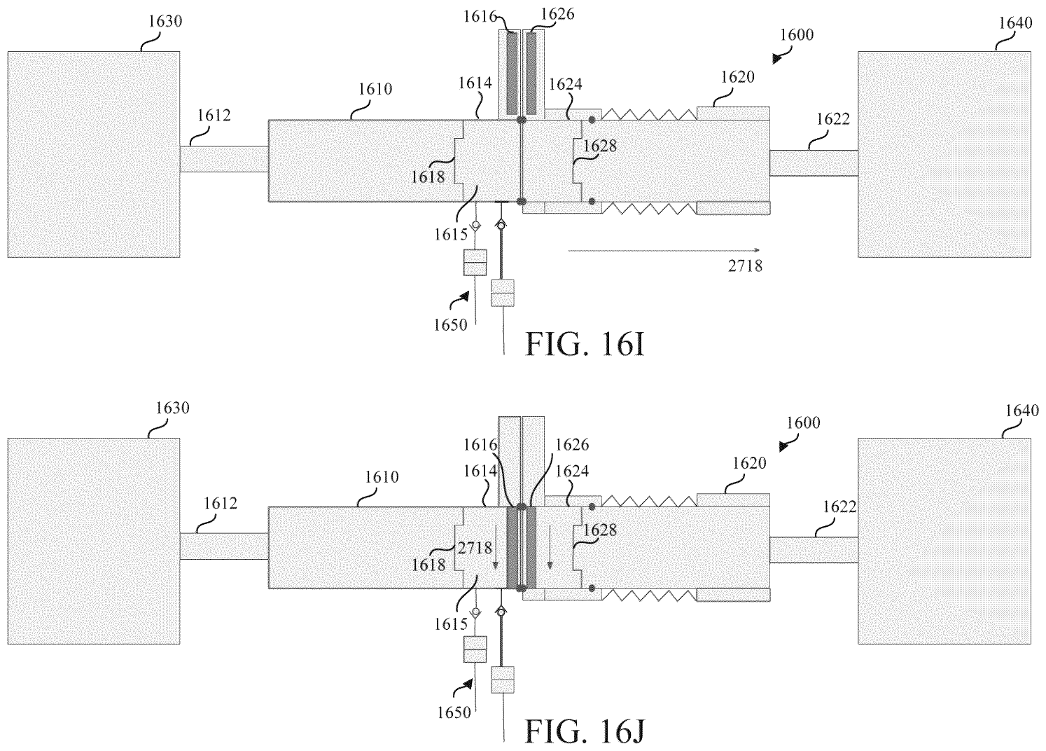

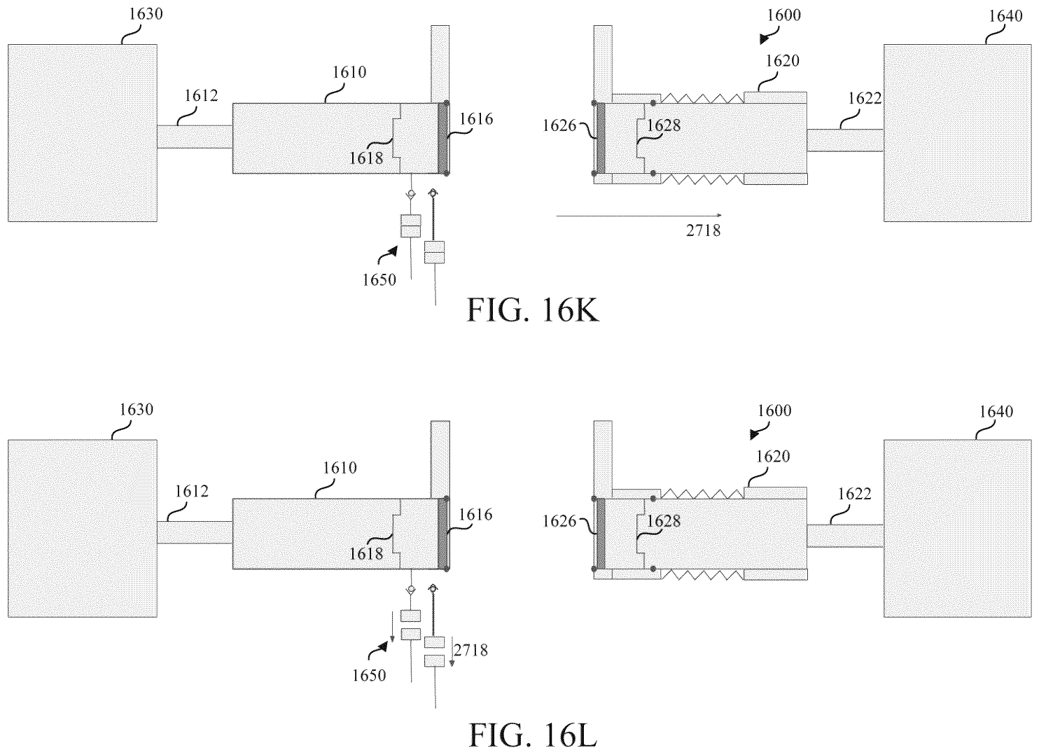

FIG. 16A is a schematic diagram of an illustrative variation of a fluid connector 1600. The fluid connector 600 may comprise a lumen extending along its length and be disposed between a first fluid device 1630 and a second fluid device 1640 to enable fluid flow through the fluid connector 1600. In some variations, the first fluid device 1630 and second fluid device 1640 may be aseptically connected and disconnected using the fluid connector 1600. The fluid devices 1630, 1640 may comprise a closed sterile device, and may be the same or different types of fluid devices. For example, the fluid devices 1630, 1640 may comprise one or more of a sterile liquid transfer device and a consumable (e.g., a cartridge). In some variations, the fluid connector 1600 may comprise a first connector 1610 including a first proximal end 1612 and a first distal end 1614. The first proximal end 1612 may be configured to couple to the first fluid device 1630. The first distal end 1614 may include a first port 1616, first housing 1617, and a first valve 1618. The first housing 1617 may be configured to receive the first port 1616 in a closed configuration as described in more detail herein.

The fluid connector 1600 may further comprise a second connector 1620 including a second proximal end 1622 and a second distal end 1624. The second proximal end 1622 may be configured to couple to the second fluid device 1640. The second distal end 1624 may include a second port 1626, second housing 1627, and a second valve 1628. The second housing 1627 may be configured to receive the second port 1626 in a closed configuration. In FIG. 16A, the first connector 1610 comprises a sterilant port 1650 configured to couple to a sterilant source (not shown). Additionally or alternatively, the second connector 1620 may comprise the sterilant port 1650. The sterilant port 1650 may be configured to be in fluid communication with the first distal end 1614 and the second distal end 1624 when the second port 1626 is coupled to the first port 1616 as described in more detail herein.

In some variations, a fluid device 1630, 1640 may comprise a sterilant chamber and a sterilant port configured to receive a sterilant. The sterilant chamber may enclose a fluid device connector (not shown) configured to couple to a proximal end of a first connector 1610 or second connector 1620. The fluid device 1630, 1640 may receive a sterilant in a similar manner as the fluid connector 1600.

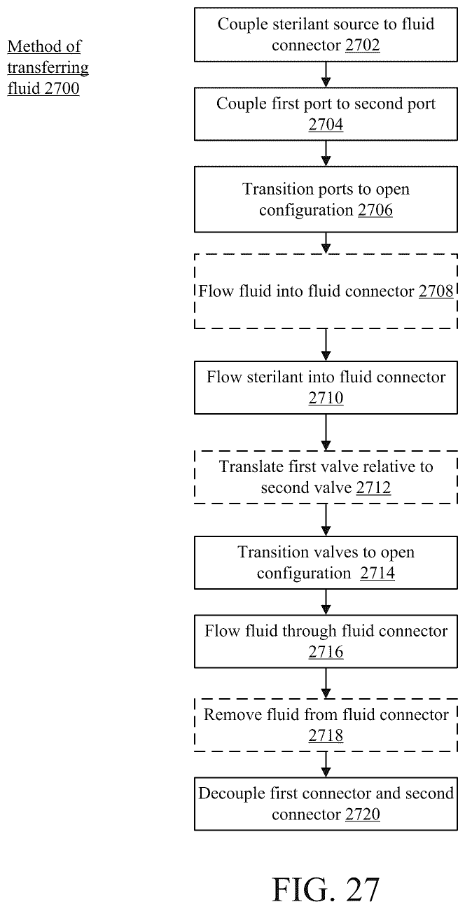

FIG. 16B is a detailed schematic diagram of the first connector 1610 including a first port housing 1617 and a chamber 1615. The chamber 1615 may be defined by the cavity enclosed by one or more of the distal ends 1614, 1624. For example, the chamber 1615 in FIG. 16B may comprise the portion of the first connector 1610 between the first valve 1618 and the first port 1616 in the closed configuration (e.g., the first distal end 1614). In some variations, the first chamber 1615 may comprise a volume of between about 1 cm³ and about 5 cm³. When the first connector 1610 is coupled to the second connector 1620 and the ports 1616, 1626 are in an open configuration (as shown in FIG. 16D), the chamber 1616 may comprise the portion of the fluid connector 1600 between the first valve 1618 and the second valve 1628 (e.g., the first distal end 1614 and second distal end 1624). The chamber 1615 may comprise an enclosed volume configured to receive a fluid such as a sterilant from the sterilant port 1650. In some variations, the sterilant port 1650 may comprise an inlet 1652 and outlet 1654. Methods of using a fluid connector are described in more detail with respect to FIGS. 16C-16L and 27.

In some variations, the fluid connector 1600 may comprise one or more alignment features and robot engagement features configured to facilitate robotic manipulation, as described in more detail herein. In some variations, the fluid connector 1600 may be coupled to one or more sensors, pumps, and valves to facilitate fluid transfer and monitoring.

In some variations, the components of the fluid connector in contact with fluid may be USP Class VI compatible for cell processing and/or GMP applications. In some variations, the components of the fluid connector may be composed of a material including, but not limited to, one or more of cyclic olefin copolymer (COC), polychlorotrifluoroethylene, polyetherimide, polysulfone, polystyrene, polycarbonate, polypropylene, silicone, polyetheretherketone, polymethylmethacrylate, nylon, acrylic, polyvinylchloride, vinyl, phenolic resin, petroleum-derived polymers, glass, polyethylene, terephthalate, metal, stainless steel, titanium, aluminum, cobalt-chromium, chrome, silicates, glass, alloys, ceramics, carbohydrate polymer, mineraloid matter, and combinations or composites thereof.

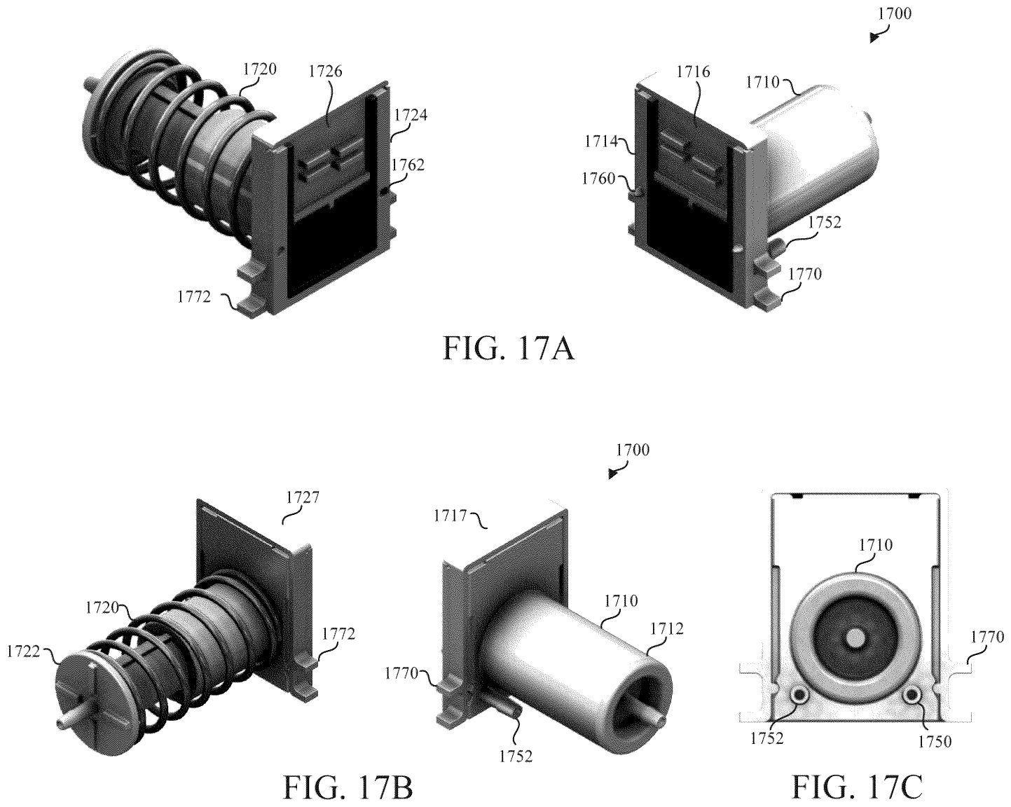

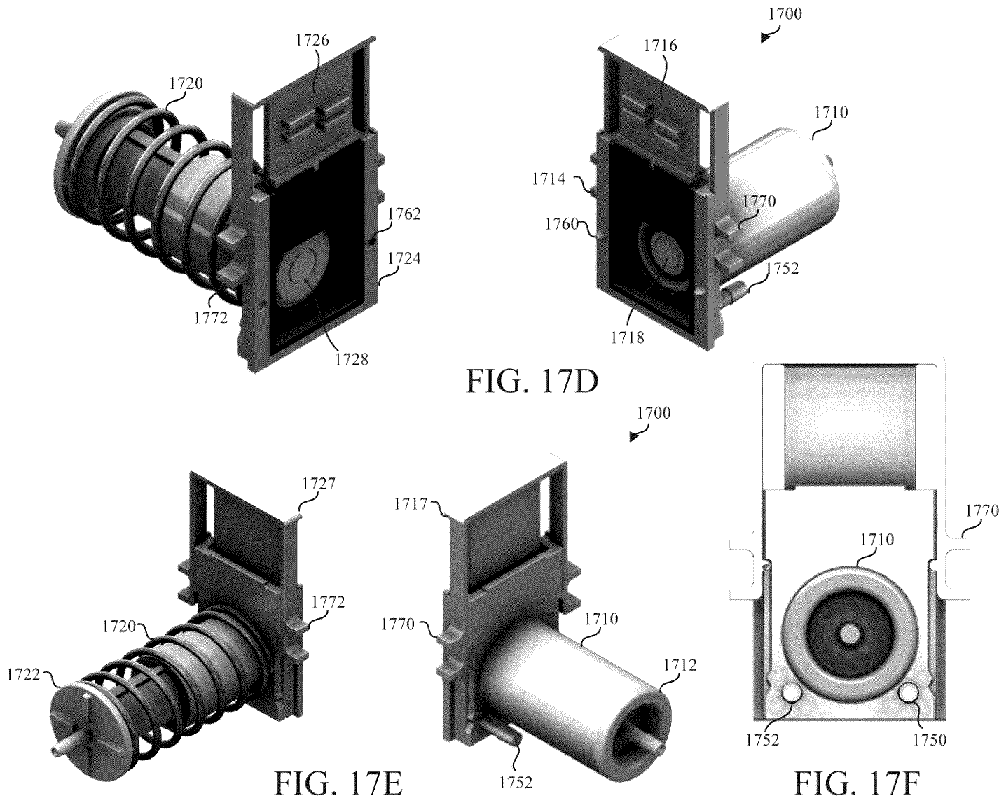

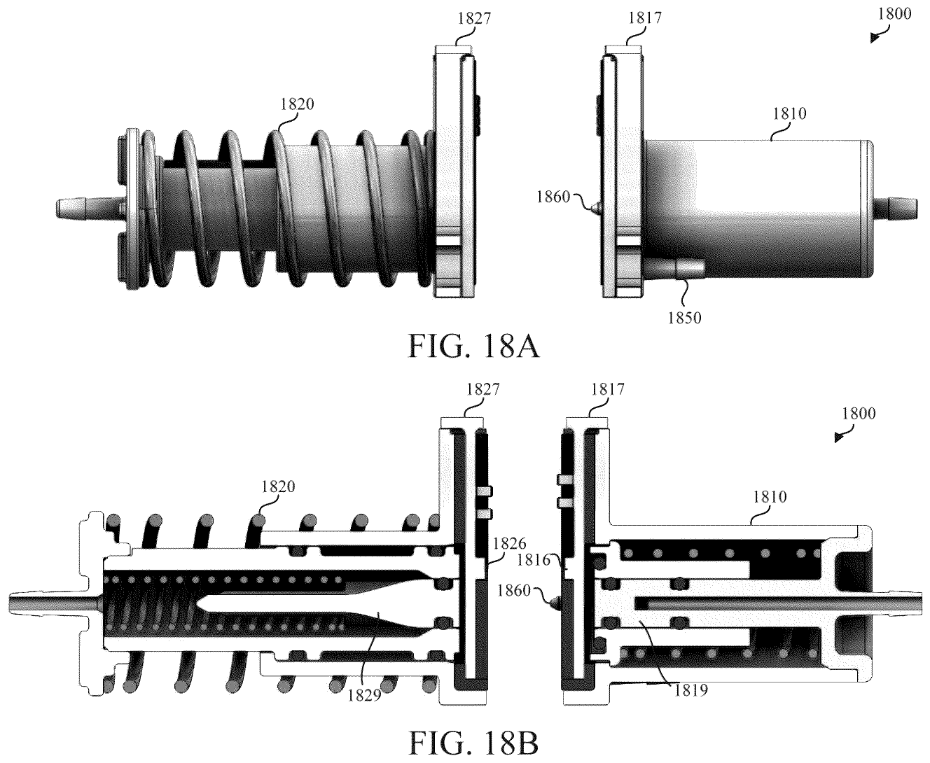

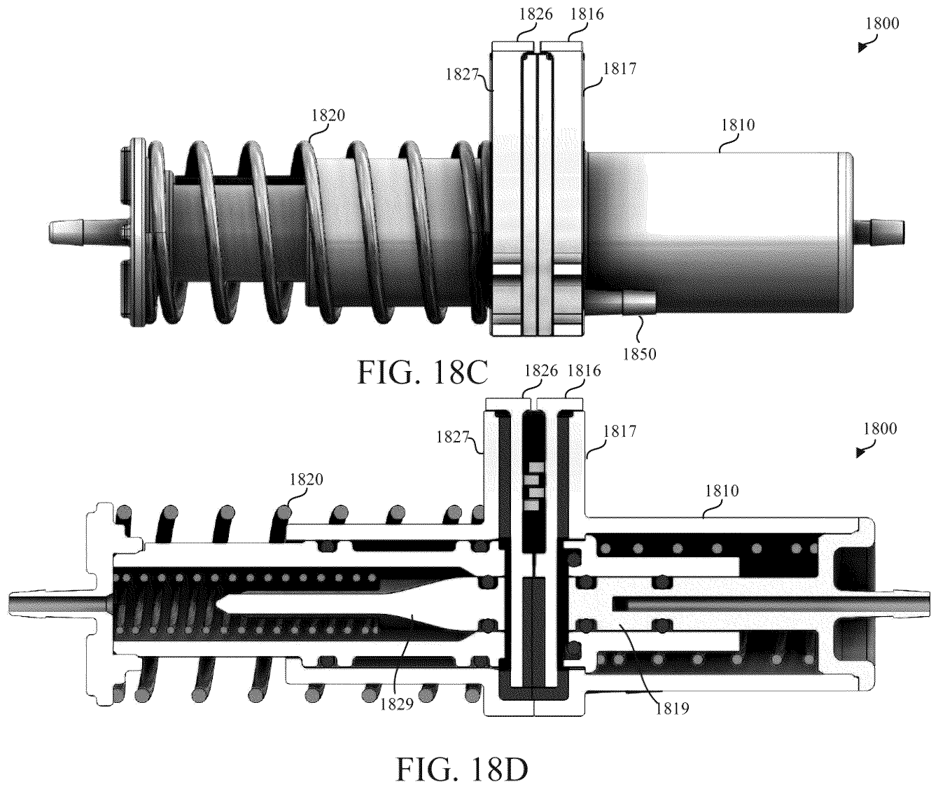

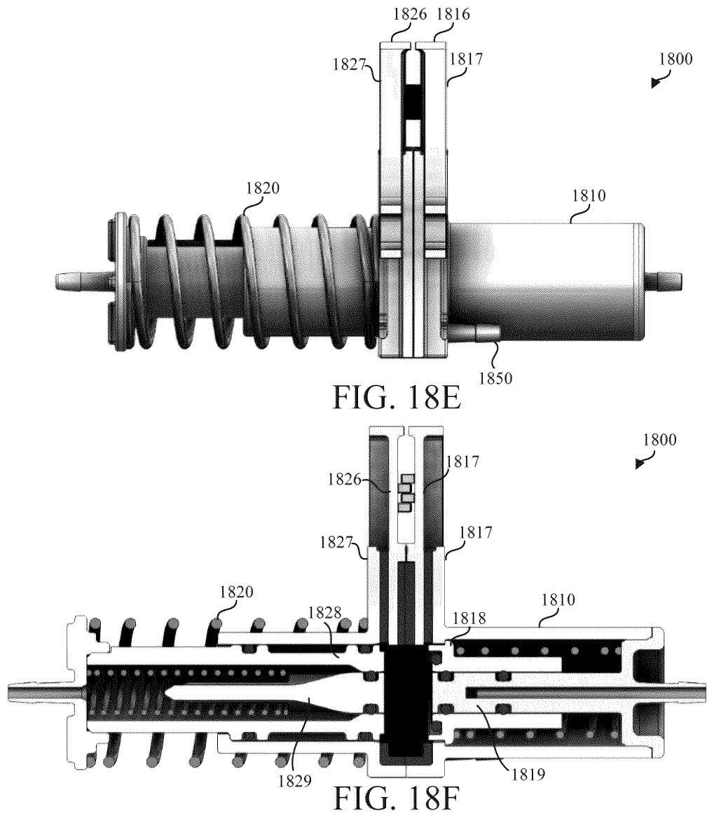

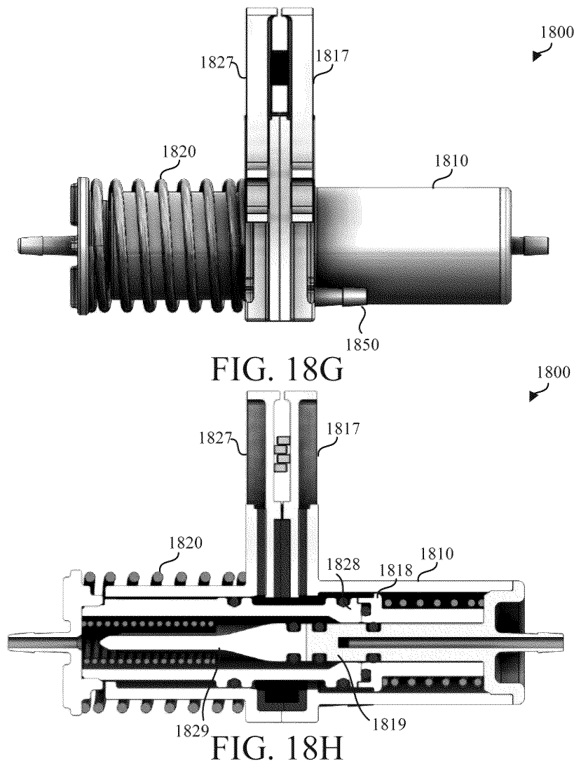

FIGS. 17A-18D depict external and internal views of variations of a fluid connector. FIG. 17A is a front perspective view of a fluid connector 1700 in a closed port configuration. FIG. 17B is a rear perspective view and FIG. 17C is a rear view of the fluid connector 1700. Generally, the fluid connector may comprise a plurality of internal seals to reduce contamination and aid sterilization, as well as alignment features to aid proper registration of the fluid connector components.

The fluid connector 1700 may comprise a lumen extending along its length. In some variations, the fluid connector 1700 may comprise a first connector 1710 including a first proximal end 1712 and a first distal end 1714. The first proximal end 1712 may be configured to couple to a first fluid device (not shown for the sake of clarity). The first proximal end 1712 may comprise a Luer connector or any other suitable connector. The first distal end 1714 may include a first port 1716 and first housing 1717. The first housing 1717 is shown in FIG. 17A holding the first port 1716 in a closed configuration. The first connector 1710 further comprises a sterilant port 1750, 1752 configured to couple to a sterilant source (not shown for the sake of clarity). In some variations, the sterilant port may comprise an inlet and outlet. In some variations, the sterilant port may optionally comprise one or more of a check valve and particle filter configured to reduce contamination into the sterilant port when not connected to a robot or actuator. The first connector 1710 may comprise a first alignment feature 1760 such as a set of protrusions on the first distal end 1714 of the first connector 1710. The alignment features may ensure that small positioning errors due to robotic manipulation do not impact the operation of the fluid connector.

The fluid connector 1700 may further comprise a second connector 1720 including a second proximal end 1722 and a second distal end 1724. The second proximal end 1722 may be configured to couple to the second fluid device (not shown for the sake of clarity). The second proximal end 1722 may comprise a Luer connector or any other suitable connector. The second distal end 1724 may include a second port 1726 and second housing 1727. The second housing 1727 is shown in FIG. 17A holding the second port 1726 in the closed configuration. The second connector 1720 may comprise a second alignment feature 1762 such as a set of holes on the second distal end 1724 of the second connector 1720. The second alignment feature 1762 may be configured to couple to the first alignment feature 1760 in a predetermined axial and rotational configuration to aid mating of the first connector 1710 and the second connector 1720.

The first port 1716 and the second port 1726 retained within respective first housing 1717 of the first distal end 1714 and second housing 1727 of the second distal end 1724 facilitates robotic control as the ports 1716, 1726 are not separable from the fluid connector 1700, and therefore reduces the risk of failure of automated handling by a robot.

In some variations, the first connector 1710 may comprise a first robot engagement feature 1770 and the second connector 1720 may comprise a second robot engagement feature 1772. The robot engagement features 1770, 1772 may be configured to be manipulated by a robot (e.g., robot 1540). In some variations, the robot engagement features 1770, 1772 may be operatively coupled to a respective first port 1716 and second port 1726 and configured to actuate the ports 1716, 1726 between a closed port configuration and an open port configuration, as shown in FIGS. 17A-17F. Additionally or alternatively, a user may manually actuate the robot engagement features 1770, 1772 to actuate respective ports 1716, 1726.

FIG. 17D is a front perspective view of the fluid connector 1700 in an open port configuration. FIG. 17E is a rear perspective view and FIG. 17F is a rear view of the fluid connector 1700 in the open port configuration. In the open port configuration, the first valve 1718 of the first connector 1710 and the second valve 1728 of the second connector 1720 are shown in FIG. 17D.

FIG. 18A is a side view and FIG. 18B is a cross-sectional side view of a fluid connector 1800 in an uncoupled configuration. In some variations, the fluid connector 1800 may comprise a first connector 1810 including a first housing 1817 comprising a first port 1816, a sterilant port 1850 configured to couple to a sterilant source (not shown), a first alignment feature 1860 configured to couple to a corresponding alignment feature (not shown) of the second connector 1820. The fluid connector 1800 may comprise a second connector 1820 including a second housing 1827 comprising a second port 1826. The first connector 1810 and second connector 1820 may be axially aligned and alignment features may aid rotational alignment of the first connector 1810 to the second connector 1820. The first valve 1818 may comprise a first valve stem 1819 and the second valve 1828 may comprise a second valve stem 1829.

FIG. 18C is a side view and FIG. 18D is a cross-sectional side view of the fluid connector 1800 in a coupled configuration where the first housing 1817 and the second housing 527 are brought together but where the first connector 1810 and the second connector 1820 are not in fluid communication since the first port 1816 and the second port 1826 are both in the closed configuration. The first alignment features on each connector 1810, 1820 may be configured to ensure axial and/or rotational alignment between the first connector 1810 and the second connector 1820.

FIG. 18E is a side view and FIG. 18F is a cross-sectional side view of the fluid connector 1800 in an open port configuration. Each of the first port 1817 and the second port 1827 are transitioned from the closed configuration to an open configuration. This creates a closed internal volume within respective distal ends of each connector 1810, 1820. Each of first valve 1818 and second valve 1828 is in a closed configuration such that fluid flow is inhibited between the first connector 1810 and the second connector 1820, still restricted on each half on account of the auto-shutoff valves in both sides.

FIG. 18G is a side view and FIG. 18H is a cross-sectional side view of the fluid connector 1800 in an open valve configuration where the first valve 1818 is coupled to the second valve 1828. For example, the second valve 1828 may be translated along a longitudinal axis of the second connector 1820 towards the first valve 1818. As shown in FIGS. 18G and 18H, the second connector 1820 may be axially compressed to translate the second valve 1828 towards the first valve 1818. The first valve 1818 coupled to the second valve 1828 may form a radial seal, and the first valve stem 1819 and the second valve stem 1829 may be in contact to enable fluid communication between the first connector 1810 and the second connector 1820.

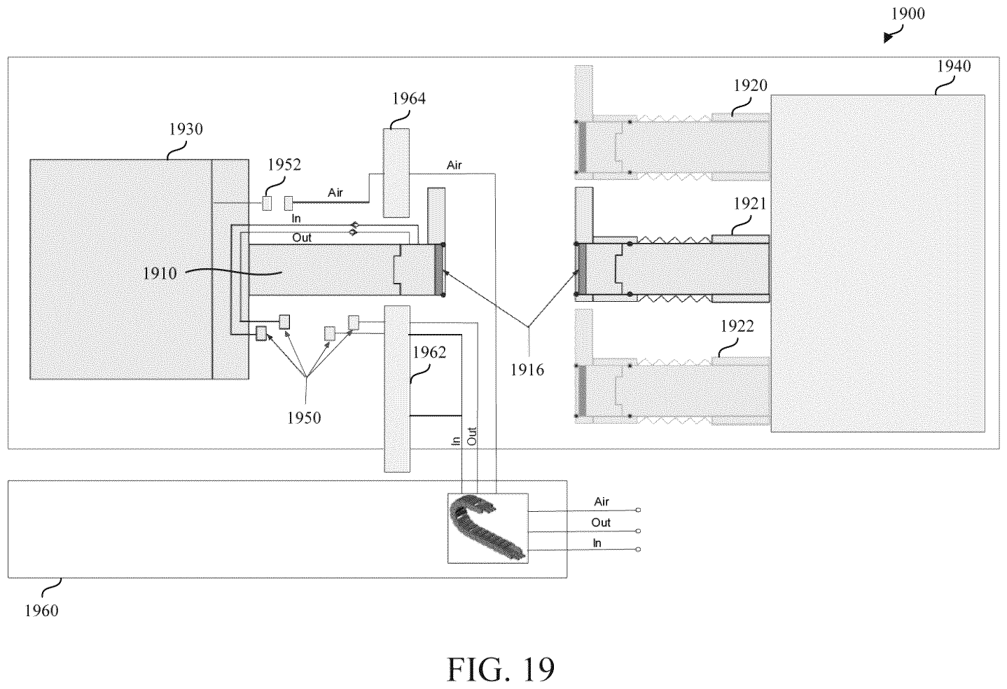

FIGS. 19-26B are schematic diagrams of variations of fluid connector systems for coupling fluid devices. In some variations, a fluid connector may comprise a first connector configured to couple to any one of a plurality of second connectors. FIG. 19 is a schematic diagram of an illustrative variation of a fluid connector system 1900 comprising a first connector 1910, a plurality of second connectors 1920, 1921, 1922, a first fluid device 1930 (e.g., sterile liquid transfer device), a second fluid device 1940 (e.g., consumable), and a robot 1960 (e.g., robotic arm, 3DOF robot). The first connector 1910 may be coupled in fluid communication with the first fluid device 1930, and the second connectors 1920, 1921, 1922 may be coupled in fluid communication with the second fluid device 1940. The first connector 1910 and the second connectors 1920, 1921, 1922 may each comprise a port 1916 configured to couple to a corresponding port as described in more detail herein. The robot 1960 may comprise one or more end effectors 1962, 1964 configured to manipulate and/or couple to one or more of the first fluid device 1930 and first connector 1910. For example, the first connector 1910 may comprise one or more sterilization ports 1950 configured to couple to an end effector 1962 (e.g., gripper). Similarly, the first fluid device 1930 may comprise one or more fluid ports 1952 configured to couple to an end effector 1964.

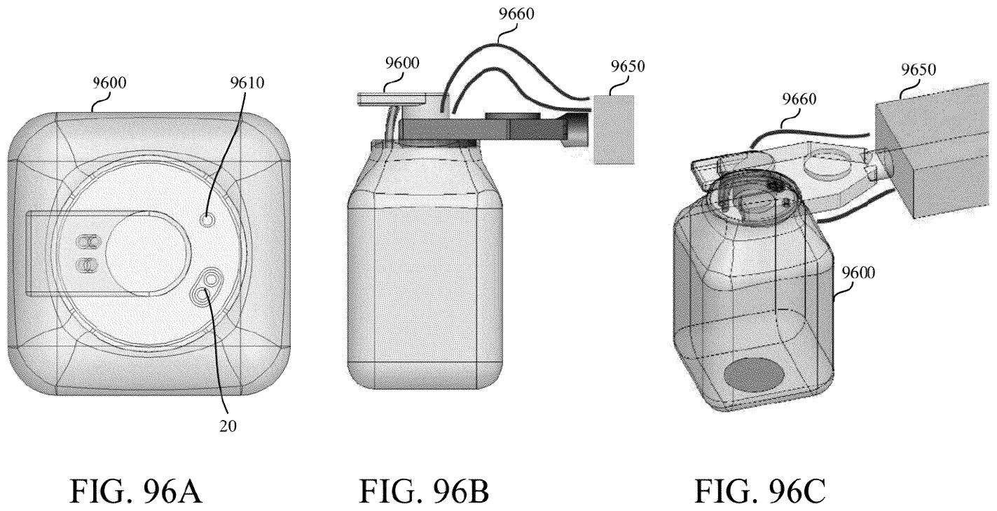

In some variations, the robot 1960 may be configured to couple to one or more of a sterilant source, fluid source, and pump in order to facilitate efficient and shared fluidic connections between the fluid device, fluid connector, and a sterilization system. For example, FIG. 96A is a plan view of a fluid device 9600 (e.g., sterile liquid transfer device) comprising a fluid port 9610 configured to couple to a fluid source (not shown) and a sterilization port 9620 configured to couple to a sterilant source (not shown). The FIGS. 96B and 96C are respective side and perspective views of a fluid device 9600 coupled to a robot 9650. In some variations, the robot 9650 may comprise one or more fluid conduits 9660 configured to couple to one or more of the fluid port 9610 and sterilization port 9620 of the fluid device 9600.

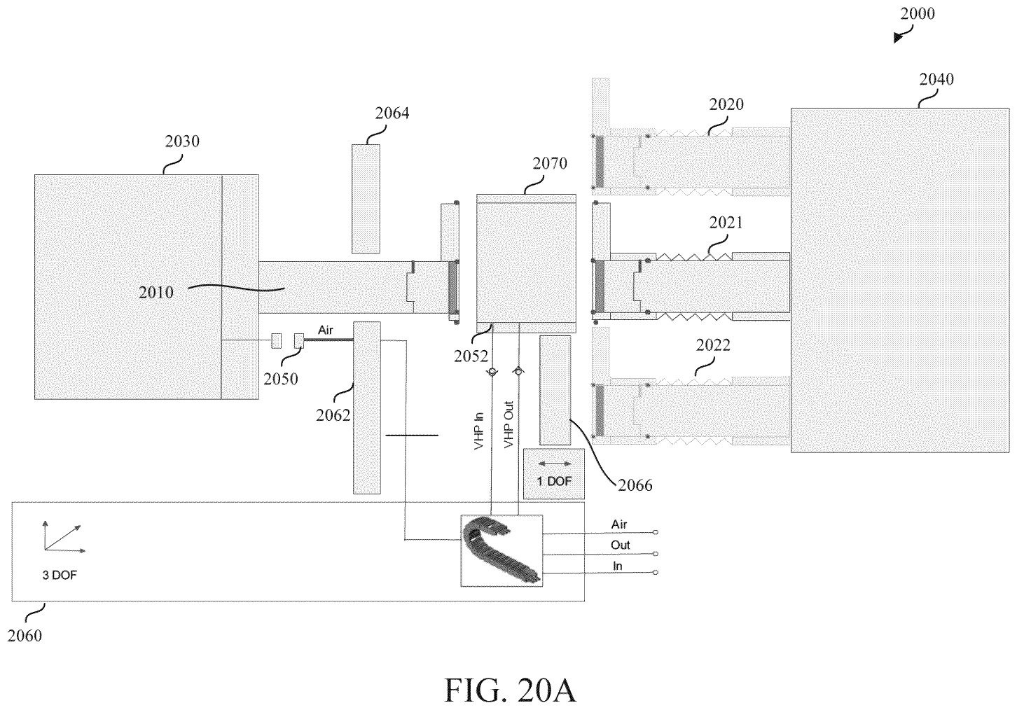

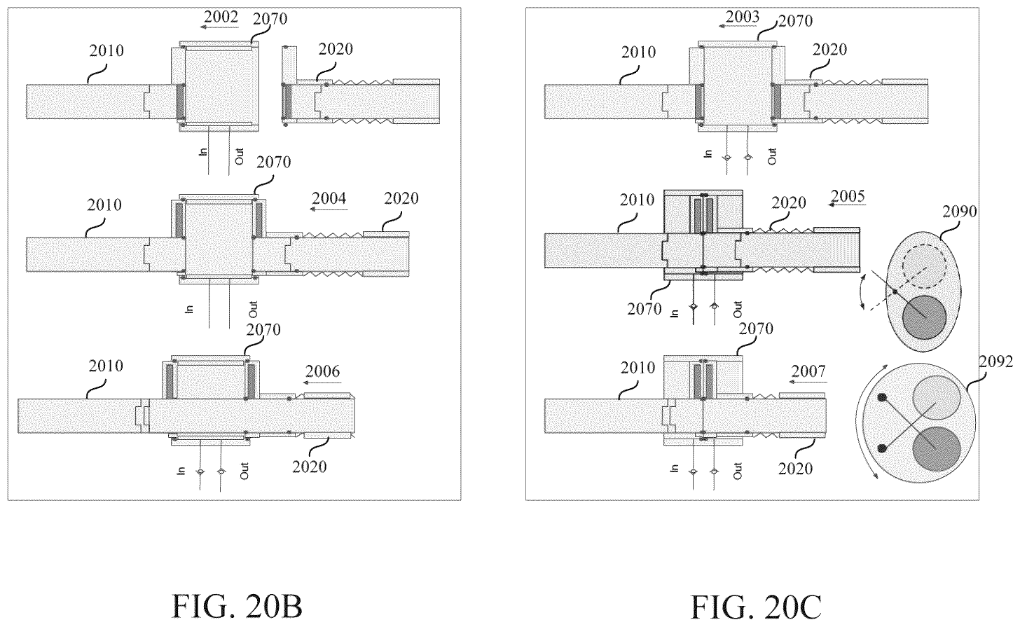

In some variations, a fluid connector may comprise a third connector disposed between a first connector and a second connector. FIG. 20A is a schematic diagram of an illustrative variation of a fluid connector system 2000 comprising a first connector 2010, a plurality of second connectors 2020, 2021, 2022, a third connector 2070 (e.g., instrument, sterilization enclosure), a first fluid device 2030 (e.g., sterile liquid transfer device), a second fluid device 2040 (e.g., a cartridge), and a robot 2060 (e.g., 3 DOF robot, 1 DOF robot). The first connector 2010 may be coupled in fluid communication with the first fluid device 2030, and the second connectors 2020, 2021, 2022 may be coupled in fluid communication with the second fluid device 2040. The third connector 2070 may be coupled between the first connector 2010 and one of the second connectors 2020, 2021, 2022. The third connector 2070 may comprise a lumen configured to receive and circulate a sterilant through one or more portions of the first connector 2010, second connector 2020, 2021, 2022, and third connector 2070. In some variations, the sterilization port 2052 may be non-removably coupled to a sterilant source and/or fluid source, thereby simplifying one or more of the first fluid device 2030 and first connector 2010.

The robot 2060 may comprise one or more end effectors 2062, 2064, 2066 configured to manipulate and/or couple to one or more of the first fluid device 2030, first connector 2010, and third connector 2070. For example, the first fluid device 2030 may comprise one or more fluid ports 2050 configured to couple to an end effector 2062. Similarly, the third connector 2070 may comprise one or more sterilization ports 2052 configured to couple to robot 2060 (e.g., end effector 2064). In some variations, the robot 2060 may be configured to couple to one or more of a sterilant source, fluid source, and pump in order to facilitate efficient and shared fluidic connections between the fluid device, fluid connector, and a sterilization system.

FIGS. 20B and 20C are schematic diagrams of a fluid connector connection process. In FIG. 20B, a third connector 2070 may be coupled to a distal end of a first connector 2010, at 2002. A distal end of the second connector 2020 may be coupled to the third connector 2070, at 2004. The second connector 2020 may be translated through the third connector 2070 to directly couple the second connector 2020 to the first connector 2010, at 2006.

In FIG. 20C, a third connector 2070 may be coupled to a distal end of the first connector 2010 and a distal end of the second connector 2020, at 2002. Each of the first connector 2010 and the second connector 2020 may be translated toward each other through the third connector 2070, at 2005. The second connector 2020 may be further translated towards the first connector 2010 to directly couple the first connector 2010 to the second connector 2010, at 2007. FIG. 20C further illustrates a first port 2090 and a second port 2092 that may transition between a closed port configuration and an open port configuration.

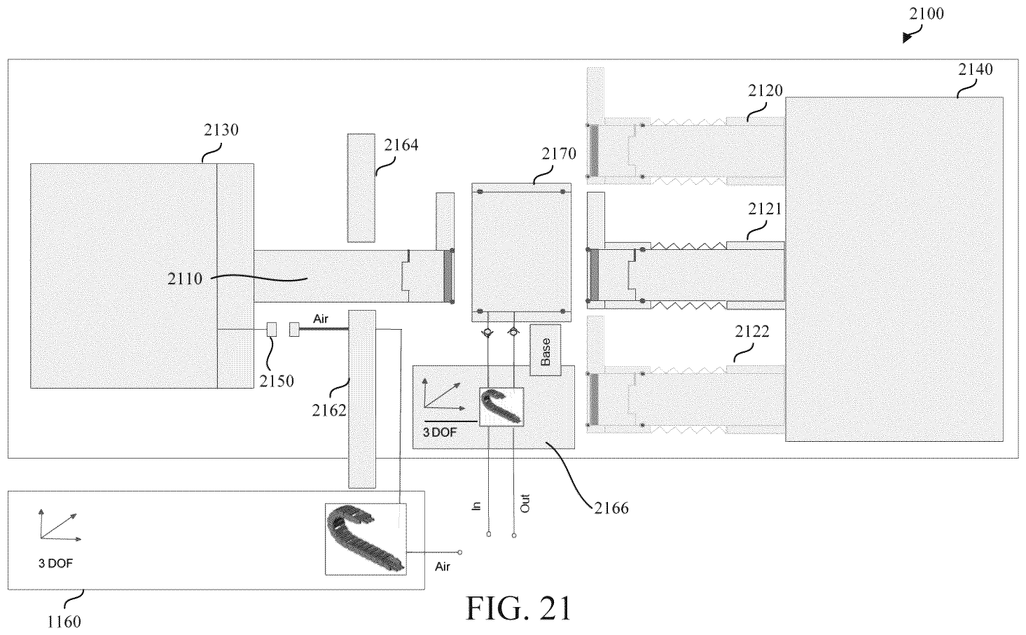

In some variations, a fluid connector may comprise a third connector disposed between a first connector and a second connector. The third connector may be coupled to a second robot different from a first robot coupled to the first connector. FIG. 21 is a block diagram of an illustrative variation of a fluid connector system 2100 comprising a first connector 2110, a plurality of second connectors 2120, 2121, 2122, a third connector 2170 (e.g., instrument, sterilization enclosure), a first fluid device 2130 (e.g., sterile liquid transfer device), a second fluid device 2140 (e.g., consumable), a first robot 2160, and a second robot 2166. The first connector 2110 may be coupled in fluid communication with the first fluid device 2130, and the second connectors 2120, 2121, 2122 may be coupled in fluid communication with the second fluid device 2140. The third connector 2170 may be coupled between the first connector 2110 and one of the second connectors 2120, 2121, 2122. The third connector 2170 may comprise a lumen configured to receive and circulate a sterilant through one or more portions of the first connector 2110, second connector 2120, 2121, 2122, and third connector 2170. In some variations, the third connector 2170 may be non-removably coupled to a sterilant source and/or fluid source, thereby simplifying one or more of the first fluid device 2130 and first connector 2110.

The first robot 2160 may comprise one or more end effectors 2162, 2164 configured to manipulate and/or couple to one or more of the first fluid device 2130 and first connector 2110. For example, the first fluid device 2130 may comprise one or more fluid ports 2150 configured to couple to an end effector 2162. The third connector 2170 may be coupled to a second robot 2166 (e.g., 3 DOF robot). In some variations, the robot 2160, 2166 may be configured to couple to one or more of a sterilant source, fluid source, and pump in order to facilitate efficient and shared fluidic connections between the fluid device, fluid connector, and a sterilization system.

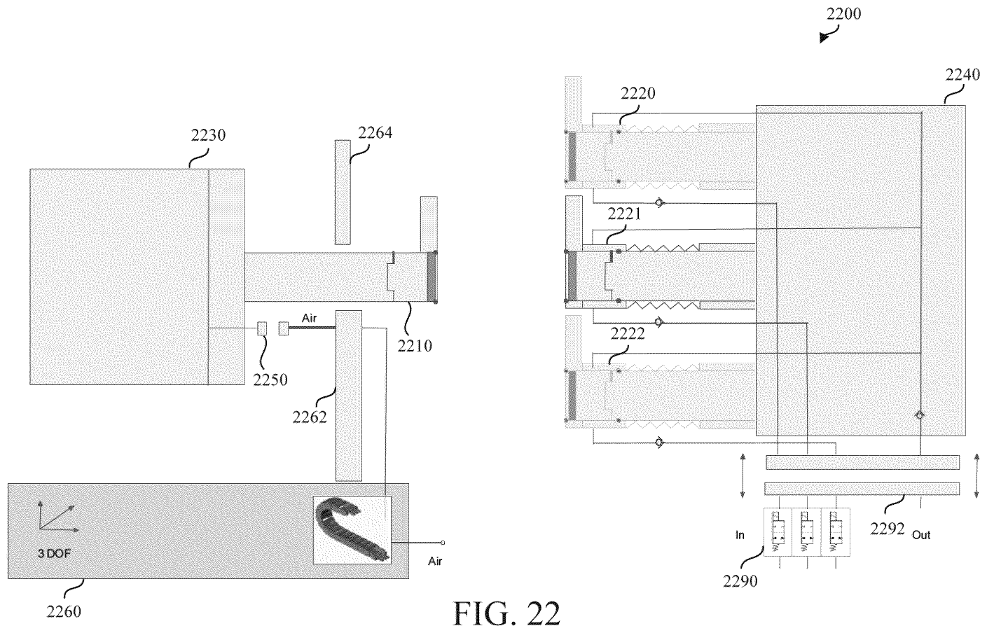

In some variations, a fluid connector may comprise a sterilant source coupled to a plurality of second connectors. FIG. 22 is a block diagram of an illustrative variation of a fluid connector system 2200 comprising a first connector 2210, a plurality of second connectors 2220, 2221, 2222, a first fluid device 2230 (e.g., sterile liquid transfer device), a second fluid device 2240 (e.g., consumable), a robot 2260, a sterilant source 2290 comprising one or more valves, and a sterilant switch 2292. The first connector 2210 may be coupled in fluid communication with the first fluid device 2230, and the second connectors 2220, 2221, 2222 may be coupled in fluid communication with the second fluid device 2240. The robot 2260 may comprise one or more end effectors 2262, 2264 configured to manipulate and/or couple to one or more of the first fluid device 2230 and first connector 2210. For example, the first fluid device 2230 may comprise one or more fluid ports 2250 configured to couple to an end effector 2262. In some variations, the sterilant source 2290 may be coupled to the switch 2292. The switch 2292 may be coupled to each of the second connectors 2220, 2221, 2222 in order to facilitate efficient and shared fluidic connections between the fluid device, fluid connector, and sterilization system. In some variations, a sterilant conduit may be routed from the switch 2292 through the second fluid device 2240 to a respective second connector 2220, 2221, 2222.

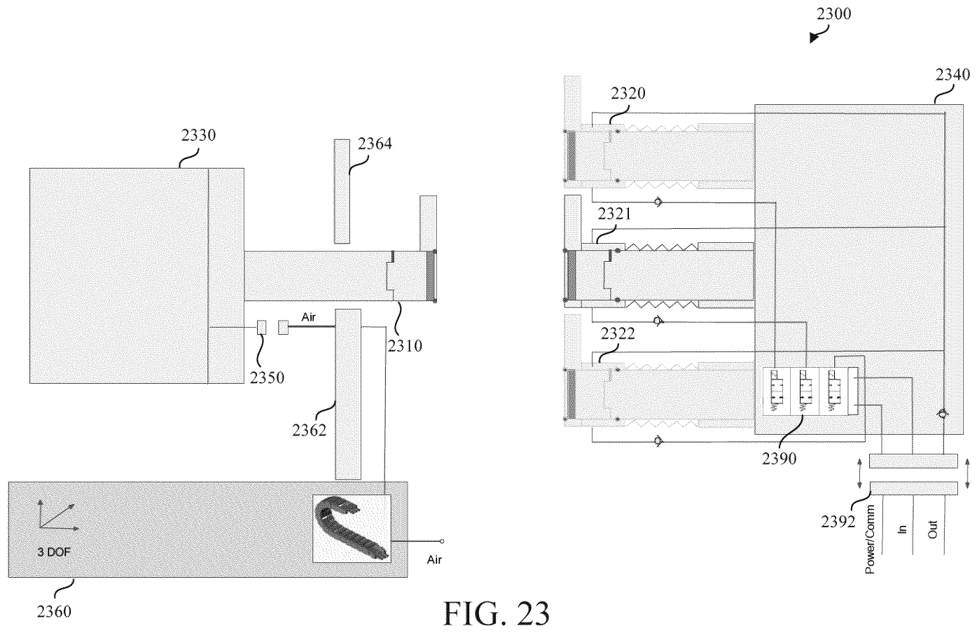

In some variations, a fluid device may comprise one or more sterilant valves coupled to a plurality of second connectors. FIG. 23 is a block diagram of an illustrative variation of a fluid connector system. FIG. 23 is a block diagram of an illustrative variation of a fluid connector system 2300 comprising a first connector 2310, a plurality of second connectors 2320, 2321, 2322, a first fluid device 2330 (e.g., sterile liquid transfer device), a second fluid device 2340 (e.g., consumable), a robot 2360, a set of sterilant valves 2390 disposed within a housing of the second fluid device 2340, and a sterilant switch 2392. The first connector 2310 may be coupled in fluid communication with the first fluid device 2330, and the second connectors 2320, 2321, 2322 may be coupled in fluid communication with the second fluid device 2340. The robot 2360 may comprise one or more end effectors 2362, 2364 configured to manipulate and/or couple to one or more of the first fluid device 2330 and first connector 2310. For example, the first fluid device 2330 may comprise one or more fluid ports 2350 configured to couple to an end effector 2362. In some variations, the sterilant valves 2390 may be coupled to the switch 2392. The switch 2392 may be coupled to each of the second connectors 2320, 2321, 2322 via the sterilant valves 2390 in order to facilitate efficient and shared fluidic connections between the fluid device, fluid connector, and sterilization system. In some variations, a sterilant conduit may be routed from the switch 2392 through the second fluid device 2340 to a respective second connector 2320, 2321, 2322.

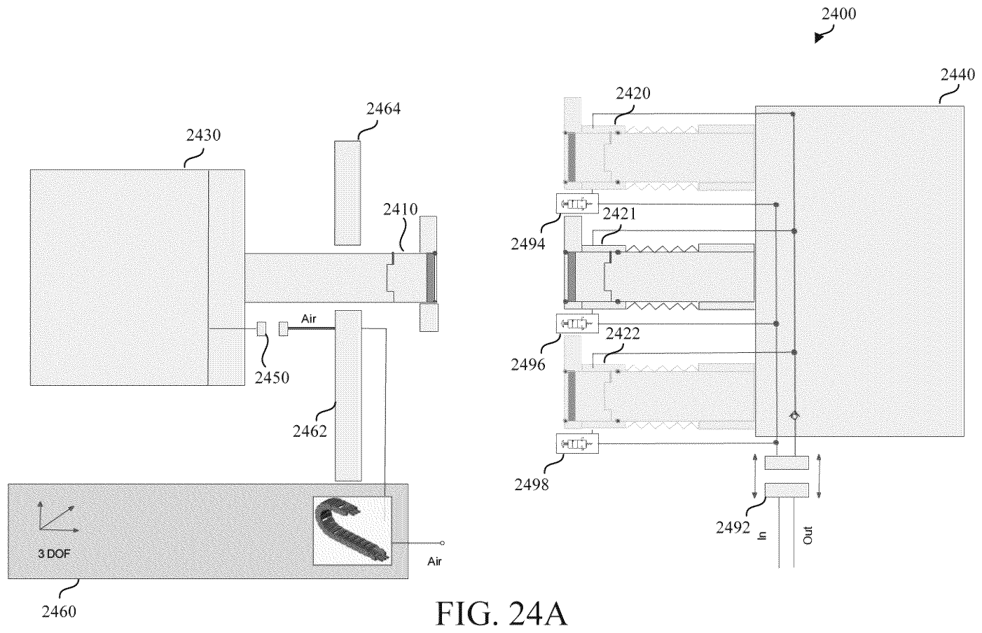

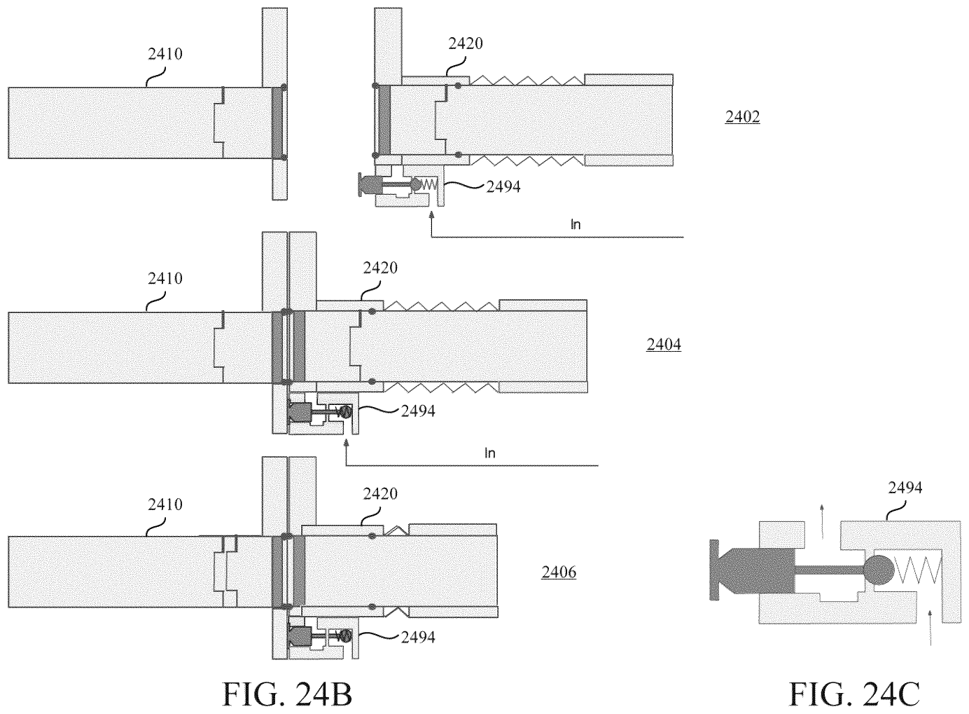

In some variations, a fluid connector may comprise a sterilant source coupled to a plurality of second connectors each having a sterilant port (e.g., sterilant valve) and a sterilant conduit through a fluid device. FIG. 24A is a block diagram of an illustrative variation of a fluid connector system 2400 comprising a first connector 2410, a plurality of second connectors 2420, 2421, 2422, a first fluid device 2430 (e.g., sterile liquid transfer device), a second fluid device 2440 (e.g., a cartridge), a robot 2460, and a sterilant switch 2492 coupled to a sterilant source (not shown). The first connector 2410 may be coupled in fluid communication with the first fluid device 2430, and the second connectors 2420, 2421, 2422 may be coupled in fluid communication with the second fluid device 2440. The robot 2460 may comprise one or more end effectors 2462, 2464 configured to manipulate and/or couple to one or more of the first fluid device 2430 and first connector 2410. For example, the first fluid device 2430 may comprise one or more fluid ports 2450 configured to couple to an end effector 2462.

In some variations, each of the second connectors 2420, 2421, 2422, may comprise a respective sterilant port 2494, 2496, 2498 comprising a valve coupled to a distal end of the second connector 2420, 2421, 2422. In some variations, a sterilant conduit may be routed from the switch 2492 through the second fluid device 2440 to a respective sterilant port 2494, 2496, 2498. In some variations, a sterilant source (not shown) may be coupled to the switch 2492. The switch 2492 may be coupled to each of the second connectors 2420, 2421, 2422 via the sterilant ports 2494, 2496, 2498 in order to facilitate efficient and shared fluidic connections between the fluid device, fluid connector, and sterilization system.

FIG. 24B are schematic diagrams of a fluid connector connection process 2402, 2404, 2406 where a first connector 2410 is coupled to a second connector 2420. For example, the sterilant port 2494 is in a closed valve configuration when the first connector 2410 and the second connector 2420 are separated and uncoupled 2402. FIG. 24C is a detailed schematic diagram of the sterilant valve 2494. In some variations, the valve 2494 may transition to an open valve configuration when the first connector 2410 is coupled to the second connector 2420, at 2404 and 2406.

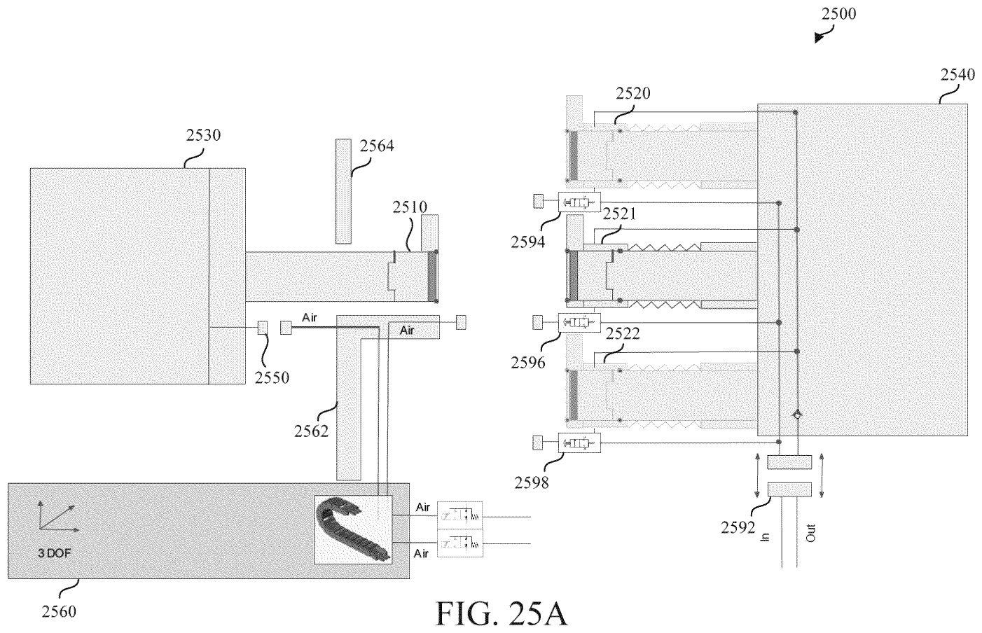

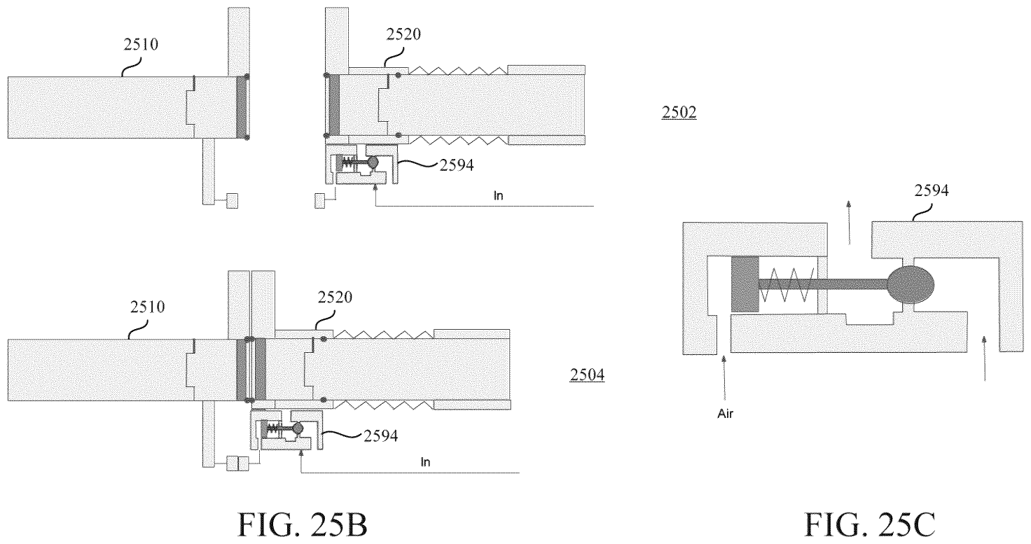

In some variations, a plurality of second connectors may comprise one or more pneumatic sterilant valves and a sterilant path through a fluid device. FIG. 25A is a block diagram of an illustrative variation of a fluid connector system 2500 comprising a first connector 2510, a plurality of second connectors 2520, 2521, 2522, a first fluid device 2530 (e.g., sterile liquid transfer device), a second fluid device 2540 (e.g., a cartridge), a robot 2560, and a sterilant switch 2592 coupled to a sterilant source (not shown). The first connector 2510 may be coupled in fluid communication with the first fluid device 2530, and the second connectors 2520, 2521, 2522 may be coupled in fluid communication with the second fluid device 2540.

In some variations, each of the second connectors 2520, 2521, 2522, may comprise a respective pneumatic sterilant port 2594, 2596, 2598 comprising a valve coupled to a distal end of the second connector 2520, 2521, 2522. In some variations, a sterilant conduit may be routed from the switch 2592 through the second fluid device 2540 to a respective sterilant port 2594, 2596, 2598. In some variations, a sterilant source (not shown) may be coupled to the switch 2592. The switch 2592 may be coupled to each of the second connectors 2520, 2521, 2522 via the sterilant ports 2594, 2596, 2598 in order to facilitate efficient and shared fluidic connections between the fluid device, fluid connector, and sterilization system.

The robot 2560 may comprise one or more end effectors 2562, 2564 configured to manipulate and/or couple to one or more of the first fluid device 2530, first connector 2510, and sterilant ports 2594, 2596, 2598. For example, the first fluid device 2530 may comprise one or more fluid ports 2550 configured to couple to an end effector 2562. Similarly, sterilant ports 2594, 2596, 2598 may be configured to couple to the end effector 2562 to pneumatically actuate the sterilant ports 2594, 2596, 2598. A pneumatically actuated sterilant port may enable the sterilant conduit to be formed with a fewer number of check valves between the sterilant ports 2594, 2596, 2598 and switch 2592.

FIG. 25B are schematic diagrams of a fluid connector connection process 2502 and 2504, where a first connector 2510 is coupled to a second connector 2520. For example, the sterilant port 2594 is in a closed valve configuration when the first connector 2510 and the second connector 2520 are separated and uncoupled 2502. FIG. 25C is a detailed schematic diagram of the sterilant valve 2594. In some variations, the valve 2594 may transition to an open valve configuration when the first connector 2510 is coupled to the second connector 2520 and the valve 2594 is pneumatically actuated, at 2504.

### Liquid Transfer Bus

Generally, to permit transfer of one or more of a cell product (that is, solution(s) containing cell product), fluids, and reagents between the modules, the modules of the cartridge may be fluidically coupled to one another either directly or via one or more liquid transfer buses. In some variations, a liquid transfer bus may comprise a portion of the cartridge configured to control the flow and distribution of the cell product between modules and reservoirs. A liquid transfer bus may comprise one or more of a fluid manifold, fluid conduit (e.g., tubing), and one or more valves (including but not limited to 2/2 valves, 3/2 valves, 3/3 valves, 4/2 valves, and rotary selector valves).

Transfer of the cell product, reagents, or fluids within the cartridge may be achieved by any pump or other structure that generates a pressure differential between fluid in one portion of the cartridge and fluid in another portion of the cartridge. For example, the cartridge may comprise one or more pumps; the cartridge may be pre-loaded with pressurized fluid contained behind a valve; the cartridge may be connected to a fluid source or a fluid sink. The cartridge may contain one or more mechanical pumps (e.g., linear pump, peristaltic pump, gear pump, screw pump, plunger pump) or portions of a pump (i.e. the pump may interface with a pump actuator). External pressure may be applied to the cartridge, to tubing within the cartridge, or to a bag within the cartridge (that is, applying pressure either to the liquid in the bag or to headspace gas of the bag). In some variations, an arrangement of the components of the cartridge may facilitate gravity-based fluid transfer within the cartridge (e.g., gravity-fed pumping). Although one advantage of the disclosed variations may be reduced operator intervention, the systems and methods of the disclosure may use manual operation in the designed workflow or as an adjunct to automated operation in case of imperfect automated system operation. For example, a process step may include manual intervention, such as fluid input or output. An operator may intervene in an automated process to correct device operation, (e.g. manually compressing a bag to flush remaining fluid into the system). Fluid may comprise liquid and/or gas, as compressed gases supplied externally or provided in pressurized chambers may be used to generate liquid flow, e.g., transfer of solution containing a cell product from one module to another.

In some variations, the liquid transfer bus may be configured to deliver the cell product(s) to each of a series of modules in an order set by the design of the cartridge, or in an order determined by operation of the system by the processor or processors. Similarly stated, some variations of the cartridge may have the advantage that the order of cell processing steps as well as the process parameters for any of the cell therapy processing steps may not be set by the cartridge but rather are controlled by the controller. In some variations, the liquid transfer bus may be controlled to deliver the cell product to the modules in any of various sequences, or to bypass one or more modules (e.g., by configuring the state of the valve(s) attached to the fluidic bus). In some variations, a module may be used more than once in a method of cell processing. Optionally, the method may comprise performing one or more wash steps. For example, a counterflow centrifugal elutriation (CCE) module may be used more than once. In an illustrative method, the method comprises culturing the cell product in a first bioreactor module, transferring the cell product to the CCE module to enrich for a desired cell type, transferring the cell product to a second bioreactor module for a second culturing step, washing the CCE module using a wash solution, and transferring the cell product to the CCE module for a second enrichment step.

In some variations, the liquid transfer bus or the liquid transfer buses may be fluidically coupled to multiple bags or reservoirs used to provide solutions or reagents, store cell products, or to collect waste solutions or reagents.

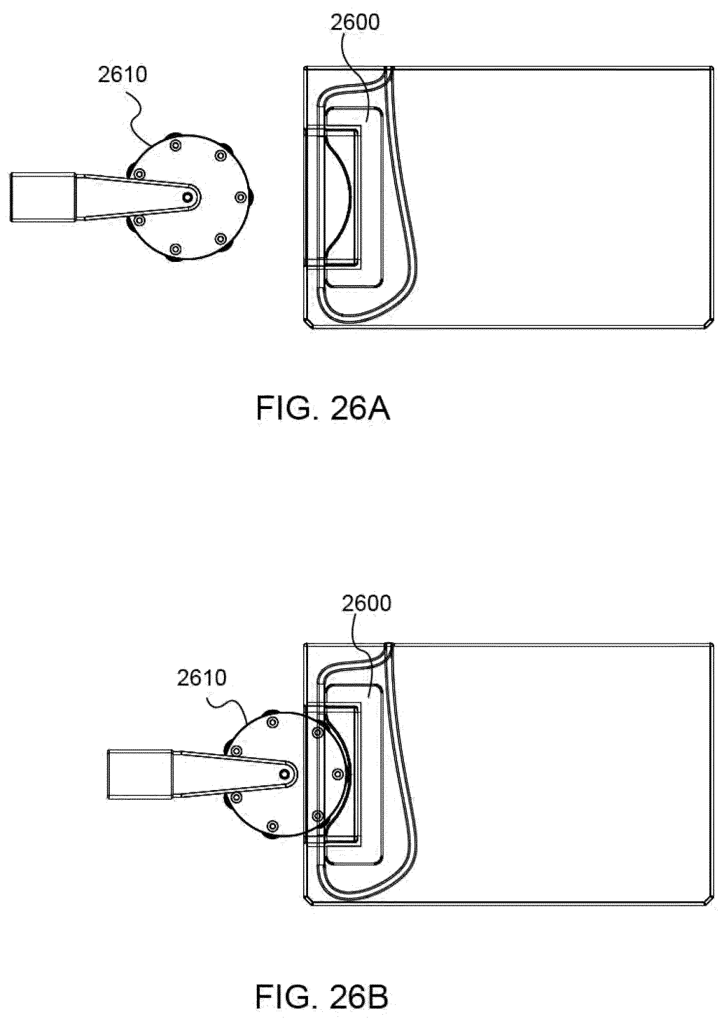

In some variations, the cartridge may comprise one or more pumps, which may be fluidically coupled to the liquid transfer bus and/or one or more modules. The pump(s) may include a motor operatively coupled to control circuits and a power source (e.g. a battery or electrical connectors for an off-cartridge power source). In some variations, the pump may be divided into a pump on the cartridge and pump actuators on one or more docking station modules of the system. The pump may be an opening in the cartridge with tubing arranged around the circumference of the opening and configured to receive a pump actuator (e.g., a peristaltic rotor). By dividing components of the pump that contact the cell product (i.e. tubing) from components of the pump that perform operations of the cell product, (i.e. the pump actuator, e.g., peristaltic rotor), the cartridge may be compact and simplified. For example, FIG. 26A and FIG. 26B illustrate a pump head 2610 and a pump 2610 of a cartridge in an uncoupled configuration (FIG. 13A) and a coupled configuration.

In some variations, one or more pumps 146 (e.g., fluid pump) may generate a predetermined fluid flow rate to circulate a sterilant and/or fluid. In some variations, a pump may comprise one or more of a positive displacement pump (e.g., peristaltic pump, diaphragm pump, syringe pump), centrifugal pump, combinations thereof, and the like. One or more fluid sources may be coupled to the pump.

In some variations, the pump may be configured to receive a pump signal (generated by a controller) configured to circulate a sterilant for a dwell time sufficient to sterilize at least a portion of a fluid connector. For example, the pump may be configured to circulate the sterilant for at least 10 seconds. In some variations, the pump may be configured to receive a pump signal configured to circulate a non-sterilant gas (e.g., inert gas, air) to remove the sterilant.

In some variations, a discontinuous flow pump (e.g., peristaltic pump) may generate pulsatile flow as, for example, a tube contracts and relaxes between rollers. In some variations, closed loop feedback from a flow sensor may be used to compensate for pulsatile flow to generate a substantially continuous flow rate. For example, a flow sensor may be coupled to a fluid conduit to measure the flow rate. A controller may receive the measured flow rate and generate a pump signal to the pump based on a proportional correction function configured to reduce the “ripples” measured by the flow sensor. Additionally or alternatively, a controller may apply periodic error correction to a pump signal to reduce periodic error that may be unique to each pump. For example, a flow sensor may measure and determine a periodic error of a pump. A pump signal comprising the periodic error correction may correspond to a waveform comprising an inverse shape of the error. The resulting pump flow may correct for fluctuations in flow rate.

### Controller

In some variations, a system 100 may comprise a controller 120 (e.g., computing device) comprising one or more of a processor 122, memory 124, communication device, 126, input device 128, and display 130. The controller 120 may be configured to control (e.g., operate) the CPS 110. The controller 120 may comprise a plurality of devices. For example, the CPS 110 may enclose one or more components of the controller 120 (e.g., processor 122, memory 124, communication device 126) while one or more components of the controller 120 may be provided remotely to the CPS 110 (e.g., input device 128, display 130).

### Processor

The processor (e.g., processor 122) described here may process data and/or other signals to control one or more components of the system (e.g., CPS 110, controller 120). The processor may be configured to receive, process, compile, compute, store, access, read, write, and/or transmit data and/or other signals. Additionally, or alternatively, the processor may be configured to control one or more components of a device and/or one or more components of controller (e.g., console, touchscreen, personal computer, laptop, tablet, server).

In some variations, the processor may be configured to access or receive data and/or other signals from one or more of the CPS 110, server, controller 120, and a storage medium (e.g., memory, flash drive, memory card, database). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor may be, for example, a general purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including structured text, typescript. C, C++, C#, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

### Memory

The cell processing and manufacturing systems and devices described here may include a memory (e.g., memory 124) configured to store data and/or information. In some variations, the memory may include one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with the device, such as image processing, image display, sensor data, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. In some variations, the memory may be configured to store any received data and/or data generated by the controller and/or the cell processing station. In some variations, the memory may be configured to store data temporarily or permanently.

### Input Device

In some variations, the display may include and/or be operatively coupled to an input device 128 (e.g., touch screen) configured to receive input data from a user. For example, user input to an input device 128 (e.g., keyboard, buttons, touch screen) may be received and processed by a processor (e.g., processor 122) and memory (e.g., memory 124) of the CPMS 100. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device including at least one switch, a switch may have, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

In some variations, the cell processing and manufacturing system may optionally include one more output devices in addition to the display, such as, for example, an audio device and haptic device. An audio device may audibly output any system data, alarms, and/or notifications. For example, the audio device may output an audible alarm when a malfunction is detected. In some variations, an audio device may include at least one of a speaker, piezoelectric audio device, magnetostrictive speaker, and/or digital speaker. In some variations, a user may communicate with other users using the audio device and a communication channel. For example, a user may form an audio communication channel (e.g., VoIP call).

Additionally or alternatively, the system may include a haptic device configured to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the processor.

### Communication Device

In some variations, the controller may include a communication device (e.g., communication device 126) configured to communicate with another controller and one or more databases. The communication device may be configured to connect the controller to another system (e.g., Internet, remote server, database, cell processing station) by wired or wireless connection. In some variations, the system may be in communication with other devices via one or more wired and/or wireless networks. In some variations, the communication device may include a radiofrequency receiver, transmitter, and/or optical (e.g., infrared) receiver and transmitter configured to communicate with one or more devices and/or networks. The communication device may communicate by wires and/or wirelessly.

The communication device may include RF circuitry configured to receive and send RF signals. The RF circuitry may convert electrical signals to/from electromagnetic signals and communicate with communications networks and other communications devices via the electromagnetic signals. The RF circuitry may include well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), EtherCAT, OPC Unified Architecture, or any other suitable communication protocol. In some variations, the devices herein may directly communicate with each other without transmitting data through a network (e.g., through NFC, Bluetooth, WiFi, RFID, and the like).

In some variations, the systems, devices, and methods described herein may be in communication with other wireless devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (MAY), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS. CDMA or GPRS, W-CDMA, EDGE or CDMA2000, LTE, WiMAX, and 5G networking standards. Some wireless network deployments combine networks from multiple cellular networks or use a mix of cellular. Wi-Fi, and satellite communication.

### Display

Image data may be output on a display e.g., display 130) of a cell processing and manufacturing system. In some variations, a display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

### II. METHODS

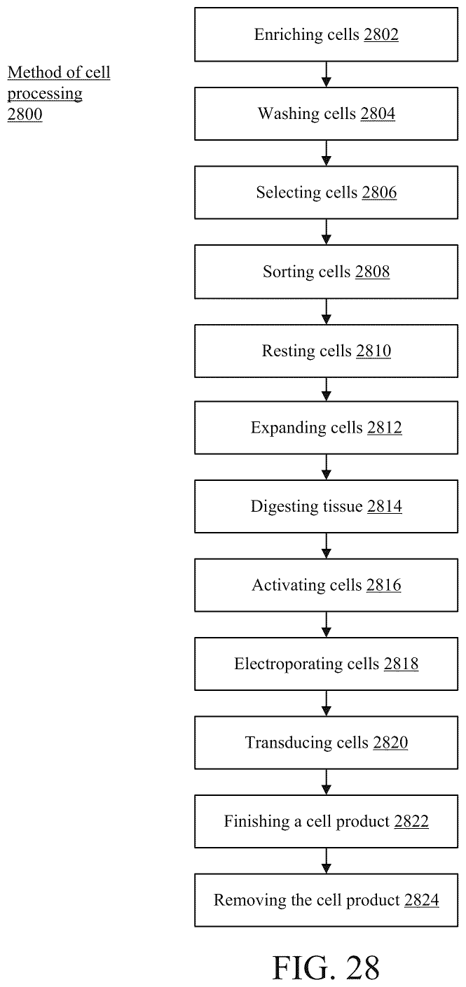

Generally, the systems and devices described herein may perform one or more cell processing steps to manufacture a cell product. FIG. 28 is a flowchart of a method of cell processing 2800. The method 2800 may include enriching a selected population of cells in a solution (e.g., fluid) 2802. For example, the solution may be conveyed to a cartridge CCE module via a liquid transfer bus. The cartridge may be moved to a docking station of a cell processing station (CPS) by a user. The docking station may comprise a plurality of docking station modules corresponding to one or more modules of the cartridge. For instance, the docking station modules may include a docking station CCE module configured to interface with the cartridge CCE module to perform cell processes on the cells within the cartridge. In this instance, the cell process includes enriching the selected population cells via counterflow centrifugal elutriation.

Additionally or alternatively, the cell product may be introduced into and out of the cartridge via a sterile liquid transfer port (either manually or automatically) for any of the steps described herein. In some variations, the cartridge may be sterilized in a feedthrough port (either manually or automatically).

In some variations, a selected population of cells in the solution may be washed 2804. For example, the solution may be conveyed to the CCE module of a cartridge within a docking station via the liquid transfer bus. The docking station CCE module may be operated to cause the cartridge CCE module to remove media from the solution, introduce media into the solution, and/or replace media in the solution.

In some variations, a population of cells in the solution may be selected 2806. For example, the solution may be conveyed to a selection module of the cartridge via the liquid transfer bus. A docking station selection module may interface with the cartridge selection module and the docking station selection module may be operated to cause the cartridge selection module to select the selected population of cells.

In some variations, a population of cells in the solution may be sorted 2808. For example, the solution may be conveyed to a sorting module of the cartridge via the liquid transfer bus. A docking station sorting module may interface with the cartridge sorting module and the docking station sorting module may be operated to cause the cartridge sorting module to sort the population of cells.

In some variations, the solution may be conveyed to a bioreactor module of the cartridge via the liquid transfer bus to rest 2810. A docking station bioreactor module may interface with the cartridge bioreactor module and the docking station bioreactor module may be operated to cause the cartridge bioreactor module to maintain the cells at a set of predetermined conditions.

In some variations, the cells may be expanded in the solution 2812. For example, the solution may be conveyed to the bioreactor module of the cartridge via the liquid transfer bus. A docking station bioreactor module may interface the cartridge bioreactor module and may be operated to cause the cartridge bioreactor module to expand the cells by cellular replication.

In some variations, tissue may be digested by conveying an enzyme reagent via the liquid transfer bus to a cartridge module containing a solution containing a tissue such that the tissue releases a select cell population into the solution 2814.

In some variations, a selected population of cells in the solution may be activated by conveying an activating reagent via the liquid transfer bus to a cartridge module containing the solution containing the cell product 2816.

In some variations, the solution may be conveyed to an electroporation module of the cartridge via the liquid transfer bus and receive an electroporation signal to electroporate the cells in the solution 2818. For example, a docking station electroporation module may interface with the cartridge electroporation module and may be operated to cause the cartridge electroporation module to electroporate the selected population of cells in the presence of genetic material.

In some variations, an effective amount of a vector may be conveyed via the liquid transfer bus to a cartridge module containing the solution containing the cell product, thereby transducing a selected population of cells in the solution 2820.

In some variations, a formulation solution may be conveyed via the liquid transfer bus to a cartridge module comprising the cell product to generate a finished cell product 2822. For example, the finished cell product may be conveyed to one or more product collection bags. In some variations, finishing a cell product may comprise one or more steps of washing cells, concentrating cells, exchanging a buffer of the cells with a formulation buffer, and dosing cells in the formulation buffer in predetermined quantities into one or more product collection bags and/or vessels.

In some variations, the cell product may be removed, either manually or automatically, from the cartridge to harvest the cells 2824.

In some variations, the cell product may comprise one or more of an immune cell genetically engineered chimeric antigen receptor T cell, a genetically engineered T cell receptor (TCR) cell, a hematopoietic stem cell (HSC), and a tumor infiltrating lymphocyte (TIL). In some variations, the immune cell may comprise a natural-killer (NK) cell.

Methods of cell processing may include a subset of cell processing steps in any suitable order. For example, the method of cell processing may include, in order, the enrichment step 2802, the selection step 2806, the activation step 2816, the transduction step 2820, the expansion step 2812, and the harvesting step 2824. In some variations, the method of cell processing may include, in order, the enrichment step 2802, the selection step 2806, the resting step 2810, the transduction step 2820, and the harvesting step 2824. In some variations, the method of cell processing may include, in order, the tissue-digestion step 2820, the washing step 2804, the activation step 2816, the expansion step 2812, and the harvesting step 2824. Moreover, one or more of the enrichment step 2802, the washing step 2804, the selecting step 2806, the sorting step 2808, the resting step 2810, the expansion step 2812, the digestion step 2814, the activation step 2816, the electroporation step 2818, the transduction step 2820, the finishing step 2822, and the removing step 2824 may be performed while the cartridge remains stationary within the docking station.

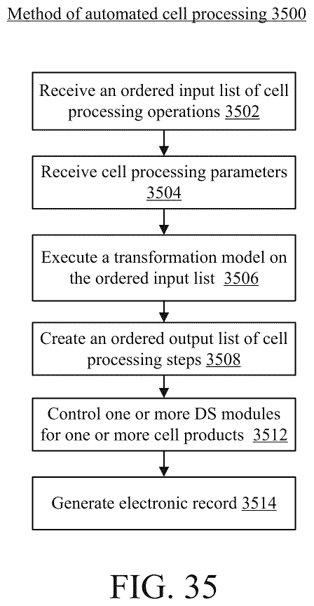

Generally, the methods described herein may offload the complex steps performed in cell processing operation to a set of docking station modules, thereby reducing the cost of the cartridge (which may be a consumable). In some variations, the cartridge may contain the cell product (e.g., solution containing cells) throughout a manufacturing process, with different docking station modules, which are interfaced with the cartridge, being engaged at appropriate times to perform one or more cell processing steps. For example, a cell processing step may comprise conveying cells and reagents to each of the modules within the cartridge. A set of docking station modules interfacing with a cartridge facilitates process flexibility where a CPS and a docking station therein may be customized with a predetermined set of docking station modules for a predetermined cell therapy product. For example, the order of cell processing steps may be customized for each cell product as described in more detail herein with respect to FIGS. 35-55.

In some variations, a cell product may be retained within the cartridge throughout a manufacturing process (e.g., workflow). Additionally or alternatively, the cell product may be removed from the cartridge for one or more cell processing steps, either manually by an operator, or automatically via a fluid connector or by other access port system on the cartridge. The cell product may then be returned to the same cartridge, transferred to another cartridge, or split among several cartridges. In some variations, one or more cell processing steps may be performed outside the cartridge. In some variations, processing within the CPS may facilitate sterile cell processing within the cartridge.

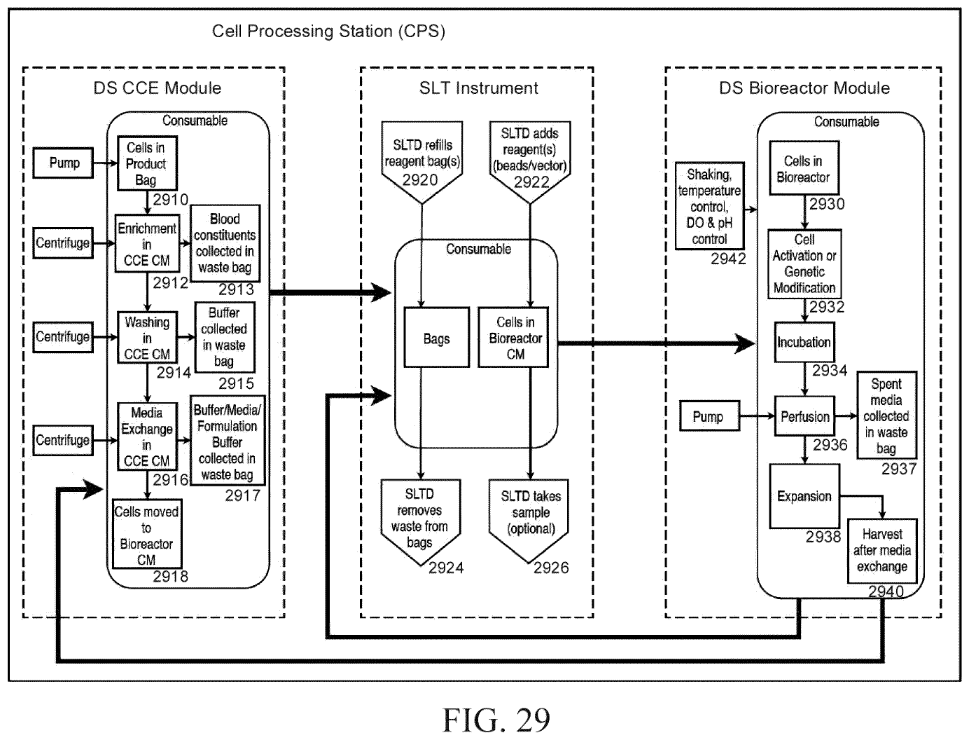

FIG. 29 is a flowchart of a method of cell processing and illustrates cell processing steps performed on a cartridge (e.g., consumable) within a CPS including a docking station (DS) CCE module, a sterile liquid transfer (SLT) instrument, and a DS bioreactor module. The cartridge may be configured to interface with any of the DS CCE module, the SLT instrument, and the DS bioreactor module to perform one or more cell processing steps.

In some variations, the DS CCE module may comprise a pump and centrifuge configured to interface with a cartridge (e.g., consumable). The SLT instrument may comprise one or more fluid connectors be configured to interface with one or more of a bag and bioreactor of a cartridge. The DS bioreactor module may comprise one or more sensors, temperature regulators, pumps, agitators, and the like, and be configured to interface with the cartridge. In some variations, the cell product may be contained within the cartridge throughout cell processing.

A method of cell processing depicted in FIG. 29 may include moving a fluid (e.g., cells in solution) in a product bag to a CCE module (e.g., rotor) of a cartridge (e.g., consumable) using a pump 2910. In some variations, the fluid may be enriched using the cartridge CCE module 2912. For example, blood constituents may be collected in a waste bag 2913. In some variations, the fluid may be washed using the cartridge CCE module 2914. For example, buffer may be collected in a waste bag 2915. In some variations, media may be exchanged using the cartridge CCE module 2916. For example, one or more of buffer (e.g., formulation buffer) and media may be collected in a waste bag 2917. In some variations, fluid may be moved to a bioreactor of the cartridge 2918.

In some variations, a fluid connector may fill a bag with a reagent 2920. In some variations, a reagent (e.g., bead, vector) may be added to a bioreactor of a cartridge 2922. In some variations, a fluid connector removes waste from a bag 2924. In some variations, a fluid connector may optionally remove a sample from a bioreactor.

In some variations, cells may be moved to a bioreactor 2930. In some variations, the cells may undergo activation or genetic modification 2932. In some variations, the cells may undergo incubation 2934. In some variations, the cells may undergo perfusion using a pump 2936. For example, spent media may be collected in a waste bag 2937. In some variations, cells may undergo expansion 2938. In some variations, cells may be harvested after media exchange 2940.

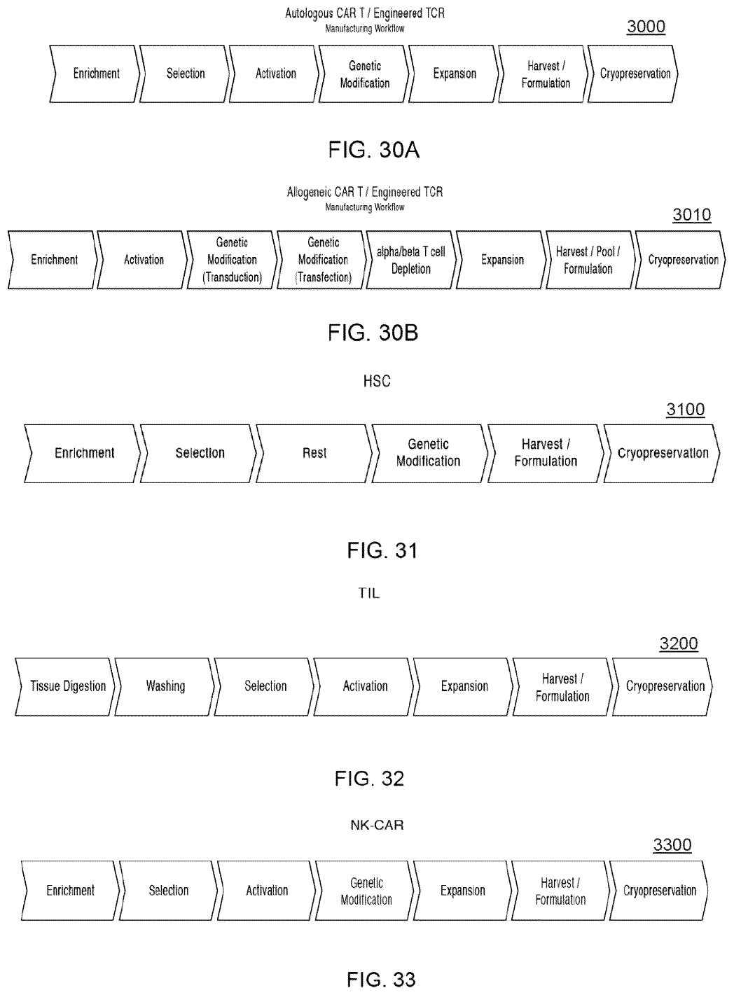

FIG. 30A is a flowchart of a method of cell processing for autologous CAR T cells or engineered TCR cells. The method 3000 may comprise the steps of enrichment, selection, activation, genetic modification, expansion, harvest/formulation, and cryopreservation. FIG. 30B is a flowchart of a method of cell processing for allogeneic CAR T cells or engineered TCR cells. The method 3010 may comprise the steps of enrichment, activation, genetic modification (e.g., transduction, transfection), alpha/beta T cell depletion, expansion, harvest/pool/formulation, and cryopreservation.

FIG. 31 is a flowchart of a method of cell processing for hematopoietic stem cell (HSC) cells. The method 3100 may comprise the steps of enrichment, selection, rest, genetic modification, harvest/formulation, and cryopreservation.

FIG. 32 is a flowchart of a method of cell processing for tumor infiltrating lymphocyte (TIL) cells. The method 3200 may comprise the steps of tissue digestion, washing, selection, activation, expansion, harvest/formulation, and cryopreservation.

FIG. 33 is a flowchart of a method of cell processing for natural killing (NK) CAR cells. The method 3300 may comprise the steps of enrichment, selection, activation, genetic modification, expansion, harvest/formulation, and cryopreservation.

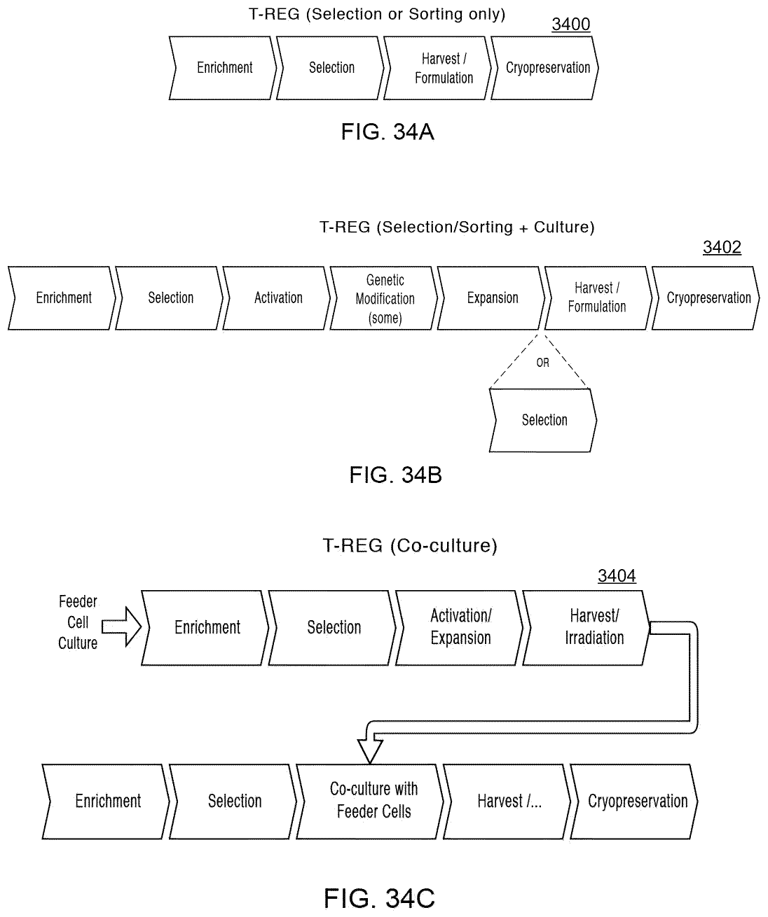

FIGS. 34A-34C are flowcharts of methods of cell processing for regulatory T (T_{reg}) cells. The method 3400 may comprise the steps of enrichment, selection, harvest/formulation, cryopreservation. The method 3402 may comprise the steps of enrichment, selection, activation, genetic modification, expansion, selection (optionally), harvest/formulation, and cryopreservation. The method 3404 may comprise the steps of introducing feeder cell culture for enrichment, selection, activation/expansion, and harvest/irradiation. Another set of cells may undergo enrichment, selection, co-culture with the processed feeder cells, harvest, and cryopreservation.

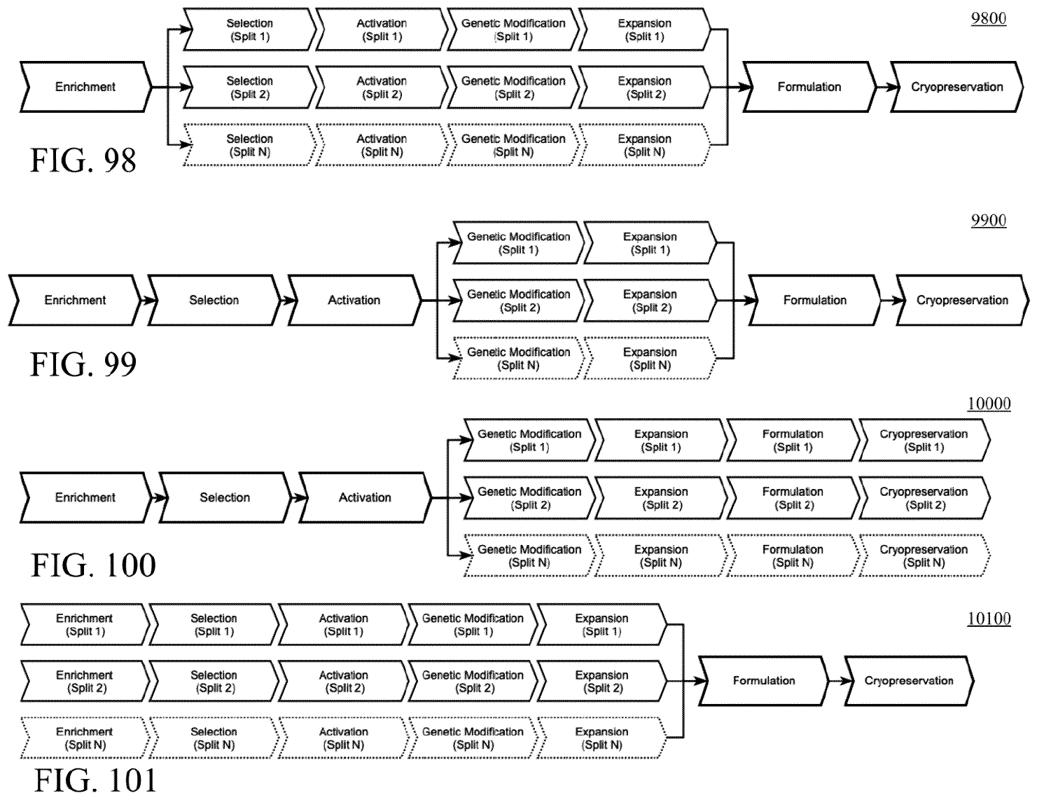

FIGS. 98-101 are flowcharts of methods of cell processing for cell therapy workflows comprising split (e.g., parallel) processing. The method 9800 may comprise the steps of enrichment, selection, activation, genetic modification, expansion, formulation, and cryopreservation. For example, a cell processing method 9800 (e.g., workflow) may comprise splitting a cell product into two or more portions after an enrichment step. The split portions may be processed in parallel within a single cartridge. In some variations, one or more split portions may be transferred to two or more cartridges and processed in parallel. One or more cell processing parameters (e.g., timing of process steps, types of reagents added, transfection constructs, and the like) may be configured independently for each split portion of the cell product. In some variations, the split portions may be pooled after the expansion step.

The method 990 may comprise the steps of enrichment, selection, activation, genetic modification, expansion, formulation, and cryopreservation. For example, a cell processing method 9900 (e.g., workflow) may comprise splitting a cell product into two or more portions after an activation step. The split portions may be processed in parallel within a single cartridge. One or more cell processing parameters (e.g., timing of process steps, types of reagents added, transfection constructs, and the like) may be configured independently for each split portion of the cell product. In some variations, the split portions may be pooled after the expansion step and/or the genetic modification step.

The method 10000 may comprise the steps of enrichment, selection, activation, genetic modification, expansion, formulation, and cryopreservation. For example, a cell processing method 10000 (e.g., workflow) may comprise splitting a cell product into two or more portions after a selection step. The split portions may be processed in parallel within a single cartridge. One or more cell processing parameters (e.g., timing of process steps, types of reagents added, transfection constructs, and the like) may be configured independently for each split portion of the cell product. In some variations, the split portions may not be pooled.

The method 10100 may comprise the steps of enrichment, selection, activation, genetic modification, expansion, formulation, and cryopreservation. For example, a cell processing method 10100 (e.g., workflow) may comprise splitting a cell product into two or more portions as starting materials. The separate products may remain segregated and processed in parallel as split portions within a single cartridge. One or more cell processing parameters (e.g., timing of process steps, types of reagents added, transfection constructs, and the like) may be configured independently for each split portion. In some variations, the split portions may be pooled after the expansion step.

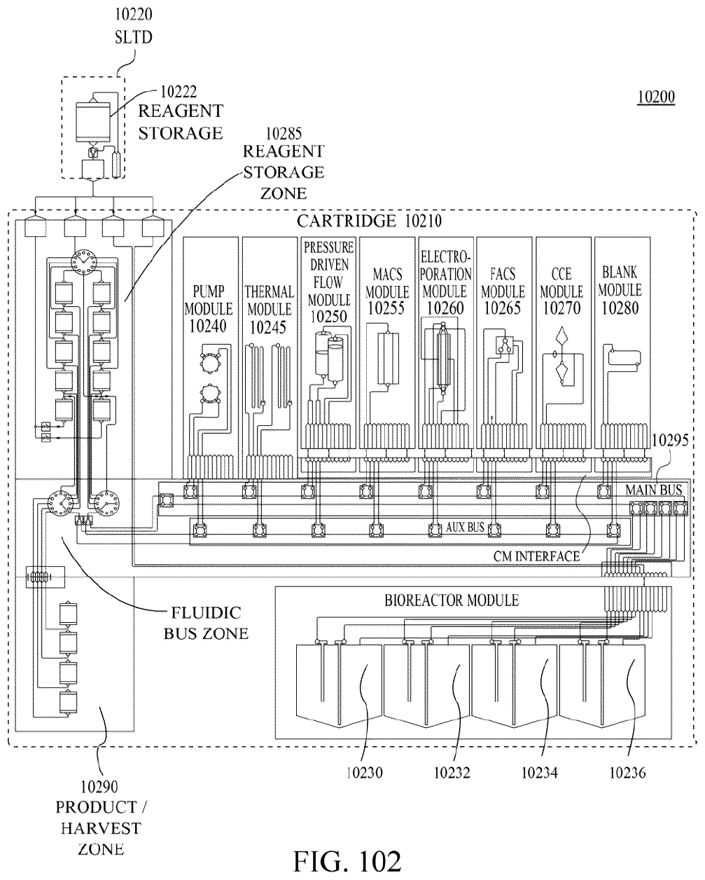

FIG. 102 is a schematic diagram of a cell processing and manufacturing system 10200 configured for split processing within a single cartridge. For example, the methods 9800-10100 described with respect to FIGS. 98-101 may be performed within the cartridge 10210. In some variations, the system 10200 may comprise a sterile liquid transfer device 10220 comprising a reagent 10222, and a cartridge 10210 comprising a plurality of bioreactor modules 10230, a pump module 10240, a thermal module 10245, a pressure driven flow module 10250, a MACS module 10255, an electroporation module 10260, a FACS module 10265, a CCE module 10270, and a blank module 10280. The cartridge 10210 may further comprise a reagent storage 10285, a plurality of product bags 10290, and a liquid transfer bus 10295. The liquid transfer bus 10295 may be configured to couple the components of the cartridge 10210 for fluid communication.

In some variations, loading and removing of cell product into and out of the cartridge may be performed in the system or outside the system. In some variations, the cartridge is loaded bedside to the patient or donor and then delivered to a cell processing and manufacturing system in or near the hospital or shipped to a facility where the cell processing and manufacturing system is installed. Likewise, the cell product may be removed from the cartridge after processing either at a facility or closer to the intended recipient of the cell product (the patient). Optionally, the cell product is frozen before, during, or after the methods of the disclosure-optionally after addition of one or more cryoprotectants to the cell product. In some variations, the system comprises a freezer and/or a liquid nitrogen source. In some variations, the system comprises a water bath or a warming chamber containing gas of controlled temperature to permit controlled thawing of the cell product, e.g. a water bath set to between about 20° C. and about 40° C. In some variations, the cartridge is made of materials that resistant mechanical damage when frozen.

### Automated Cell Processing

Described here are methods of transforming user-defined cell processing operations into cell processing steps using the automated cell processing and manufacturing systems and devices described herein. In some variations, cell processing operations are received and transformed into cell processing steps to be performed by the system given a set of predetermined constraints. For example, a user may input a set of biologic process steps and corresponding biologic process parameters to be executed by a cell processing and manufacturing system. Optionally, process parameters may be customized for each cartridge or sets of cartridges.

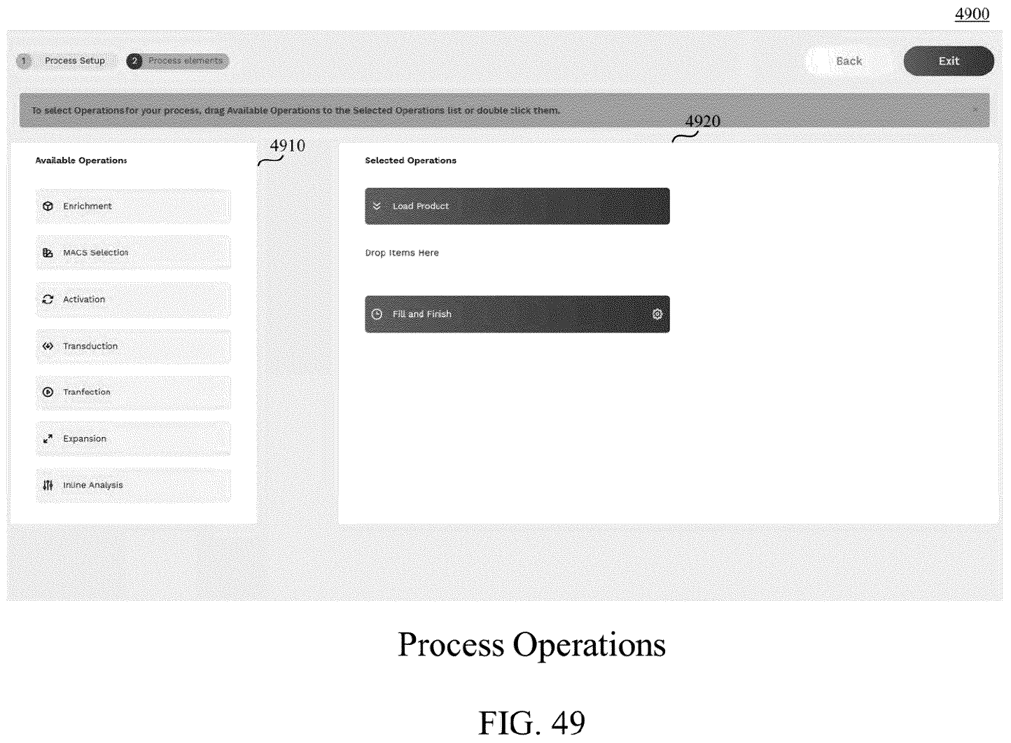

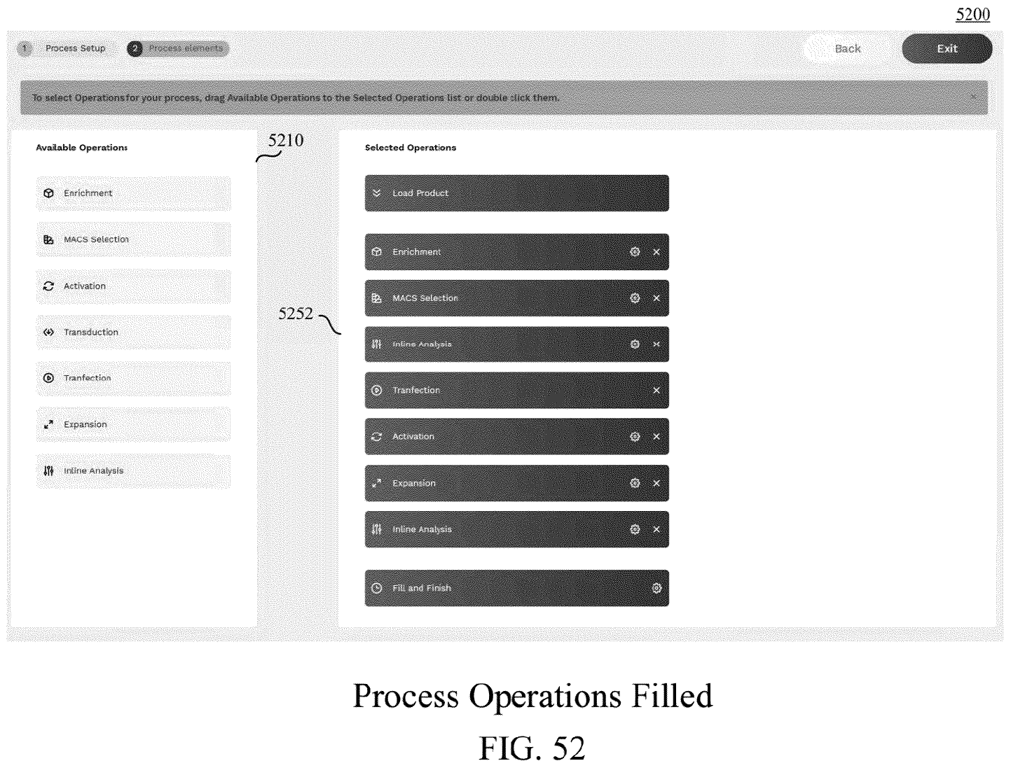

FIG. 35 is a flowchart that generally describes a variation of a method of automated cell processing. The method 3500 may include receiving an ordered input list of cell processing operations 3502. For example, a set of more than one ordered input list of cell processing operations may be received to be performed on more than one cartridge on an automated cell processing and manufacturing system. For example, as shown in the GUI 4900 of FIG. 49 and described in more detail herein, one or more biologic process inputs (e.g., available operations) such as enrichment, MACS selection, activation, transduction, transfection, expansion, and inline analysis may be selected as an ordered input list of cell processing operations. Furthermore. GUI 5200 of FIG. 52 illustrates a complete ordered input list of cell processing operations (e.g., set of selected operations) 5220 selected by a user.

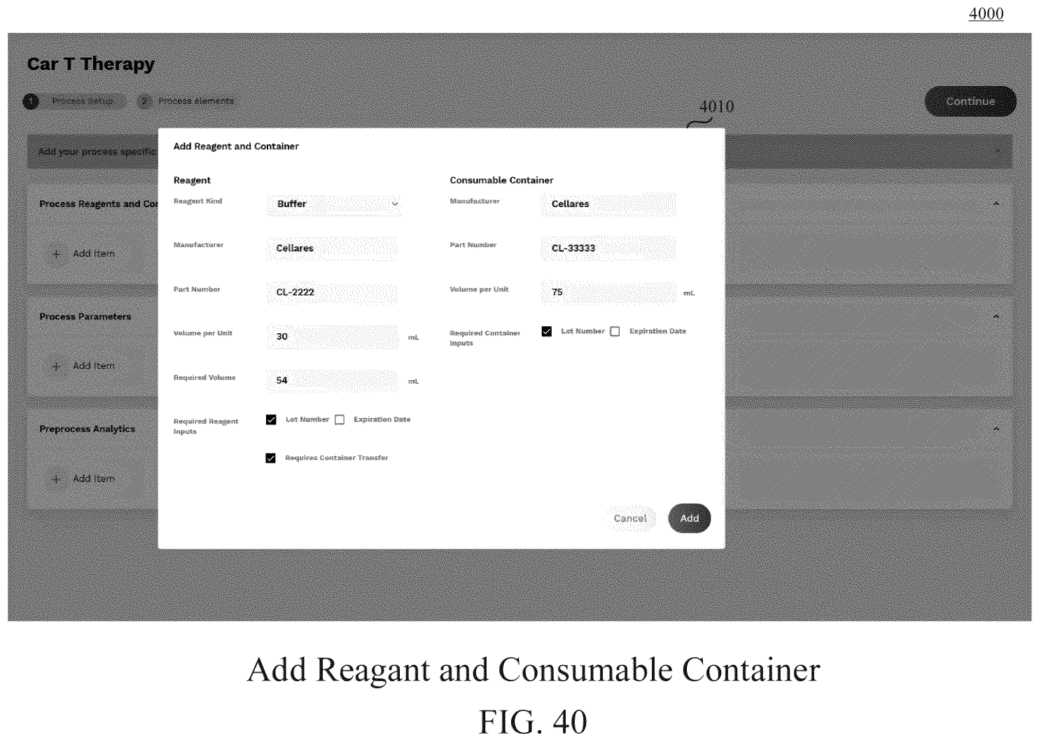

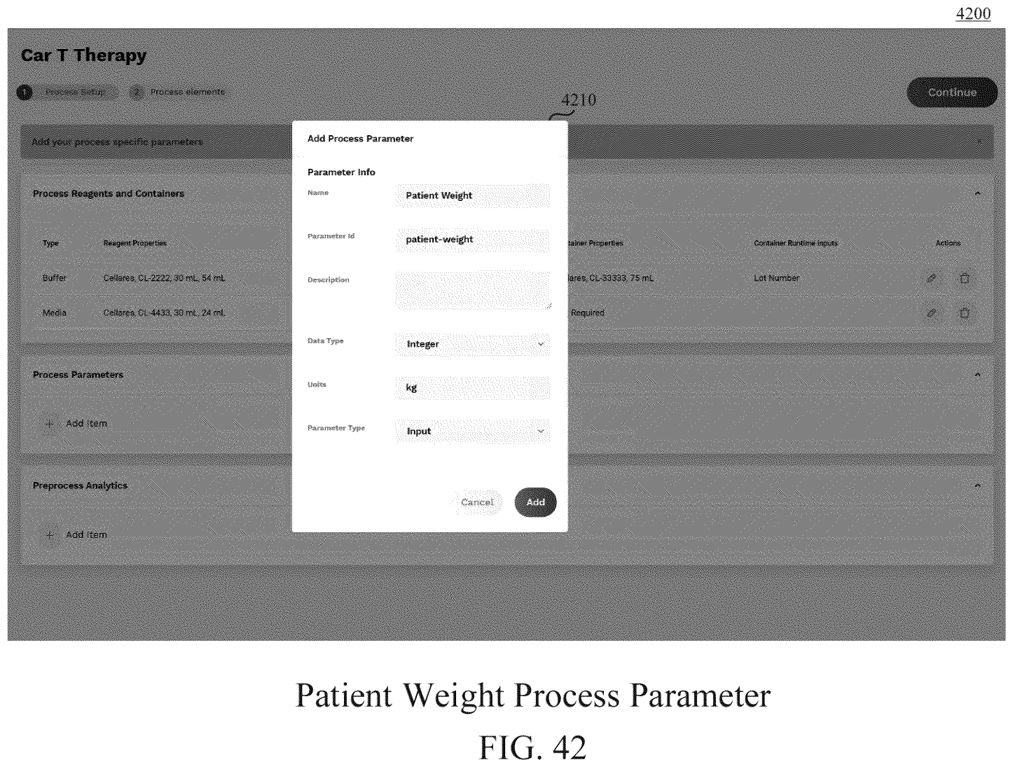

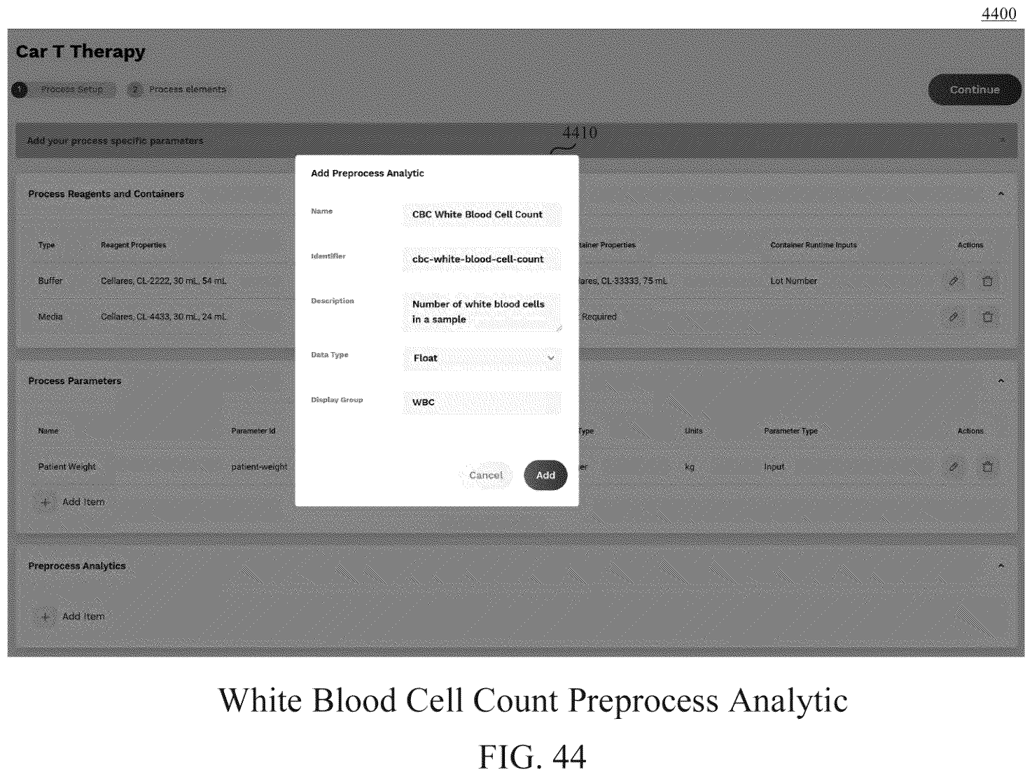

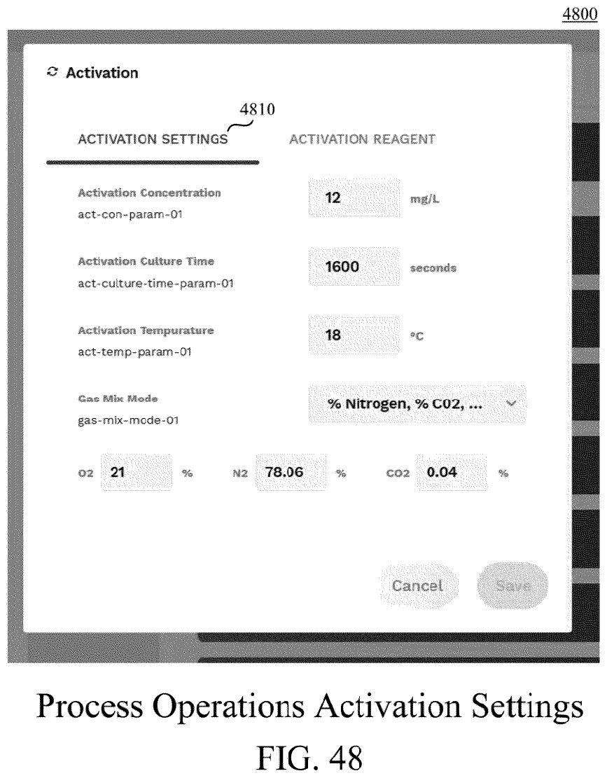

In some variations, one or more sets of cell processing parameters may be received 3504. Each set of cell processing parameters may be associated with one of the cell processing operations. Each set of cell processing parameters may specify characteristics of the cell processing step to be performed by the docking station module at that cell processing step. For example, the GUI 4000 of FIG. 40 illustrates reagent and container parameters, the GUI 4200 of FIG. 42 illustrates an example of a process parameter, the GUI 4400 of FIG. 44 illustrates an example of a preprocess analytic, and the GUI 4800 of FIG. 48 illustrates an example of a set of activation settings.

In some variations, a transformation model may be executed on the ordered input list 3506. In some variations, the transformation model may comprise constraints on the ordered output list determined by a predetermined configuration of the automated cell processing and manufacturing system. For example, the constraints may comprise information on the configuration of the automated cell processing and manufacturing system.

In some variations, the constraints may comprise one or more of a type and/or number and/or state of docking station modules, a type and/or number and/or state of modules on the cartridge, a type and/or number of reservoirs on the cartridge, a type and/or number of sterile liquid transfer ports on the cartridge, and number and position of fluid paths between the modules, reservoirs, and sterile liquid transfer ports on the cartridge.

In some variations, a set of predetermined constraints may be placed on a set of the process control parameters. For example, the volume and/or the type of reagents used may be constrained based on the size of the system and/or products manufactured. Other process parameter constraints may include, but is not limited to, one or more or temperature, volume, time, pH, cell size, cell number, cell density, cell viability, dissolved oxygen, glucose levels, volumes of onboard reagent storage and waste, combinations thereof, and the like. For example, the GUI 4000 of FIG. 40 depicts that a reagent has a volume per unit of 30 ml and a required volume of 54 ml, and a consumable container has a volume per unit of 75 ml. The GUI 4800 of FIG. 48 depicts that an activation concentration is 12 mg/L, an activation culture time is 1600 seconds, activation temperature is 18° C., and a gas mix includes 21% oxygen, 78.06% nitrogen, and 0.04% of carbon dioxide. These constraints may be applied by a transformation model to generate an ordered output list of cell processing steps that affect how one or more of a robot, docking station modules, a cartridge, and cartridge modules are operated and the cell product manufactured.

In some variations, the order of operations may be constrained based on hardware constraints.

In some variations, as illustrated in the GUI 4900 of FIG. 49, a load product operation must be the first operation performed and may be performed once for each process. A fill and finish operation may always be the last operation performed before product completion and may be performed once for each process.

In some variation, the system may prevent the user from executing a set of operations in an order that may not be performed by the system.

In some variations, a notification (e.g., warning, alert) may be output if a user orders a set of operations in a “non-standard” manner. For example, a notification may be output if the same type of operation is repeated sequentially (e.g., enrichment immediately followed by enrichment). Similarly, a notification may be output if an operation (e.g., selection, activation) is used two times or more within a given process when such an operation is typically used just once in a given process.

In some variations, an output of the transformation model may correspond to an ordered output list of cell processing steps capable of being performed by the system 3508. For example, the transformation model may be executed on the sets of ordered input lists to create the ordered output list of cell processing steps. The output list of cell processing steps may control a robot, a cartridge, cartridge modules, and one or more docking station modules.

In some variations, the ordered output list is further performed by the system to control one or more of the docking station modules to perform one or more cell processing steps on one or more cell products 3512 of a respective cartridge. For example, the compute server rack 210 (e.g., controller 120) may be configured to control a cartridge electroporation module 220 configured to apply a pulsed electric field to a cell suspension of a cartridge 250. In some variations, the ordered output list may comprise instructions for a docking station module (e.g., bioreactor) to process the product (e.g., transfer the cell product from a small cartridge bioreactor module to a large cartridge bioreactor module). Furthermore, the docking station module may be further configured to operate under a set of process parameters (e.g., 9 hour duration, pH of 6.7, temperature between 37.3° C. and 37.8° C., mixing mode 3). As another example, the ordered output list may comprise instructions to operate a sterile liquid transfer module to perform one or more of removing waste from a cartridge, adding media to the cartridge, and adding a MACS reagent to the cartridge.

In some variations, one or more electronic batch records may be generated 3514 based on the process parameters and data collected from sensors during process execution. Batch records generated by the system may include process parameters, time logging, sensor measurements from the docking station modules. QC parameters determined by QC instrumentation, and other records.

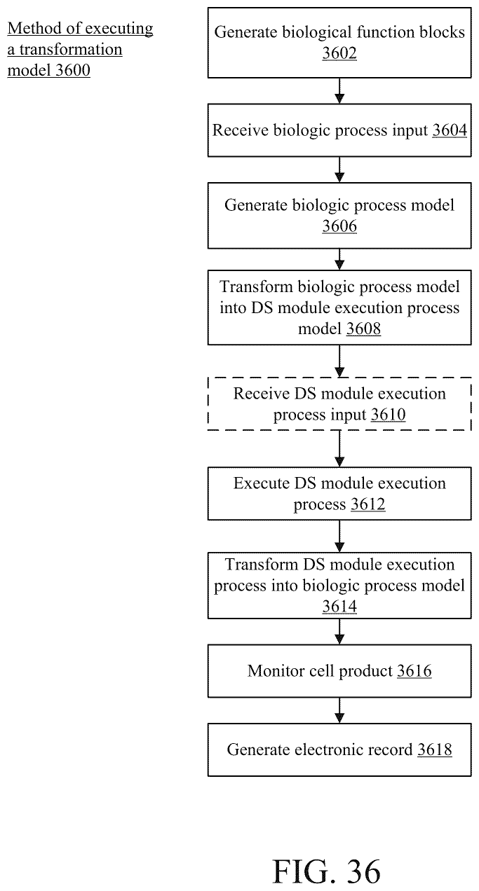

FIG. 36 is a flowchart that generally describes a variation of a method of executing a transformation model 3600. In some variations, one or more biological functions may be generated and output to a user. For example, a set of configurable biological function blocks may be displayed on a graphical user interface for user selection. The GUI may enable a user to select and order the biological function blocks and define biological control parameters. One or more control parameters of the biologic function blocks may be modified by a user if desired. In some variations, one or more biologic function templates may be generated comprising a predefined sequence of biological function blocks. One or more biological control parameters of the biologic function templates may be modified by a user if desired.

In some variations, a cell processing and manufacturing system may be configured to receive and/or store one or more biologic function (e.g., process) inputs from the user 3604. For example, a user may select one or more predefined biological function templates.

In some variations, a biologic process model (e.g., process definition) may be generated based on the biologic process inputs 3606. In some variations, a biologic process model may include one or more of enrichment, isolation, MACS selection, FACS selection, activation, genetic modification, gene transfer, transduction, transfection, expansion, formulation (e.g., harvest, pool), cryopreservation, T cell depletion, rest, tissue digestion, washing, irradiation, co-culture, combinations thereof, and the like.

In some variations, the biologic process model may be transformed into a docking station (DS) module execution process model 3608. For example, each biological function block in the biological process model may correspond to an ordered list of cell processing and manufacturing system operations with corresponding hardware control parameters. The DS module execution process model may comprise the sequence of hardware operations corresponding to the biologic process model. As described herein, the transformation model may comprise one or more constraints.

Optionally, in some variations, a cell processing and manufacturing system may be configured to receive and/or store one or more DS module execution process inputs from the user 3610. For example, a user may modify the transformed DS module execution process model if desired. The user may select specific hardware components to perform certain steps, modify timing parameters, and the like.

In some variations, the DS module execution process may be executed to generate the cell product 3612. For example, the cell processing and manufacturing system at run-time may process the cell product through the system as defined by the DS module execution process model.

In some variations, a DS module execution process may be executed 3612. In some variations, a DS module execution process model may be transformed back into a biologic process model 3614. This progress of the biologic process model may be output (e.g., displayed) to a user for monitoring. For example, the DS module execution process model may comprise one or more references (e.g., pointers) back to the biological process model so that run-time execution progress may be reported against the biological process model.

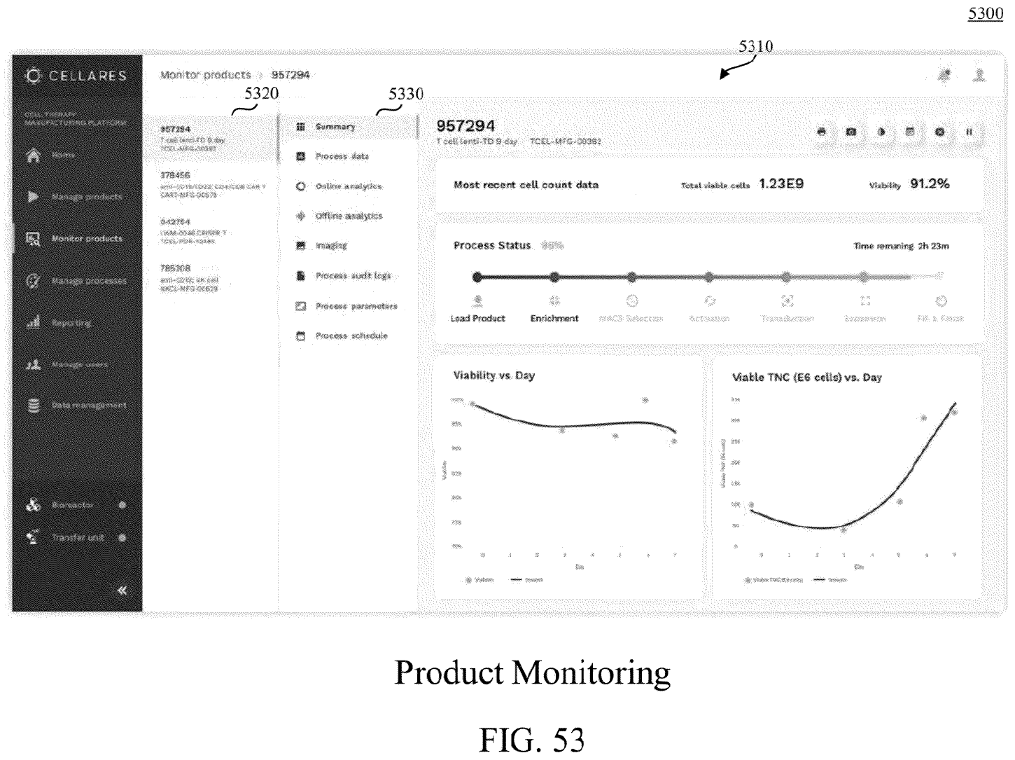

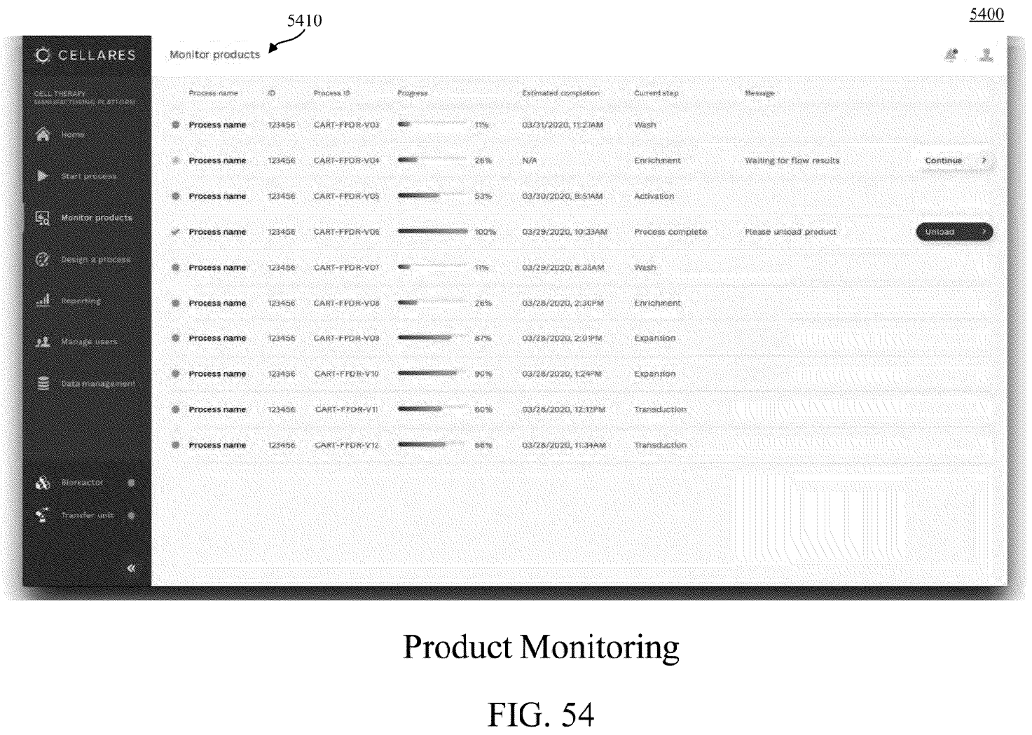

In some variations, a cell product may be monitored 3616. For example, the GUIs 5300 and 5400 of respective FIGS. 53 and 54 illustrate sensor data monitored by the system for a plurality of products. For example, a number of viable cells and a status of a process (e.g., as a function of percentage completion) may be graphically illustrated for a user.

In some variations, an electronic record may be generated based on the monitored data 3618. For example, one or more electronic batch records may be generated in compliance with, for example, 21 CFR regulations.

### Graphical User Interface

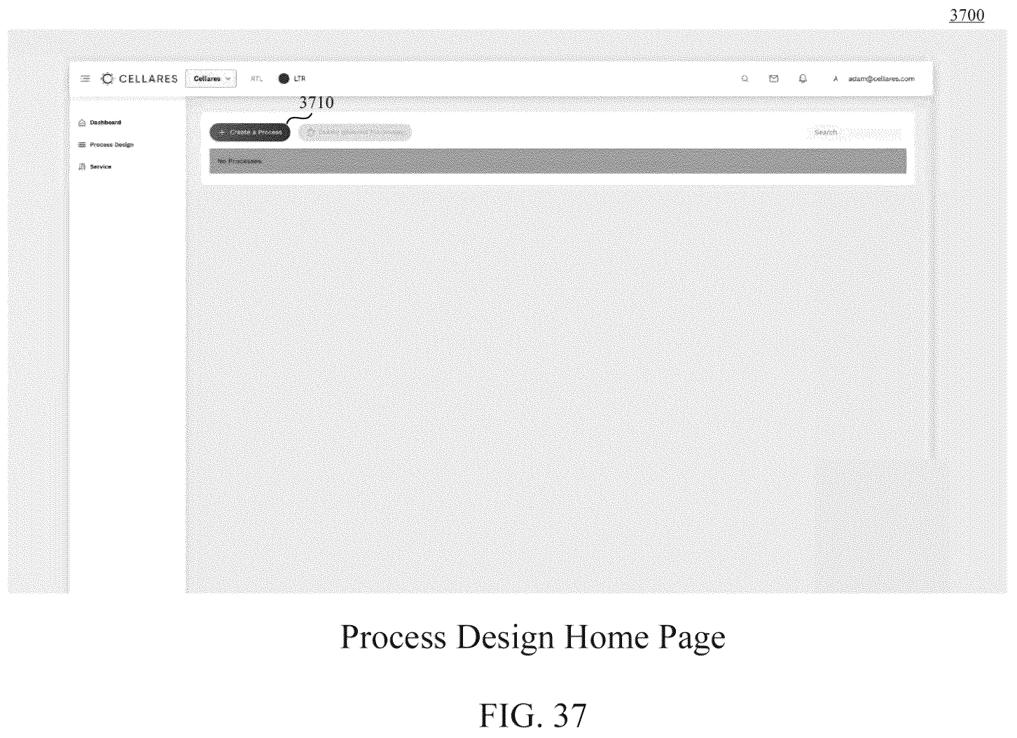

In some variations, a graphical user interface (GUI) may be configured for designing a process and monitoring a product. FIG. 37 is a variation of a GUI 3700 comprising an initial process design interface. For example, GUI 3700 may be a process design home page. The GUI 3700 may indicate that no processes have been selected or loaded. A create icon 3710 (e.g., “Create a Process”) may be selectable for a user to begin a process design process. In some variations, one or more of the GUIs described herein may include a search bar.

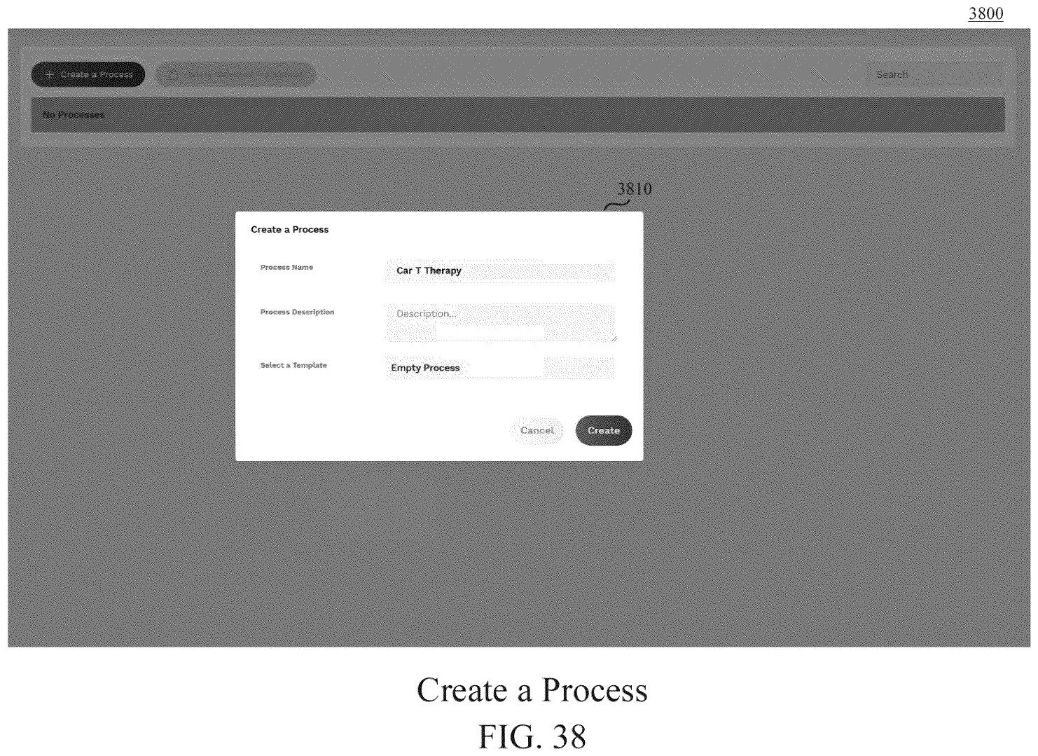

FIG. 38 is a variation of a GUI 3800 relating to creating a process. GUI 3800 may be displayed following selection of the create icon 3710 in FIG. 37. For example, GUI 3800 may comprise a process creation window 3810 allowing a user to input and/or select one or more of a process name, process description, and template. In some variations, a user may select from a list of predetermined templates. For example, a user may create a process and save it as a template for later selection.

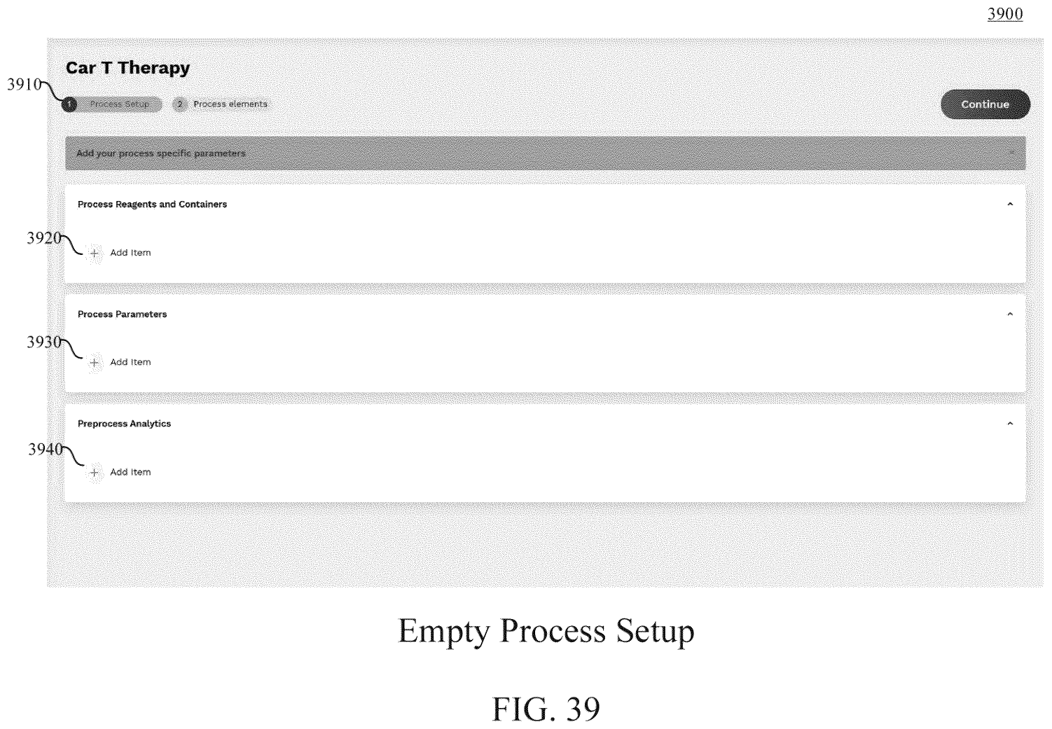

FIG. 39 is a variation of a GUI 3900 comprising relating to an empty process. GUI 3900 may be displayed following confirmation in GUI 3800 that a process is to be created. GUI 3900 may indicate the process name (e.g., Car T Therapy) and may highlight Process Setup icon 3910 and allow process specific parameters to be added such as process reagents and containers, process parameters, and preprocess analytics. GUI 3900 may further comprise an Add Process Reagents and Containers icon 3920, Add Process Parameters icon 3930, and Add Preprocess Analytics icon 3940. Once process setup is completed, one or more process elements may be specified.

In some variations, the GUI 3900 may comprise one or more predetermined templates for a set of biological processes (e.g., CAR-T, NK cells, HSC, TIL, etc.). For example, the templates may aid process development and be validated starting points for process development. The templates may be further modified (e.g., customized) based on user requirements.

FIG. 40 is a variation of a GUI 4000 comprising relating to adding a reagent and a consumable container. GUI 4000 may be displayed following selection of an Add Process Reagents and Containers icon 3920 in FIG. 39. For example, GUI 4000 may comprise an Add Reagent and Container window 4010 enabling a user to input and/or select one or more reagents comprising a reagent kind, manufacturer, part number, volume per unit, required volume and required reagent inputs (e.g., lot number, expiration date, requires container transfer). Add Reagent and Container window 4010 may comprise one or more of an input field, selection box, drop-down selector, and the like. Furthermore, the Add Reagent and Container window 3810 may enable a user to input and/or select one or more consumable containers comprising a manufacturer, part number, volume per unit, and required container inputs (e.g., lot number, expiration date). In some variations, a user may select from a list of predetermined templates. For example, a user may create a process and save it as a template.

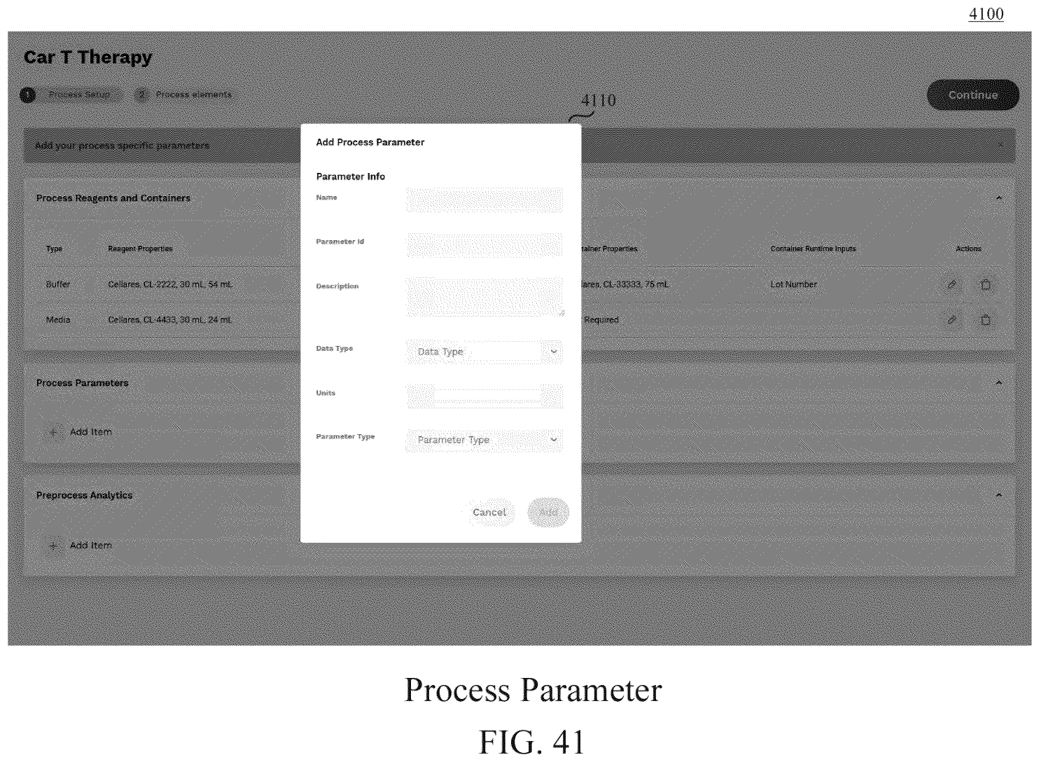

FIG. 41 is a variation of a GUI 4100 comprising relating to a process parameter. GUI 4100 may be displayed following selection of an Add Process Reagents and Containers icon 3930 in FIG. 39. For example, GUI 4100 may comprise an Add Process Parameter window 4110 enabling a user to input and/or select one or more parameters comprising a name, parameter identification, description, data type, units, and parameter type. Add Process Parameter window 4010 may comprise one or more of an input field, selection box, drop-down selector, and the like. In some variations, a user may select from a list of predetermined templates. For example, a user may create a parameter and save it as a template. FIG. 42 is a variation of a GUI 4200 comprising relating to a patient weight process parameter. For example, GUI 4200 may comprise an Add Process Parameter window 4110 having filled in parameter information including patient weight, data type (e.g., integer), units (e.g., kg), and parameter type (e.g., input).

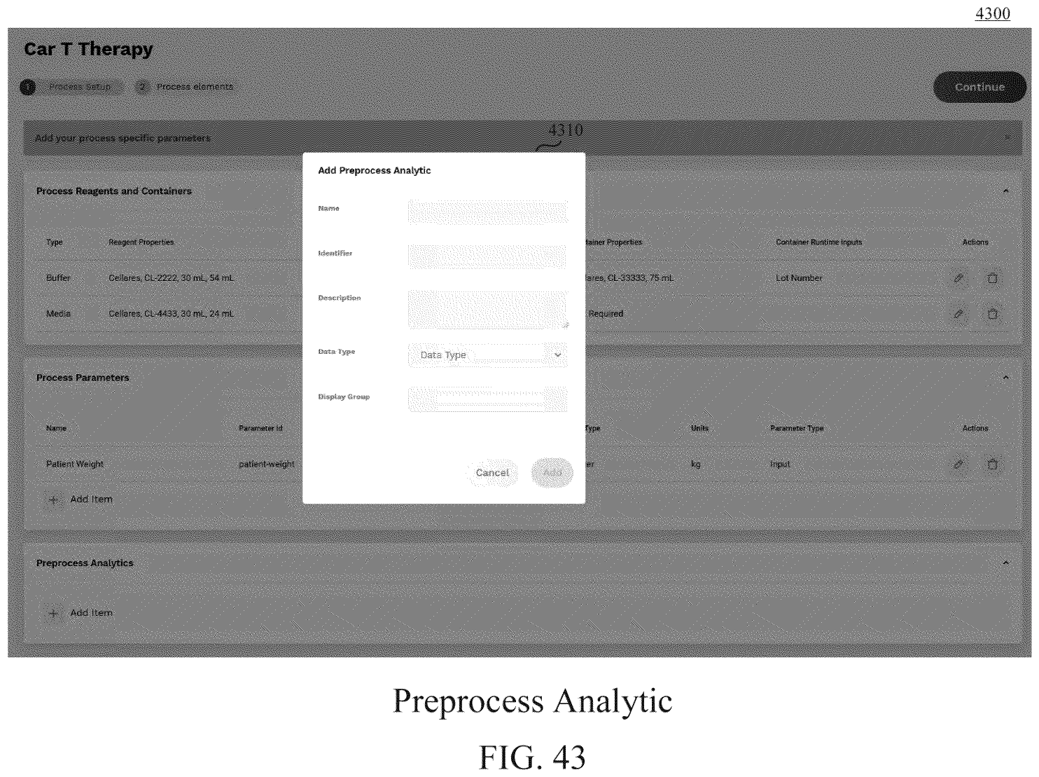

FIG. 43 is a variation of a GUI 4300 relating to a preprocess analytic. GUI 4300 may be displayed following selection of an Add Preprocess analytics icon 3940 in FIG. 39. For example, GUI 4300 may comprise an Add Preprocess Analytic window 4310 enabling a user to input and/or select one or more parameters comprising a name, identifier, description, data type, and display group. Add Preprocess Analytic window 4310 may comprise one or more of an input field, selection box, drop-down selector, and the like. In some variations, a user may select from a list of predetermined templates. For example, a user may create a parameter and save it as a template.

FIG. 44 is a variation of a GUI 4400 relating to a white blood cell count preprocess analytic. For example, GUI 4400 may comprise an Add Preprocess Analytic window 4410 having filled in preprocess analytic information including name (e.g., CBC White Blood Cell Count), identifier (e.g., CBC-white-blood-cell-count), description (e.g., Number of white blood cells in a sample), data type (e.g., float), and display group (e.g., WBC).

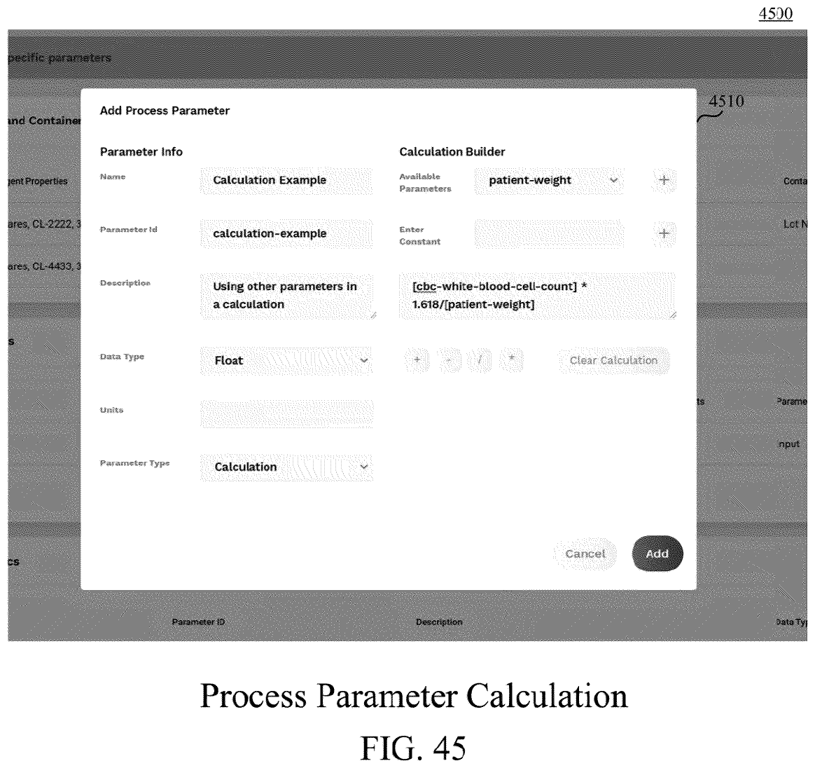

FIG. 45 is a variation of a GUI 4500 relating to a process parameter calculation. GUI 4500 may be displayed following selection of an Add Preprocess analytics icon 3940 in FIG. 39 and selection of a “Calculation” parameter type. For example, GUI 4500 may comprise an Add Preprocess Analytic window 4510 enabling a user to input and/or select one or more parameters comprising a name, identifier, description, data type, display group, units, and parameter type. Furthermore, a Calculation Builder may enable a user to define a formula (e.g., algorithm, equation) to perform a predetermined calculation. For example, a Calculation Builder may comprise one or more of a set of available parameters (e.g., patient weight), constant value, equation, and operands.

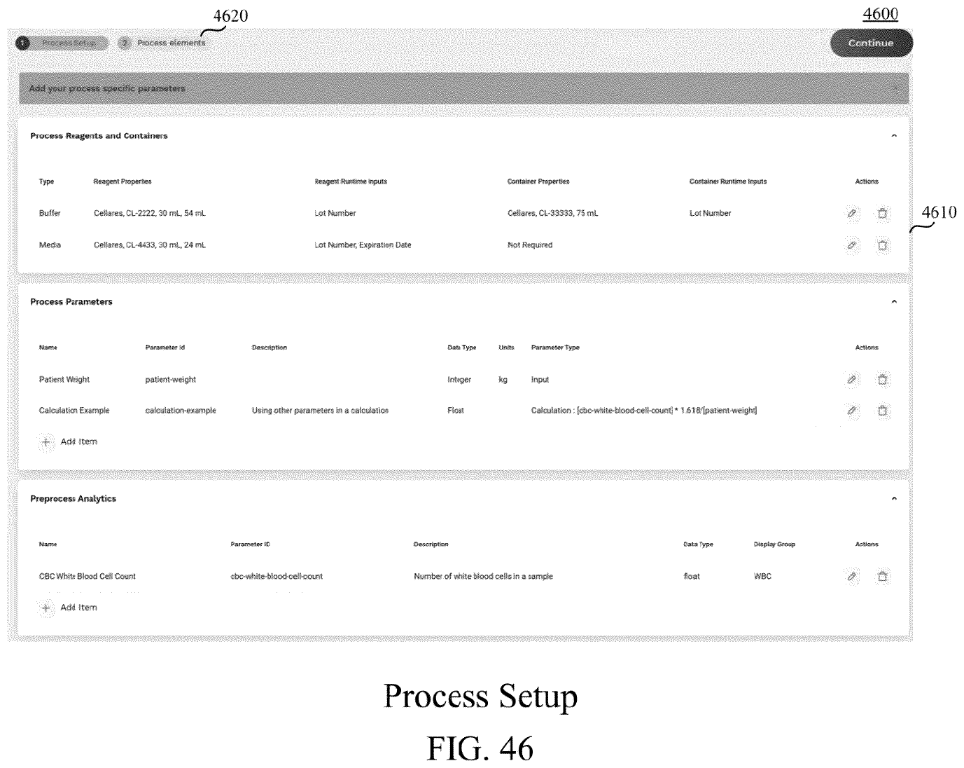

FIG. 46 is a variation of a GUI 4600 relating to a completed process setup. For example, GUI 4600 may comprise a Process Setup window 4610 having a filled in process reagents, containers, process parameters, and preprocess analytics. Once process setup is completed, one or more process elements may be specified.

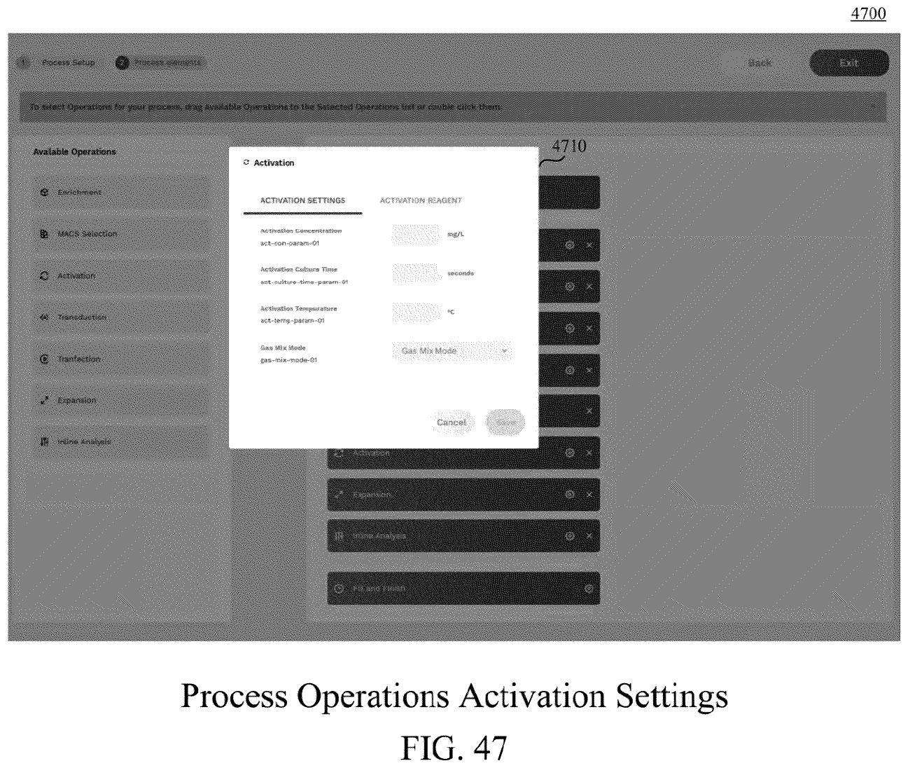

FIG. 47 is a variation of a GUI 4700 relating to process operations activation settings. GUI 4700 may be displayed following selection of a Process elements icon 4620 in FIG. 46. For example, GUI 4700 may comprise an Activation settings window 4710 allowing a user to input and/or select one or more of activation concentration (e.g., mg/L), activation culture time (e.g., seconds), activation temperature (e.g., ° C.), and gas mix mode. In some variations, a user may select from a list of predetermined templates. For example, a user may create a set of activation settings and save it as a template for later selection.

FIG. 48 is a variation of a GUI 4800 relating to a filled process operations activation settings. For example, GUI 4800 may comprise an Activation settings window 4810 having filled in Activation setting information. In some variations, a set of gases (e.g., 02, N₂, CO₂) and corresponding concentrations may be specified.

FIG. 49 is a variation of a GUI 4900 relating to a process operations interface. The GUI 4900 may comprise an Available Operations window 4910 and a Selected Operations window 4920. The available options for selection may include one or more biologic process inputs as described herein including, but not limited to, enrichment, MACS selection, activation, transduction, transfection, expansion, and inline analysis. One or more of the operations may be selected and dragged into the Selected Operations window 4920. The selected operations may be reordered within the Selected Operations window 4920.

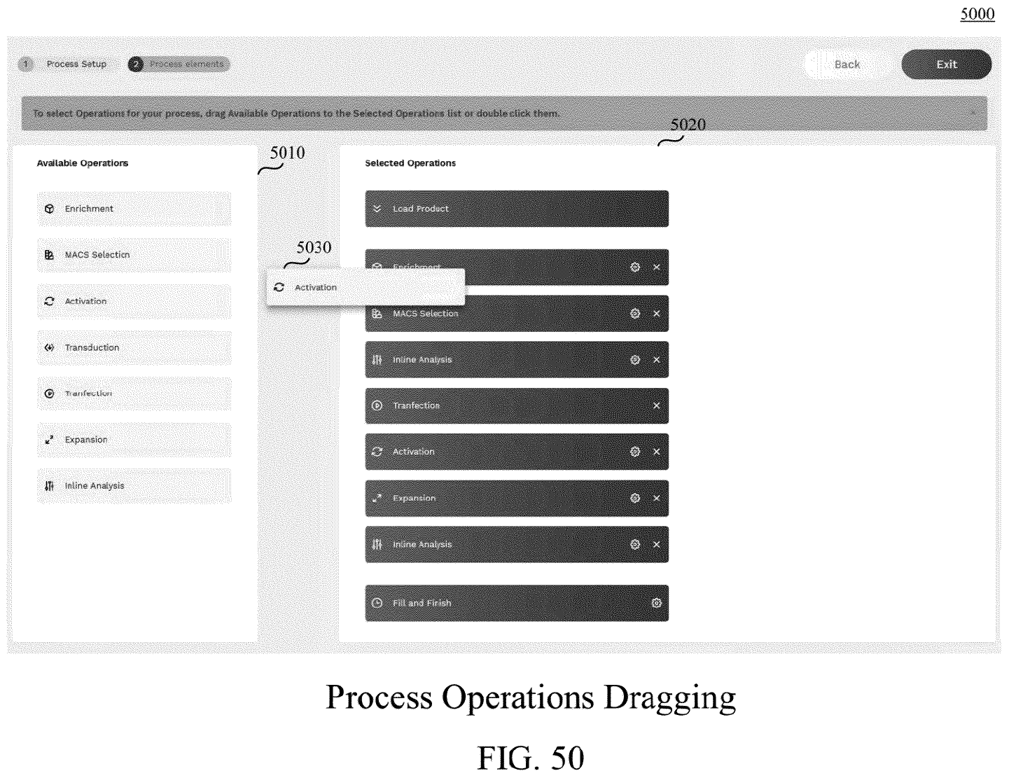

FIG. 50 is a variation of a GUI 5000 relating to dragging process operations. The GUI 5000 may comprise an Available Operations window 5010, a Selected Operations window 5020, and a selected (e.g., dragged) operation 5030 that may be drag and dropped between the Available Operations window 5010 and the Selected Operations window 5020. The Selected Operations window 5020 may comprise a plurality of selected operations.

FIG. 51 is a variation of a GUI 5100 relating to dragging process operations. The GUI 5100 may comprise an Available Operations window 5110, a Selected Operations window 5120, and a selected (e.g., dragged) operation 5130 that may be drag and dropped between the Available Operations window 5110 and the Selected Operations window 5120. The Selected Operations window 5120 may comprise a plurality of selected operations.

FIG. 52 is a variation of a GUI 5200 relating to a filled process operations. For example, the GUI 5200 may comprise an Available Operations window 5210 and a Selected Operations window 5220 comprising a completed set of selected operations. In some variations, the settings (e.g., parameters) of each operation may be selectively modified by the user by selecting a corresponding icon (e.g., gear icon).

FIGS. 53 and 54 are variations of a GUI 5300 and 5400 relating to product monitoring. The GUI 5300 and 5400 may comprise respective monitoring windows 5310, 5410. For example, the GUI 5310 may monitor a plurality of products 5320 and output one or more product characteristics 5330 including, but not limited to, a summary, process data, online analytics, imaging, process audit logs, process parameters, and process schedule. The monitoring window 5410 may monitor one or more product characteristics of one or more products. For example, the product characteristics may include, but is not limited to, one or more of a process name, identification, process identification, progress, estimated completion, current step, and message.

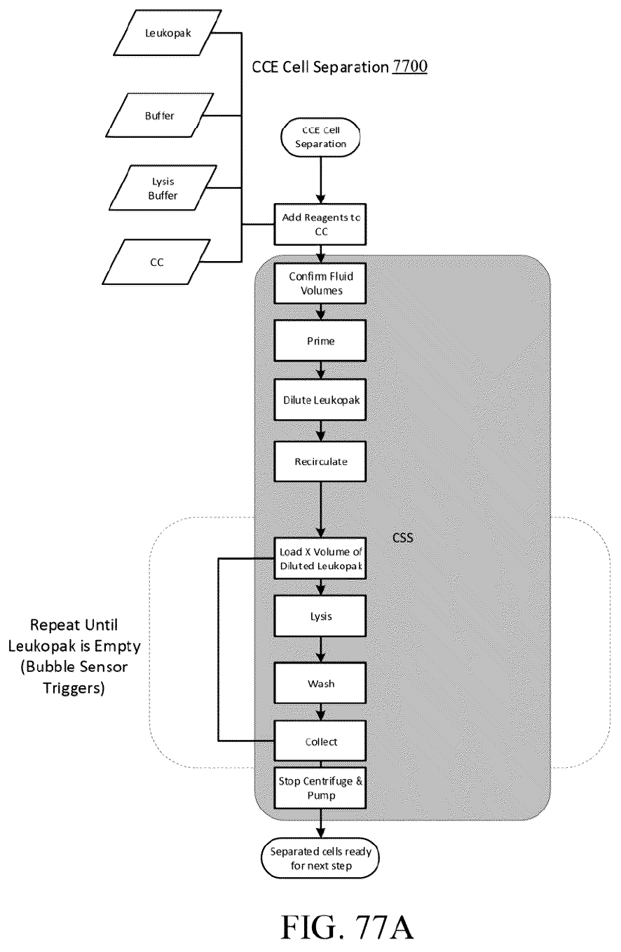

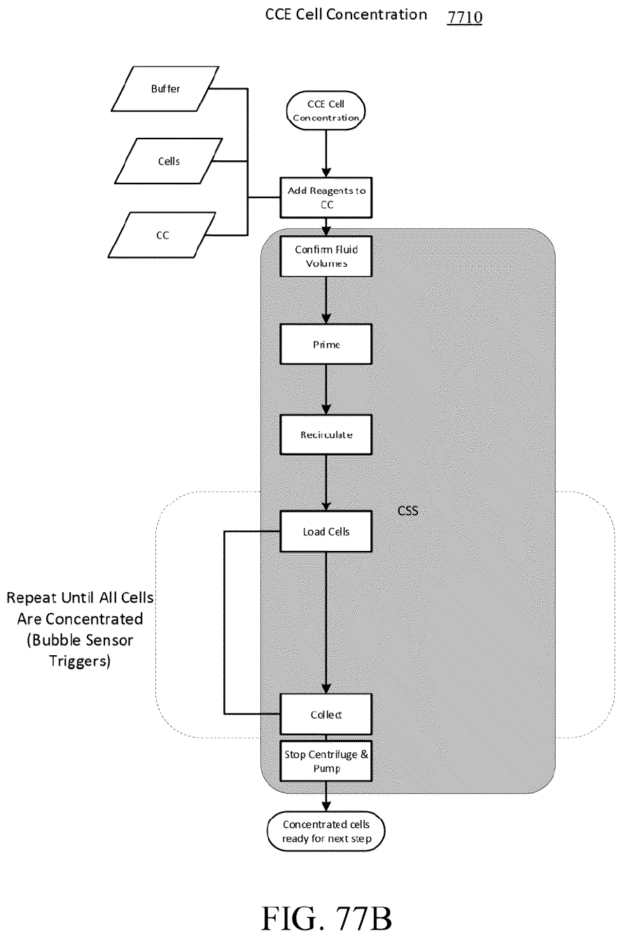

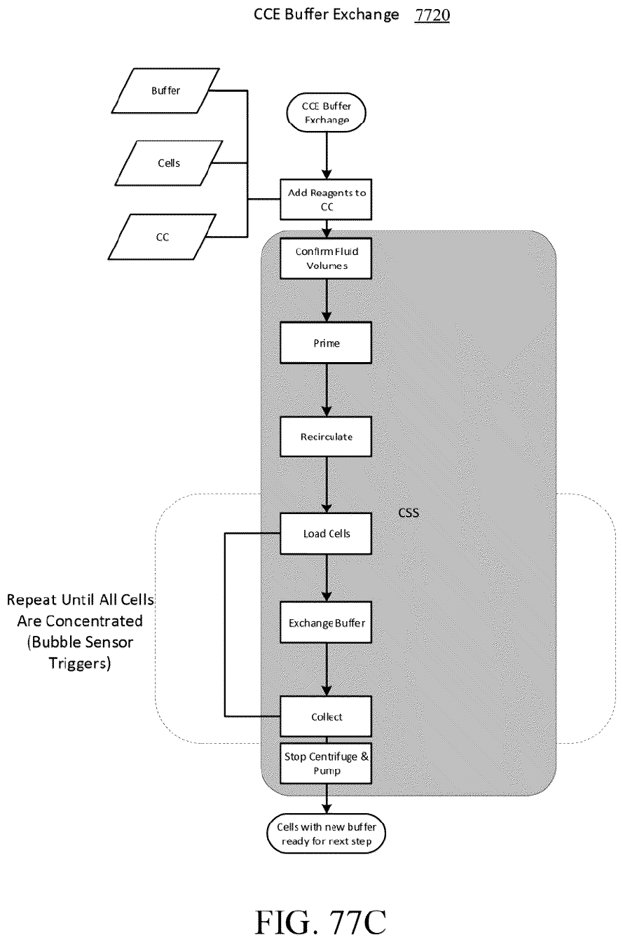

FIG. 77A is a flowchart of a method of separating cells 7700 using a cartridge CCE module. FIG. 77B is a flowchart of a method of concentrating cells 7710 using a cartridge CCE module. FIG. 77C is a flowchart of a method of buffer exchange 7720 using a cartridge CCE module.

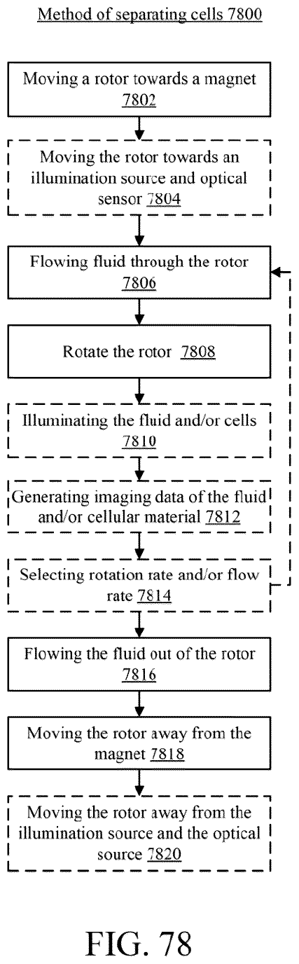

FIG. 78 is a flowchart of a method of separating cells 7800. A method of counterflow centrifugal elutriation (CCE) 7800 may comprise the step moving a rotor towards a magnet 7802. The rotor may define a rotational axis. In some variations, moving the rotor comprises advancing and withdrawing the magnet relative to the rotor using a robot. The rotor may be optionally moved towards an illumination source and an optical sensor 7804. Fluid may be flowed through the rotor 7806. In some variations, flowing the fluid comprises a flow rate of up to about 150 ml/min while rotating the rotor. The rotor may be magnetically rotated about the rotational axis using the magnet while flowing the fluid through the rotor 7808. In some variations, rotating the rotor comprises a rotation rate of up to 6,000 RPM. One or more of the fluid and the cells may be optionally illuminated using an illumination source 7810. Image data of one or more of the fluid and biological material (e.g., particles, cellular material) in the rotor may optionally be generated using an optical sensor 7812. One or more of a rotation rate of the rotor and a flow rate of the fluid may optionally be selected based at least in part on the image data 7814. The fluid may be flowed out of the rotor 7816. The rotor may be moved away from the magnet 7818. The rotor may optionally be moved away from the illumination source and the optical sensor 7820.

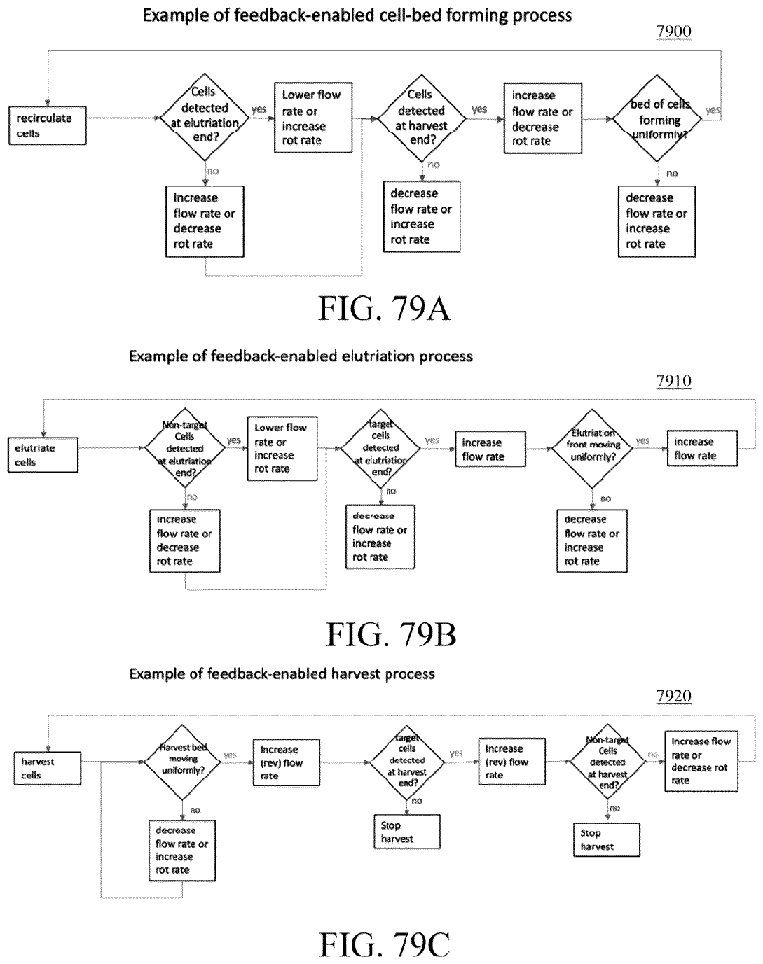

FIG. 79A is a flowchart of a closed-loop method of separating cells 7900. FIG. 79B is a flowchart of a closed-loop method of elutriating cells 7910. FIG. 79C is a flowchart of a closed-loop method of harvesting cells 7920.

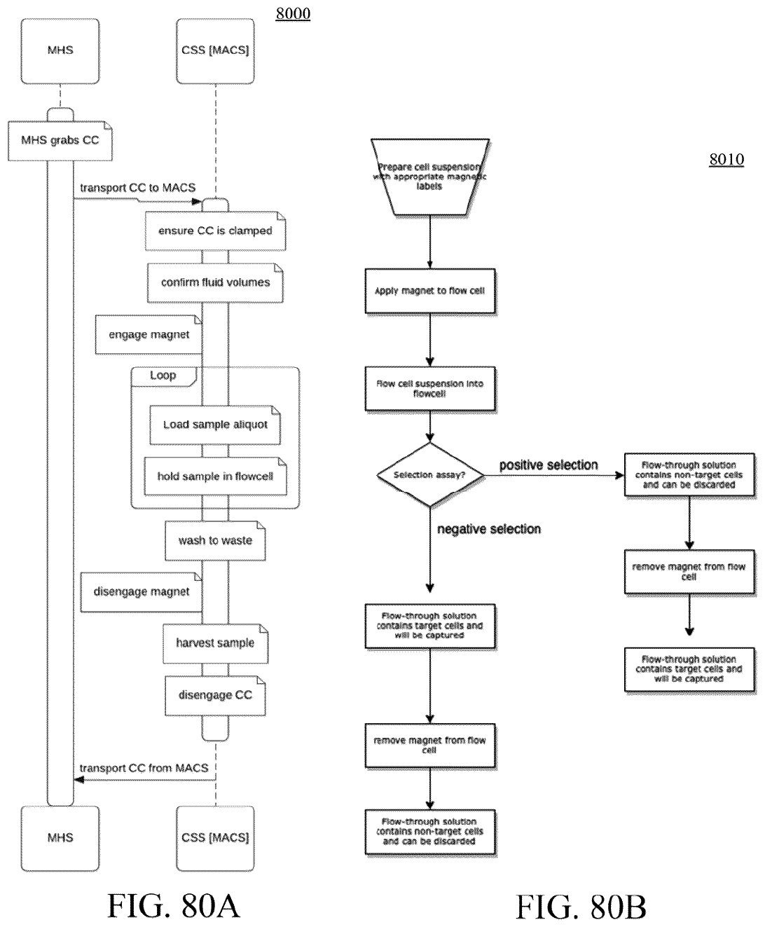

FIG. 80A is a flowchart of a method of separating cells 8000. FIG. 80B is a flowchart of a method of selecting cells 8010.

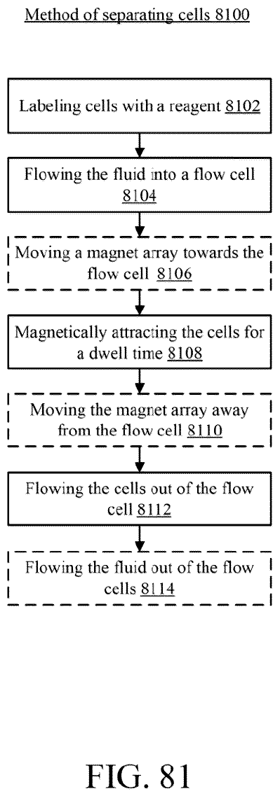

FIG. 81 is a flowchart of a method of separating cells 8100. A method of magnetic-activated cell selection (MACS) may comprise labeling cells with a reagent 8102. In some variations, a magnetic-activated cell selection (MACS) reagent may be incubated with the input cells to label the set of cells with the MACS reagent. In some variations, incubating the MACS reagent comprises a temperature between about ° C. and about 10° C. The fluid comprising input cells may be flowed into a flow cell 8104. A set of the cells are labeled with the MACS reagent. In some variations, the magnet array may optionally be moved relative to the flow cell 8106. In some variations, the set of cells may be magnetically attracted towards a magnet array for a dwell time 8108. In some variations, the dwell time may be at least about one minute. In some variations, the magnet array may be disposed external to the flow cell. In some variations, a longitudinal axis of the flow cell is perpendicular to ground. In some variations, the flow cell may be absent beads. In some variations, the magnet array may optionally be moved away from the flow cell to facilitate flowing the set of cells out of the flow cell 8110. The set of cells may be flowed out of the flow cell after the dwell time 8112. For example, flowing the set of cells out of the flow cell may comprise flowing a gas through the flow cell. The fluid without the set of cells may optionally be flowed out of the flow cell after the dwell time 8114.

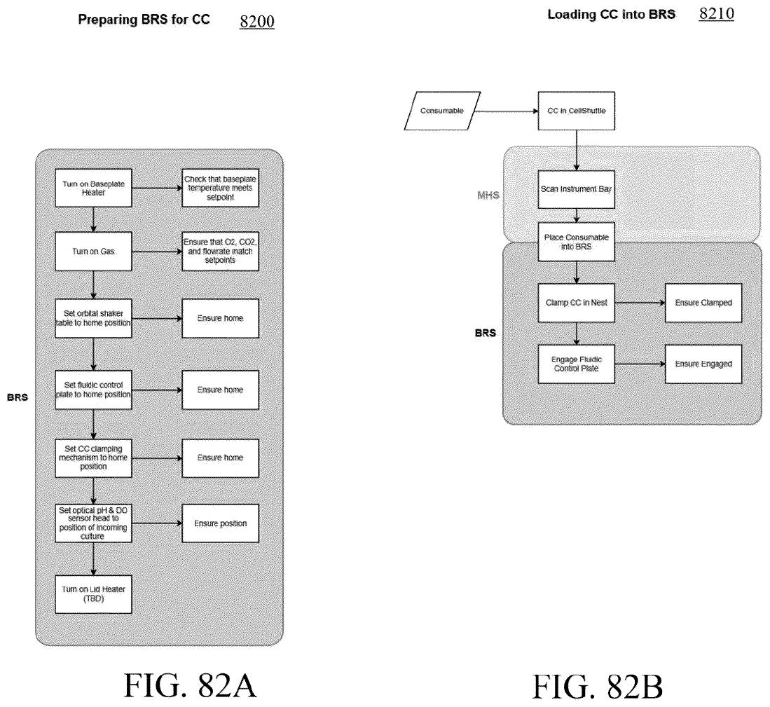

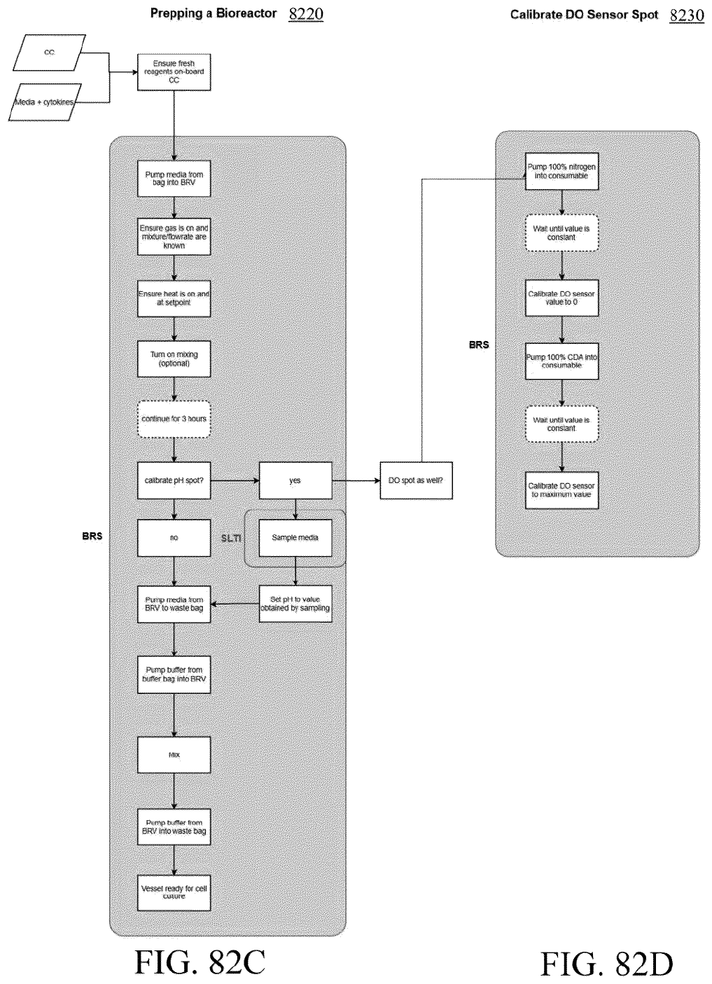

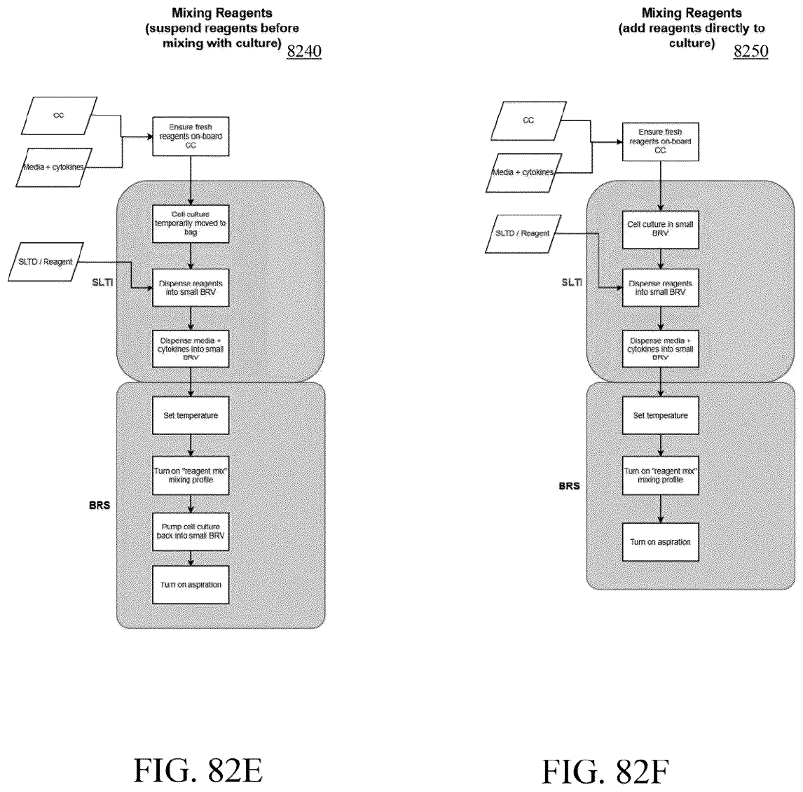

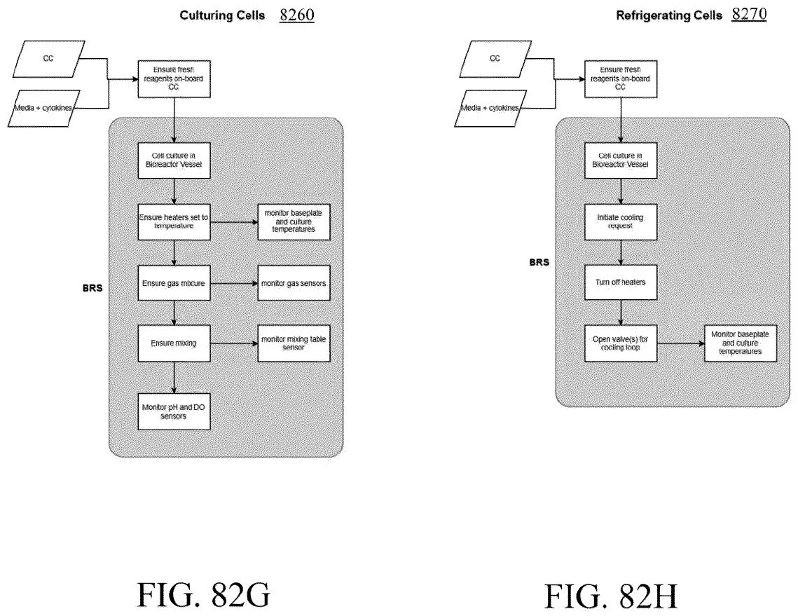

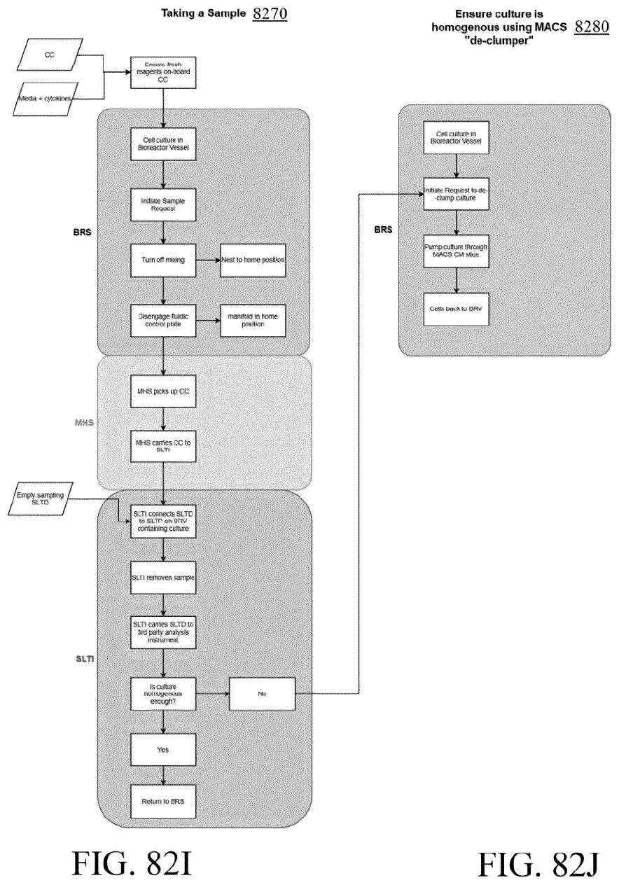

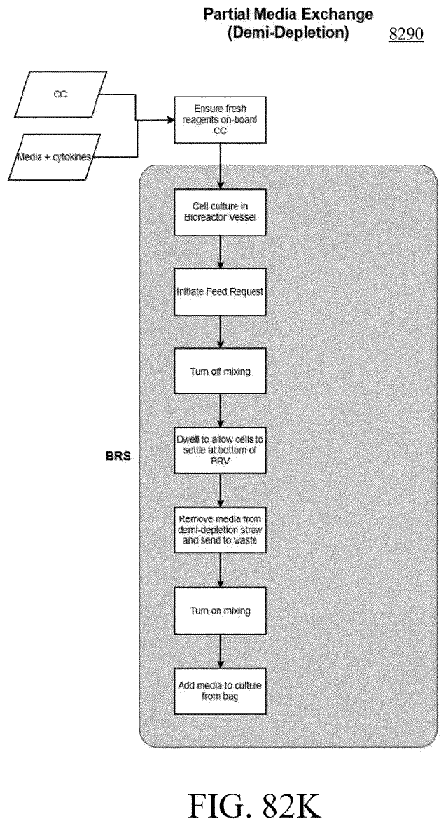

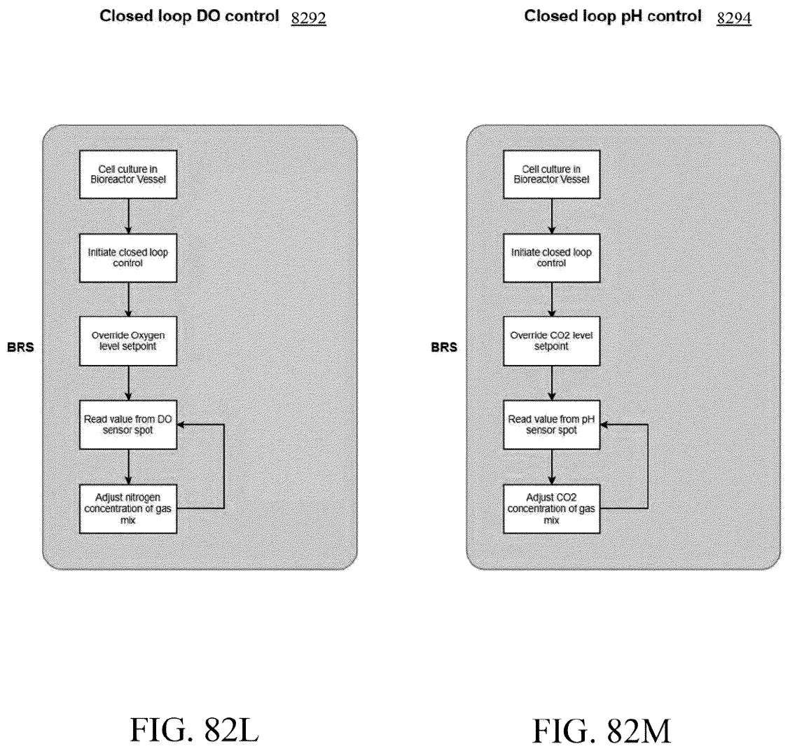

FIG. 82A is a flowchart of a method of preparing a bioreactor 8200. FIG. 82B is a flowchart of a method of loading a bioreactor 8210. FIG. 82C is a flowchart of a method of preparing a bioreactor 8220. FIG. 82D is a flowchart of a method of calibration for a bioreactor 8230. FIG. 82E is a flowchart of a method of mixing reagents 8240. FIG. 82F is a flowchart of a method of mixing reagents 8250. FIG. 82G is a flowchart of a method of culturing cells 8260. FIG. 82H is a flowchart of a method of refrigerating cells 8270. FIG. 82I is a flowchart of a method of taking a sample 8270. FIG. 82J is a flowchart of a method of culturing cells 8280. FIG. 82K is a flowchart of a method of media exchange 8290. FIG. 82L is a flowchart of a method of controlling gas 8292. FIG. 82M is a flowchart of a method of controlling pH 8294.

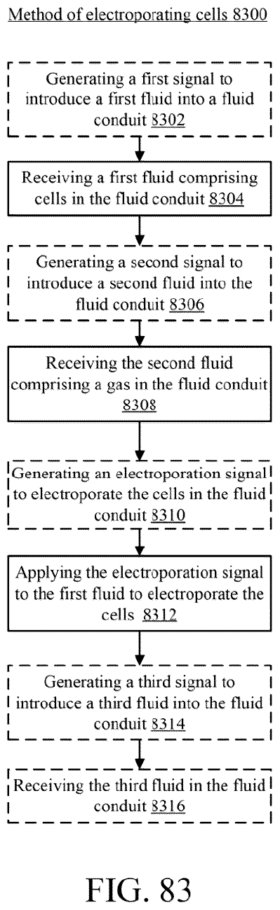

FIG. 83 is a flowchart of a method of electroporating cells 8300 using a cartridge electroporation module. In some variations, a cartridge electroporation module may comprise a fluid conduit configured to receive a first fluid comprising cells and a second fluid, a set of electrodes coupled to the fluid conduit, a pump coupled to the fluid conduit, and a controller comprising a processor and memory.

A method of electroporating cells may optionally comprise generating a first signal to introduce the first fluid into the fluid conduit using the pump 8302. A first fluid comprising cells in a fluid conduit may be received 8304. In some variations, a second signal may optionally be generated to introduce the second fluid into the fluid conduit such that the second fluid separates the first fluid from a third fluid 8306. In some variations, the second fluid may comprise a gas or oil. A second fluid in the fluid conduit may be received to separate the first fluid from a third fluid 8308. An electroporation signal may optionally be generated to electroporate the cells in the fluid conduit using the set of electrodes 8310. An electroporation signal may be applied to the first fluid to electroporate the cells 8312. In some variations, the first fluid may be substantially static when applying the electroporation signal. In some variations, a third signal may optionally be generated to introduce the third fluid into the fluid conduit 8314. The third fluid may be separated from the first fluid by the second fluid. The third fluid may optionally be received in the fluid conduit separated from the first fluid by the second fluid 8316.

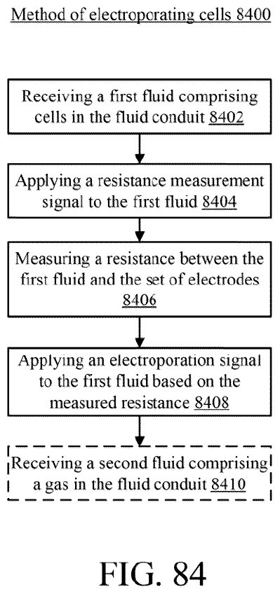

FIG. 84 is a flowchart of a method of electroporating cells 8400. A method of electroporating cells may comprise receiving a first fluid comprising cells in a fluid conduit 8402. A resistance measurement signal may be applied to the first fluid using a set of electrodes 8404. A resistance may be measured between the first fluid and the set of electrodes 8406. An electroporation signal may be applied to the first fluid based on the measured resistance 8408. In some variations, a second fluid comprising a gas may optionally be received in the fluid conduit before applying the electroporation signal to the fluid. The first fluid may be separated from a third fluid by the second fluid.

### Fluid Connector

A method of transferring fluid using a fluid connector 2700 is described in the flowchart of FIG. 27 and illustrated schematically in the corresponding steps depicted in FIGS. 16B-16L. The method 2700 may comprise the step of coupling a sterilant source to a fluid connector 2702. For example, as shown in FIG. 16B, the inlet 1652 and outlet 1654 is coupled to a sterilant source to form a fluid pathway or connection. In some variations, a robot may be configured to couple and decouple the sterilant source to the sterilant port 1650 using a fluid conduit such as a tube. In some variations, the fluid connector 1600 may comprise a plurality of sterilant ports 1650. As described herein, in some variations, a sterilant port may optionally comprise one or more of a check valve and a particle filter configured to reduce ingress of debris (e.g., after disconnecting the fluid connector). In some variations, the sterilant source may comprise or be coupled to a pump configured to circulate a sterilant through the sterilant port 1650. In some variations, the sterilant port 1650 may be coupled to one or more of a sterilant source and a fluid source such as a heated air source. For example, a first sterilant port may be configured to couple to a first sterilant source, a second sterilant port may be configured to couple to a second sterilant source, and a third sterilant source may be configured to couple to an air source.

The separate portions of the fluid connector 1600 may be brought together and mated. The method 2700 may comprise coupling a first port of a first connector to a second port of a second connector 2704. FIG. 16C is a schematic diagram of the fluid connector 1600 where the first port 216 and second port 226 are in a coupled configuration (e.g. docked position) that forms a first seal. In some variations, the first connector 1610 and the second connector 1620 may be axially and/or rotationally aligned, and one or more of the connectors 1610, 1620 may be translated to couple the connectors 1610, 1620 together. In FIG. 16C, the first port 1616 and the second port 1626 are each in a closed configuration where the lumens of the respective first connector 1610 and second connector 1620 are sealed from the external environment to maintain sterility of the lumen of the fluid connector 1600. Furthermore, the first valve 1618 and the second valve 1628 are each in a closed configuration that seals the proximal and distal ends of the connectors from each other. For example, the first valve 1618 in the closed configuration forms a seal (e.g., barrier) between the first proximal end 1612 and the first distal end 1614. Similarly, the second valve 1628 in the closed configuration forms a seal between the second proximal end 1622 and the second distal end 1624. In this manner, even if a portion of a connector is contaminated (e.g., first distal end 1614), then the other portions of the fluid connector 1600 (e.g., first proximal end 1612, second connector 1620) may remain sterile by virtue of one or more of the port seals and valve seals.

The ports may be transitioned to an open configuration such that a distal end of the connectors may be in fluid communication. The method 2700 may comprise transitioning the ports to an open configuration 2706. FIG. 16D is a schematic diagram of the fluid connector 1600 where the first port 1616 and the second port 1626 are transitioned into an open port configuration to create a shared volume between the valves 1618, 1628 that is isolated from the external environment. In FIG. 16D, the first valve 1618 and the second valve 1628 are in the closed configuration such that the chamber 1615 defines the volume (e.g., cavity) of the fluid connector 1600 between the first valve 1618 and the second valve 1628. That is, the first distal end 1614 is in fluid communication with the second distal end 1624. The ports 1616, 1627 may be received and/or held in respective housings 1617, 1627 in the closed configuration. In some variations, a robot may be configured to transition the ports 1616, 1626 between the open configuration and the closed configuration as described in more detail herein. Additionally or alternatively, the first port 1616 and second port 1626 may automatically transition (e.g., mechanically actuate) from the closed configuration to the open configuration upon mating the first port 1616 to the second port 1626.

In some variations, a fluid may be flowed into the fluid connector to aid sterilization. The method 2700 may comprise flowing fluid (e.g., liquid, gas) into the fluid connector through the sterilant port 2708. FIG. 16E is a schematic diagram of the fluid connector 1600 where the first chamber 1615 receives a fluid such as air at a predetermined temperature, pressure, and/or humidity. In some variations, one or more portions of the fluid connector 1600 may be dehumidified. For example, pressurized hot air may optionally be circulated within chamber 1615 in order to remove residual fluid, moisture, and raise a temperature of the inner surfaces of the chamber 1615. The circulated fluid may flow through housings 1617, 1627 and over inner and/or outer surfaces of the ports 1616, 1626.

Generally, sterilization of a fluid connector may comprise one or more steps of dehumidification, conditioning, decontamination, and aeration (e.g., ventilation). Dehumidification may include removing moisture from the fluid connector. Conditioning may include heating the surfaces of the fluid connector to be decontaminated in order to prevent condensation and aid sterilization. Decontamination may include circulating a sterilant through the fluid connector at a predetermined concentration, rate, and exposure time. Aeration may include removing the sterilant from the fluid connector by circulating a gas (e.g., sterile air) through the fluid connector.

A sterilant may be flowed into the fluid connector to sterilize one or more portions of the fluid connector. As described in more detail herein, the sterilant may be, for example, vaporized hydrogen peroxide (VHP) and/or ionized hydrogen peroxide (IHP). The method 2700 may comprise flowing a sterilant into the fluid connector through the sterilant port 2710. FIG. 16F is a schematic diagram of the fluid connector 1600 where the first chamber 1615 receives the sterilant for a predetermined amount of time (e.g., dwell time). For example, the sterilant may be circulated within the chamber 1615 to sterilize the chamber 1615 of the fluid connector 1600 and any contents disposed therein (e.g., other fluid, biological material). In some variations, the dwell time may be up to about 10 minutes, and between about 1 minute to about 10 minutes, including all ranges and sub-values in-between. In some variations, the vaporized hydrogen peroxide may comprise a concentration between about 50% and about 70%, including all ranges and sub-values in-between. Additionally or alternatively, one or more of the first valve 1618 and the second valve 1628 may be in the open configuration such that the sterilant may be circulated through other portions of the fluid connector 1600 such as first proximal end 1612 and second proximal end 1622.

In some variations, the valves may be translated relative to each other. The method 2700 may comprise translating a first valve relative to a second valve 2712. FIG. 16G is a schematic diagram of the fluid connector 16) where the first valve 1618 and second valve 1628 are coupled to each other (e.g., transfer position). The first valve 1618 coupled to the second valve 1628 forms a second seal between the first connector 1610 and the second connector 1620.

The valves may be transitioned to an open configuration such that each end of the fluid connector is in fluid communication. The method 2700 may comprise transitioning the first valve and the second valve from a closed configuration to an open configuration 2714. In some variations, the first valve and the second valve may comprise a spring-loaded shutoff configured to actuate to the open configuration, thereby allowing for fluidic communication between the sterile lumens of the first connector 1610 and the second connector 1620. In some variations, each of the first valve 1618 of a first connector 1610 and the second valve 1628 of a second connector 1620 may comprise an engagement feature such as threading configured to facilitate coupling between the first valve 1618 and the second valve 1628. For example, once the second valve 1628 is translated to contact the first valve 1618, the engagement features of the valves 1618, 1628 may be coupled (e.g., locked) by rotating (e.g., twisting) one of the first valve 1618 and the second valve 1628 to engage their respective threads to each other. Conversely, one of the first valve 1618 and the second valve 1628 may be rotated in the opposite direction to uncouple (e.g., unlock) the first valve 1618 from the second valve 1628.

In some variations, fluid may flow through the fluid connector 2716. FIG. 16H is a schematic diagram of the fluid connector depicted in FIG. 16A transferring fluid between fluid devices coupled to the fluid connector. For example, the contents (e.g., fluid, biological material) of the first fluid device 1630 and the second fluid device 1640 may be transferred through the fluid connector 1600. In some variations, one or more of a pump, gravity feed, and the like may aid transfer through the fluid connector 1600.

In some variations, another fluid may be flowed into the fluid connector after fluid transfer between a first fluid device and a second fluid device has been completed. The method 2700 may comprise flowing fluid (e.g., liquid, gas, sterilant) into the fluid connector through the sterilant port 2708 to remove a fluid and/or biological material from the fluid connector 2718. For example, flowing an inert gas into the fluid connector may reduce drops of liquid from forming when the first connector and second connector are separated. If a sterilant is flowed through the fluid connector, another fluid such as an inert gas may be flowed to aerate the fluid connector and ensure that the sterilant is removed.

To begin decoupling the fluid connector, the valves may be translated away from each other. The method 2700 may comprise decoupling the first connector and the second connector 2720. In some variations, a robot may be configured to manipulate the fluid connector 1600 to transition the valves 1618, 1628 to a closed configuration and to translate the valves 1618, 1628 away from each other, which may occur simultaneously or independently. The valves 1618, 1628 in the closed configuration inhibit fluid flow between the first connector 1610 and the second connector 1620. FIG. 16I is a schematic diagram of the fluid connector 1600 in a closed valve configuration where the second valve 1628 is translated away from the first valve 1618. Accordingly, the fluid connector 1600 returns to the docked position. For example, the first valve 1618 and the second valve 1628 may be configured to engage their respective spring-loaded shutoff features to form a seal and reduce drips and/or leaks. In some variations, one or more of a fluid and sterilant may optionally be configured to circulate through the chamber 1615 to remove moisture and/or sterilize the chamber 1615.

FIG. 163 is a schematic diagram of the fluid connector 1600 where the first port 1616 and the second port 1626 are transitioned from the open port configuration to the closed port configuration. In some variations, a robot may be configured to manipulate the fluid connector 1600 to transition the ports 1616, 1626 to a closed position to seal a lumen of the first connector 1610 from a lumen of the second connector 1620. In some variations, the ports 1616, 1626 may be configured to automatically transition to the closed port configuration when the first valve 1618 separates from the second valve 1628.

FIG. 16K is a schematic diagram of the fluid connector 1600 where the second connector 1620 is translated away from the first connector 1610. In some variations, a robot may be configured to manipulate the fluid connector 1600 to separate the first connector 1610 from the second connector 1620. FIG. 16K depicts the fluid connector 1600 in a disengaged configuration.

FIG. 16L is a schematic diagram of the fluid connector 1600 decoupled from the sterilant source. In some variations, a robot may be configured to manipulate the fluid connector 1600 and/or sterilant source to separate the sterilant source 1650 from the sterilant source. In some variations, the sterilant source may be decoupled from the fluid connector 1600 at any point after completing a sterilization process.

In some variations, the cartridge comprises one or more Sterile Liquid Transfer Ports (SLTPs) configured for use with a Sterile Liquid Transfer Device (SLTD). In some variations, the SLTP comprises one or more of a cap, a fitting, and a tube fluidically coupled to the fitting. The cap may be removable or pierceable. The fitting may be a push-to-connect fitting (PTCF) or a threaded fitting. PTCF include male-to-female, female-to-male, and androgynous fittings. Illustrative SLTPs and SLTDs suitable for use in the systems of the disclosure may include, for example, AseptiQuik® S connectors, Lynx® CDR connectors, Kleenpak™ connectors, Intact™ connectors, GE LifeScience®, ReadyMate connectors.

When the disclosure refers to sterile liquid transfer devices, sterile liquid transfer ports, and sterile liquid transfer, the word “sterile” should be understood as a non-limiting description of some variations—an optional feature providing advantages in operation of certain systems and methods of the disclosure. Maintaining sterility is typically desirable for cell processing but may be achieved in various ways, including but not limited to providing sterile reagents, media, cells, and other solutions; sterilizing cartridge(s) and/or cartridge component(s) after loading (preserving the cell product from destruction); and/or operating the system in a sterile enclosure, environment, building, room, or the like. Such operator performed or system performed sterilization steps may make the cartridge or cartridge components sterile and/or preserve the sterility of the cartridge or cartridge components.

### III. EXAMPLES

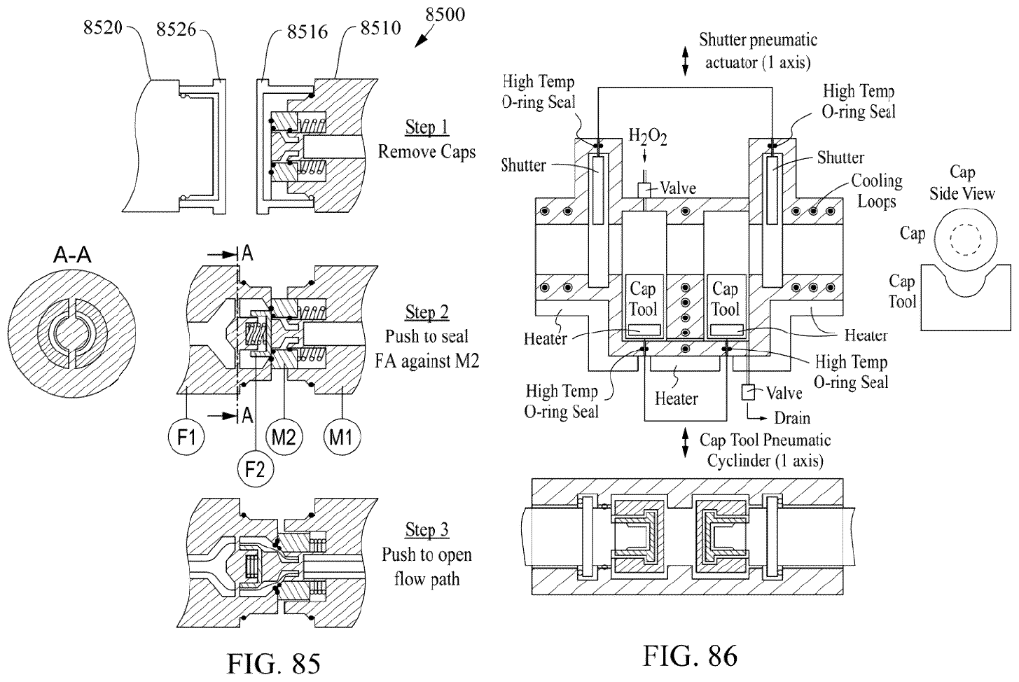

FIGS. 85-96D are diagrams of other variations of a fluid connector. FIG. 85 depicts a fluid connector 8500 comprising a first connector 8510 including a first cap 8516 and a second connector 8520 including a second cap 8526. Fluid connector 8500 may comprise a male connector and a female connector, each with a removable cap and internal self-shutoff valve configured to reduce leaks and drips. The first cap 8516 and the second cap 8526 may be removable from their respective connectors 8510, 8520.

In some variations, the fluid connector may be used with a self-sterilizing cap and decap tool 8600 depicted in FIG. 86. The cap/decap tool 8600 may be configured to facilitate a sterile environment (e.g., ISO5) where the caps may be removed and the connectors pressed together, first sealing the connectors to each other, and then pressed further to transition the internal self-shutoff valves to an open configuration.

In some variations, the tool 8600 may be configured to remove and re-apply caps to the fluid connector 8500, and to provide a sterile volume for aseptic connection and disconnection of the fluid connector 8500 pair. In some variations, a method of using the tool 8600 may comprise inserting both capped connectors in a first configuration (e.g., where the caps approach the closed shutters) such that the fluid connectors form a seal within a lumen of the decap tool 8600. In some variations the shutters may be opened to ensure a decap mechanism is retracted. Both capped connectors may be pushed to form a second configuration. The decap mechanism may be engaged to lock into features on the caps. Both capped connectors may be retracted to the first configuration where the caps are retained in the decap mechanism. The decap mechanism may be retracted such that the caps are held within a recess in the tool 8600. The internal volume may optionally be decontaminated with sterilant or heat. Both connectors may be advanced to connect and perform the transfer. The steps described herein may be sequentially reversed.

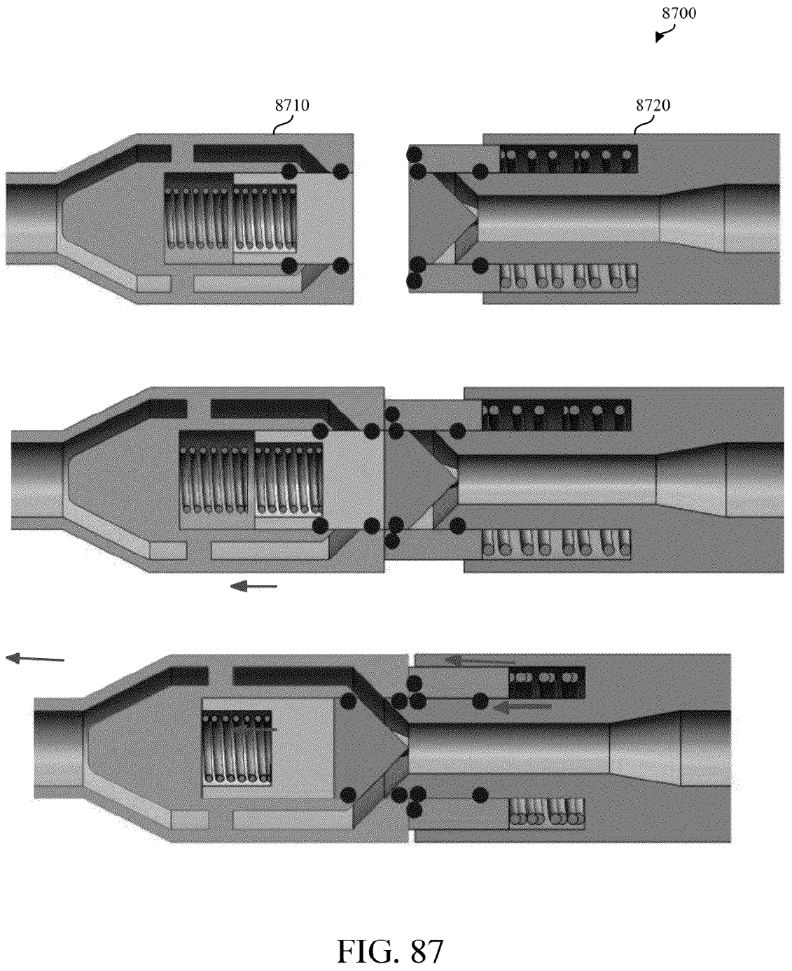

FIG. 87 depict a coupling sequence for a self-sealing fluid connector 8700 comprising a first connector 8710 and a second connector 8720. The fluid connector 8700 may be configured to reduce leaks and drips and may facilitate smoother fluid flow path by removing spring elements from contact with fluid.

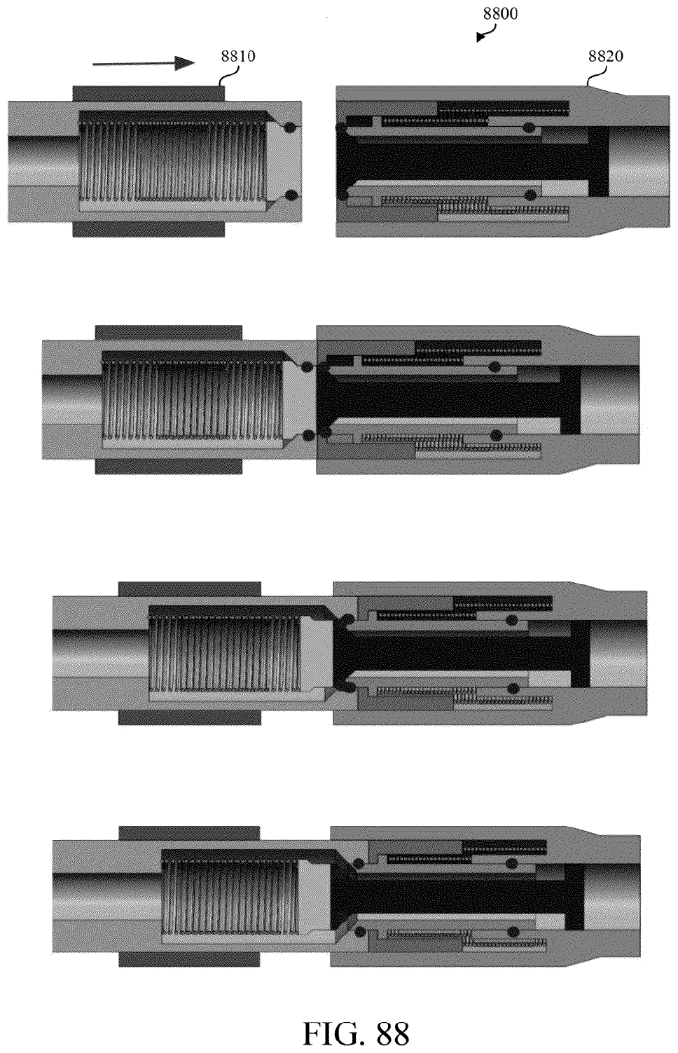

FIG. 88 depict a coupling sequence for a self-sealing fluid connector 8800 comprising a first connector 8810 and a second connector 8820.

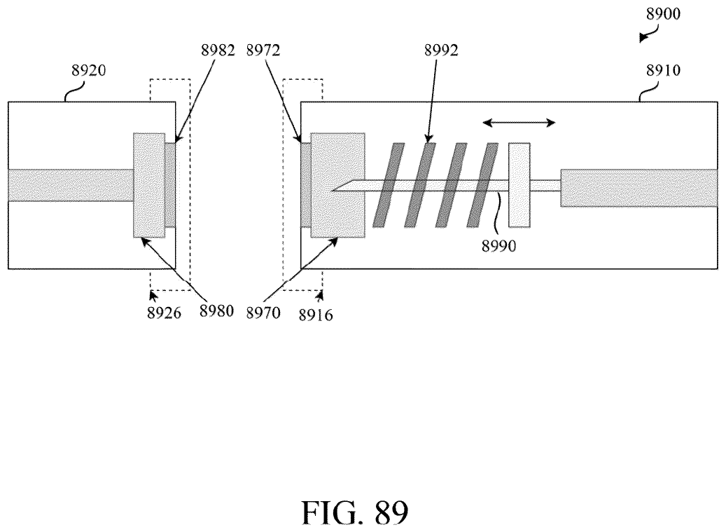

In some variations, a fluid connector may transfer fluids in a sterile manner using a retractable needle. FIG. 89 depicts a fluid connector 8900 comprising a first connector 8910 and a second connector 8920. The first connector 8910 may comprise a first cap 8916 configured to removably couple to a distal end of the first connector 8910. The first connector 8910 may comprise a first elastomeric member 8970 (e.g., sealing septum) and a first thermal member 8972 (e.g., thermally resealable septum) disposed at a distal end of the first connector 8910. The first connector 8910 may further comprise a needle 8990 and a spring 8992 coupled to the first elastomeric member 8970 and the needle 8990. The second connector 8920 may comprise a second cap 8926 configured to removably couple to a distal end of the second connector 8920. The second connector 8920 may comprise a second elastomeric member 8980 (e.g., sealing septum) and a second thermal member 8982 (e.g., thermally resealable septum) disposed at a distal end of the second connector 8920.

In some variations, the needle 8990 may be advanced through each of the first elastomeric member 8970, first thermal member 8972, second thermal member 8982, and second elastomeric member 8980 to form a fluid pathway between the first connector 8910 and the second connector 8920. Fluid may flow through the first connector 8910 and into the second connector 8920 via a lumen of needle 8990. Each of the elastomeric members 8970, 8980 and thermal members 8972, 8982 may seal once the needle 8990 is withdrawn from a distal end of the first connector 8910. For example, the thermal member 8972, 8982 may be configured to thermally seal at a predetermined temperature and the elastomeric members 8970, 8980 may self-seal once the needle 8990 has been withdrawn. In some variations, the fluid connector 8900 may be thermally decontaminated and resealed after fluid transfer. For example, the fluid connector 8900 (e.g., thermal members 8972, 8982) may be heated using one or more of a laser, contact heating, heated air, combinations thereof, and the like.

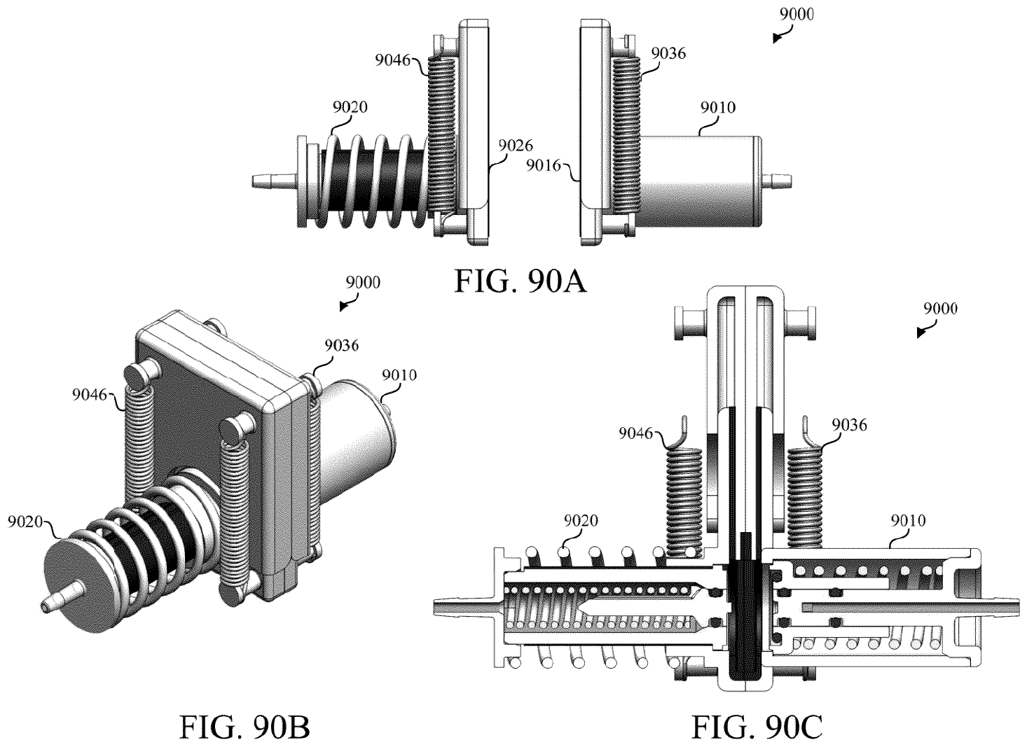

In some variations, a fluid connector may comprise a port comprising an actuator configured to transition the port between a closed port configuration and an open port configuration. In some variations, the actuator may comprise a spring such as an external spring, a rotary spring, and a linear spring, as described in more detail with respect to FIGS. 90A-96D.

FIGS. 90A-90C depict a fluid connector having an external spring actuator. FIG. 90A is a side view, FIG. 90B is a perspective view, and FIG. 90C is a cross-sectional side view of a fluid connector 9000 comprising a first connector 9010 and second connector 9020. The first connector 9010 may comprise a first port 9016 comprising a first spring 9036, and the second connector 9020 may comprise a second port 9026 comprising a second spring 9046. The springs 9036, 9046 may be configured to actuate respective ports 9016, 9026 between a closed port configuration and an open port configuration. Although not shown in FIG. 90C, springs 9036, 9046 may be coupled in an extended configuration to the pin in the open port configuration.

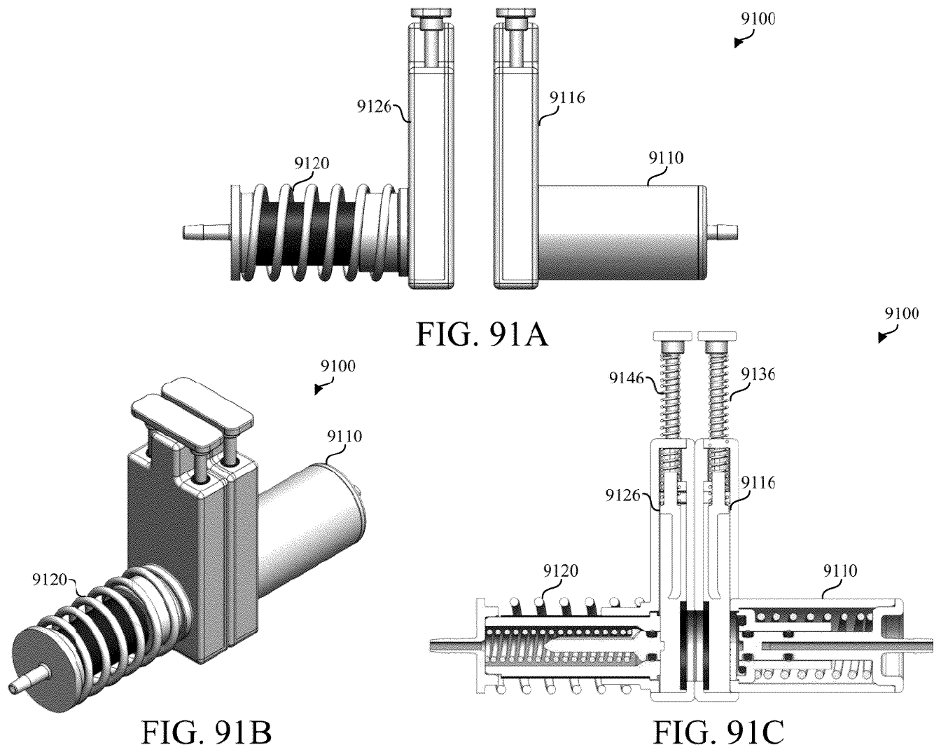

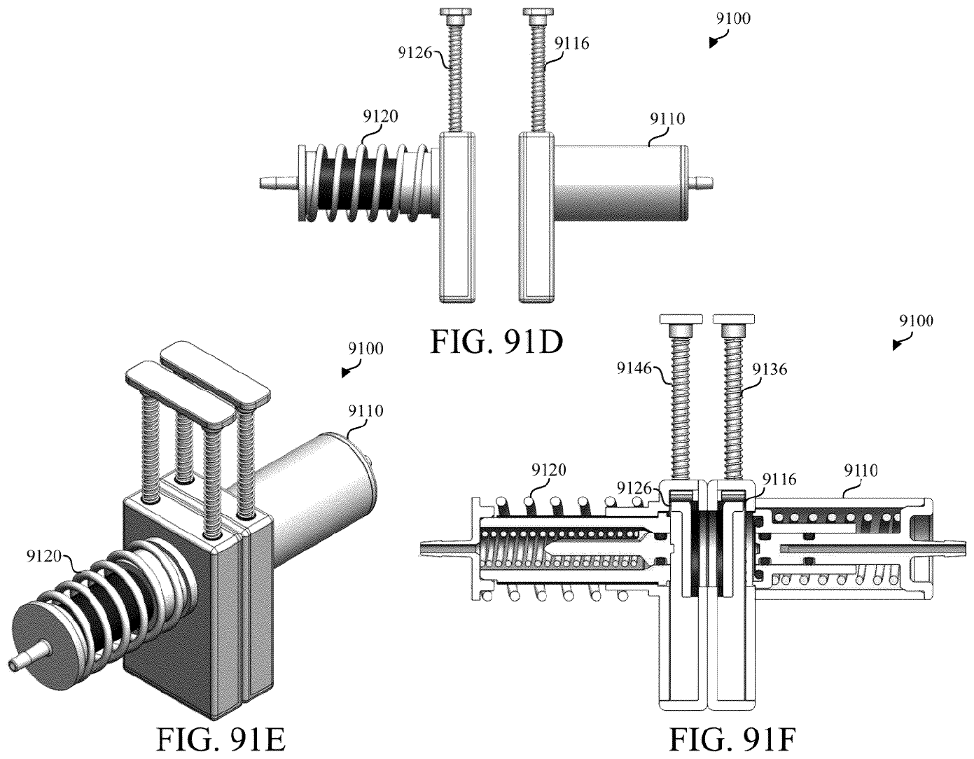

FIGS. 91A-91F depict a fluid connector having a linear spring actuator. FIG. 91A is a side view, FIG. 91B is a perspective view, and FIG. 91C is a cross-sectional side view of the fluid connector 9100 in an open port configuration. The fluid connector 9100 may comprise a first connector 9110 and second connector 9120. The first connector 9110 may comprise a first port 9116 comprising a first spring 9136, and the second connector 9120 may comprise a second port 9126 comprising a second spring 9146. The springs 9136, 9146 may be configured to actuate respective ports 9116, 9126 between a closed port configuration and an open port configuration. FIG. 91D is a side view, FIG. 91E is a perspective view, and FIG. 91F is a cross-sectional side view of the fluid connector 9100 in a closed configuration.

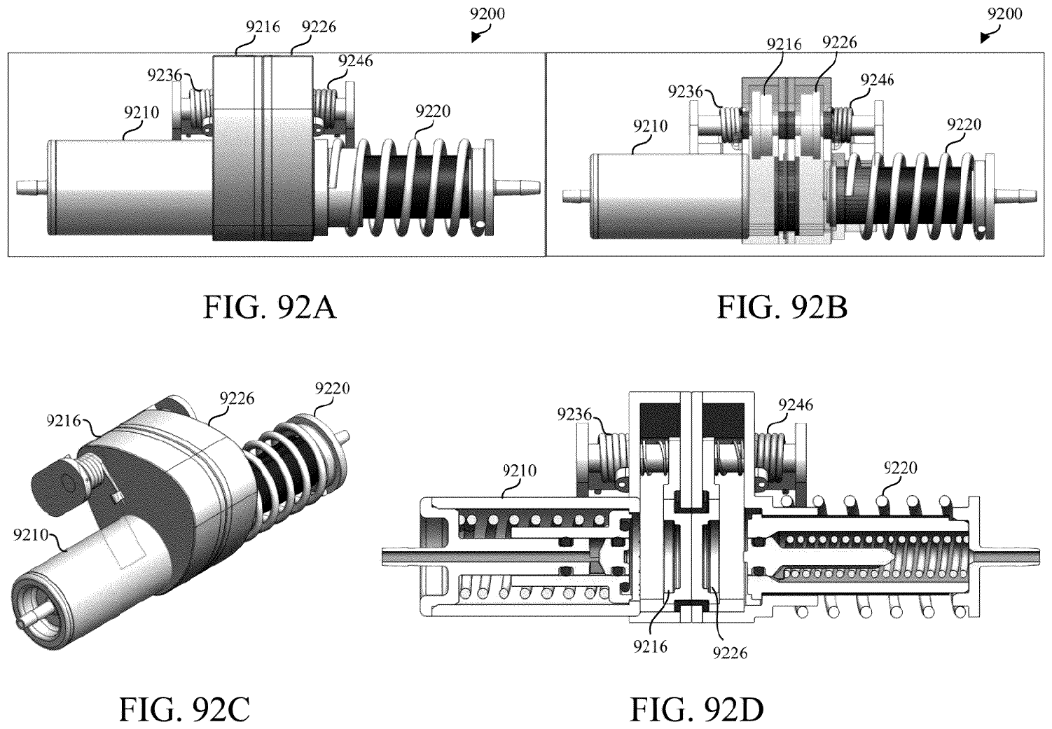

FIGS. 92A-92D depict a fluid connector having a rotary spring actuator. FIG. 92A is a side view, FIG. 92B is a transparent side view, FIG. 92C is a perspective view, and FIG. 92D is a cross-sectional side view of a fluid connector 9200 comprising a first connector 9210 and second connector 9220. The first connector 9210 may comprise a first port 9216 comprising a first spring 9236, and the second connector 9220 may comprise a second port 9226 comprising a second spring 9246. The springs 9236, 9246 may be configured to actuate respective ports 9216, 9226 between a closed port configuration and an open port configuration. FIG. 92B shows the ports 9216, 9226 in an open port configuration and FIG. 92D shows the ports 9216, 9226 in a closed port configuration.

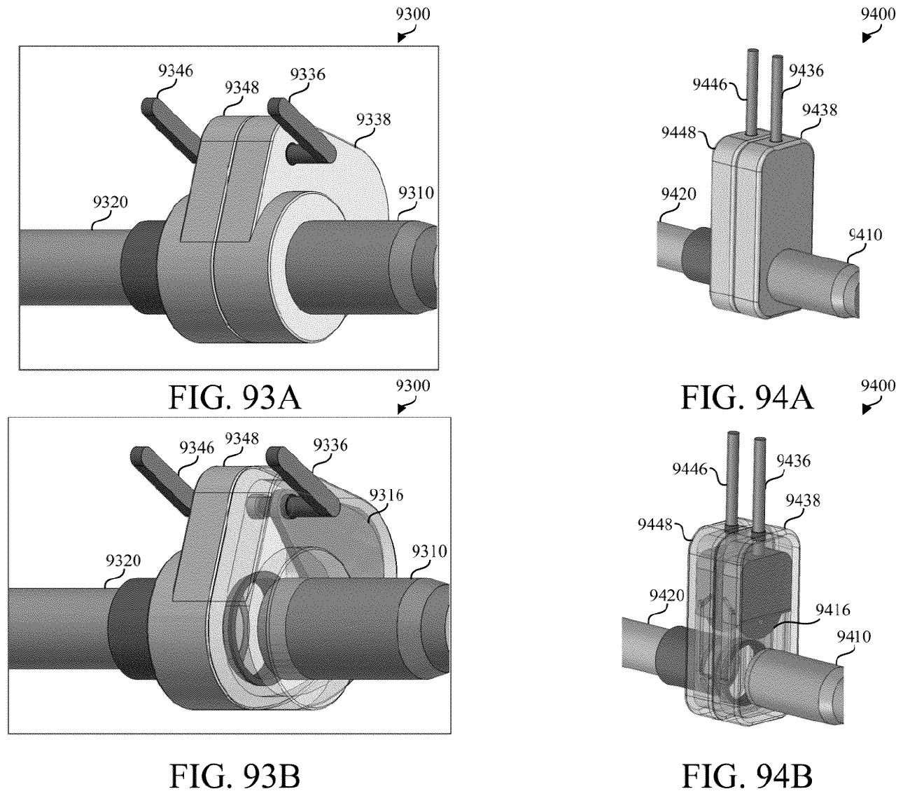

FIGS. 93A-94B depict fluid connectors having ports enclosed within a housing (e.g., enclosure). FIG. 93A is a perspective view and FIG. 93B is a transparent perspective view of a fluid connector 9300 comprising a first connector 9310 having a first housing 9338 and first actuator 9336, and a second connector 9320 having a second housing 9348 and a second actuator 9346. FIG. 93B shows a first port 9316 enclosed within first 9338 housing. The first port 9316 is coupled to the first actuator 9336 configured to transition the first port 9316 between an open port configuration (shown in FIG. 93B) and a closed port configuration.

FIG. 94A is a perspective and FIG. 94B is a transparent perspective view of a fluid connector 9400 comprising a first connector 9410 having a first housing 9438 and a first actuator 9436, and a second connector 9420 having a second housing 9448 and a second actuator 9446. FIG. 94B shows a first port 9416 enclosed within first 9438 housing. The first actuator 9436 coupled to the first port 9416 may be configured to transition the first port 9416 between an open port configuration (shown in FIG. 94B) and a closed port configuration.

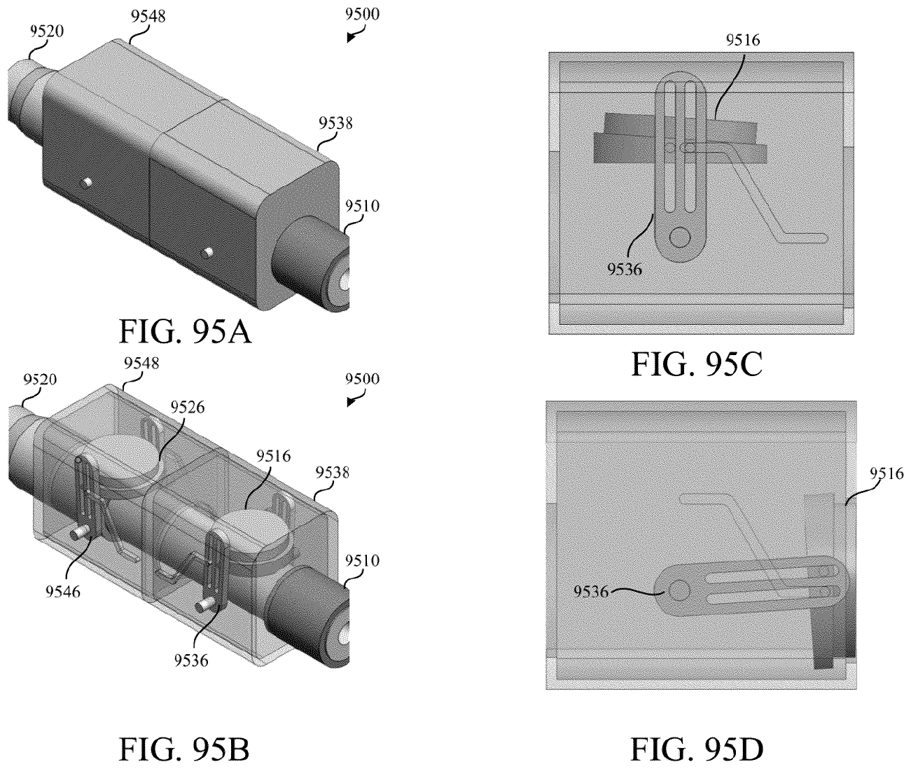

FIG. 95A is a perspective view and FIG. 95B is a transparent perspective view of a fluid connector 9500 comprising a first connector 9510 having a first housing 9538, first port 9516, and a first actuator 9536. A second connector 9520 may comprise a second housing 9548, second port 9526, and a second actuator 9546. FIG. 95B shows the first port 9516 and the second port 9526 each in an open port configuration. For example, the first actuator 9536 coupled to the first port 9516 may be configured to transition the first port 9516 between an open port configuration and a closed port configuration. FIG. 95C is a detailed side view of the first port 9516 and first actuator 9536 in an open port configuration, and FIG. 95D is a detailed side view of the first port 9516 and first actuator 9536 in a closed port configuration.

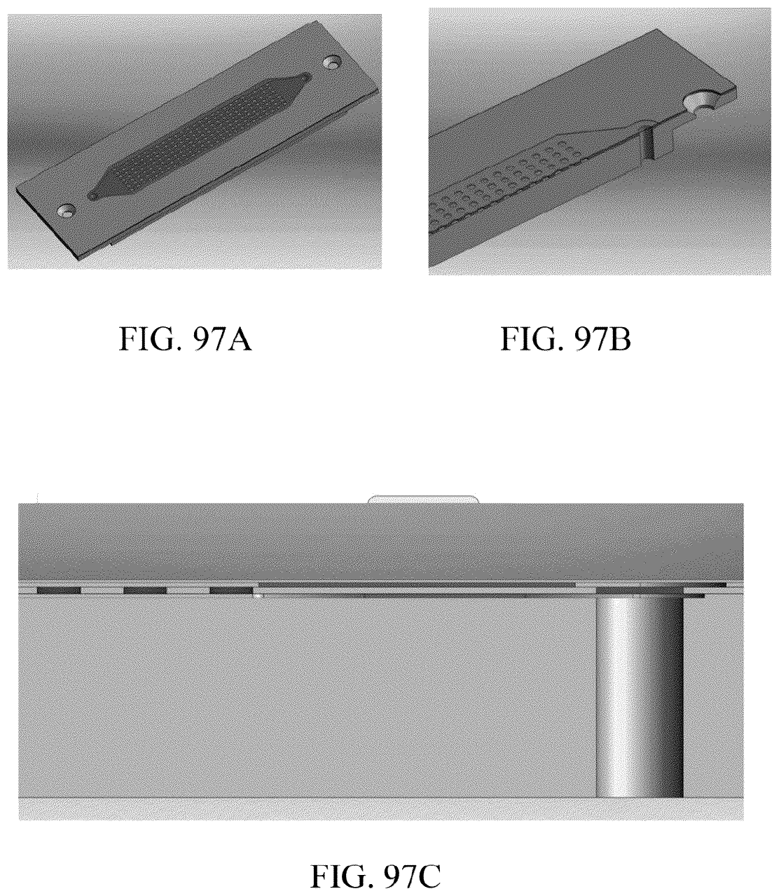

FIG. 97A is a perspective view of a cartridge MACS module. FIG. 97B is a cross-sectional perspective view of a cartridge MACS module. FIG. 97C is a cross-sectional side view of a cartridge MACS module.

All variations of any aspect of the disclosure may be used in combination, unless the context clearly dictates otherwise.

While variations of the present invention have been shown and described herein, those skilled in the art will understand that such variations are provided by way of example only. Numerous variations, changes, and substitutions will now occur to those skilled in the art without departing from the invention. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed. It should be understood that various alternatives to the variations of the invention described herein may be employed in practicing the invention. It is intended that the following claims define the scope of the invention and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE INVENTION

Notwithstanding the appended claims, the disclosure sets forth the following numbered embodiments:

(1) An automated system for cell processing, comprising a cartridge comprising a plurality of cartridge modules including a bioreactor module, a counterflow centrifugal elutriation module, and at least one of an electroporation module, a magnetic-activated cell selection module, a fluorescence-activated cell selection module, or a spinoculation module, at least one sterile liquid transfer port, and a liquid transfer bus fluidically coupled to each cartridge module, and a docking station comprising a plurality of docking station modules corresponding to the plurality of cartridge modules, each of the plurality of docking station modules being independently configured to perform one or more cell processing operations upon the cartridge in coordination with a respective cartridge module, wherein the docking station is sized and shaped to receive a single cartridge.

(2) The system of (1), wherein the liquid transfer bus is fluidically coupled to each cartridge module by fluid conduits arranged between ports on the liquid transfer bus and respective ports of each cartridge module.

(3) The system of either (1) or (2), wherein the fluid conduits comprise tubing or channeling.

(4) The system of any one of (1) to (3), further comprising a sterile liquid transfer device configured to facilitate transfer of liquid between the cartridge and a closed volume fluid device.

(5) The system of any one of (1) to (4), wherein the closed volume fluid device is a fluid vessel.

(6) The system of any one of (1) to (5), wherein the sterile liquid transfer device comprises at least one actuator.

(7) The system of any one of (1) to (6), wherein the actuator actuates one of the at least one sterile liquid transfer port of the cartridge and a corresponding sterile liquid transfer port of a closed volume fluid device.

(8) The system of any one of (1) to (7), further comprising a fluid vault disposed within a housing of the system, the fluid vault being accessible by a user to insert and/or remove one or more closed volume fluid devices.

(9) The system of any one of (1) to (8), wherein each of the one or more cell processing operations performed upon the cartridge are performed on cells within the cartridge.

(10) The system of any one of (1) to (9), wherein the plurality of cartridge modules comprises the electroporation module.

(11) The system of any one of (1) to (10), wherein the plurality of cartridge modules comprises the magnetic-activated cell selection module.

(12) The system of any one of (1) to (11), wherein the plurality of cartridge modules comprises the fluorescence-activated cell selection module.

(13) The system of any one of (1) to (12), wherein the plurality of cartridge modules comprises the spinoculation module.

(14) The system of any one of (1) to (13), wherein the plurality of docking station modules comprises a corresponding bioreactor module.

(15) The system of any one of (1) to (14), wherein the plurality of docking station modules comprises a corresponding magnetic-activated cell selection module.

(16) The system of any one of (1) to (15), wherein the plurality of docking station modules comprises a corresponding fluorescence-activated cell selection module.

(17) The system of any one of (l) to (16), wherein the plurality of docking station modules comprises a corresponding electroporation module.

(18) The system of any one of (1) to (17), wherein the plurality of docking station modules comprises a corresponding counterflow centrifugal elutriation module.

(19) The system of any one of (1) to (18), wherein the plurality of docking station modules comprises a corresponding spinoculation module.

(20) The system of any one of (1) to (19), wherein the cartridge comprises a pump fluidically coupled to the liquid transfer bus.

(21) The system of any one of (1) to (20), further comprising a pump actuator configured to interface with the pump.

(22) The system of any one of (1) to (21), further comprising a waste reservoir to capture waste generated by the cartridge.

(23) The system of any one of (1) to (22), wherein the transfer of liquid between the cartridge and the closed volume fluid device comprises extraction of at least a portion of a cell processing sample from the cartridge.

(24) An automated cell processing method comprising performing at least two cell processing operations within a cartridge positioned in a docking station, wherein the cartridge comprises a plurality of cartridge modules, at least one sterile liquid transfer port, and a liquid transfer bus fluidically coupled to each cartridge module, and wherein the docking station comprising a plurality of docking station modules corresponding to the plurality of cartridge modules and is sized and shaped to receive a single cartridge, wherein the plurality of cartridge modules comprises a bioreactor module, a counterflow centrifugal elutriation module, and at least one of an electroporation module, a magnetic-activated cell selection module, a fluorescence-activated cell selection module, or a spinoculation module, and wherein the cell processing operations are automatic upon execution of a set of received instructions.

(25) The method of (24), further comprising actuating one of the at least one sterile liquid transfer port of the cartridge and a corresponding sterile liquid transfer port of a closed volume fluid device to facilitate transfer of liquid therebetween.

(26) The method of either (24) or (25), wherein facilitating the transfer of liquid between the cartridge and the closed volume fluid device comprises actuating the one of the at least one sterile liquid transfer port and the corresponding sterile liquid transfer port.

(27) The method of any one of (24) to (26), wherein the transfer of liquid between the cartridge and the closed volume fluid device comprises extraction of at least a portion of a cell processing sample from the cartridge.

(28) The method of any one of (24) to (27), wherein the plurality of cartridge modules comprises the electroporation module.

(29) The method of any one of (24) to (28), wherein the plurality of cartridge modules comprises the magnetic-activated cell selection module.

(30) The method of any one of (24) to (29), wherein the plurality of cartridge modules comprises the fluorescence-activated cell selection module.

(31) The method of any one of (24) to (30), wherein the plurality of cartridge modules comprises the spinoculation module.

(32) The method of any one of (24) to (31), wherein the plurality of docking station modules comprises a corresponding bioreactor module.

(33) The method of any one of (24) to (32), wherein the plurality of docking station modules comprises a corresponding counterflow centrifugal elutriation module.

(34) The method of any one of (24) to (33), wherein the plurality of docking station modules comprises a corresponding electroporation module.

(35) The method of any one of (24) to (34), wherein the plurality of docking station modules comprises a corresponding magnetic-activated cell selection module.

(36) The method of any one of (24) to (35), wherein the plurality of docking station modules comprises a corresponding fluorescence-activated cell selection module.

(37) The method of any one of (24) to (36), wherein the plurality of docking station modules comprises a corresponding spinoculation module.

(38) The method of any one of (24) to (37), wherein the two or more cell processing performed within the cartridge are performed upon cells within the cartridge.

1. An automated system for cell processing, comprising:
a cartridge comprising
a plurality of cartridge modules including
a bioreactor module, a counterflow centrifugal elutriation module, and at least one of an electroporation module, a magnetic-activated cell selection module, a fluorescence-activated cell selection module, or a spinoculation module,
at least one sterile liquid transfer port, and
a liquid transfer bus fluidically coupled to each cartridge module; and
a docking station comprising a plurality of docking station modules corresponding to the plurality of cartridge modules, each of the plurality of docking station modules being independently configured to perform one or more cell processing operations upon the cartridge in coordination with a respective cartridge module, wherein the docking station is sized and shaped to receive a single cartridge.

2. The system of claim 1, wherein the liquid transfer bus is fluidically coupled to each cartridge module by fluid conduits arranged between ports on the liquid transfer bus and respective ports of each cartridge module.

3. (canceled)

4. The system of claim 1, further comprising a sterile liquid transfer device configured to facilitate transfer of liquid between the cartridge and a closed volume fluid device.

5. (canceled)

6. The system of claim 4, wherein the sterile liquid transfer device comprises at least one actuator.

7. The system of claim 6, wherein the actuator actuates one of the at least one sterile liquid transfer port of the cartridge and a corresponding sterile liquid transfer port of a closed volume fluid device.

8. (canceled)

9. (canceled)

10. The system of claim 1, wherein the plurality of cartridge modules comprises the electroporation module.

11. The system of claim 1, wherein the plurality of cartridge modules comprises the magnetic-activated cell selection module.

12. The system of claim 1, wherein the plurality of cartridge modules comprises the fluorescence-activated cell selection module.

13. The system of claim 1, wherein the plurality of cartridge modules comprises the spinoculation module.

14. The system of claim 1, wherein the plurality of docking station modules comprises a corresponding bioreactor module.

15. The system of claim 1, wherein the plurality of docking station modules comprises a corresponding magnetic-activated cell selection module.

16. The system of claim 1, wherein the plurality of docking station modules comprises a corresponding fluorescence-activated cell selection module.

17. The system of claim 1, wherein the plurality of docking station modules comprises a corresponding electroporation module.

18. The system of claim 1, wherein the plurality of docking station modules comprises a corresponding counterflow centrifugal elutriation module.

19. The system of claim 1, wherein the plurality of docking station modules comprises a corresponding spinoculation module.

20. The system of claim 1, wherein the cartridge comprises a pump fluidically coupled to the liquid transfer bus.

21. (canceled)

22. (canceled)

23. (canceled)

24. An automated cell processing method comprising:
performing at least two cell processing operations within a cartridge positioned in a docking station, wherein the cartridge comprises a plurality of cartridge modules, at least one sterile liquid transfer port, and a liquid transfer bus fluidically coupled to each cartridge module, and wherein the docking station comprising a plurality of docking station modules corresponding to the plurality of cartridge modules and is sized and shaped to receive a single cartridge,
wherein the plurality of cartridge modules comprises a bioreactor module, a counterflow centrifugal elutriation module, and at least one of an electroporation module, a magnetic-activated cell selection module, a fluorescence-activated cell selection module, or a spinoculation module, and
wherein the cell processing operations are automatic upon execution of a set of received instructions.

25. The method of claim 24, further comprising:
actuating one of the at least one sterile liquid transfer port of the cartridge and a corresponding sterile liquid transfer port of a closed volume fluid device to facilitate transfer of liquid therebetween.

26. The method of claim 25, wherein facilitating the transfer of liquid between the cartridge and the closed volume fluid device comprises actuating the one of the at least one sterile liquid transfer port and the corresponding sterile liquid transfer port.

27. The method of claim 25, wherein the transfer of liquid between the cartridge and the closed volume fluid device comprises extraction of at least a portion of a cell processing sample from the cartridge.

28.-37. (canceled)

38. The method of claim 24, wherein the two or more cell processing performed within the cartridge are performed upon cells within the cartridge.
